(19) United States
(12) Patent Application Publication
(10) Pub. No.: US 2019/0312210 A1
(43) Pub. Date: Oct. 10, 2019

(54) ORGANIC ELECTROLUMINESCENCE DEVICE AND POLYCYCLIC COMPOUND FOR ORGANIC ELECTROLUMINESCENCE DEVICE

(71) Applicant: Samsung Display Co., Ltd., Yongin-si (KR)

(72) Inventors: Nobutaka AKASHI, Yokohama (JP); Yuji SUZAKI, Yokohama (JP)

(21) Appl. No.: 16/298,942

(22) Filed: Mar. 11, 2019

(30) Foreign Application Priority Data
Apr. 5, 2018 (KR) 10-2018-0039925

(51) Int. Cl. H01L 51/00; C07F 5/02; C07D 519/00; C07F 7/08; C09K 11/06

(52) U.S. Cl. CPC H01L 51/0072; C07F 5/027; C07D 519/00; H01L 51/5012; C07F 7/081; C09K 11/06; H01L 51/0094

(57) ABSTRACT

An organic electroluminescence device includes a first electrode, a hole transport region on the first electrode, an emission layer on the hole transport region, an electron transport region on the emission layer, and a second electrode on the electron transport region, wherein the emission layer includes a polycyclic compound represented by Formula 1. In Formula 1, X₁ may be C, Si or Ge; and AC may be an electron acceptor.

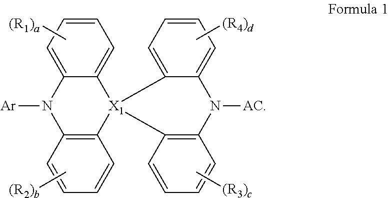

### CROSS-REFERENCE TO RELATED APPLICATION

This application claims priority to and the benefit of Korean Patent Application No. 10-2018-0039925, filed on Apr. 5, 2018, the entire content of which is hereby incorporated by reference.

### BACKGROUND

One or more aspects of embodiments of the present disclosure are related to an organic electroluminescence device (OLED), and a polycyclic compound used for the organic electroluminescence device.

The development of an organic electroluminescence device as an image display device is being actively conducted. The organic electroluminescence device differs from a liquid crystal display device, and is a so-called self-luminescent display device. In a self-luminescent display device, holes and electrons respectively injected from a first electrode and a second electrode recombine in an emission layer, and an organic light-emitting material included in the emission layer may emit light to attain display (e.g., have a display function).

An organic electroluminescence device may include, for example, a first electrode, a hole transport layer on the first electrode, an emission layer on the hole transport layer, an electron transport layer on the emission layer, and a second electrode on the electron transport layer. Holes are injected from the first electrode via the hole transport layer into the emission layer. Electrons are injected from the second electrode via the electron transport layer into the emission layer. The holes and electrons injected into the emission layer may recombine to produce excitons in the emission layer. The organic electroluminescence device may emit light that is generated by excitons transitioning to the ground state. Embodiments of the configuration of the organic electroluminescence device are not limited thereto, and various modifications may be possible.

### SUMMARY

One or more aspects of embodiments of the present disclosure provide an organic electroluminescence device (OLED), and a polycyclic compound used in the organic electroluminescence device.

One or more aspects of embodiments of the present disclosure provide an organic electroluminescence device including a first electrode, a hole transport region on the first electrode, an emission layer on the hole transport region, an electron transport region on the emission layer, and a second electrode on the electron transport region, wherein the emission layer includes a polycyclic compound represented by Formula 1:

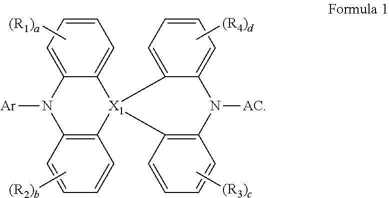

In Formula 1, X₁ may be carbon (C), silicon (Si), or germanium (Ge); Ar may be a substituted or unsubstituted aryl group having 6 to 30 carbon atoms for forming a ring or a substituted or unsubstituted heteroaryl group having 2 to 30 carbon atoms for forming a ring; R₁ to R₄ may each independently be a hydrogen atom (H), a deuterium atom (D), a substituted or unsubstituted alkyl group having 1 to 10 carbon atoms, a substituted or unsubstituted aryl group having 6 to 30 carbon atoms for forming a ring, or a substituted or unsubstituted heteroaryl group having 2 to 30 carbon atoms for forming a ring; “a” to “d” may each independently be an integer of 0 to 4, and AC may be represented by one of Formulae 2 to 9:

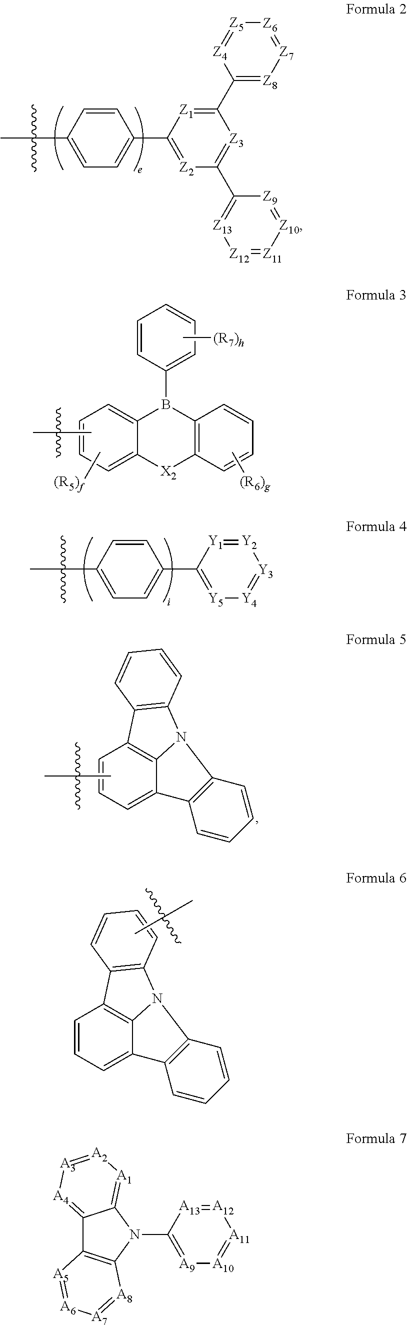

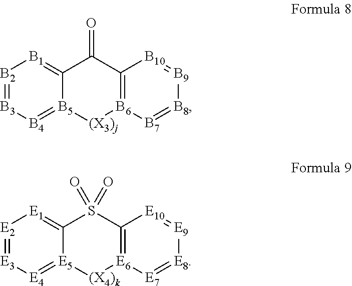

In Formula 2, Z₁ to Z₁₃ may each independently be CH or nitrogen (N), where two or three of Z₁ to Z₃ may each be N, at least one of Z₄ to Z₁₃ may be N, and “e” may be an integer of 0 to 2.

In Formula 3, X₂ may be oxygen (O), sulfur (S), or CR₈R₉, where R₅ to R₉ may each independently be a hydrogen atom, a deuterium atom, or a substituted or unsubstituted alkyl group having 1 to 5 carbon atoms, or may be combined with an adjacent group to form a hydrocarbon ring or a heterocycle. “f” may be an integer of 0 to 3, “g” may be an integer of 0 to 4, and “h” may be an integer of 0 to 5.

In Formula 4, “i” may be an integer of 0 to 2; Y₁ may be N or CR₁₀; Y₂ may be N or CR₁₁; Y₃ may be N or CR₁₂; Y₄ may be N or CR₁₃; and Y₅ may be N or CR₁₄, where at least one of Y₁ to Y₅ may be N; CR₁₀ to CR₁₄ may each independently be a hydrogen atom, a deuterium atom, a cyano group, a substituted or unsubstituted alkyl group having 1 to 10 carbon atoms, or a trifluoromethyl group. When X₁ in Formula 1 is C, at least one of R₁₀ to R₁₄ is not a hydrogen atom, and when at least one of Y₂ and Y₄ in Formula 4 is N, Y₃ is CR₁₂, and R₁₂ is not a cyano group.

In Formula 7, A₁ to A₁₃ may each independently be N or CR₁₅; at least one of A₁ to A₈ may be N, at least one of A₉ to A₁₃ may be N, and at least one of A₁ to A₁₃ may be a part (moiety) that is connected with Formula 1; and R₁₅ may be a hydrogen atom, a deuterium atom, a substituted or unsubstituted alkyl group having 1 to 10 carbon atoms, a substituted or unsubstituted aryl group having 6 to 30 carbon atoms for forming a ring, or a substituted or unsubstituted heteroaryl group having 2 to 30 carbon atoms for forming a ring.

In Formula 8, B₁ to B₁₀ may each independently be N or CR₁₆, and one of B₁ to B₁₀ may be a part (moiety) that is connected with Formula 1; X₃ may be a direct linkage, O, S, CR₁₇R₁₈, or SiR₁₉R₂₀; and “j” may be 0 or 1, where if “j” is 1, B₅ and B₆ may be C; and R₁₆ to R₂₀ may each independently be a hydrogen atom, a deuterium atom, a substituted or unsubstituted alkyl group having 1 to 10 carbon atoms, a substituted or unsubstituted aryl group having 6 to 30 carbon atoms for forming a ring, or a substituted or unsubstituted heteroaryl group having 2 to 30 carbon atoms for forming a ring.

In Formula 9, E₁ to E₁₀ may each independently be N or CR₂₁, and one of E₁ to E₁₀ may be a part (moiety) that is connected with Formula 1; X₄ may be a direct linkage, O, S, CR₂₂R₂₃, or SiR₂₄R₂₅; and “k” may be 0 or 1, where if “k” is 1, E₅ and E₆ may each be C; and R₂₂ to R₂₅ may each independently be a hydrogen atom, a deuterium atom, a substituted or unsubstituted alkyl group having 1 to 10 carbon atoms, a substituted or unsubstituted aryl group having 6 to 30 carbon atoms for forming a ring, or a substituted or unsubstituted heteroaryl group having 2 to 30 carbon atoms for forming a ring.

In some embodiments, Formula 3 may be further represented by Formula 3-1 or Formula 3-2:

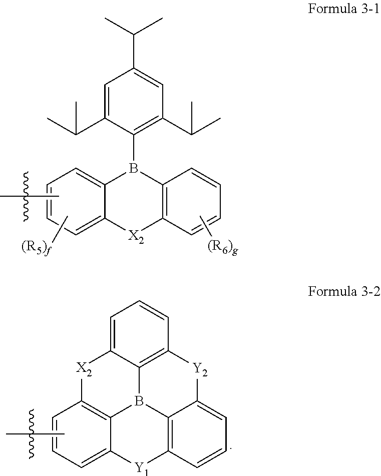

In Formulae 3-1 and 3-2, X₂, R₅, R₆, “f” and “g” may each independently be the same as defined in Formula 3; Y₁ and Y₂ may each independently be O, S, or CR₂₆R₂₇; and R₂₆ and R₂₇ may each independently be a hydrogen atom, a deuterium atom, or a substituted or unsubstituted alkyl group having 1 to 5 carbon atoms.

In some embodiments, Formula 4 may be further represented by one of the following formulae:

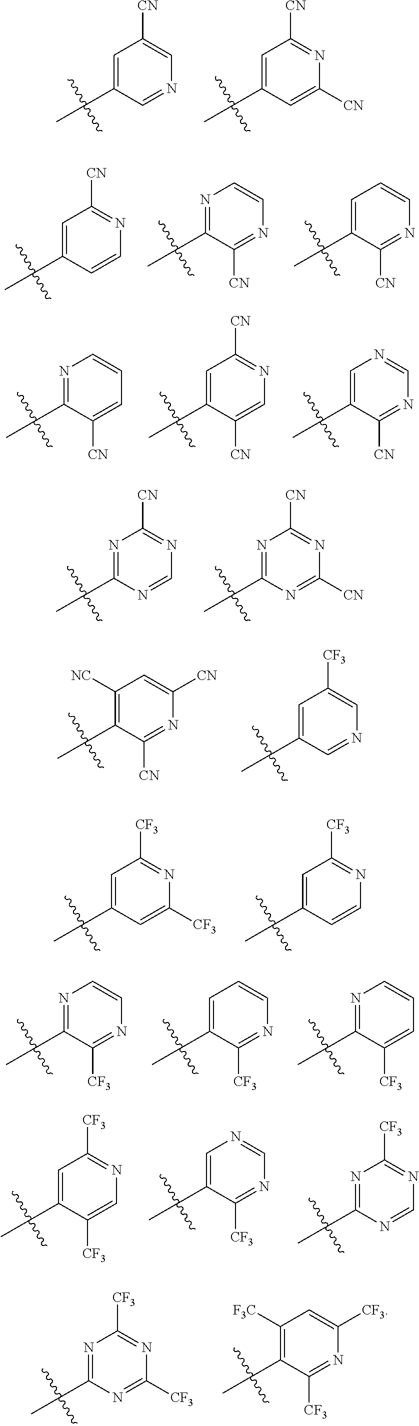

In some embodiments, Formula 7 may be further represented by one of the following formulae:

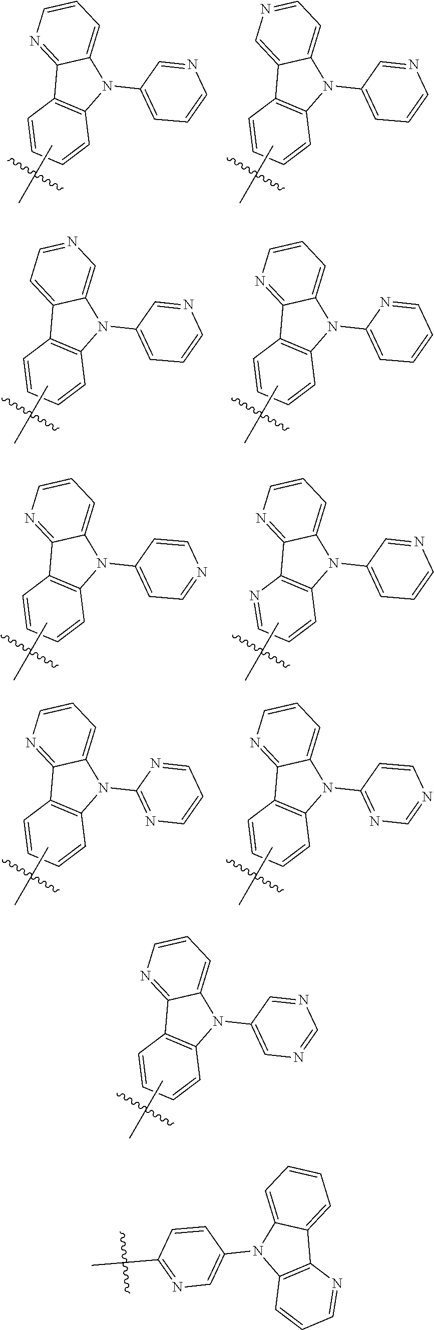

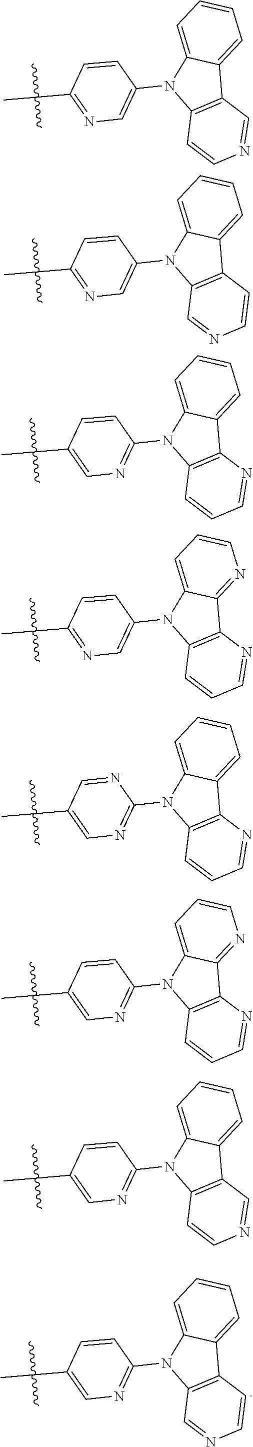

In some embodiments, Formula 8 may be further represented by one of the following formulae:

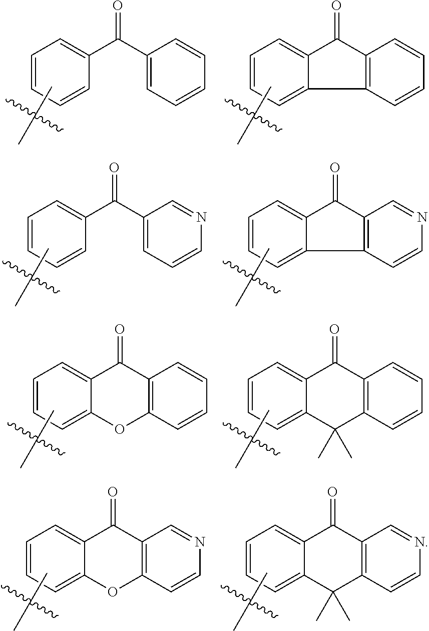

In some embodiments, Formula 9 may be further represented by one of the following formulae:

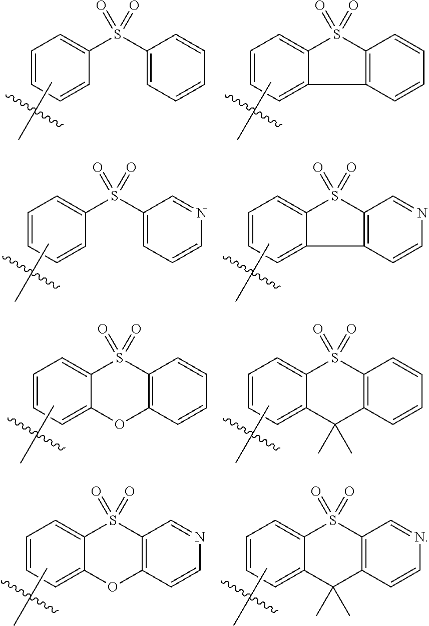

In some embodiments, in Formula 1, Ar may be a substituted or unsubstituted phenyl group.

In some embodiments, the emission layer may include a host and a dopant, and the dopant may include the polycyclic compound represented by Formula 1.

In some embodiments, the polycyclic compound represented by Formula 1 may be a dopant for thermally activated delayed fluorescence.

In some embodiments, the polycyclic compound represented by Formula 1 may be a blue dopant having a wavelength of less than about 470 nm.

In some embodiments, the hole transport region may include a hole injection layer on the first electrode, a hole transport layer on the hole injection layer, and an electron blocking layer on the hole transport layer.

In some embodiments, the electron transport region may include a hole blocking layer on the emission layer, an electron transport layer on the hole blocking layer, and an electron injection layer on the electron transport layer.

One or more aspects of embodiments of the present disclosure provide the polycyclic compound represented by Formula 1.

### BRIEF DESCRIPTION OF THE DRAWINGS

The accompanying drawings are included to provide a further understanding of the present disclosure, and are incorporated in and constitute a part of this specification. The drawings illustrate example embodiments of the present disclosure and, together with the description, serve to explain principles of the present disclosure. In the drawings:

FIG. 1 is a cross-sectional view schematically illustrating an organic electroluminescence device according to one or more embodiments of the present disclosure;

FIG. 2 is a cross-sectional view schematically illustrating an organic electroluminescence device according to one or more embodiments of the present disclosure; and

FIG. 3 is a cross-sectional view schematically illustrating an organic electroluminescence device according to one or more embodiments of the present disclosure.

### DETAILED DESCRIPTION

The above objects, other objects, features and advantages of the present disclosure will be better understood from example embodiments with reference to the accompanying drawings. The present disclosure may, however, be embodied in different forms and should not be construed as being limited to the embodiments set forth herein. Rather, example embodiments are provided so that the contents disclosed herein may be thorough and complete, and so that the spirit of the present disclosure is sufficiently understood a person skilled in the art.

Like reference numerals refer to like elements in the drawings, and duplicative descriptions may not be provided. In the drawings, the sizes and thicknesses of elements may be enlarged or modified for clarity. It will be understood that although the terms first, second, etc. may be used herein to describe various elements, the elements should not be limited by those terms. These terms are only used to distinguish one element from another element. For example, a first element could be alternatively termed a second element, and similarly, a second element could be alternatively termed a first element. Singular forms used herein are intended to also include the plural forms, unless the context clearly indicates otherwise.

It will be further understood that the terms “comprises” or “comprising,” when used in this specification, specify the presence of stated features, numerals, steps, operations, elements, parts, or a combination thereof, but do not preclude the presence or addition of one or more other features, numerals, steps, operations, elements, parts, or a combination thereof. It will be understood that when a layer, a film, a region, a plate, etc. is referred to as being “on” another part, it can be “directly on” the other part, or intervening layers may also be present. When a layer, a film, a region, a plate, etc. is referred to as being “under” another part, it can be “directly under” the other part, or intervening layers may also be present. Further, the use of “may” when describing embodiments of the present disclosure refers to “one or more embodiments of the present disclosure”.

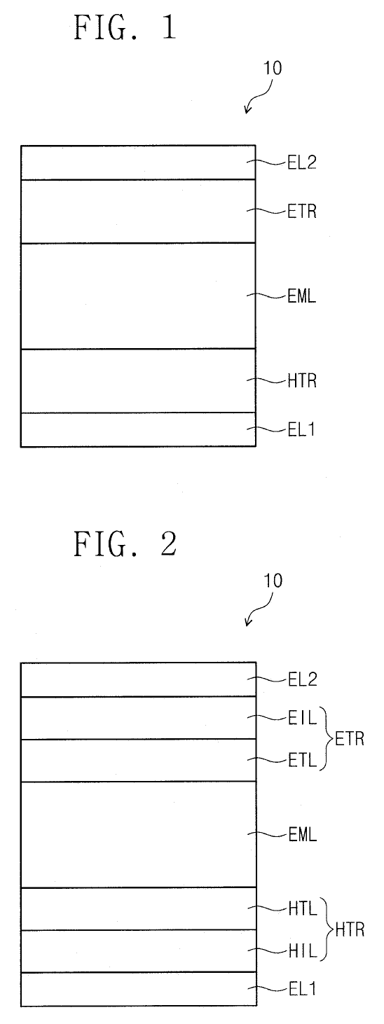

First, organic electroluminescence devices according to example embodiments of the present disclosure will be explained with reference to FIGS. 1-3.

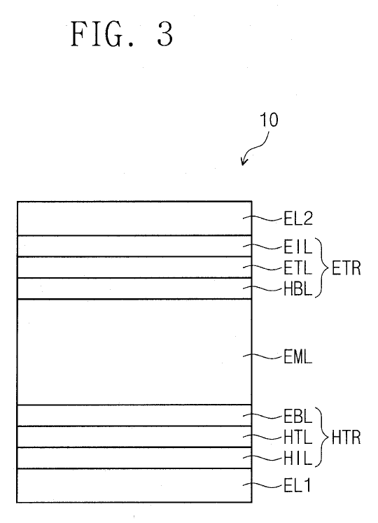

FIG. 1 is a cross-sectional view schematically illustrating an organic electroluminescence device according to one or more embodiments of the present disclosure: FIG. 2 is a cross-sectional view schematically illustrating an organic electroluminescence device according to one or more embodiments of the present disclosure. FIG. 3 is a cross-sectional view schematically illustrating an organic electroluminescence device according to one or more embodiments of the present disclosure.

Referring to FIGS. 1-3, an organic electroluminescence device (OLED) 10 according to one or more embodiments of the present disclosure may include a first electrode EL1, a hole transport region HTR, an emission layer EML, an electron transport region ETR, and a second electrode EL2.

The first electrode EL1 has conductivity (e.g., may be conductive). The first electrode EL1 may be a pixel electrode or an anode. The first electrode EL1 may be a transmissive electrode, a transflective (e.g., semi-transmissive) electrode, or a reflective electrode. When the first electrode EL1 is a transmissive electrode, the first electrode EL1 may include a transparent metal oxide (such as indium tin oxide (ITO), indium zinc oxide (IZO), zinc oxide (ZnO), and/or indium tin zinc oxide (ITZO)). When the first electrode EL1 is a transflective electrode or a reflective electrode, the first electrode EL1 may include silver (Ag), magnesium (Mg), copper (Cu), aluminum (Al), platinum (Pt), palladium (Pd), gold (Au), nickel (Ni), neodymium (Nd), iridium (Ir), chromium (Cr), lithium (Li), calcium (Ca), LiF/Ca, LiF/Al, molybdenum (Mo), titanium (Ti), a compound thereof, or a mixture thereof (for example, a mixture of Ag and Mg). The first electrode EL1 may include a plurality of layers including a reflective layer or transflective layer formed using the above materials, or a transparent layer formed using ITO, IZO, ZnO, or ITZO. For example, the first electrode EL1 may have a three-layer structure of ITO/Ag/ITO, without limitation.

The thickness of the first electrode EL1 may be about 1,000 Å to about 10,000 Å, for example, about 1,000 Å to about 3,000 Å.

The hole transport region HTR may be on the first electrode EL1. The hole transport region HTR may include at least one of a hole injection layer HIL, a hole transport layer HTL, a hole buffer layer, and an electron blocking layer EBL. The thickness of the hole transport region HTR may be, for example, about 1.00 Å to about 1,500 Å. In some embodiments, for example, the hole transport region HTR may include a hole injection layer HIL and a hole transport layer HTL, as shown in FIG. 2. In some embodiments, the hole transport region HTR may include a hole injection layer HIL, a hole transport layer HTL, and an electron blocking layer EBL, as shown in FIG. 3.

The hole transport region HTR may be a single layer formed using a single material, a single layer formed using a plurality of different materials, or a multilayer structure including a plurality of layers formed using a plurality of different materials.

For example, the hole transport region HTR may have the structure of a single layer (such as a hole injection layer HIL and a hole transport layer HTL, e.g., together), and may have a structure of a single layer formed using a hole injection material and a hole transport material. In some embodiments, the hole transport region HTR may have a structure of a single layer formed using a plurality of different materials, or a structure laminated on the first electrode EL1, for example, including a hole injection layer HIL/hole transport layer HTL, a hole injection layer HIL/hole transport layer HTL/hole buffer layer, a hole injection layer HIL/hole buffer layer, a hole transport layer HTL/hole buffer layer, or a hole injection layer HIL/hole transport layer HTL/electron blocking layer EBL, without limitation.

The hole transport region HTR may be formed using one or more suitable methods (such as a vacuum deposition method, a spin coating method, a cast method, a Langmuir-Blodgett (LB) method, an inkjet printing method, a laser printing method, and/or a laser induced thermal imaging (LITI) method).

The hole injection layer HIL may include, for example, a phthalocyanine compound (such as copper phthalocyanine); N,N′-diphenyl-N,N′-bis-[4-(phenyl-m-tolyl-amino)-phenyl]-biphenyl-4,4′-diamine (DNTPD), 4,4′,4″-tris(3-methylphenylphenylamino) triphenylamine (m-MTDATA), 4,4′,4″-tris(N,N-diphenylamino)triphenylamine (TDATA), 4,4′,4″-tris{N-(2-naphthyl)-N-phenylamino}-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-dinaphthalene-1-yl)-N,N′-diphenyl-benzidine (NPB), triphenylamine-containing polyether ketone (TPAPEK), 4-isopropyl-4′-methyldiphenyliodonium tetrakis(pentafluorophenyl)borate, dipyrazino[2,3-f: 2′,3′-h]quinoxaline-2,3,6,7,10,11-hexacarbonitrile (HAT-CN), etc.

The hole transport layer HTL may include, for example, carbazole derivatives (such as N-phenyl carbazole and/or polyvinyl carbazole), fluorine-based derivatives, N,N′-bis(3-methylphenyl)-N,N′-diphenyl-[1,1-biphenyl]-4,4′-diamine (TPD), triphenylamine-based derivatives (such as 4,4′,4″-tris(N-carbazolyl)triphenylamine (TCTA)), N,N′-di(1-naphthalene-1-yl)-N,N′-diphenyl-benzidine (NPB), 4,4′-cyclohexylidene bis[N,N-bis(4-methylphenyl)benzenamine] (TAPC), 4,4′-bis[N,N′-(3-tolyl)amino]-3,3′-dimethylbiphenyl (HMTPD), 1,3-bis(N-carbazolyl)benzene (mCP), etc.

The thickness of the hole transport region HTR may be about 100 Å to about 10,000 Å, for example, about 100 Å to about 1,000 Å. When the hole transport region HTR includes both (e.g., simultaneously) a hole injection layer HIL and a hole transport layer HTL, the thickness of the hole injection layer HIL may be about 100 Å to about 10,000 Å, for example, about 100 Å to about 1,000 Å, and the thickness of the hole transport layer HTL may be about 30 Å to about 1,000 Å. When the thicknesses of the hole transport region HTR, the hole injection layer HIL, and the hole transport layer HTL each satisfy the above-described ranges, satisfactory hole transport properties may be obtained without a substantial increase in driving voltage.

The hole transport region HTR may further include a charge generating material in addition to the above-described materials to increase conductivity. The charge generating material may be dispersed uniformly or non-uniformly in the hole transport region HTR. The charge generating material may be, for example, a p-dopant. The p-dopant may be a quinone derivative, a metal oxide, or a cyano group-containing compound, without limitation. Non-limiting examples of the p-dopant may include quinone derivatives (such as tetracyanoquinodimethane (TCNQ) and/or 2,3,5,6-tetrafluoro-7,7,8,8-tetracyanoquinodimethane (F4-TCNQ)), metal oxides (such as tungsten oxide and/or molybdenum oxide).

As described above, the hole transport region HTR may further include at least one of a hole buffer layer and an electron blocking layer, in addition to the hole injection layer HIL and the hole transport layer HTL. The hole buffer layer may compensate for a resonance distance according to the wavelength of light emitted from the emission layer EML (e.g., be used to adjust the optical resonance distance to match the wavelength of light emitted from the emission layer) and to increase the light emission efficiency of the device. Materials included in the hole transport region HTR may also be included in the hole buffer layer. The electron blocking layer may prevent or reduce electron injection from the electron transport region ETR to the hole transport region HTR.

The emission layer EML may be on the hole transport region HTR. The emission layer EML may have a thickness of about 100 Å to about 1,000 Å, or about 100 Å to about 300 Å. The emission layer EML may be a single layer formed using a single material, a single layer formed using a plurality of different materials, or a multilayer structure having a plurality of layers formed using a plurality of different materials.

The emission layer EML may include a polycyclic compound according to one or more embodiments of the present disclosure. Hereinafter, the polycyclic compound according to one or more embodiments of the present disclosure will be explained in more detail.

In the description,

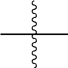

refers to a part to be connected (e.g., a connection point to another moiety or formula structure).

In the description, “substituted or unsubstituted” may refer to substitution with at least one substituent selected from the group consisting of a deuterium atom, a halogen atom, a cyano group, a nitro group, a nitro group, an amino group, a silyl group, a boron group, a phosphine oxide group, a phosphine sulfide group, an alkyl group, an alkenyl group, an aryl group, and a heterocycle, or absence of the above substituents (e.g., substitution with hydrogen atoms only). In some embodiments, each of the above substituents may be further substituted or unsubstituted. For example, a biphenyl group may be interpreted as an aryl group in itself, or as a phenyl group substituted with a phenyl group. The term “heterocycle” as used herein may refer to aliphatic heterocycles and aromatic heterocycles (heteroaryl group).

In some embodiments, “substituted or unsubstituted” may refer to being substituted with one or more substituents selected from the group consisting of a deuterium atom, a halogen atom, a cyano group, an alkyl group, an aryl group, and a heterocycle, as well as being unsubstituted.

In the description, the term “halogen atom” may refer to a fluorine atom (F), a chlorine atom (Cl), a bromine atom (Br), and/or an iodine atom (I).

In the description, the term “alkyl” (e.g., “alkyl group”) may refer to a linear, branched, or cyclic alkyl group. The carbon number of the alkyl may be 1 to 30, 1 to 20, 1 to 10, or 1 to 5. Non-limiting examples of an alkyl group may include methyl, ethyl, n-propyl, isopropyl, n-butyl, s-butyl, t-butyl, i-butyl, 2-ethylbutyl, 3,3-dimethylbutyl, n-pentyl, i-pentyl, neopentyl, t-pentyl, cyclopentyl, 1-methylpentyl, 3-methylpentyl, 2-ethylpentyl, 4-methyl-2-pentyl, n-hexyl, 1-methylhexyl, 2-ethylhexyl, 2-butylhexyl, cyclohexyl, 4-methylcyclohexyl, 4-t-butylcyclohexyl, n-heptyl, 1-methylheptyl, 2,2-dimethylheptyl, 2-ethylheptyl, 2-butylheptyl, n-octyl, t-octyl, 2-ethyloctyl, 2-butyloctyl, 2-hexyloctyl, 3,7-dimethyloctyl, cyclooctyl, n-nonyl, n-decyl, adamantyl, 2-ethyldecyl, 2-butyldecyl, 2-hexyldecyl, 2-octyldecyl, n-undecyl, n-dodecyl, 2-ethyldodecyl, 2-butyldodecyl, 2-hexyldocecyl, 2-octyldodecyl, n-tridecyl, n-tetradecyl, c-pentadecyl, n-hexadecyl, 2-ethylhexadecyl, 2-butylhexadecyl, 2-hexylhexadecyl, 2-octylhexadecyl, n-heptadecyl, n-octadecyl, n-nonadecyl, n-eicosyl, 2-ethyleicosyl, 2-butyleicosyl, 2-hexyleicosyl, 2-octyleicosyl, n-henicosyl, n-docosyl, n-tricosyl, n-tetracosyl, n-pentacosyl, n-hexacosyl, n-heptacosyl, n-octacosyl, n-nonacosyl, n-triacontyl, etc.

In the description, the term “alkenyl group” may refer to a linear chain (e.g., linear alkene) or a branched chain (e.g., branched alkene). The carbon number is not specifically limited, and for example, may be 2 to 30, 2 to 20, or 2 to 10. Non-limiting examples of the alkenyl group may include vinyl, 1-butenyl, 1-pentenyl, 1,3-butadienyl aryl, styrenyl, styrylvinyl, etc. However, embodiments of the present disclosure are not limited thereto.

In the description, the term “aryl group” refers to a functional group or substituent derived from an aromatic hydrocarbon ring. The aryl group may be a monocyclic aryl group or a polycyclic aryl group. The number of carbon atoms for forming a ring in the aryl group may be 6 to 60, 6 to 30, 6 to 20, or 6 to 15. Non-limiting examples of the aryl group may include phenyl, naphthyl, fluorenyl, anthracenyl, phenanthryl, biphenyl, terphenyl, quaterphenyl, quinqphenyl, sexiphenyl, biphenylene, triphenylene, pyrenyl, benzofluoranthenyl, chrysenyl, etc.

In the description, the fluorenyl group may be substituted (e.g., at the 9H position), and two substituents of the fluorenyl group may be combined with each other to form a spiro structure. Non-limiting examples of substituted fluorenyl groups are shown below. However, embodiments of the present disclosure are not limited thereto.

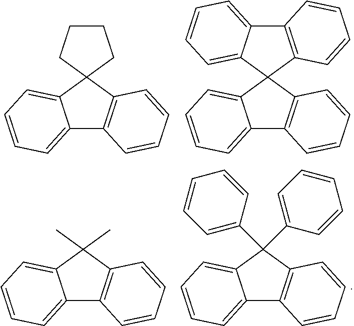

In the description, the term “heterocycle” may refer to a cyclic structure including at least one of boron (B), O, N, phosphorus (P), silicon (Si), or S as a heteroatom. When the heterocycle includes two or more heteroatoms, the two or more heteroatoms may be the same or different. The heterocycle may be heteroaromatic, and may be a monocyclic heteroaryl group or a polycyclic heteroaryl group. The number of carbons for forming a ring in the heterocycle may be 2 to 30, 2 to 20, or 2 to 10. Non-limiting examples of the polycycle (e.g., heterocycle) may include thiophene, furan, pyrrole, imidazole, thiazole, oxazole, oxadiazole, triazole, pyridine, bipyridine, pyrimidine, triazine, triazole, acridyl, pyridazine, pyrazinyl, quinoline, quinazoline, quinoxaline, phenoxazine, phthalazine, pyrido pyrimidine, pyrido pyrazine, pyrazino pyrazine, isoquinoline, indole, carbazole, N-arylcarbazole, N-heteroarylcarbazole, N-alkylcarbazole, benzoxazole, benzimidazole, benzothiazole, benzocarbazole, benzothiophene, dibenzothiophene, thienothiophene, benzofuran, phenanthroline, thiazole, isooxazole, oxadiazole, thiadiazole, phenothiazine, dibenzosilole, dibenzofuran, etc.

In the description, the term “silyl group” may refer to an alkyl silyl group and/or an aryl silyl group. Non-limiting examples of the silyl group may include trimethylsilyl, triethylsilyl, t-butyldimethylsilyl, vinyldimethylsilyl, propyldimethylsilyl, triphenylsilyl, diphenylsilyl, phenylsilyl, etc. However, embodiments of the present disclosure are not limited thereto.

In the description, the boron group includes an alkyl boron group and an aryl boron group. Non-limiting examples of the boron group may include trimethylboron, triethylboron, t-butyldimethylboron, triphenylboron, diphenylboron, phenylboron, etc. However, embodiments of the present disclosure are not limited thereto.

In the description, the carbon number of the amino group is not specifically limited, and for example, may be 1 to 30. The amino group may include an alkyl amino group and an aryl amino group. Non-limiting examples of the amino group include a methylamino group, a dimethylamino group, a phenylamino group, a diphenylamino group, a naphthylamino group, a 9-methyl-anthracenylamino group, a triphenylamino group, etc.

In the description, the phosphine oxide group may be substituted with at least one of an alkyl group or an aryl group. Non-limiting examples of the phosphine oxide group include a phenyl phosphine oxide group, diphenyl phosphine oxide group, etc.

In the description, the phosphine sulfide group may be substituted with at least one of an alkyl group or an aryl group.

The polycyclic compound according to one or more embodiments of the present disclosure may be represented by Formula 1:

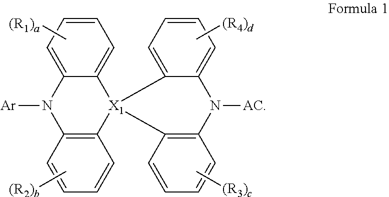

In Formula 1, X₁ may be C, Si, or Ge; Ar may be a substituted or unsubstituted aryl group having 6 to 30 carbon atoms for forming a ring, or a substituted or unsubstituted heteroaryl group having 2 to 30 carbon atoms for forming a ring; R₁ to R₄ may each independently be a hydrogen atom (H), a deuterium atom (D), a substituted or unsubstituted alkyl group having 1 to 10 carbon atoms, a substituted or unsubstituted aryl group having 6 to 30 carbon atoms for forming a ring, or a substituted or unsubstituted heteroaryl group having 2 to 30 carbon atoms for forming a ring; “a” to “d” may each independently be an integer of 0 to 4, and AC may be represented by one of Formulae 2 to 9:

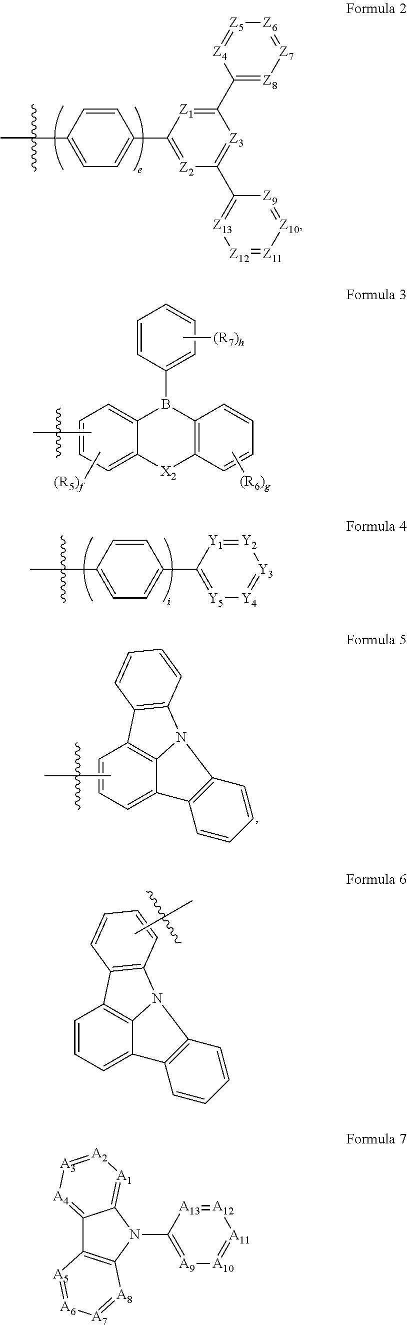

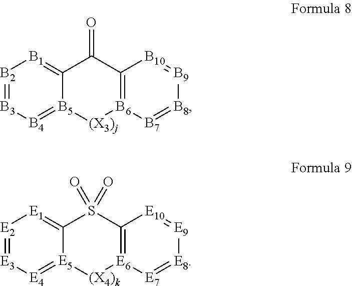

In Formula 2, Z₁ to Z₁₃ may each independently be CH or N, where two or three among Z₁ to Z₃ may each be N and at least one of Z₄ to Z₁₃ may be N; and “e” may be an integer of 0 to 2.

In Formula 3, X₂ may be O, S or CR₈R₉; R₅ to R₉ may each independently be a hydrogen atom, a deuterium atom, or a substituted or unsubstituted alkyl group having 1 to 5 carbon atoms, or may be combined with an adjacent group to form a hydrocarbon ring or a heterocycle; “f” may be an integer of 0 to 3, “g” may be an integer of 0 to 4, and “h” may be an integer of 0 to 5. In Formula 4, “i” may be an integer of 0 to 2, Y₁ may be N or CR₁₀, Y₂ may be N or CR₁₁, Y₃ may be N or CR₁₂, Y₄ may be N or CR₁₃, and Y₅ may be N or CR₁₄, where at least one of Y₁ to Y₅ may be N; and CR₁₀ to CR₁₄ may each independently be a hydrogen atom, a deuterium atom, a cyano group, or a trifluoromethyl group. When X₁ of Formula 1 is C, at least one of R₁₀ to R₁₄ may not be a hydrogen atom, and when at least one of Y₂ and Y₄ is N, Y₃ may be CR₁₂ and R₁₂ may not be a cyano group.

In Formula 7, A₁ to A₁₃ may each independently be N or CR₁₅, at least one of A₁ to A₈ may be N, at least one of A₉ to A₁₃ may be N, and at least one of A₁ to A₁₃ may be a part that is connected with Formula 1; and R₁₅ may be a hydrogen atom, a deuterium atom, a substituted or unsubstituted alkyl group having 1 to 10 carbon atoms, a substituted or unsubstituted aryl group having 6 to 30 carbon atoms for forming a ring, or a substituted or unsubstituted heteroaryl group having 2 to 30 carbon atoms for forming a ring.

In Formula 8, B₁ to B₁₀ may each independently be N or CR₁₆, and one of B₁ to B₁₀ may be a part that is connected with Formula 1; X₃ may be a direct linkage, O, S, CR₁₇R₁₈, or SiR₁₉R₂₀; “j” may be 0 or 1, where if “j” is 1, B₅ and B₆ may be C; and R₁₆ to R₂₀ may each independently be a hydrogen atom, a deuterium atom, a substituted or unsubstituted alkyl group having 1 to 10 carbon atoms, a substituted or unsubstituted aryl group having 6 to 30 carbon atoms for forming a ring, or a substituted or unsubstituted heteroaryl group having 2 to 30 carbon atoms for forming a ring.

In Formula 9, E₁ to E₁₀ may each independently be N or CR₂₁; one of E₁ to E₁₀ may be a part that is connected with Formula 1; X₄ may be a direct linkage, O, S, CR₂₂R₂₃, or SiR₂₄R₂₅; “k” may be 0 or 1, where if “k” is 1, E₅ and E₆ may be C; and R₂₂ to R₂₅ may each independently be a hydrogen atom, a deuterium atom, a substituted or unsubstituted alkyl group having 1 to 10 carbon atoms, a substituted or unsubstituted aryl group having 6 to 30 carbon atoms for forming a ring, or a substituted or unsubstituted heteroaryl group having 2 to 30 carbon atoms for forming a ring.

In Formula 1, if “a” is 2 or more, a plurality of R₁ groups may be the same or different; if “b” is 2 or more, a plurality of R₂ groups may be the same or different; if “c” is 2 or more, a plurality of R₃ groups may be the same or different; and if “d” is 2 or more, a plurality of R₄ groups may be the same or different.

In Formula 1, if “a” is 0, R₁ may not be a hydrogen atom; if “b” is 0, R₂ may not be a hydrogen atom; if “c” is 0, R₃ may not be a hydrogen atom; and if “d” is 0, R₄ may not be a hydrogen atom.

For example, each of “a” to “d” may be 0. However, embodiments of the present disclosure are not limited thereto. In order to control the energy level of a polycyclic compound, etc., at least one of “a” to “d” may be 1 or more, and a substituent other than a hydrogen atom may be introduced.

In Formula 1, Ar may be a substituted or unsubstituted aryl group having 6 to 20 carbon atoms. For example, Ar may be a substituted or unsubstituted phenyl group, a substituted or unsubstituted naphthyl group, a substituted or unsubstituted biphenyl group, a substituted or unsubstituted anthracenyl group, a substituted or unsubstituted phenanthryl group, or a substituted or unsubstituted terphenyl group. For example, Ar may be a substituted or unsubstituted phenyl group. For example, Ar may be an unsubstituted phenyl group.

AC in Formula 1 may be an electron acceptor, and the structure excluding AC in Formula 1 may be an electron donor. For example, the polycyclic compound according to one or more embodiments of the present disclosure may include an electron acceptor and an electron donor in one molecule.

In Formula 2, at least one of Z₅ to Z₇ and Z₁₀ to Z₁₂ may be N.

In Formula 3, if “f” is 2 or more, a plurality of R₅ groups may be the same or different; if “g” is 2 or more, a plurality of R₆ groups may be the same or different; and if “h” is 2 or more, a plurality of R₇ groups may be the same or different.

In Formula 3, if “f” is 0, R₅ may not be a hydrogen atom; if “g” is 0, R₆ may not be a hydrogen atom; and if “h” is 0, R₇ may not be a hydrogen atom.

For example, “h” may be 1 or more, and R₇ may be a substituted or unsubstituted alkyl group having 1 to 5 carbon atoms. In some embodiments, Formula 3 may be further represented by Formula 3-1:

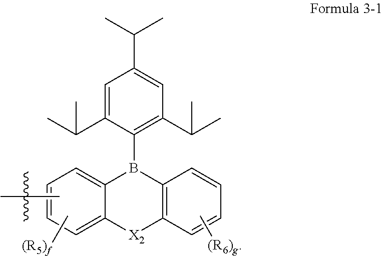

In Formula 3-1, X₂, R₅, R₆, “f” and “g” may each independently be the same as defined in Formula 3. In some embodiments, Formula 3-1 may be further represented by Formula 3-1-1 or 3-1-2:

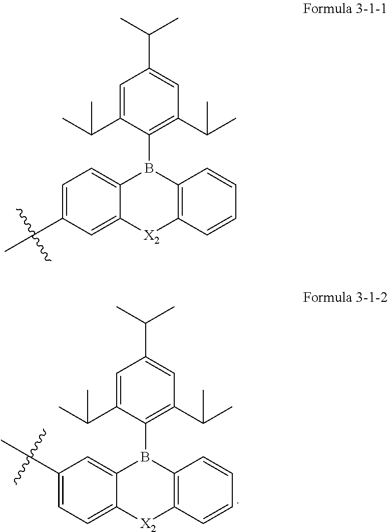

In Formulae 3-1-1 and 3-1-2, X₂ may be the same as defined in Formula 3.

In Formula 3, X₂ may be O or S. In Formula 3, X₂ may be CR₈R₉, where R₈ and R₉ may each independently be a substituted or unsubstituted methyl group.

As described above, in Formula 3, R₅ to R₇ may be combined with an adjacent group to form a hydrocarbon ring or a heterocycle. In some embodiments, for example, Formula 3 may be further represented by Formula 3-2:

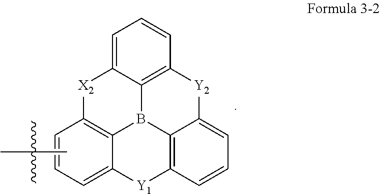

In Formula 3-2, X₂ may be the same as defined in Formula 3; Y₁ and Y₂ may each independently be O, S or CR₂₆R₂₇; and R₂₆ and R₂₇ may each independently be a hydrogen atom, a deuterium atom, or a substituted or unsubstituted alkyl group having 1 to 5 carbon atoms. For example, each of R₂₆ and R₂₇ may be a substituted or unsubstituted methyl group.

In Formula 3-2, at least one of Y₁ and Y₂ may be CR₂₆R₂₇.

In some embodiments, Formula 3-2 may be further represented by one of Formulae 3-2-1 to 3-2-5:

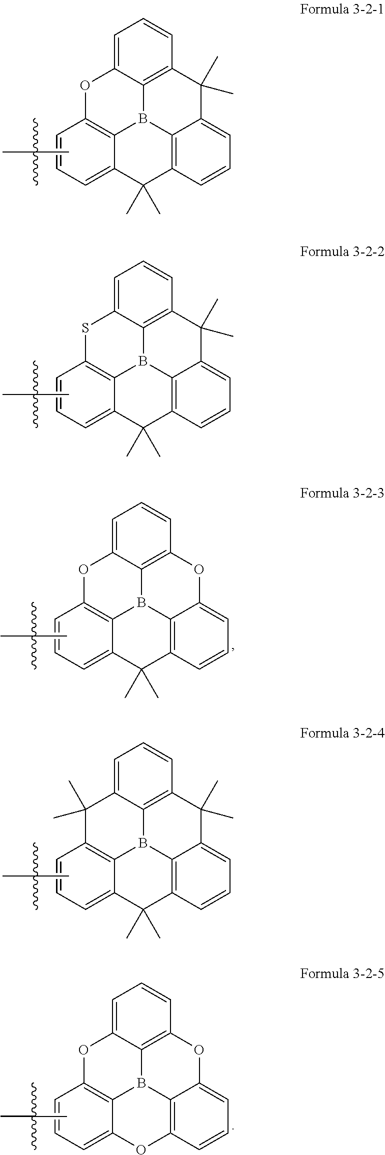

In Formula 4, “i” may be 0.

Formula 4 may be, for example, further represented by one of the following formulae, but embodiments of the present disclosure are not limited thereto:

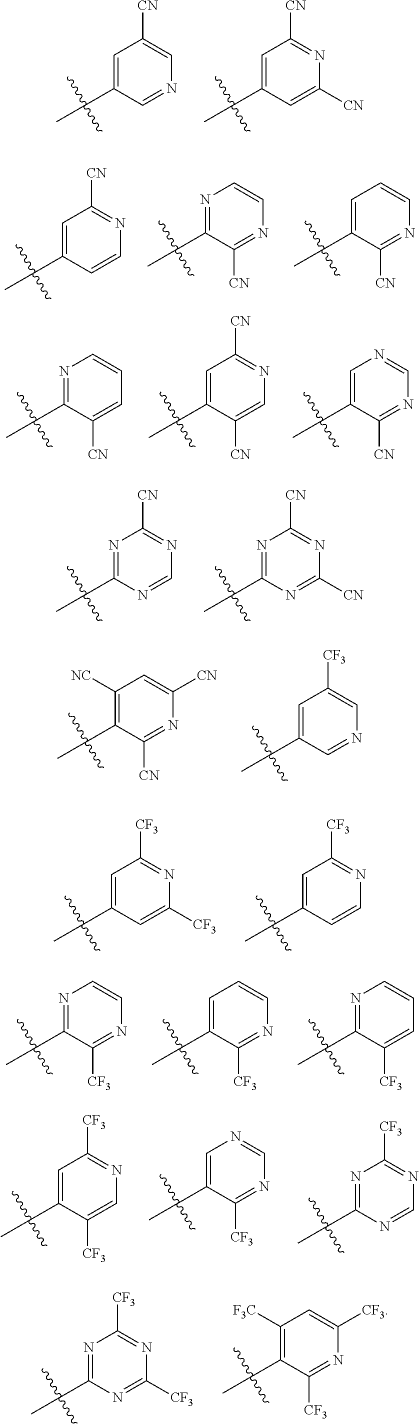

Formula 7 may be further represented by one of the following formulae, but embodiments of the present disclosure are not limited thereto:

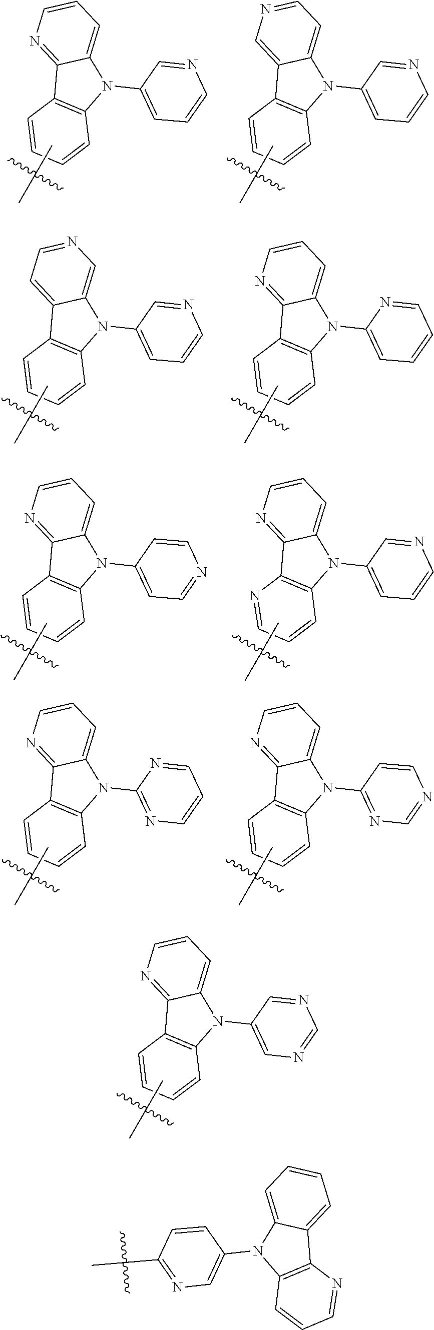

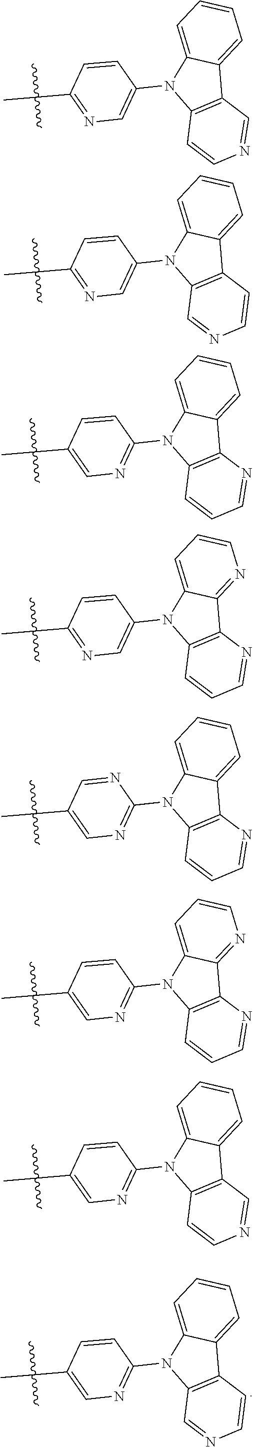

Formula 8 may be, for example, further represented by one of the following formulae, but embodiments of the present disclosure are not limited thereto:

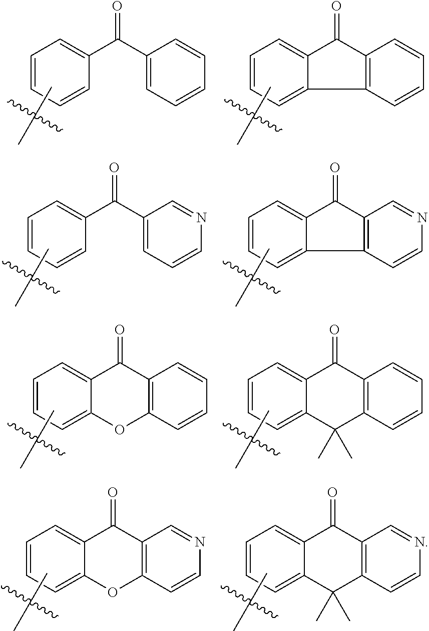

Formula 9 may be, for example, further represented by one of the following formulae, but embodiments of the present disclosure are not limited thereto:

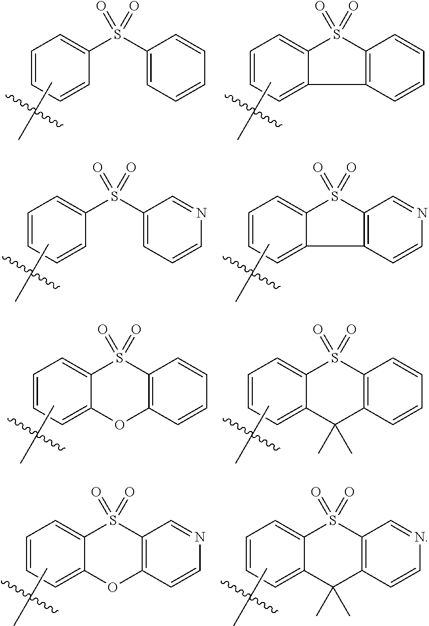

The polycyclic compound represented by Formula 1 may be at least one selected from the compounds represented in Compound Group 1, but embodiments of the present disclosure are not limited thereto:

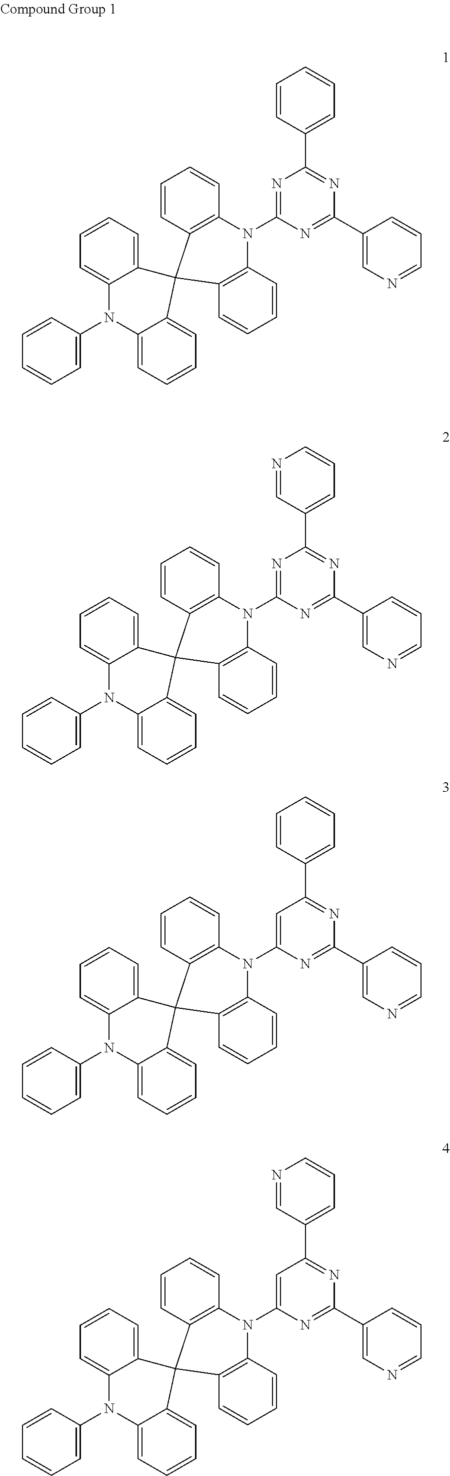 [SMILES: c1ccc(-c2nc(-c3cccnc3)nc(N3c4ccccc4C4(c5ccccc5N(c5ccccc5)c5ccccc54)c4ccccc43)n2)cc1 ; c1ccc(N2c3ccccc3C3(c4ccccc42)c2ccccc2N(c2nc(-c4cccnc4)nc(-c4cccnc4)n2)c2ccccc23)cc1 ; c1ccc(-c2cc(N3c4ccccc4C4(c5ccccc5N(c5ccccc5)c5ccccc54)c4ccccc43)nc(-c3cccnc3)n2)cc1 ; c1ccc(N2c3ccccc3C3(c4ccccc42)c2ccccc2N(c2cc(-c4cccnc4)nc(-c4cccnc4)n2)c2ccccc23)cc1]

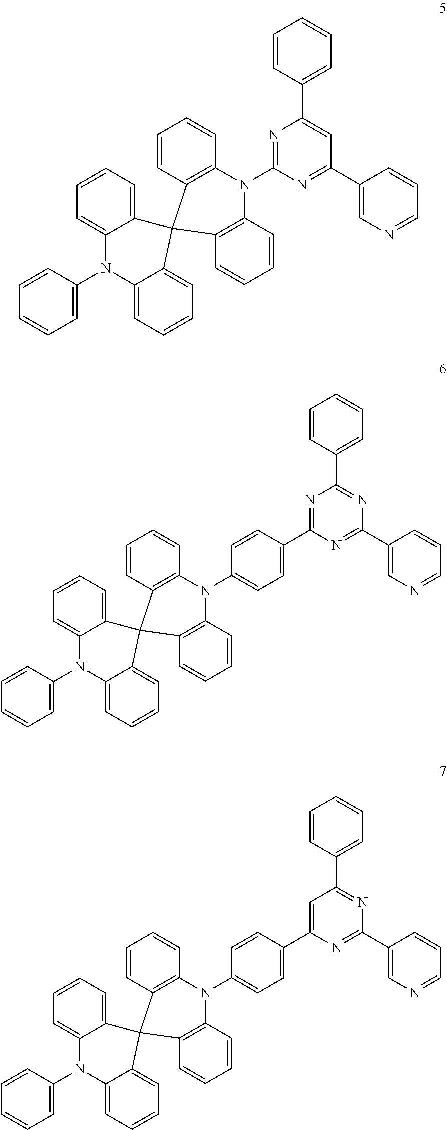 [SMILES: c1ccc(-c2cc(-c3cccnc3)nc(N3c4ccccc4C4(c5ccccc5N(c5ccccc5)c5ccccc54)c4ccccc43)n2)cc1 ; c1ccc(-c2nc(-c3ccc(N4c5ccccc5C5(c6ccccc6N(c6ccccc6)c6ccccc65)c5ccccc54)cc3)nc(-c3cccnc3)n2)cc1 ; c1ccc(-c2cc(-c3ccc(N4c5ccccc5C5(c6ccccc6N(c6ccccc6)c6ccccc65)c5ccccc54)cc3)nc(-c3cccnc3)n2)cc1]

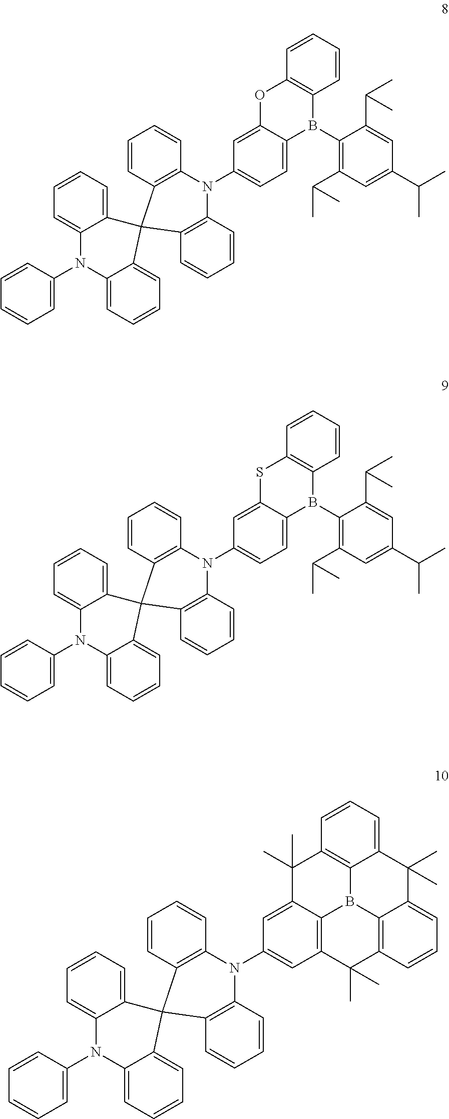 [SMILES: CC(C)c1cc(C(C)C)c(B2c3ccccc3Oc3cc(N4c5ccccc5C5(c6ccccc6N(c6ccccc6)c6ccccc65)c5ccccc54)ccc32)c(C(C)C)c1 ; CC(C)c1cc(C(C)C)c(B2c3ccccc3Sc3cc(N4c5ccccc5C5(c6ccccc6N(c6ccccc6)c6ccccc65)c5ccccc54)ccc32)c(C(C)C)c1 ; CC1(C)c2cccc3c2B2c4c1cccc4C(C)(C)c1cc(N4c5ccccc5C5(c6ccccc6N(c6ccccc6)c6ccccc65)c5ccccc54)cc(c12)C3(C)C]

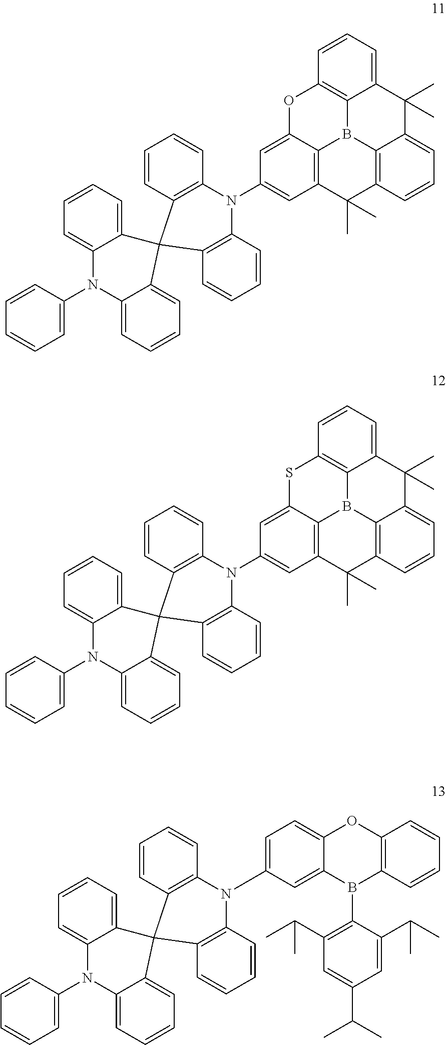 [SMILES: CC1(C)c2cccc3c2B2c4c(cc(N5c6ccccc6C6(c7ccccc7N(c7ccccc7)c7ccccc76)c6ccccc65)cc4C(C)(C)c4cccc1c42)O3 ; CC1(C)c2cccc3c2B2c4c(cc(N5c6ccccc6C6(c7ccccc7N(c7ccccc7)c7ccccc76)c6ccccc65)cc4C(C)(C)c4cccc1c42)S3 ; CC(C)c1cc(C(C)C)c(B2c3ccccc3Oc3ccc(N4c5ccccc5C5(c6ccccc6N(c6ccccc6)c6ccccc65)c5ccccc54)cc32)c(C(C)C)c1]

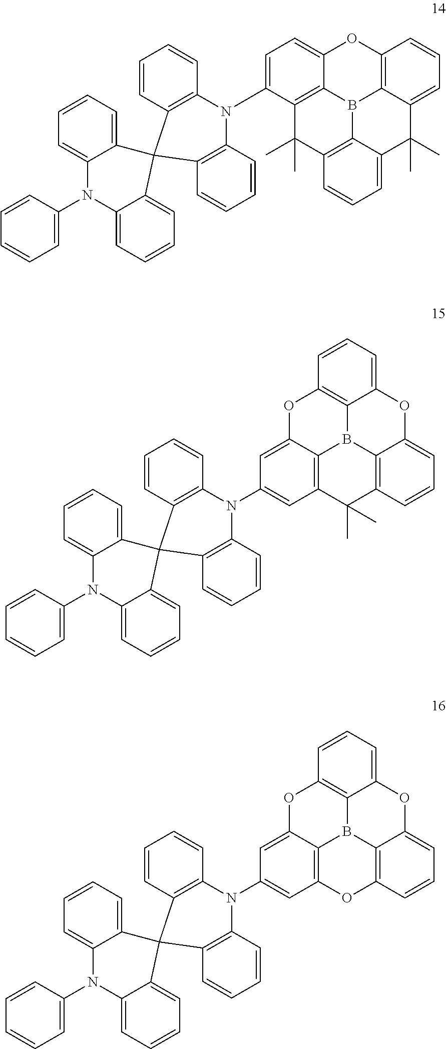 [SMILES: CC1(C)c2cccc3c2B2c4c1cccc4C(C)(C)c1c(N4c5ccccc5C5(c6ccccc6N(c6ccccc6)c6ccccc65)c5ccccc54)ccc(c12)O3 ; CC1(C)c2cccc3c2B2c4c(cccc4Oc4cc(N5c6ccccc6C6(c7ccccc7N(c7ccccc7)c7ccccc76)c6ccccc65)cc1c42)O3 ; c1ccc(N2c3ccccc3C3(c4ccccc42)c2ccccc2N(c2cc4c5c(c2)Oc2cccc6c2B5c2c(cccc2O4)O6)c2ccccc23)cc1]

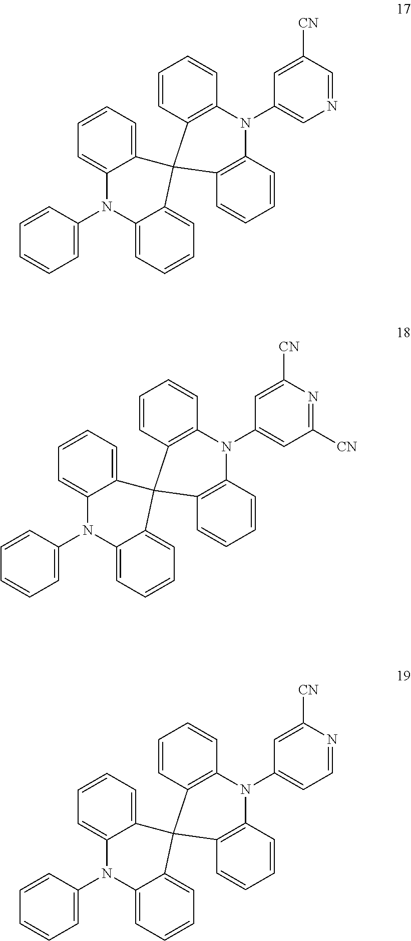 [SMILES: N#Cc1cncc(N2c3ccccc3C3(c4ccccc4N(c4ccccc4)c4ccccc43)c3ccccc32)c1 ; N#Cc1cc(N2c3ccccc3C3(c4ccccc4N(c4ccccc4)c4ccccc43)c3ccccc32)cc(C#N)n1 ; N#Cc1cc(N2c3ccccc3C3(c4ccccc4N(c4ccccc4)c4ccccc43)c3ccccc32)ccn1]

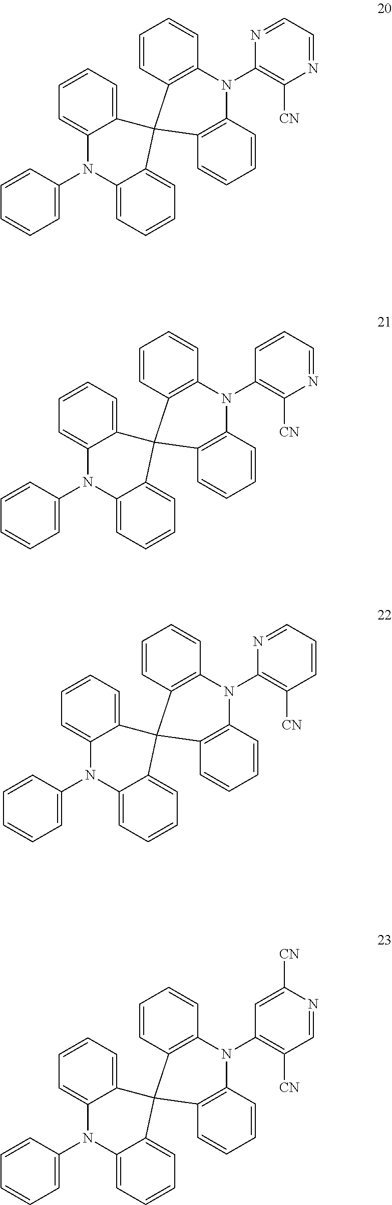 [SMILES: N#Cc1nccnc1N1c2ccccc2C2(c3ccccc3N(c3ccccc3)c3ccccc32)c2ccccc21 ; N#Cc1ncccc1N1c2ccccc2C2(c3ccccc3N(c3ccccc3)c3ccccc32)c2ccccc21 ; N#Cc1cccnc1N1c2ccccc2C2(c3ccccc3N(c3ccccc3)c3ccccc32)c2ccccc21 ; N#Cc1cc(N2c3ccccc3C3(c4ccccc4N(c4ccccc4)c4ccccc43)c3ccccc32)c(C#N)cn1]

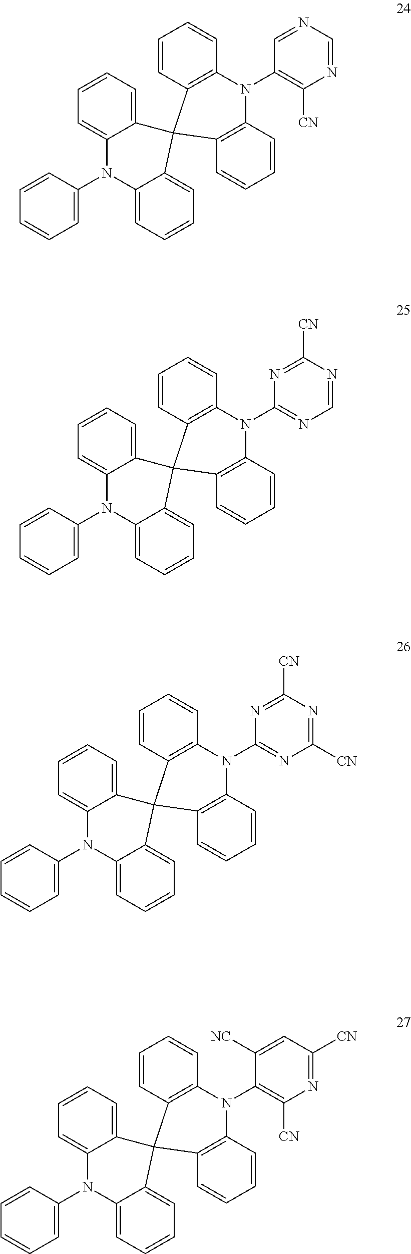 [SMILES: N#Cc1ncncc1N1c2ccccc2C2(c3ccccc3N(c3ccccc3)c3ccccc32)c2ccccc21 ; N#Cc1ncnc(N2c3ccccc3C3(c4ccccc4N(c4ccccc4)c4ccccc43)c3ccccc32)n1 ; N#Cc1nc(C#N)nc(N2c3ccccc3C3(c4ccccc4N(c4ccccc4)c4ccccc43)c3ccccc32)n1 ; N#Cc1cc(C#N)c(N2c3ccccc3C3(c4ccccc4N(c4ccccc4)c4ccccc43)c3ccccc32)c(C#N)n1]

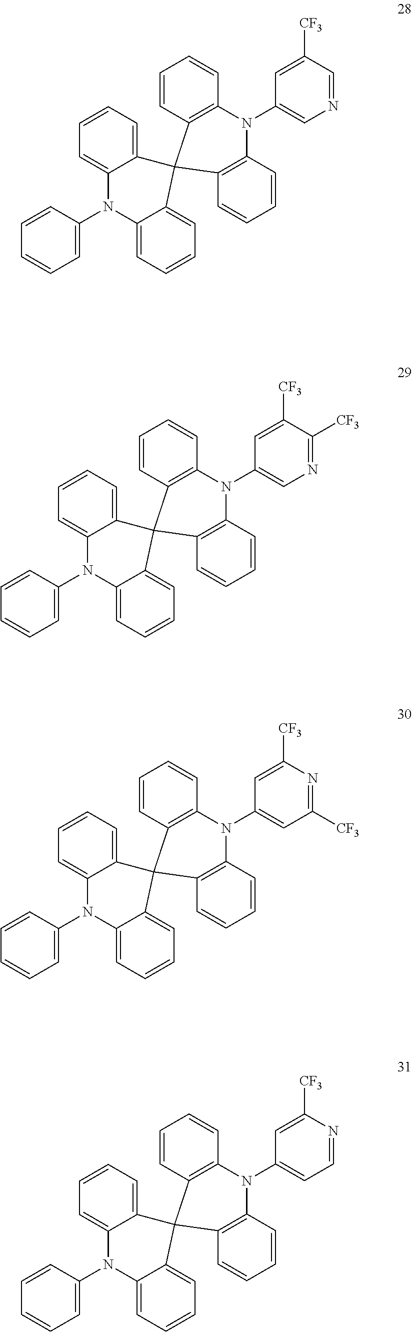 [SMILES: FC(F)(F)c1cncc(N2c3ccccc3C3(c4ccccc4N(c4ccccc4)c4ccccc43)c3ccccc32)c1 ; FC(F)(F)c1cc(N2c3ccccc3C3(c4ccccc4N(c4ccccc4)c4ccccc43)c3ccccc32)cnc1C(F)(F)F ; FC(F)(F)c1cc(N2c3ccccc3C3(c4ccccc4N(c4ccccc4)c4ccccc43)c3ccccc32)cc(C(F)(F)F)n1 ; FC(F)(F)c1cc(N2c3ccccc3C3(c4ccccc4N(c4ccccc4)c4ccccc43)c3ccccc32)ccn1]

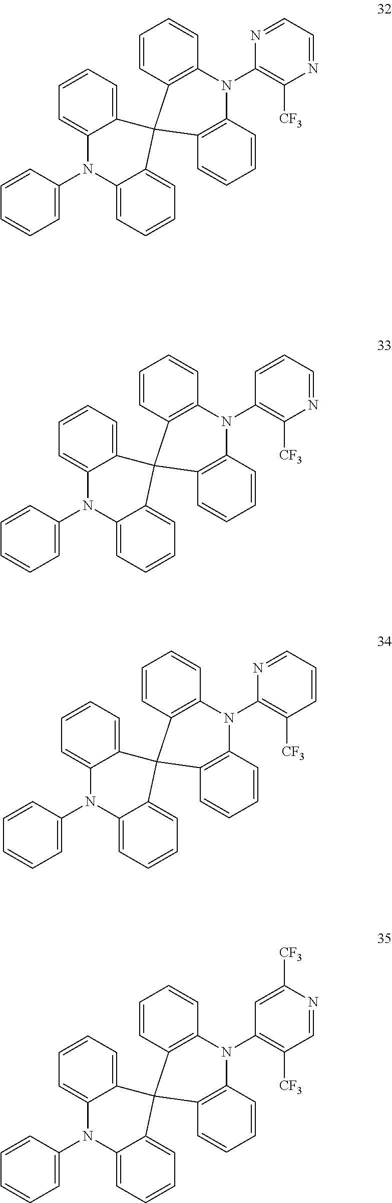 [SMILES: FC(F)(F)c1nccnc1N1c2ccccc2C2(c3ccccc3N(c3ccccc3)c3ccccc32)c2ccccc21 ; FC(F)(F)c1ncccc1N1c2ccccc2C2(c3ccccc3N(c3ccccc3)c3ccccc32)c2ccccc21 ; FC(F)(F)c1cccnc1N1c2ccccc2C2(c3ccccc3N(c3ccccc3)c3ccccc32)c2ccccc21 ; FC(F)(F)c1cc(N2c3ccccc3C3(c4ccccc4N(c4ccccc4)c4ccccc43)c3ccccc32)c(C(F)(F)F)cn1]

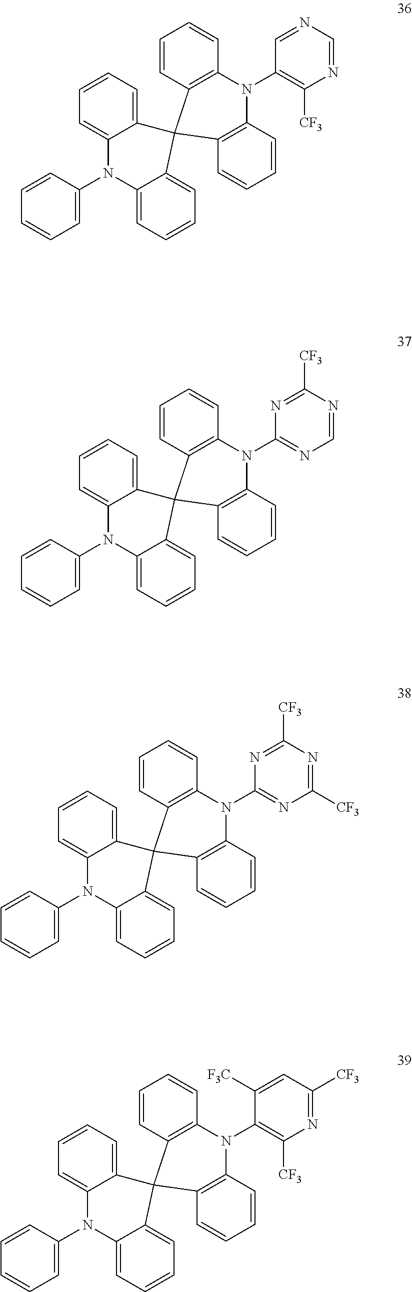 [SMILES: FC(F)(F)c1ncncc1N1c2ccccc2C2(c3ccccc3N(c3ccccc3)c3ccccc32)c2ccccc21 ; FC(F)(F)c1ncnc(N2c3ccccc3C3(c4ccccc4N(c4ccccc4)c4ccccc43)c3ccccc32)n1 ; FC(F)(F)c1nc(N2c3ccccc3C3(c4ccccc4N(c4ccccc4)c4ccccc43)c3ccccc32)nc(C(F)(F)F)n1 ; FC(F)(F)c1cc(C(F)(F)F)c(N2c3ccccc3C3(c4ccccc4N(c4ccccc4)c4ccccc43)c3ccccc32)c(C(F)(F)F)n1]

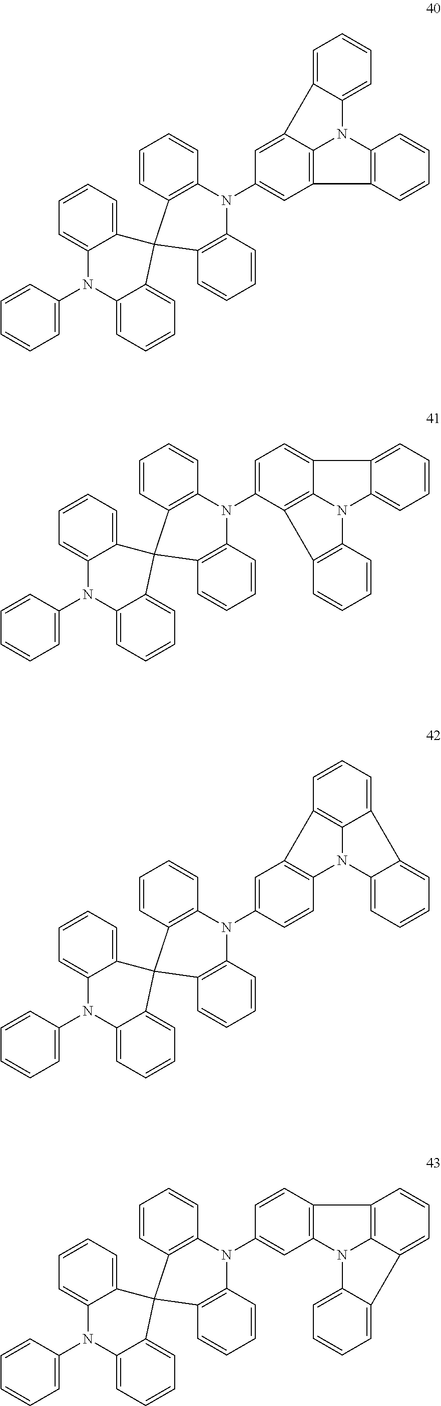 [SMILES: c1ccc(N2c3ccccc3C3(c4ccccc42)c2ccccc2N(c2cc4c5ccccc5n5c6ccccc6c(c2)c45)c2ccccc23)cc1 ; c1ccc(N2c3ccccc3C3(c4ccccc42)c2ccccc2N(c2ccc4c5ccccc5n5c6ccccc6c2c45)c2ccccc23)cc1 ; c1ccc(N2c3ccccc3C3(c4ccccc42)c2ccccc2N(c2ccc4c(c2)c2cccc5c6ccccc6n4c52)c2ccccc23)cc1 ; c1ccc(N2c3ccccc3C3(c4ccccc42)c2ccccc2N(c2ccc4c5cccc6c7ccccc7n(c4c2)c65)c2ccccc23)cc1]

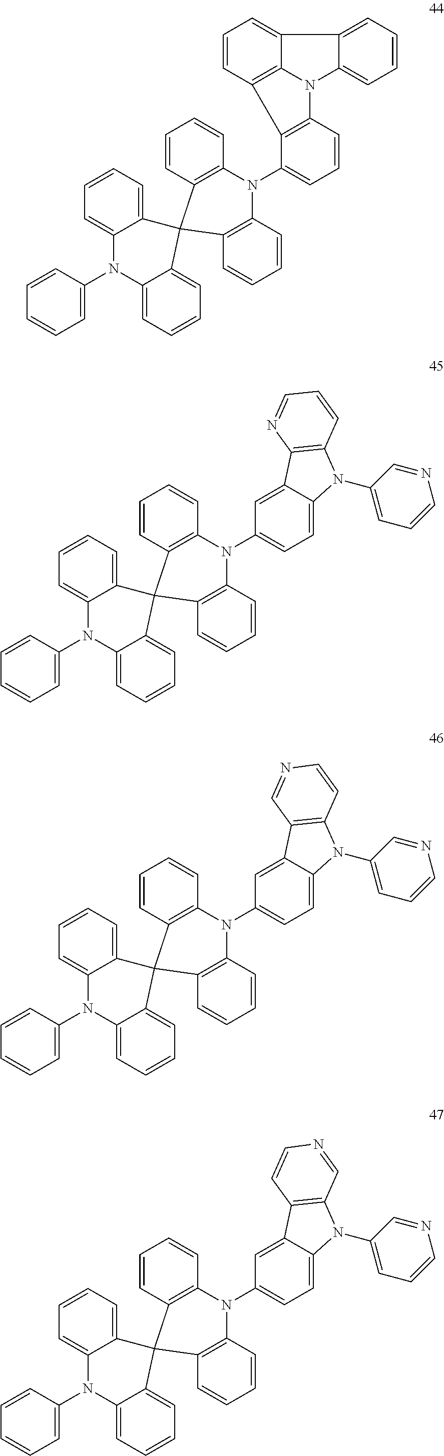 [SMILES: c1ccc(N2c3ccccc3C3(c4ccccc42)c2ccccc2N(c2cccc4c2c2cccc5c6ccccc6n4c52)c2ccccc23)cc1 ; c1ccc(N2c3ccccc3C3(c4ccccc42)c2ccccc2N(c2ccc4c(c2)c2ncccc2n4-c2cccnc2)c2ccccc23)cc1 ; c1ccc(N2c3ccccc3C3(c4ccccc42)c2ccccc2N(c2ccc4c(c2)c2cnccc2n4-c2cccnc2)c2ccccc23)cc1 ; c1ccc(N2c3ccccc3C3(c4ccccc42)c2ccccc2N(c2ccc4c(c2)c2ccncc2n4-c2cccnc2)c2ccccc23)cc1]

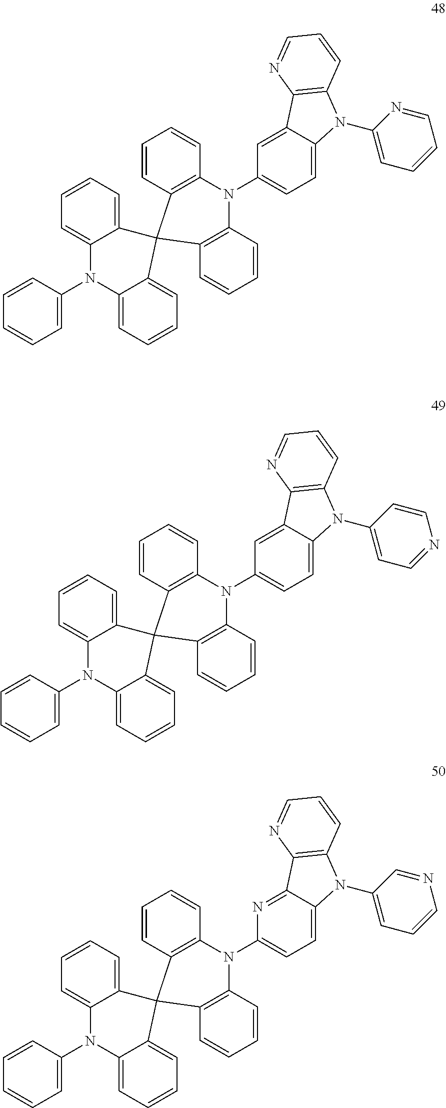 [SMILES: c1ccc(N2c3ccccc3C3(c4ccccc42)c2ccccc2N(c2ccc4c(c2)c2ncccc2n4-c2ccccn2)c2ccccc23)cc1 ; c1ccc(N2c3ccccc3C3(c4ccccc42)c2ccccc2N(c2ccc4c(c2)c2ncccc2n4-c2ccncc2)c2ccccc23)cc1 ; c1ccc(N2c3ccccc3C3(c4ccccc42)c2ccccc2N(c2ccc4c(n2)c2ncccc2n4-c2cccnc2)c2ccccc23)cc1]

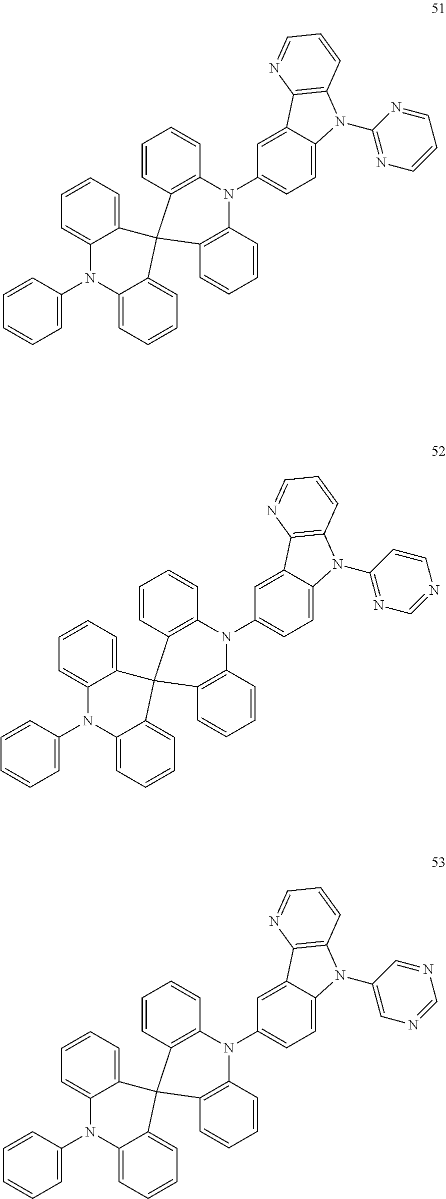 [SMILES: c1ccc(N2c3ccccc3C3(c4ccccc42)c2ccccc2N(c2ccc4c(c2)c2ncccc2n4-c2ncccn2)c2ccccc23)cc1 ; c1ccc(N2c3ccccc3C3(c4ccccc42)c2ccccc2N(c2ccc4c(c2)c2ncccc2n4-c2ccncn2)c2ccccc23)cc1 ; c1ccc(N2c3ccccc3C3(c4ccccc42)c2ccccc2N(c2ccc4c(c2)c2ncccc2n4-c2cncnc2)c2ccccc23)cc1]

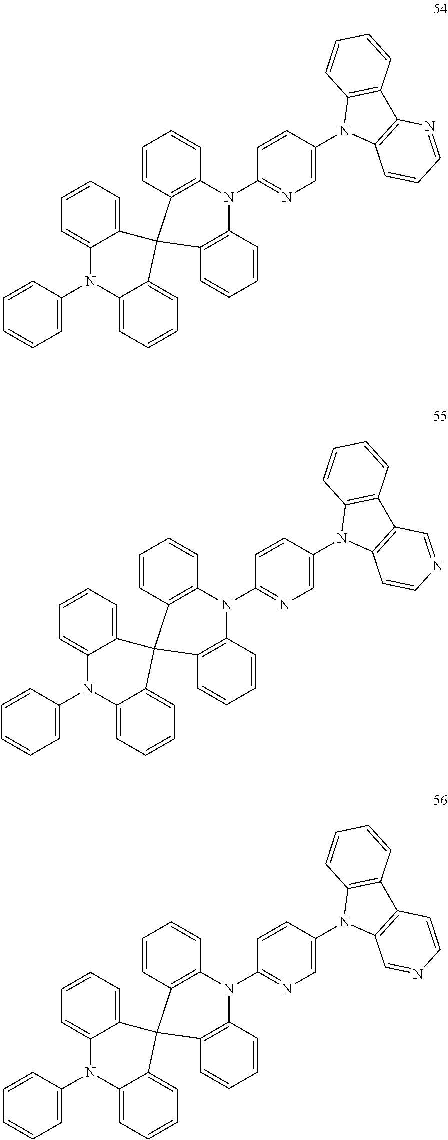 [SMILES: c1ccc(N2c3ccccc3C3(c4ccccc42)c2ccccc2N(c2ccc(-n4c5ccccc5c5ncccc54)cn2)c2ccccc23)cc1 ; c1ccc(N2c3ccccc3C3(c4ccccc42)c2ccccc2N(c2ccc(-n4c5ccccc5c5cnccc54)cn2)c2ccccc23)cc1 ; c1ccc(N2c3ccccc3C3(c4ccccc42)c2ccccc2N(c2ccc(-n4c5ccccc5c5ccncc54)cn2)c2ccccc23)cc1]

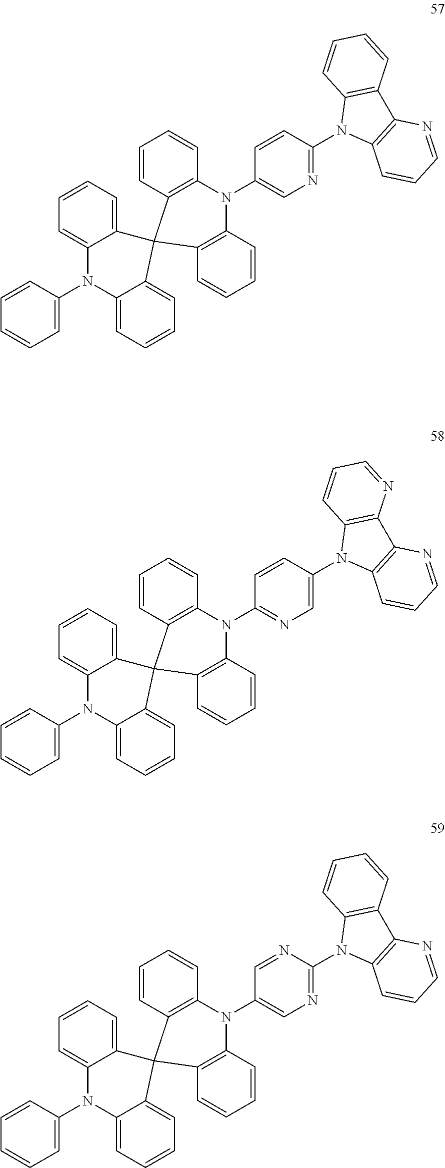 [SMILES: c1ccc(N2c3ccccc3C3(c4ccccc42)c2ccccc2N(c2ccc(-n4c5ccccc5c5ncccc54)nc2)c2ccccc23)cc1 ; c1ccc(N2c3ccccc3C3(c4ccccc42)c2ccccc2N(c2ccc(-n4c5cccnc5c5ncccc54)cn2)c2ccccc23)cc1 ; c1ccc(N2c3ccccc3C3(c4ccccc42)c2ccccc2N(c2cnc(-n4c5ccccc5c5ncccc54)nc2)c2ccccc23)cc1]

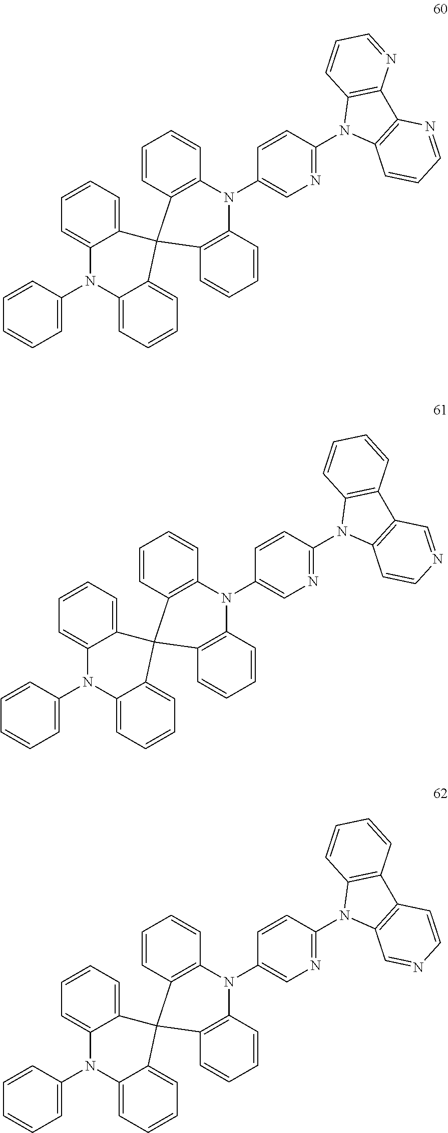 [SMILES: c1ccc(N2c3ccccc3C3(c4ccccc42)c2ccccc2N(c2ccc(-n4c5cccnc5c5ncccc54)nc2)c2ccccc23)cc1 ; c1ccc(N2c3ccccc3C3(c4ccccc42)c2ccccc2N(c2ccc(-n4c5ccccc5c5cnccc54)nc2)c2ccccc23)cc1 ; c1ccc(N2c3ccccc3C3(c4ccccc42)c2ccccc2N(c2ccc(-n4c5ccccc5c5ccncc54)nc2)c2ccccc23)cc1]

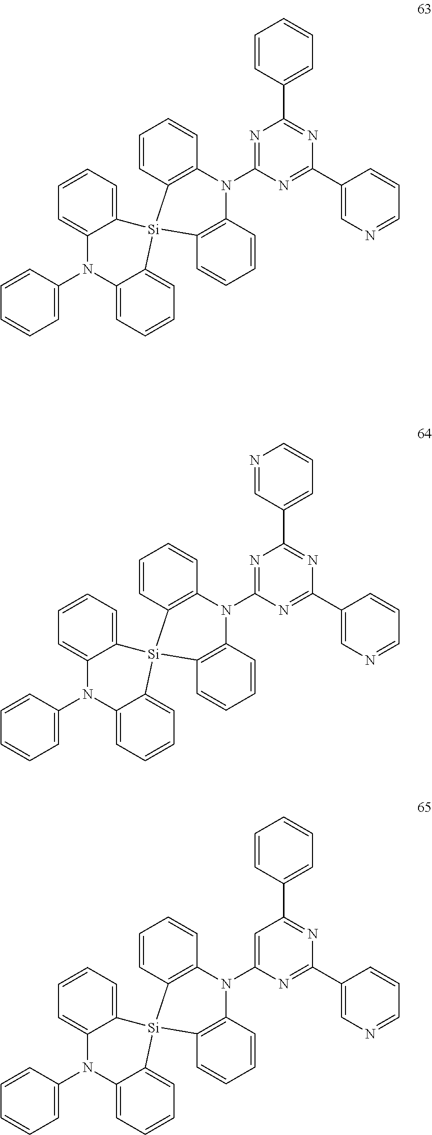 [SMILES: c1ccc(-c2nc(-c3cccnc3)nc(N3c4ccccc4[Si]4(c5ccccc5N(c5ccccc5)c5ccccc54)c4ccccc43)n2)cc1 ; c1ccc(N2c3ccccc3[Si]3(c4ccccc42)c2ccccc2N(c2nc(-c4cccnc4)nc(-c4cccnc4)n2)c2ccccc23)cc1 ; c1ccc(-c2cc(N3c4ccccc4[Si]4(c5ccccc5N(c5ccccc5)c5ccccc54)c4ccccc43)nc(-c3cccnc3)n2)cc1]

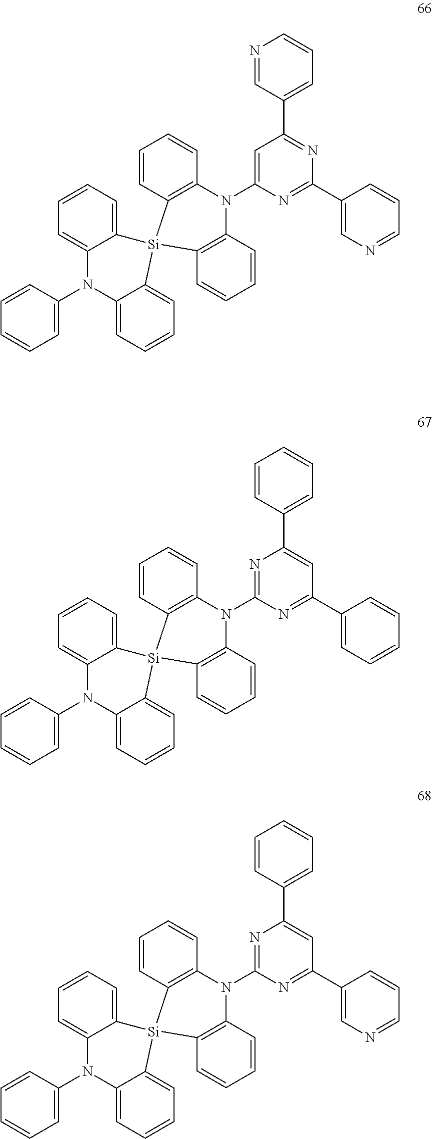 [SMILES: c1ccc(N2c3ccccc3[Si]3(c4ccccc42)c2ccccc2N(c2cc(-c4cccnc4)nc(-c4cccnc4)n2)c2ccccc23)cc1 ; c1ccc(-c2cc(-c3ccccc3)nc(N3c4ccccc4[Si]4(c5ccccc5N(c5ccccc5)c5ccccc54)c4ccccc43)n2)cc1 ; c1ccc(-c2cc(-c3cccnc3)nc(N3c4ccccc4[Si]4(c5ccccc5N(c5ccccc5)c5ccccc54)c4ccccc43)n2)cc1]

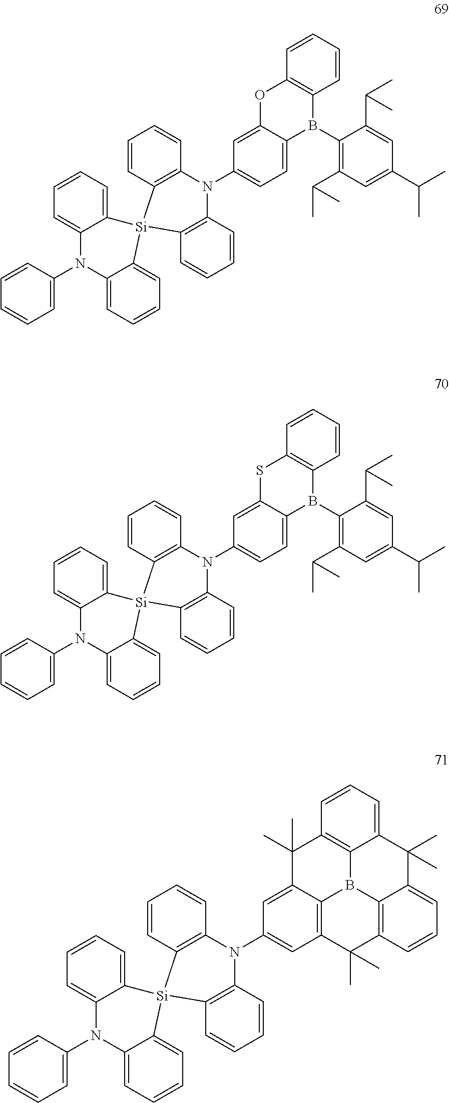 [SMILES: CC(C)c1cc(C(C)C)c(B2c3ccccc3Oc3cc(N4c5ccccc5[Si]5(c6ccccc6N(c6ccccc6)c6ccccc65)c5ccccc54)ccc32)c(C(C)C)c1 ; CC(C)c1cc(C(C)C)c(B2c3ccccc3Sc3cc(N4c5ccccc5[Si]5(c6ccccc6N(c6ccccc6)c6ccccc65)c5ccccc54)ccc32)c(C(C)C)c1 ; CC1(C)c2cccc3c2B2c4c1cccc4C(C)(C)c1cc(N4c5ccccc5[Si]5(c6ccccc6N(c6ccccc6)c6ccccc65)c5ccccc54)cc(c12)C3(C)C]

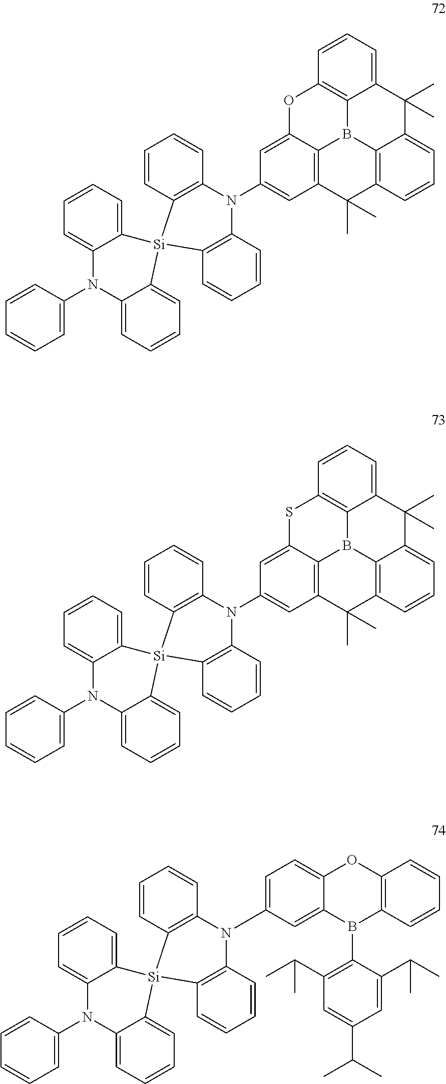 [SMILES: CC1(C)c2cccc3c2B2c4c(cc(N5c6ccccc6[Si]6(c7ccccc7N(c7ccccc7)c7ccccc76)c6ccccc65)cc4C(C)(C)c4cccc1c42)O3 ; CC1(C)c2cccc3c2B2c4c(cc(N5c6ccccc6[Si]6(c7ccccc7N(c7ccccc7)c7ccccc76)c6ccccc65)cc4C(C)(C)c4cccc1c42)S3 ; CC(C)c1cc(C(C)C)c(B2c3ccccc3Oc3ccc(N4c5ccccc5[Si]5(c6ccccc6N(c6ccccc6)c6ccccc65)c5ccccc54)cc32)c(C(C)C)c1]

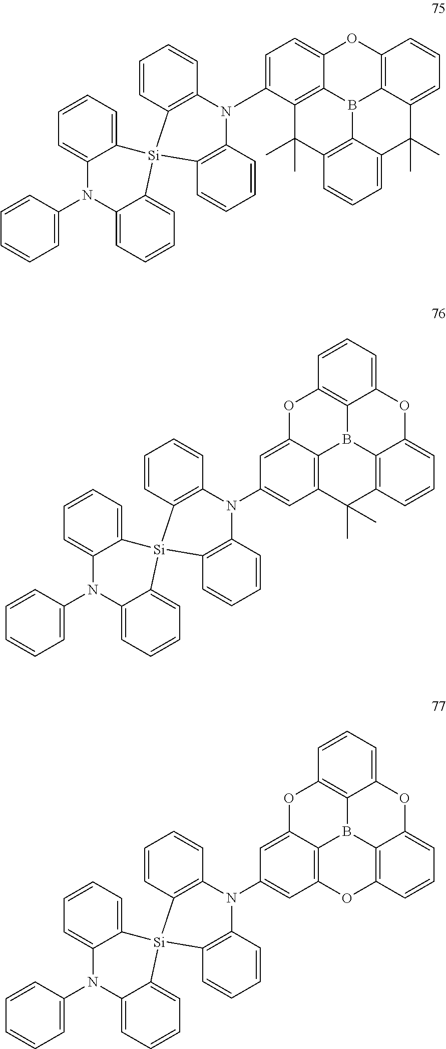 [SMILES: CC1(C)c2cccc3c2B2c4c1cccc4C(C)(C)c1c(N4c5ccccc5[Si]5(c6ccccc6N(c6ccccc6)c6ccccc65)c5ccccc54)ccc(c12)O3 ; CC1(C)c2cccc3c2B2c4c(cccc4Oc4cc(N5c6ccccc6[Si]6(c7ccccc7N(c7ccccc7)c7ccccc76)c6ccccc65)cc1c42)O3 ; c1ccc(N2c3ccccc3[Si]3(c4ccccc42)c2ccccc2N(c2cc4c5c(c2)Oc2cccc6c2B5c2c(cccc2O4)O6)c2ccccc23)cc1]

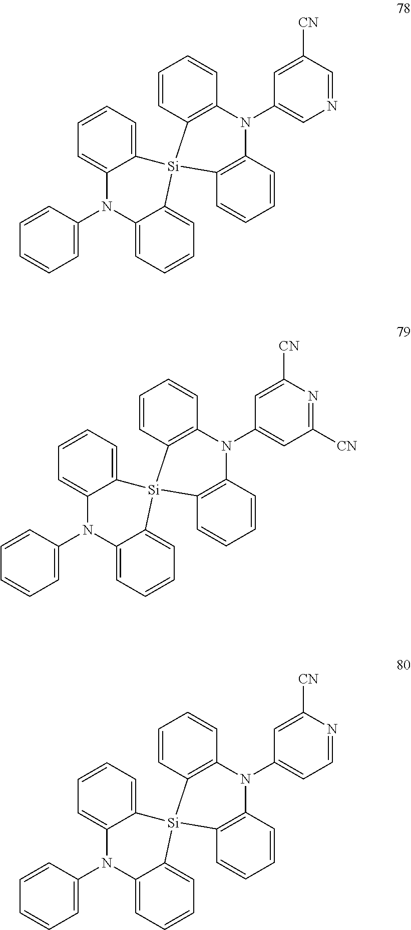 [SMILES: N#Cc1cncc(N2c3ccccc3[Si]3(c4ccccc4N(c4ccccc4)c4ccccc43)c3ccccc32)c1 ; N#Cc1cc(N2c3ccccc3[Si]3(c4ccccc4N(c4ccccc4)c4ccccc43)c3ccccc32)cc(C#N)n1 ; N#Cc1cc(N2c3ccccc3[Si]3(c4ccccc4N(c4ccccc4)c4ccccc43)c3ccccc32)ccn1]

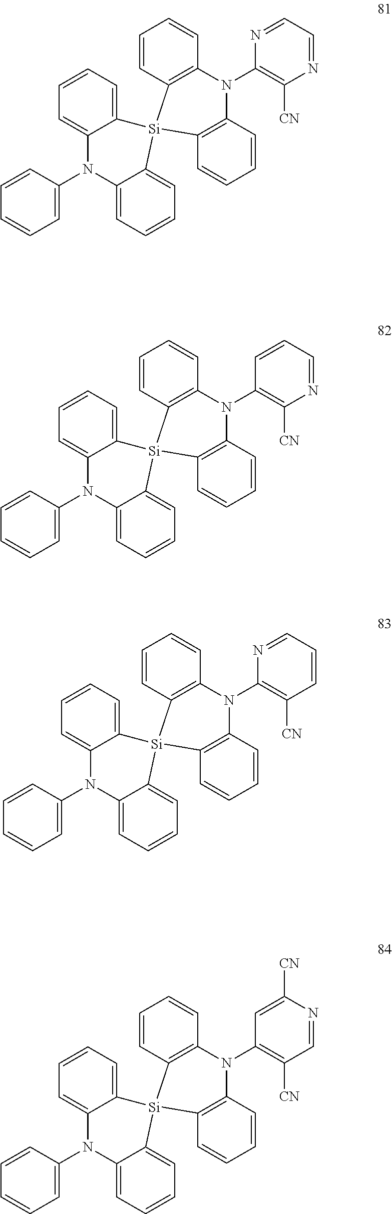 [SMILES: N#Cc1nccnc1N1c2ccccc2[Si]2(c3ccccc3N(c3ccccc3)c3ccccc32)c2ccccc21 ; N#Cc1ncccc1N1c2ccccc2[Si]2(c3ccccc3N(c3ccccc3)c3ccccc32)c2ccccc21 ; N#Cc1cccnc1N1c2ccccc2[Si]2(c3ccccc3N(c3ccccc3)c3ccccc32)c2ccccc21 ; N#Cc1cc(N2c3ccccc3[Si]3(c4ccccc4N(c4ccccc4)c4ccccc43)c3ccccc32)c(C#N)cn1]

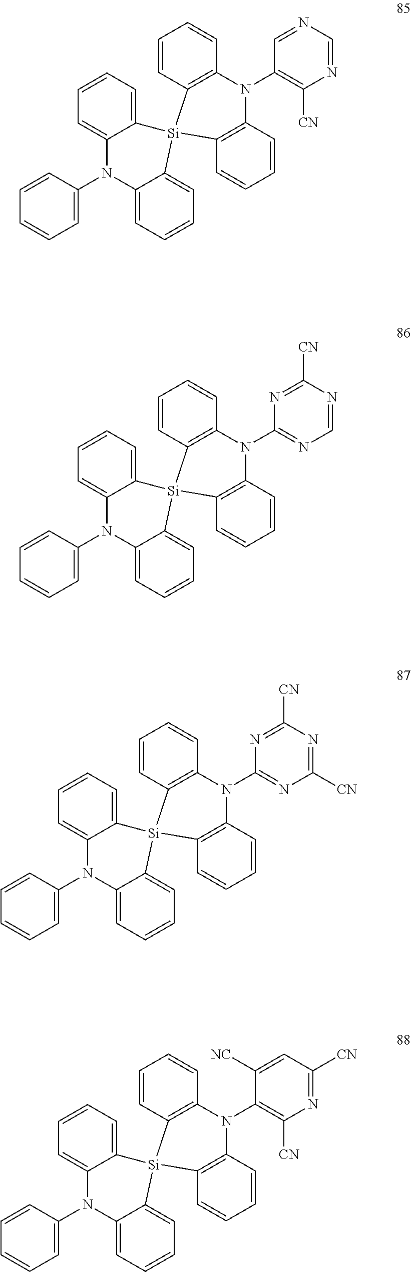 [SMILES: N#Cc1ncncc1N1c2ccccc2[Si]2(c3ccccc3N(c3ccccc3)c3ccccc32)c2ccccc21 ; N#Cc1ncnc(N2c3ccccc3[Si]3(c4ccccc4N(c4ccccc4)c4ccccc43)c3ccccc32)n1 ; N#Cc1nc(C#N)nc(N2c3ccccc3[Si]3(c4ccccc4N(c4ccccc4)c4ccccc43)c3ccccc32)n1 ; N#Cc1cc(C#N)c(N2c3ccccc3[Si]3(c4ccccc4N(c4ccccc4)c4ccccc43)c3ccccc32)c(C#N)n1]

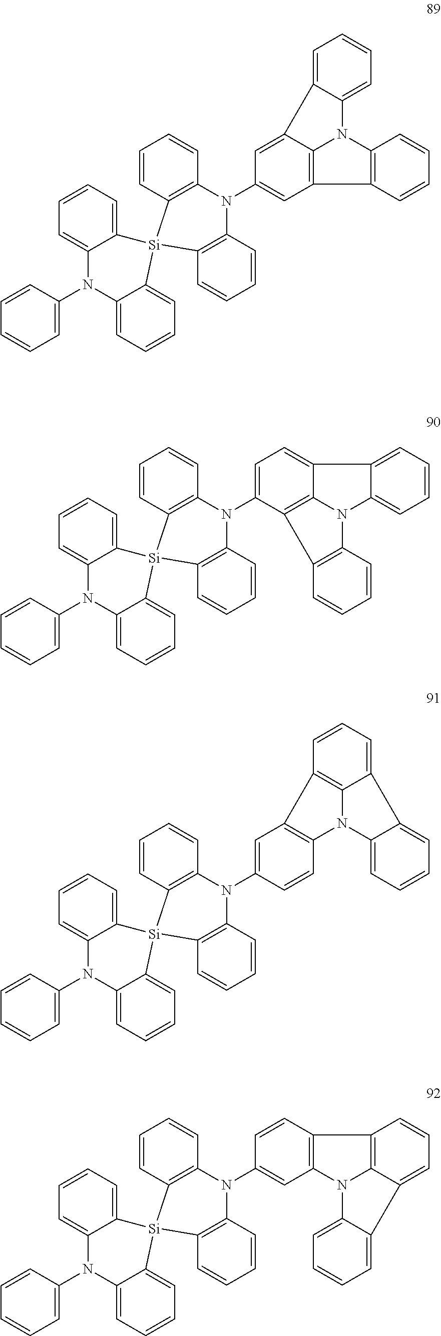 [SMILES: c1ccc(N2c3ccccc3[Si]3(c4ccccc42)c2ccccc2N(c2cc4c5ccccc5n5c6ccccc6c(c2)c45)c2ccccc23)cc1 ; c1ccc(N2c3ccccc3[Si]3(c4ccccc42)c2ccccc2N(c2ccc4c5ccccc5n5c6ccccc6c2c45)c2ccccc23)cc1 ; c1ccc(N2c3ccccc3[Si]3(c4ccccc42)c2ccccc2N(c2ccc4c(c2)c2cccc5c6ccccc6n4c52)c2ccccc23)cc1 ; c1ccc(N2c3ccccc3[Si]3(c4ccccc42)c2ccccc2N(c2ccc4c5cccc6c7ccccc7n(c4c2)c65)c2ccccc23)cc1]

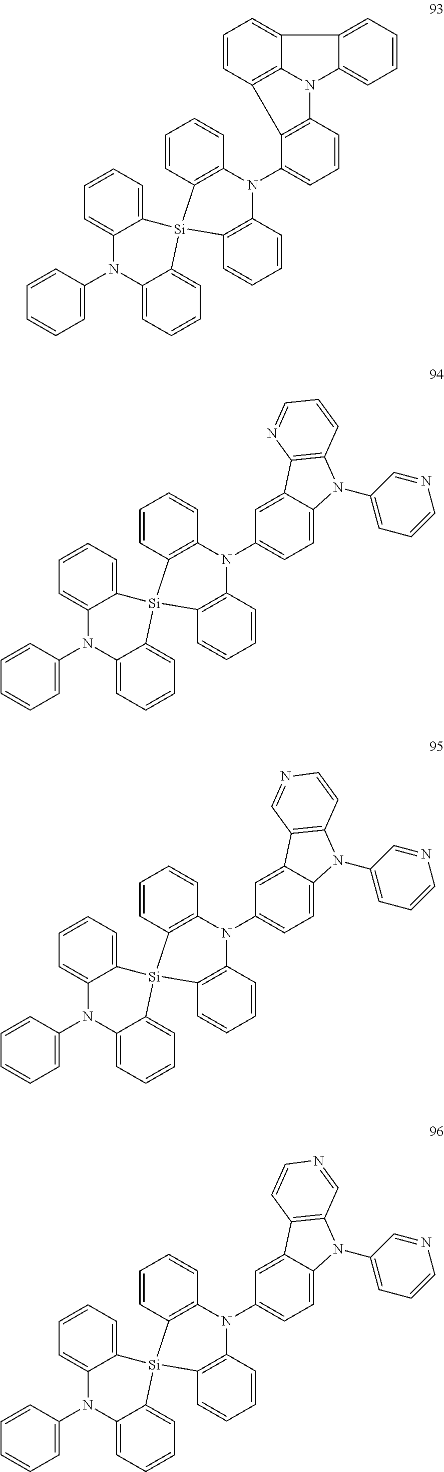 [SMILES: c1ccc(N2c3ccccc3[Si]3(c4ccccc42)c2ccccc2N(c2cccc4c2c2cccc5c6ccccc6n4c52)c2ccccc23)cc1 ; c1ccc(N2c3ccccc3[Si]3(c4ccccc42)c2ccccc2N(c2ccc4c(c2)c2ncccc2n4-c2cccnc2)c2ccccc23)cc1 ; c1ccc(N2c3ccccc3[Si]3(c4ccccc42)c2ccccc2N(c2ccc4c(c2)c2cnccc2n4-c2cccnc2)c2ccccc23)cc1 ; c1ccc(N2c3ccccc3[Si]3(c4ccccc42)c2ccccc2N(c2ccc4c(c2)c2ccncc2n4-c2cccnc2)c2ccccc23)cc1]

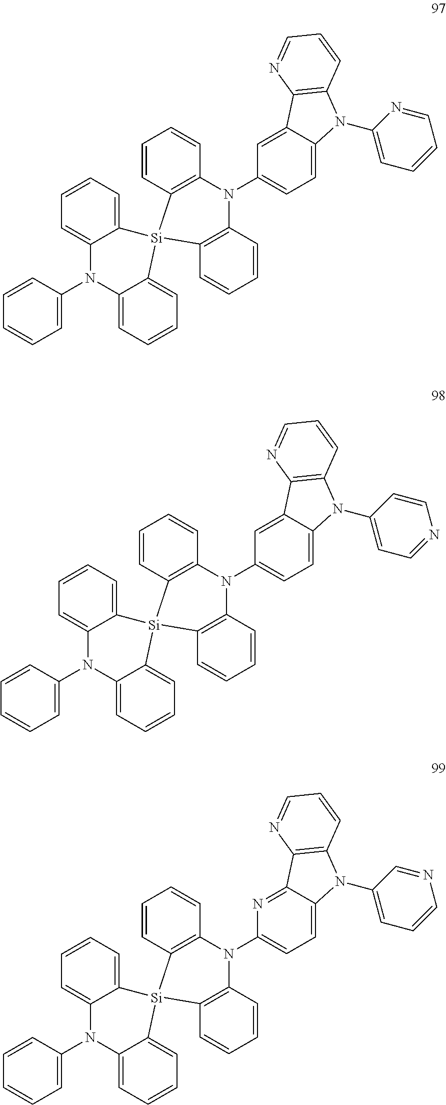 [SMILES: c1ccc(N2c3ccccc3[Si]3(c4ccccc42)c2ccccc2N(c2ccc4c(c2)c2ncccc2n4-c2ccccn2)c2ccccc23)cc1 ; c1ccc(N2c3ccccc3[Si]3(c4ccccc42)c2ccccc2N(c2ccc4c(c2)c2ncccc2n4-c2ccncc2)c2ccccc23)cc1 ; c1ccc(N2c3ccccc3[Si]3(c4ccccc42)c2ccccc2N(c2ccc4c(n2)c2ncccc2n4-c2cccnc2)c2ccccc23)cc1]

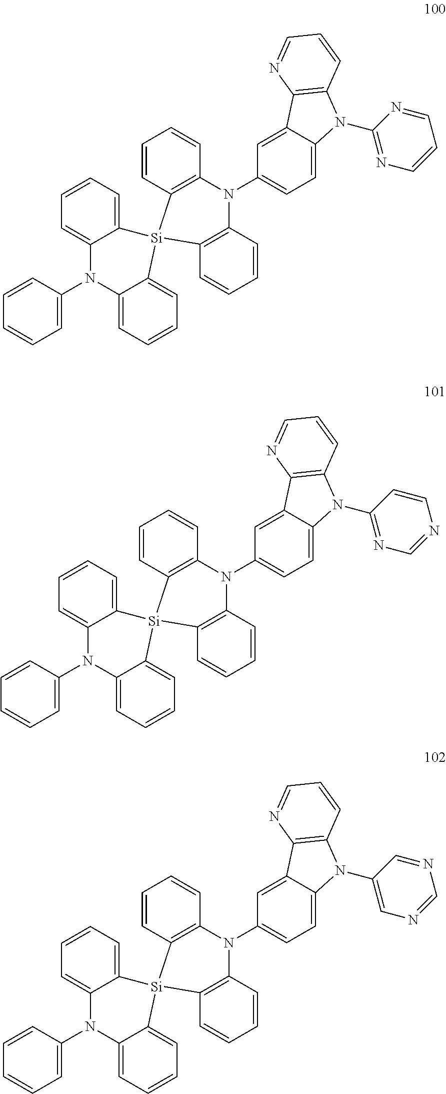 [SMILES: c1ccc(N2c3ccccc3[Si]3(c4ccccc42)c2ccccc2N(c2ccc4c(c2)c2ncccc2n4-c2ncccn2)c2ccccc23)cc1 ; c1ccc(N2c3ccccc3[Si]3(c4ccccc42)c2ccccc2N(c2ccc4c(c2)c2ncccc2n4-c2ccncn2)c2ccccc23)cc1 ; c1ccc(N2c3ccccc3[Si]3(c4ccccc42)c2ccccc2N(c2ccc4c(c2)c2ncccc2n4-c2cncnc2)c2ccccc23)cc1]

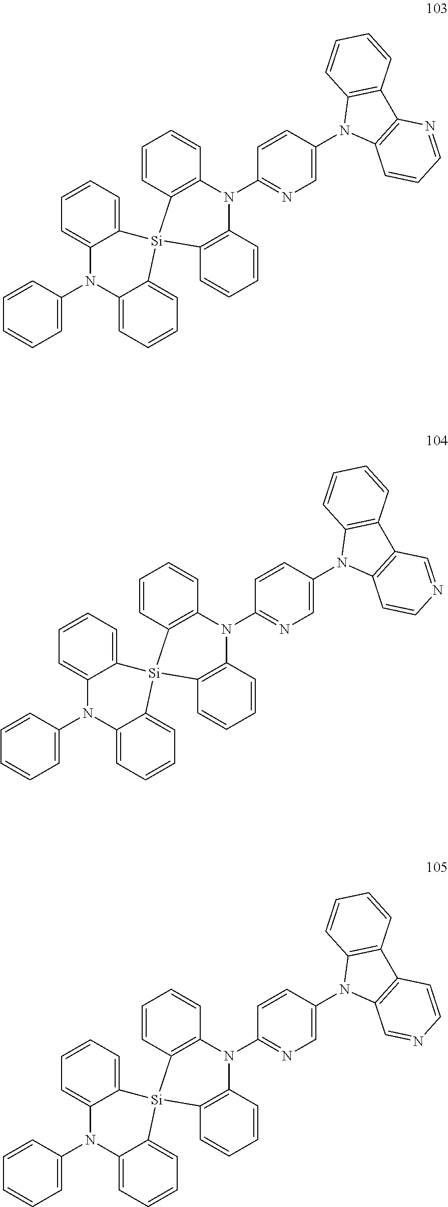 [SMILES: c1ccc(N2c3ccccc3[Si]3(c4ccccc42)c2ccccc2N(c2ccc(-n4c5ccccc5c5ncccc54)cn2)c2ccccc23)cc1 ; c1ccc(N2c3ccccc3[Si]3(c4ccccc42)c2ccccc2N(c2ccc(-n4c5ccccc5c5cnccc54)cn2)c2ccccc23)cc1 ; c1ccc(N2c3ccccc3[Si]3(c4ccccc42)c2ccccc2N(c2ccc(-n4c5ccccc5c5ccncc54)cn2)c2ccccc23)cc1]

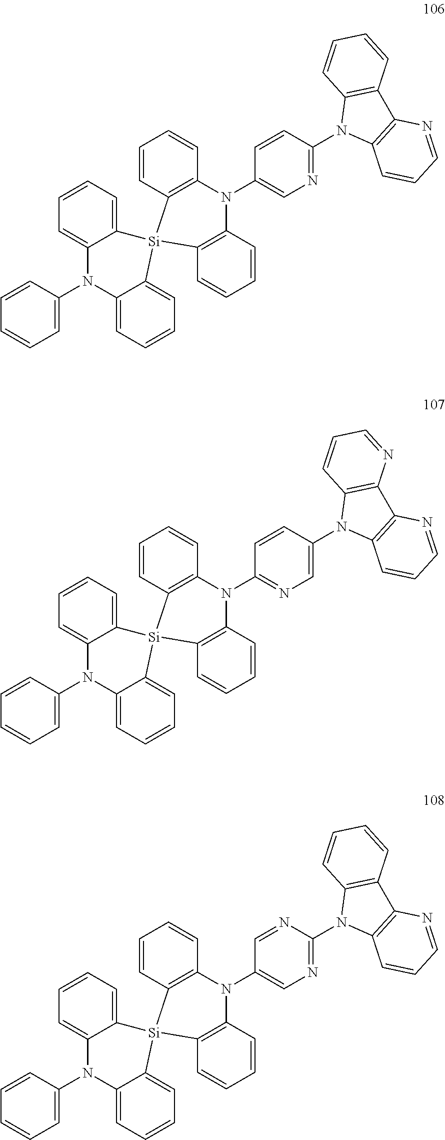 [SMILES: c1ccc(N2c3ccccc3[Si]3(c4ccccc42)c2ccccc2N(c2ccc(-n4c5ccccc5c5ncccc54)nc2)c2ccccc23)cc1 ; c1ccc(N2c3ccccc3[Si]3(c4ccccc42)c2ccccc2N(c2ccc(-n4c5cccnc5c5ncccc54)cn2)c2ccccc23)cc1 ; c1ccc(N2c3ccccc3[Si]3(c4ccccc42)c2ccccc2N(c2cnc(-n4c5ccccc5c5ncccc54)nc2)c2ccccc23)cc1]

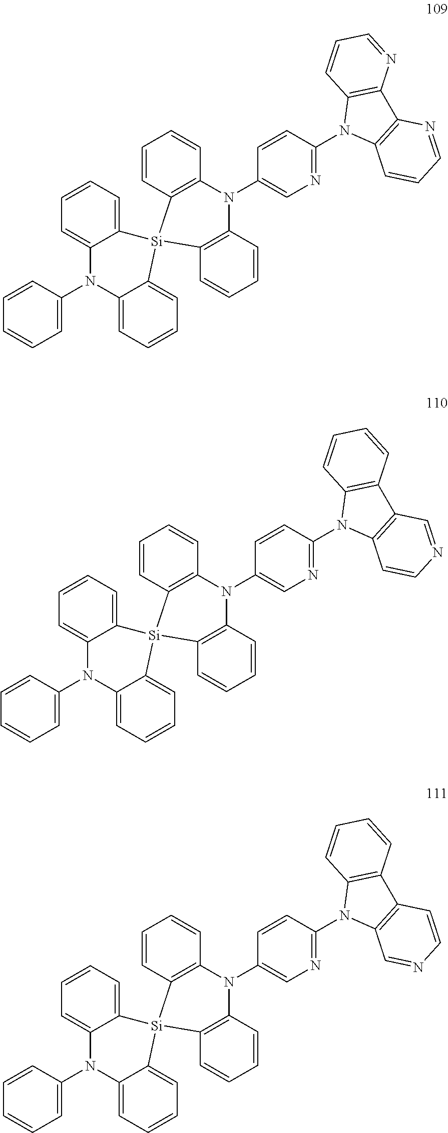 [SMILES: c1ccc(N2c3ccccc3[Si]3(c4ccccc42)c2ccccc2N(c2ccc(-n4c5cccnc5c5ncccc54)nc2)c2ccccc23)cc1 ; c1ccc(N2c3ccccc3[Si]3(c4ccccc42)c2ccccc2N(c2ccc(-n4c5ccccc5c5cnccc54)nc2)c2ccccc23)cc1 ; c1ccc(N2c3ccccc3[Si]3(c4ccccc42)c2ccccc2N(c2ccc(-n4c5ccccc5c5ccncc54)nc2)c2ccccc23)cc1]

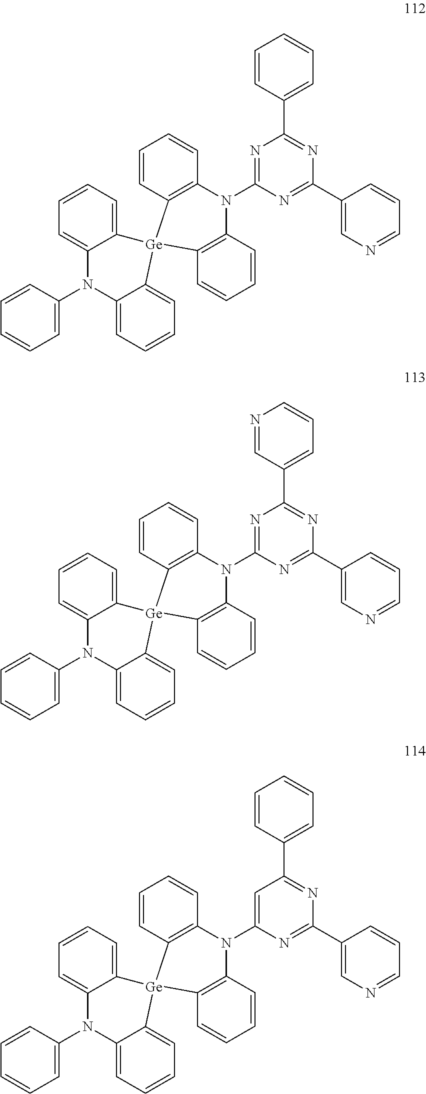

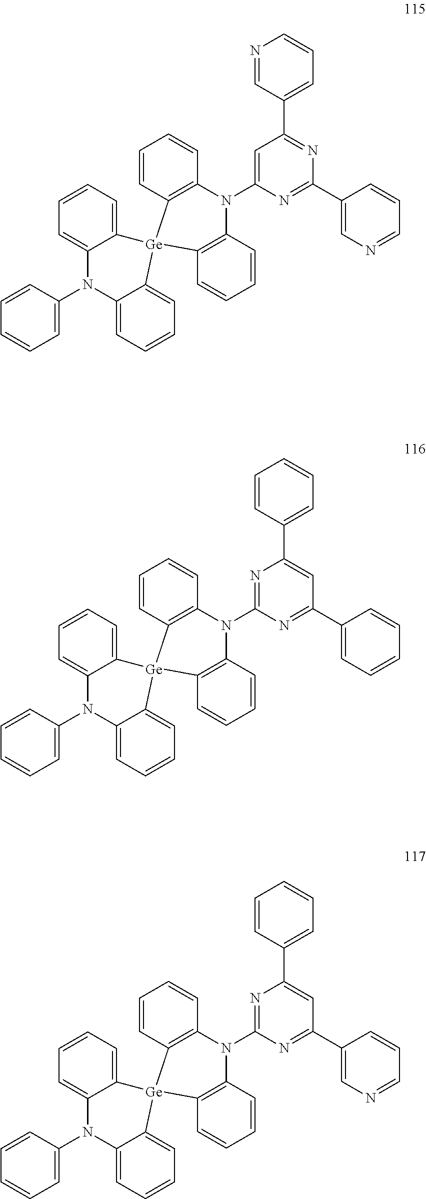

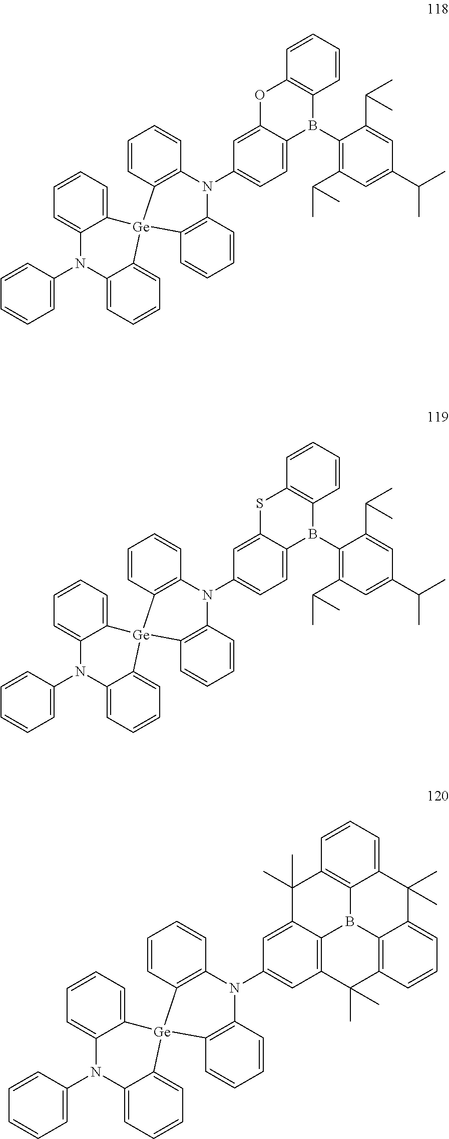

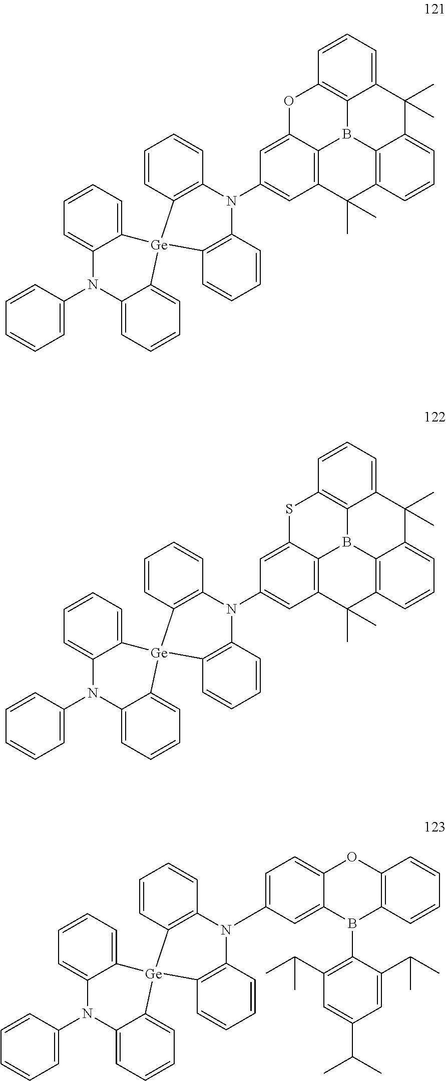

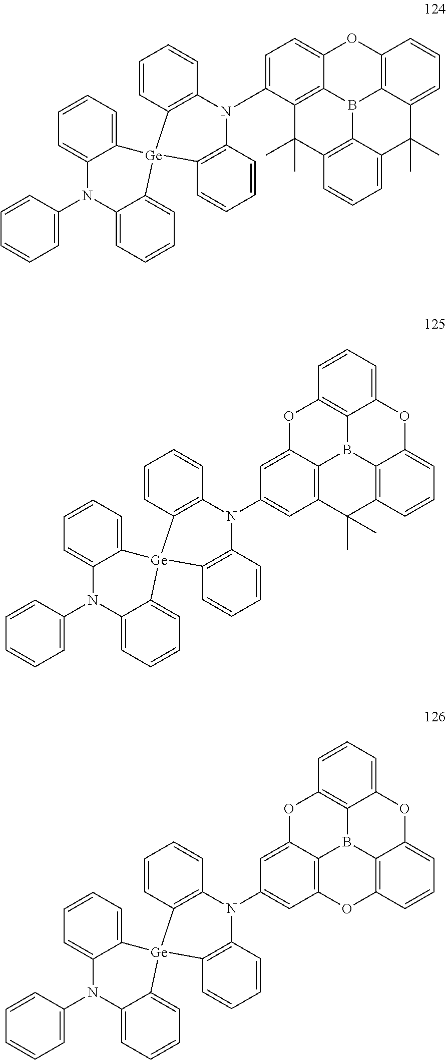

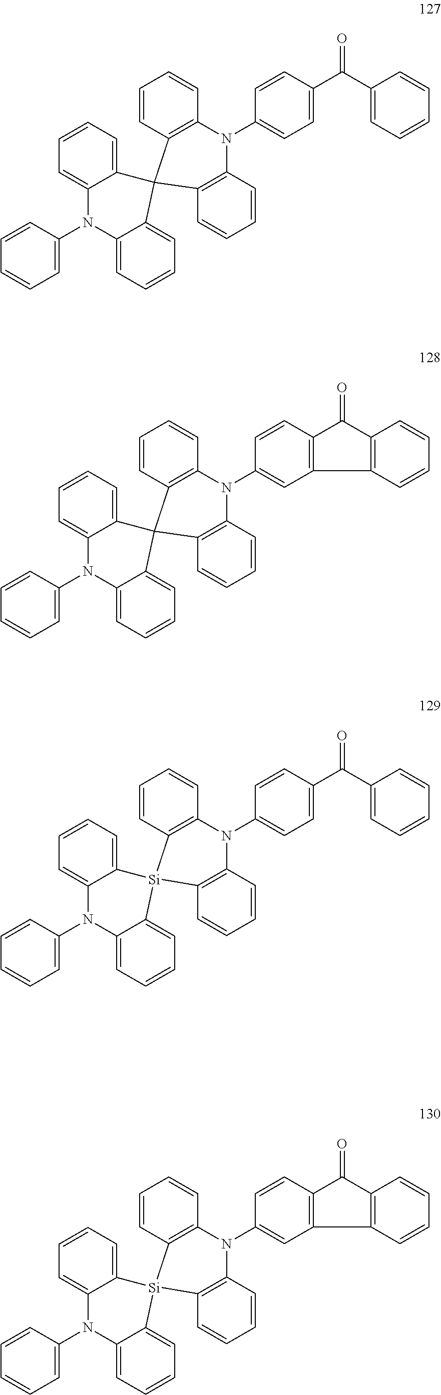 [SMILES: O=C(c1ccccc1)c1ccc(N2c3ccccc3C3(c4ccccc4N(c4ccccc4)c4ccccc43)c3ccccc32)cc1 ; O=C1c2ccccc2-c2cc(N3c4ccccc4C4(c5ccccc5N(c5ccccc5)c5ccccc54)c4ccccc43)ccc21 ; O=C(c1ccccc1)c1ccc(N2c3ccccc3[Si]3(c4ccccc4N(c4ccccc4)c4ccccc43)c3ccccc32)cc1 ; O=C1c2ccccc2-c2cc(N3c4ccccc4[Si]4(c5ccccc5N(c5ccccc5)c5ccccc54)c4ccccc43)ccc21]

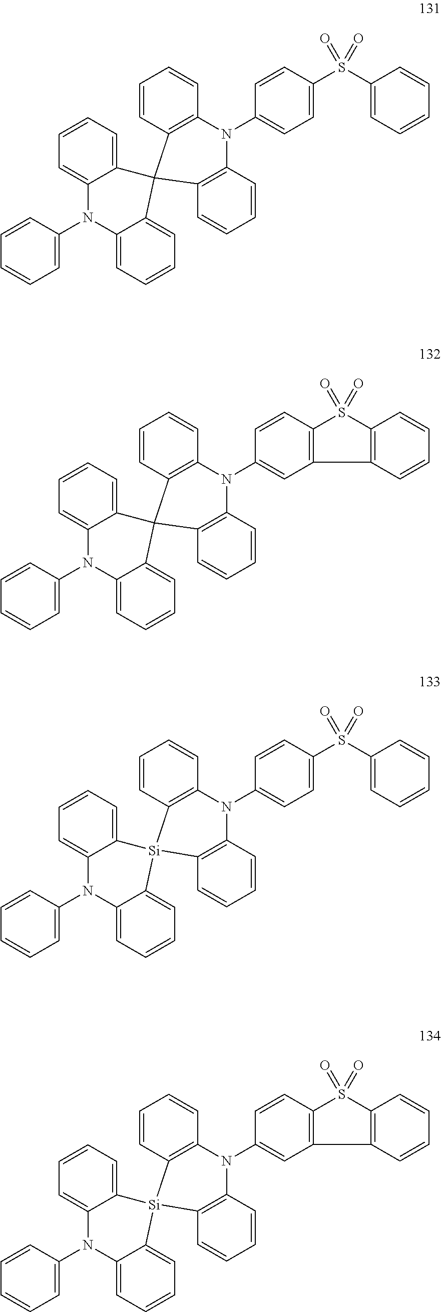 [SMILES: O=S(=O)(c1ccccc1)c1ccc(N2c3ccccc3C3(c4ccccc4N(c4ccccc4)c4ccccc43)c3ccccc32)cc1 ; O=S1(=O)c2ccccc2-c2cc(N3c4ccccc4C4(c5ccccc5N(c5ccccc5)c5ccccc54)c4ccccc43)ccc21 ; O=S(=O)(c1ccccc1)c1ccc(N2c3ccccc3[Si]3(c4ccccc4N(c4ccccc4)c4ccccc43)c3ccccc32)cc1 ; O=S1(=O)c2ccccc2-c2cc(N3c4ccccc4[Si]4(c5ccccc5N(c5ccccc5)c5ccccc54)c4ccccc43)ccc21]

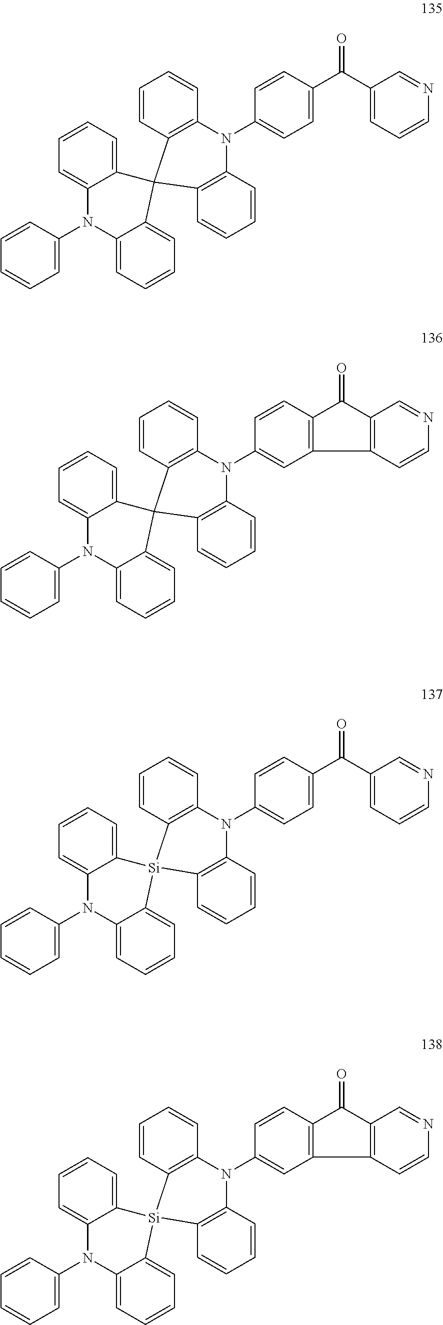 [SMILES: O=C(c1ccc(N2c3ccccc3C3(c4ccccc4N(c4ccccc4)c4ccccc43)c3ccccc32)cc1)c1cccnc1 ; O=C1c2cnccc2-c2cc(N3c4ccccc4C4(c5ccccc5N(c5ccccc5)c5ccccc54)c4ccccc43)ccc21 ; O=C(c1ccc(N2c3ccccc3[Si]3(c4ccccc4N(c4ccccc4)c4ccccc43)c3ccccc32)cc1)c1cccnc1 ; O=C1c2cnccc2-c2cc(N3c4ccccc4[Si]4(c5ccccc5N(c5ccccc5)c5ccccc54)c4ccccc43)ccc21]

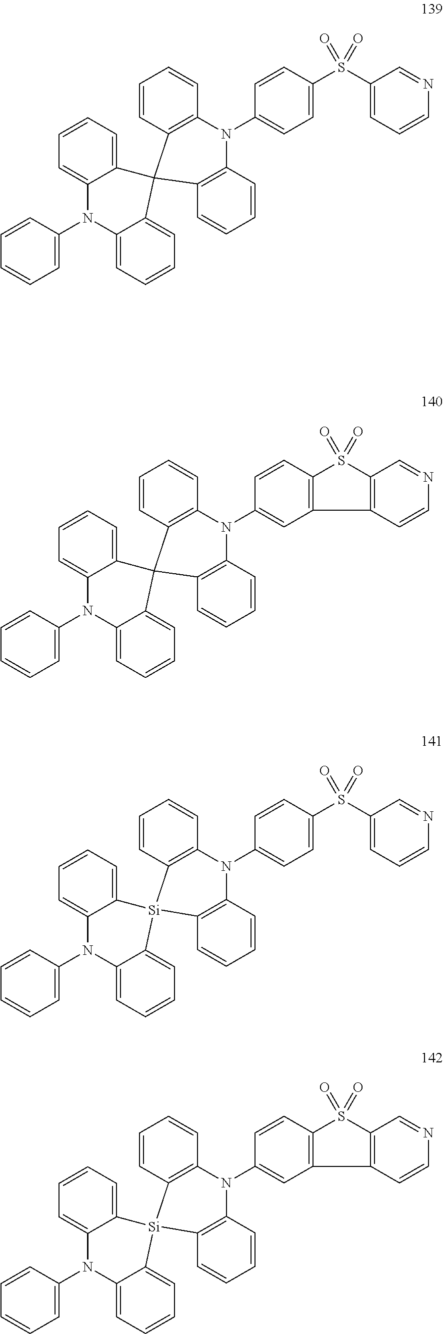 [SMILES: O=S(=O)(c1ccc(N2c3ccccc3C3(c4ccccc4N(c4ccccc4)c4ccccc43)c3ccccc32)cc1)c1cccnc1 ; O=S1(=O)c2ccc(N3c4ccccc4C4(c5ccccc5N(c5ccccc5)c5ccccc54)c4ccccc43)cc2-c2ccncc21 ; O=S(=O)(c1ccc(N2c3ccccc3[Si]3(c4ccccc4N(c4ccccc4)c4ccccc43)c3ccccc32)cc1)c1cccnc1 ; O=S1(=O)c2ccc(N3c4ccccc4[Si]4(c5ccccc5N(c5ccccc5)c5ccccc54)c4ccccc43)cc2-c2ccncc21]

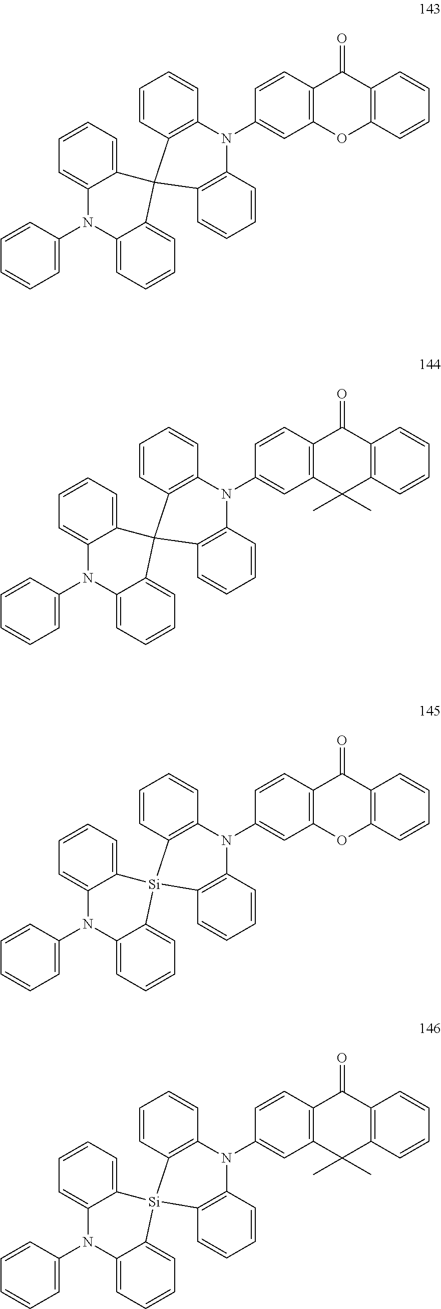 [SMILES: O=c1c2ccccc2oc2cc(N3c4ccccc4C4(c5ccccc5N(c5ccccc5)c5ccccc54)c4ccccc43)ccc12 ; CC1(C)c2ccccc2C(=O)c2ccc(N3c4ccccc4C4(c5ccccc5N(c5ccccc5)c5ccccc54)c4ccccc43)cc21 ; O=c1c2ccccc2oc2cc(N3c4ccccc4[Si]4(c5ccccc5N(c5ccccc5)c5ccccc54)c4ccccc43)ccc12 ; CC1(C)c2ccccc2C(=O)c2ccc(N3c4ccccc4[Si]4(c5ccccc5N(c5ccccc5)c5ccccc54)c4ccccc43)cc21]

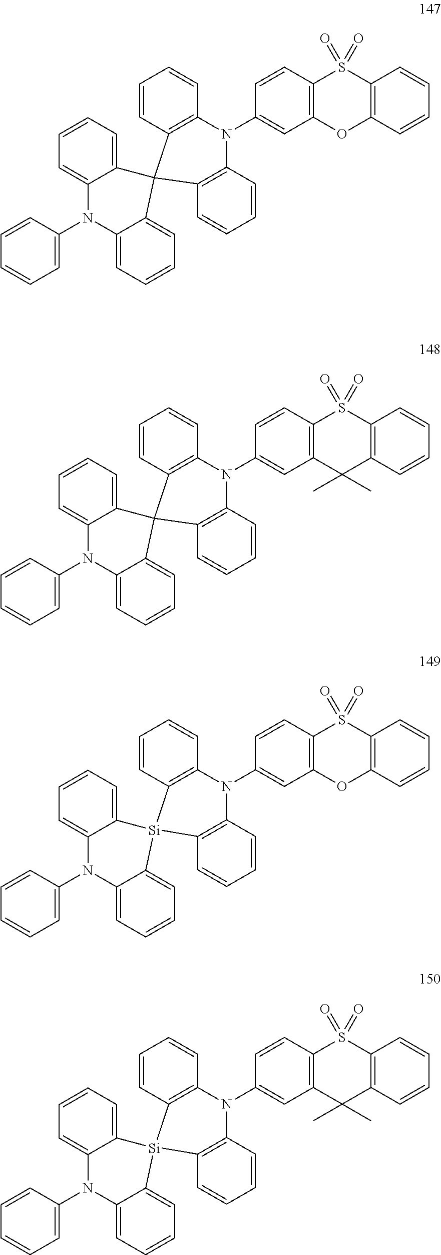 [SMILES: O=S1(=O)c2ccccc2Oc2cc(N3c4ccccc4C4(c5ccccc5N(c5ccccc5)c5ccccc54)c4ccccc43)ccc21 ; CC1(C)c2ccccc2S(=O)(=O)c2ccc(N3c4ccccc4C4(c5ccccc5N(c5ccccc5)c5ccccc54)c4ccccc43)cc21 ; O=S1(=O)c2ccccc2Oc2cc(N3c4ccccc4[Si]4(c5ccccc5N(c5ccccc5)c5ccccc54)c4ccccc43)ccc21 ; CC1(C)c2ccccc2S(=O)(=O)c2ccc(N3c4ccccc4[Si]4(c5ccccc5N(c5ccccc5)c5ccccc54)c4ccccc43)cc21]

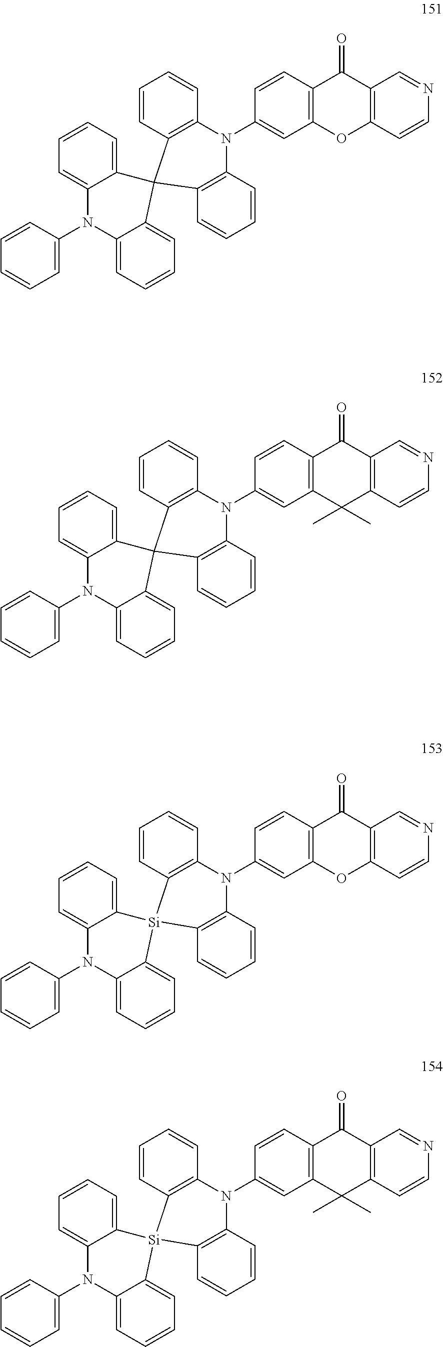 [SMILES: O=c1c2ccc(N3c4ccccc4C4(c5ccccc5N(c5ccccc5)c5ccccc54)c4ccccc43)cc2oc2ccncc12 ; CC1(C)c2ccncc2C(=O)c2ccc(N3c4ccccc4C4(c5ccccc5N(c5ccccc5)c5ccccc54)c4ccccc43)cc21 ; O=c1c2ccc(N3c4ccccc4[Si]4(c5ccccc5N(c5ccccc5)c5ccccc54)c4ccccc43)cc2oc2ccncc12 ; CC1(C)c2ccncc2C(=O)c2ccc(N3c4ccccc4[Si]4(c5ccccc5N(c5ccccc5)c5ccccc54)c4ccccc43)cc21]

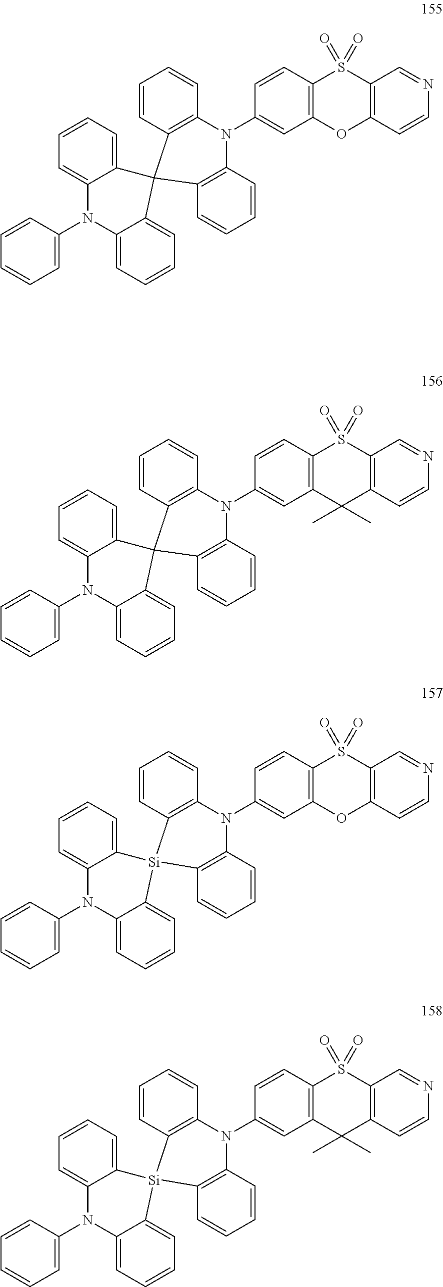 [SMILES: O=S1(=O)c2ccc(N3c4ccccc4C4(c5ccccc5N(c5ccccc5)c5ccccc54)c4ccccc43)cc2Oc2ccncc21 ; CC1(C)c2cc(N3c4ccccc4C4(c5ccccc5N(c5ccccc5)c5ccccc54)c4ccccc43)ccc2S(=O)(=O)c2cnccc21 ; O=S1(=O)c2ccc(N3c4ccccc4[Si]4(c5ccccc5N(c5ccccc5)c5ccccc54)c4ccccc43)cc2Oc2ccncc21 ; CC1(C)c2cc(N3c4ccccc4[Si]4(c5ccccc5N(c5ccccc5)c5ccccc54)c4ccccc43)ccc2S(=O)(=O)c2cnccc21]

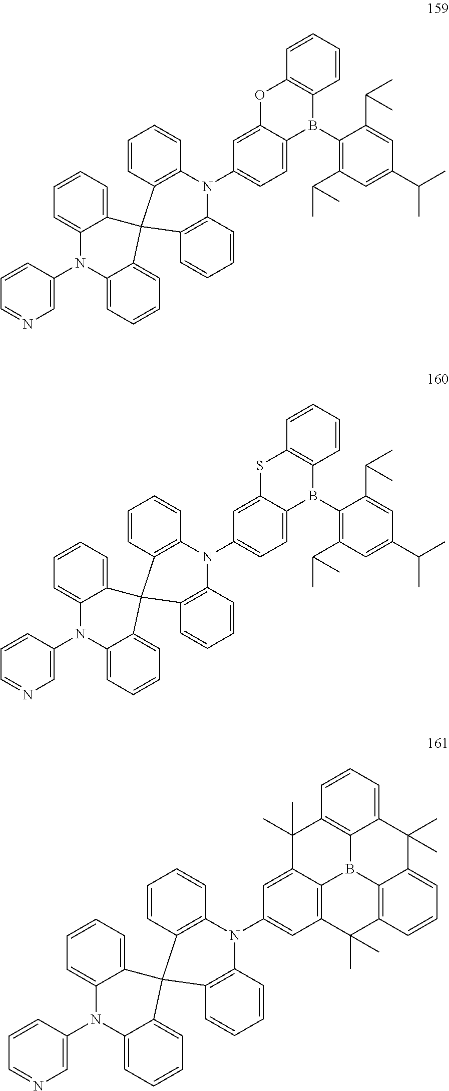 [SMILES: CC(C)c1cc(C(C)C)c(B2c3ccccc3Oc3cc(N4c5ccccc5C5(c6ccccc6N(c6cccnc6)c6ccccc65)c5ccccc54)ccc32)c(C(C)C)c1 ; CC(C)c1cc(C(C)C)c(B2c3ccccc3Sc3cc(N4c5ccccc5C5(c6ccccc6N(c6cccnc6)c6ccccc65)c5ccccc54)ccc32)c(C(C)C)c1 ; CC1(C)c2cccc3c2B2c4c1cccc4C(C)(C)c1cc(N4c5ccccc5C5(c6ccccc6N(c6cccnc6)c6ccccc65)c5ccccc54)cc(c12)C3(C)C]

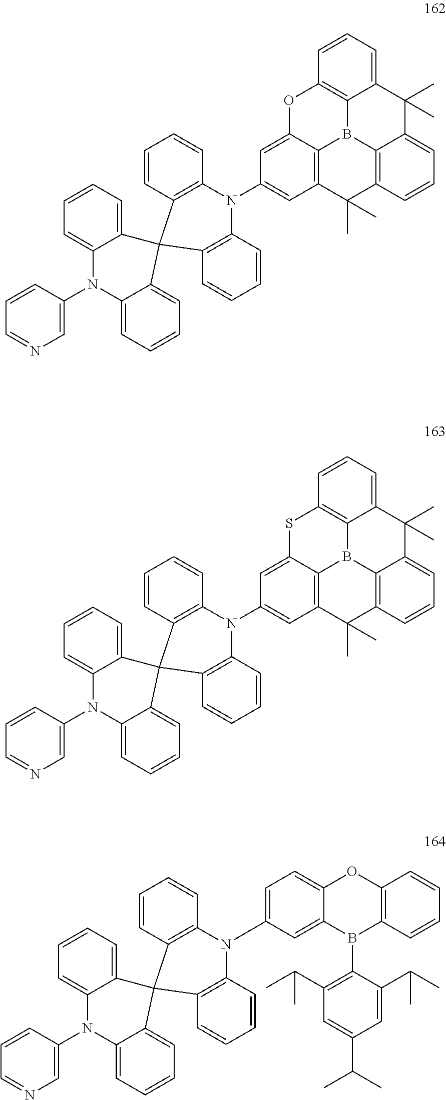 [SMILES: CC1(C)c2cccc3c2B2c4c(cc(N5c6ccccc6C6(c7ccccc7N(c7cccnc7)c7ccccc76)c6ccccc65)cc4C(C)(C)c4cccc1c42)O3 ; CC1(C)c2cccc3c2B2c4c(cc(N5c6ccccc6C6(c7ccccc7N(c7cccnc7)c7ccccc76)c6ccccc65)cc4C(C)(C)c4cccc1c42)S3 ; CC(C)c1cc(C(C)C)c(B2c3ccccc3Oc3ccc(N4c5ccccc5C5(c6ccccc6N(c6cccnc6)c6ccccc65)c5ccccc54)cc32)c(C(C)C)c1]

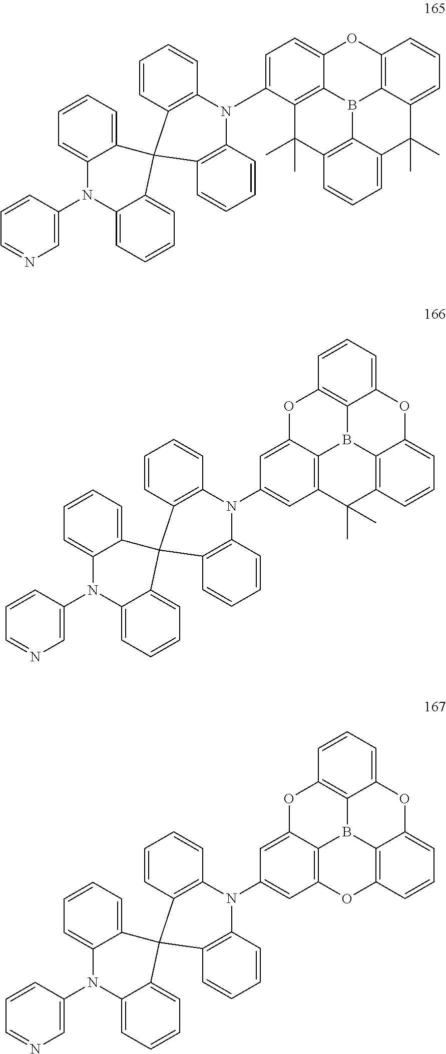 [SMILES: CC1(C)c2cccc3c2B2c4c1cccc4C(C)(C)c1c(N4c5ccccc5C5(c6ccccc6N(c6cccnc6)c6ccccc65)c5ccccc54)ccc(c12)O3 ; CC1(C)c2cccc3c2B2c4c(cccc4Oc4cc(N5c6ccccc6C6(c7ccccc7N(c7cccnc7)c7ccccc76)c6ccccc65)cc1c42)O3 ; c1cncc(N2c3ccccc3C3(c4ccccc42)c2ccccc2N(c2cc4c5c(c2)Oc2cccc6c2B5c2c(cccc2O4)O6)c2ccccc23)c1]

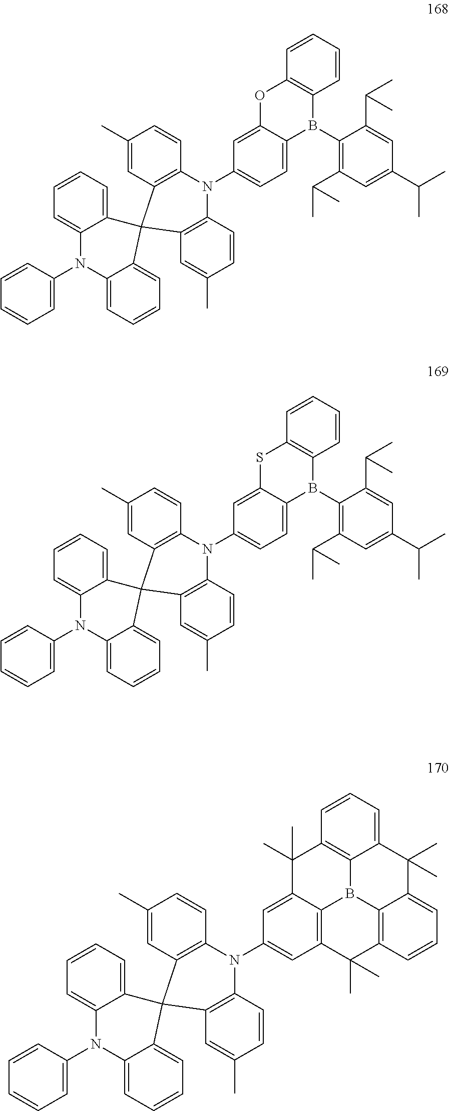 [SMILES: Cc1ccc2c(c1)C1(c3ccccc3N(c3ccccc3)c3ccccc31)c1cc(C)ccc1N2c1ccc2c(c1)Oc1ccccc1B2c1c(C(C)C)cc(C(C)C)cc1C(C)C ; Cc1ccc2c(c1)C1(c3ccccc3N(c3ccccc3)c3ccccc31)c1cc(C)ccc1N2c1ccc2c(c1)Sc1ccccc1B2c1c(C(C)C)cc(C(C)C)cc1C(C)C ; Cc1ccc2c(c1)C1(c3ccccc3N(c3ccccc3)c3ccccc31)c1cc(C)ccc1N2c1cc2c3c(c1)C(C)(C)c1cccc4c1B3c1c(cccc1C2(C)C)C4(C)C]

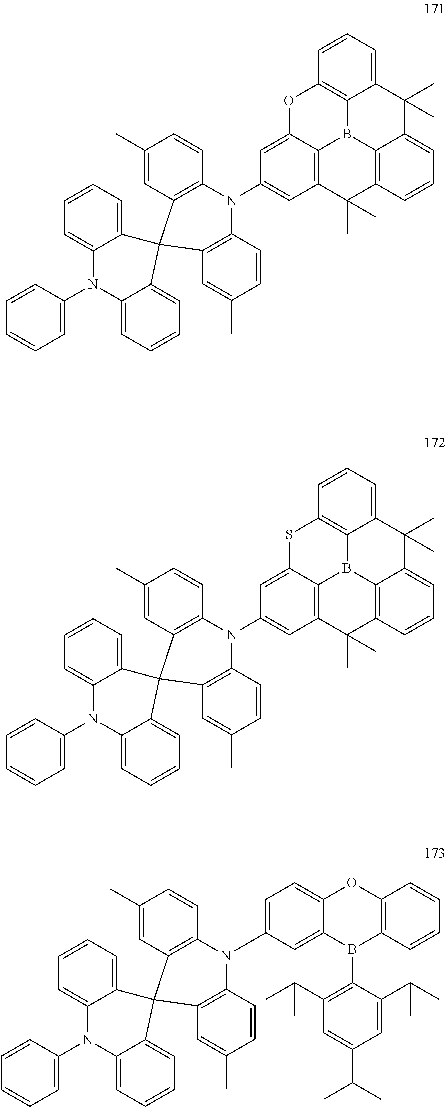 [SMILES: Cc1ccc2c(c1)C1(c3ccccc3N(c3ccccc3)c3ccccc31)c1cc(C)ccc1N2c1cc2c3c(c1)C(C)(C)c1cccc4c1B3c1c(cccc1C4(C)C)O2 ; Cc1ccc2c(c1)C1(c3ccccc3N(c3ccccc3)c3ccccc31)c1cc(C)ccc1N2c1cc2c3c(c1)C(C)(C)c1cccc4c1B3c1c(cccc1C4(C)C)S2 ; Cc1ccc2c(c1)C1(c3ccccc3N(c3ccccc3)c3ccccc31)c1cc(C)ccc1N2c1ccc2c(c1)B(c1c(C(C)C)cc(C(C)C)cc1C(C)C)c1ccccc1O2]

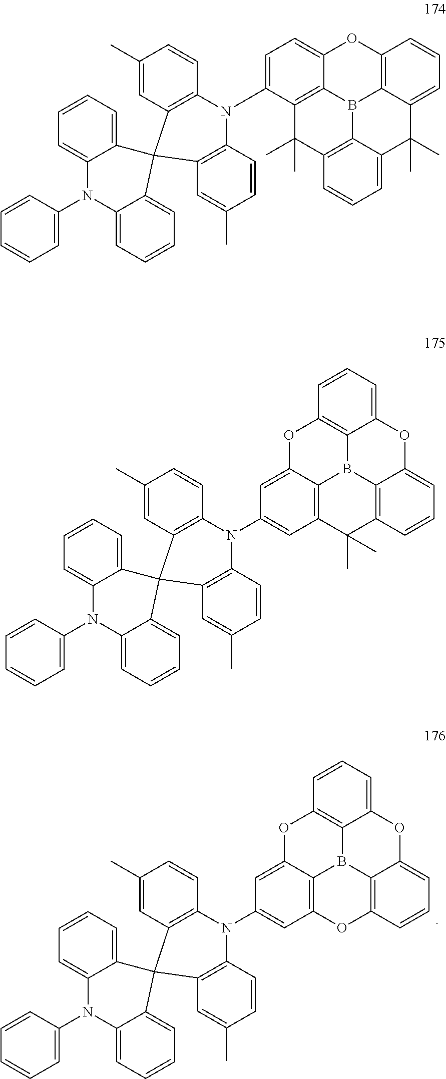 [SMILES: Cc1ccc2c(c1)C1(c3ccccc3N(c3ccccc3)c3ccccc31)c1cc(C)ccc1N2c1ccc2c3c1C(C)(C)c1cccc4c1B3c1c(cccc1C4(C)C)O2 ; Cc1ccc2c(c1)C1(c3ccccc3N(c3ccccc3)c3ccccc31)c1cc(C)ccc1N2c1cc2c3c(c1)C(C)(C)c1cccc4c1B3c1c(cccc1O2)O4 ; Cc1ccc2c(c1)C1(c3ccccc3N(c3ccccc3)c3ccccc31)c1cc(C)ccc1N2c1cc2c3c(c1)Oc1cccc4c1B3c1c(cccc1O2)O4]

The polycyclic compounds according to one or more embodiments of the present disclosure may have an energy level difference between a singlet energy level and a triplet energy level of about 0.2 eV or less, and as a result, may be utilized as a material for thermally activated delayed fluorescence. The polycyclic compound according to one or more embodiments of the present disclosure may be used as a material for an organic electroluminescence device to contribute to an increase in efficiency.

An organic electroluminescence device will be explained by reference to FIGS. 1-3.

The emission layer EML may include one or two or more kinds or structures of the polycyclic compound represented by Formula 1. The emission layer EML may further include other suitable materials in addition to the polycyclic compound represented by Formula 1.

The emission layer EML may include a host and a dopant, and the dopant may include the polycyclic compound represented by Formula 1. The polycyclic compound according to one or more embodiments of the present disclosure may be included in the emission layer EML as a dopant for thermally activated delayed fluorescence. The polycyclic compound according to one or more embodiments of the present disclosure may be included in the emission layer EML as a dopant for emitting blue light having a wavelength of about 470 nm or less, and for example, may be included in the emission layer EML as a dopant for emitting deep blue light having a wavelength of about 440 nm to about 470 nm, or about 450 nm to about 470 nm.

The host material may include suitable materials available in the art, without limitation. For example, at least one of bis[2-(diphenylphosphino)phenyl] ether oxide (DPEPO), 4,4′-bis(carbazol-9-yl)biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TcTa), and 1,3,5-tris(N-phenylbenzimidazole-2-yl)benzene (TPBi) may be included. However, embodiments of the present disclosure are not limited thereto. For example, tris(8-hydroxyquinolino)aluminum (Alq₃), 4,4′-bis(N-carbazolyl)-1,1′-biphenyl (CBP), poly(N-vinylcarbazole) (PVK), 9,10-di(naphthalene-2-yl)anthracene (ADN), 4,4′,4″-tris(carbazol-9-yl)-triphenylamine (TCTA), 1,3,5-tris(N-phenylbenzim idazole-2-yl)benzene (TPBi), 3-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), bis[2-(diphenylphosphino)phenyl]ether oxide (DPEPO), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), 2,8-bis(diphenylphosphoryl)dibenzofuran (PPF), etc., may be used or included as the host material.

For example, the emission layer EML may further include at least one of N,N,N′,N′-tetraphenyl-pyrene-1,6-diamine (TPD), 4,4′-bis(2-(9-ethyl-9H-carbazol-3-yl)vinyl)-1,1′-biphenyl (BCzVBi); 4,4′-bis(9-ethyl-3-carbazovinylene)-1,1′-biphenyl, 10-phenyl-10H,10′H-spiro[acridine-9,9′-anthracene]-10′-one (ACRSA), 3,4,5,6-tetra-9H-carbazol-9-yl-1,2-benzenedicarbonitrile (4CzPN), 2,4,5,6-tetra-9H-carbazol-9-yl-isophthalonitrile (4CzlPN), bis[4-9,9-dimethyl-9,10-dihydroacridine]phenyl]sulfone (DMAC-DPS), and 2-phenoxazine-4,6-diphenyl-1,3,5-triazine (PSZ-TRZ) as a dopant. In some embodiments, the emission layer EML may further include styryl derivatives (for example, 1,4-bis[2-(3-N-ethylcarbazoryl)vinyl]benzene (BCzVB), 4-(di-p-tolylamino)-4′-[(di-p-tolylamino)styryl]stilbene (DPAVB), and N-(4-((E)-2-(6-((E)-4-(diphenylamino)styryl)naphthalen-2-yl)vinyl)phenyl)-N-phenylbenzenamine (N-BDAVBi)), perylene and derivatives thereof (for example, 2,5,8,11-tetra-t-butylperylene (TBP)), pyrene and the derivatives thereof (for example, 1,1-dipyrene, 1,4-dipyrenylbenzene, 1,4-bis(N,N-diphenylamino)pyrene), etc., as dopant materials.

The emission layer EML may be a blue emission layer to emit blue light. The emission layer EML may be an emission layer to emit light in a wavelength of about 480 nm or less, or about 470 nm or less. The emission layer EML may be a fluorescence emission layer radiating fluorescence. The emission layer EML may be a delayed fluorescence emission layer radiating delayed fluorescence.

The electron transport region ETR may be on the emission layer EML. The electron transport region ETR may include at least one of a hole blocking layer HBL, an electron transport layer ETL, or an electron injection layer EIL, without limitation. In some embodiments, for example, the electron transport region ETR may include an electron injection layer EIL and an electron transport layer ETL, as shown in FIG. 2. In some embodiments, the electron transport region ETR may include an electron injection layer EIL, an electron transport layer ETL, and hole blocking layer EBL, as shown in FIG. 3.

The electron transport region ETR may have or be a single layer formed using a single material, a single layer formed using a plurality of different materials, or a multilayer structure having a plurality of layers formed using a plurality of different materials.

For example, the electron transport region ETR may have a single layer structure of an electron injection layer EIL or an electron transport layer ETL, or a single layer structure formed using an electron injection material and an electron transport material. Further, the electron transport region ETR may have a single layer structure having a plurality of different materials, or a structure laminated from the emission layer EML of electron transport layer ETL/electron injection layer EIL, or hole blocking layer HBL/electron transport layer ETL/electron injection layer EIL, without limitation. The thickness of the electron transport region ETR may be, for example, from about 1,000 Å to about 1,500 Å.

The electron transport region ETR may be formed using one or more suitable methods (such as a vacuum deposition method, a spin coating method, a cast method, a Langmuir-Blodgett (LB) method, an inkjet printing method, a laser printing method, and/or a laser induced thermal imaging (LITI) method).

If the electron transport region ETR includes an electron transport layer ETL, the electron transport region ETR may include an anthracene-based compound. However, embodiments of the present disclosure are not limited thereto. The electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazolyl-1-ylphenyl)-9,10-dinaphthylanthracene, 1,3,5-tri(1-phenyl-1H-benzo[d]imidazol-2-yl)phenyl (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphehyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), beryllium bis(benzoquinolin-10-olate (Bebq₂), 9,10-di(naphthalene-2-yl)anthracene (ADN), and/or a mixture thereof. The thickness of the electron transport layer ETL may be about 100 Å to about 1,000 Å, for example, about 150 Å to about 500 Å. When the thickness of the electron transport layers ETL satisfies the above-described range, satisfactory electron transport properties may be obtained without a substantial increase in driving voltage.

When the electron transport region ETR includes an electron injection layer EIL, the electron transport layer ETL may use LiF, lithium quinolate (LiQ), Li₂O, BaO, NaCl, CsF, a lanthanide metal (such as ytterbium (Yb)), or a metal halide (such as RbCl and/or Rbl). However, embodiments of the present disclosure are not limited thereto. The electron injection layer EIL may also be formed using a mixture of an electron transport material and an insulating organ metal salt. The organo metal salt may be a material having an energy band gap of about 4 eV or more. Non-limiting examples of the organo metal salt may include metal acetates, metal benzoates, metal acetoacetates, metal acetylacetonates, and metal stearates. The thickness of the electron injection layer EIL may be about 1 Å to about 100 Å, or about 3 Å to about 90 Å. When the thickness of the electron injection layer EIL satisfies the above-described range, satisfactory electron injection properties may be obtained without substantial increase of a driving voltage.

The electron transport region ETR may include a hole blocking layer as described above. The hole blocking layer may include, for example, 2,9-dimethyl-4,7-diphenyl-1,10-phenanthroline (BCP), and/or 4,7-diphenyl-1,10-phenanthroline (Bphen). However, embodiments of the present disclosure are not limited thereto.

The second electrode EL2 may be on the electron transport region ETR. The second electrode EL2 may be a common electrode or a cathode. The second electrode EL2 may be a transmissive electrode, a transflective (e.g., semi-transmissive) electrode, or a reflective electrode. When the second electrode EL2 is a transmissive electrode, the second electrode EL2 may include a transparent metal oxide, for example, ITO, IZO, ZnO, ITZO, etc.

When the second electrode EL2 is a transflective electrode or a reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, a compound including one or more of the listed metals, or a mixture thereof (for example, a mixture of Ag and Mg). The second electrode EL2 may have a multilayered structure including a reflective layer or a transflective layer formed using the above-described materials and a transparent conductive layer formed using ITO, IZO, ZnO, ITZO, etc.

In some embodiments, the second electrode EL2 may be connected with (to) an auxiliary electrode. When the second electrode EL2 is connected with the auxiliary electrode, the resistance of the second electrode EL2 may decrease.

In the organic electroluminescence device 10, during application of a voltage to each of the first electrode EL1 and second electrode EL2, holes injected from the first electrode EL1 may move via the hole transport region HTR to the emission layer EML, and electrons injected from the second electrode EL2 may move via the electron transport region ETR to the emission layer EML. The electrons and the holes may recombine in the emission layer EML to produce excitons, and the excitons may emit light when they transition from an excited state to the ground state.

When the organic electroluminescence device 10 is a top emission type (top emission OLED), the first electrode EL1 may be a reflective electrode and the second electrode EL2 may be a transmissive electrode or a transflective electrode. When the organic electroluminescence device 10 is a bottom emission type (bottom emission OLED), the first electrode EL1 may be a transmissive electrode or a transflective electrode and the second electrode EL2 may be a reflective electrode.

The organic electroluminescence device 10 according to one or more embodiments of the present disclosure may include the polycyclic compound as a material for an emission layer EML, and thus, high efficiency may be achieved.

Hereinafter, the present disclosure will be explained in more detail with reference to example preparation methods, examples, and comparative examples. The following examples are illustrations to assist in the understanding of the present disclosure, and the scope of embodiments of the present disclosure is not limited thereto.

### Synthetic Examples

The polycyclic compound according to one or more embodiments of the present disclosure may be synthesized, for example, as follows. However, embodiments of the present disclosure are not limited thereto.

### 1. Synthesis of Compound 8

Compound 8 according to one or more embodiments of the present disclosure may be synthesized, for example, as follows.

### Synthesis of Intermediate 1A

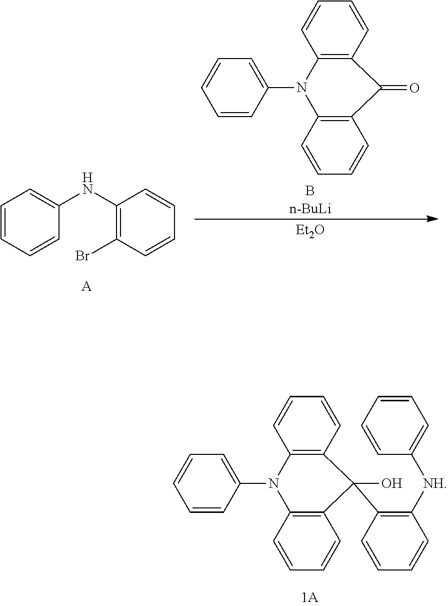

2.0 g of Compound A and 30 mL of dehydrated diethyl ether were added to a 200 mL, three-neck flask under an argon (Ar) atmosphere, and 10 mL of 1.6 M n-BuLi was added dropwise thereto at room temperature, followed by stirring at room temperature for about 2 hours. Then, Compound B dissolved in 20 mL of dehydrated diethyl ether was added dropwise thereto, followed by stirring overnight. After the reaction was complete, water was added, an organic layer was isolated, and the solvents were removed via distillation. The crude product thus obtained was separated by silica gel chromatography (using toluene) to obtain 2.20 g (yield 62%) of Intermediate 1A as a white solid.

The molecular weight of Intermediate 1A, as measured by FAB-MS, was 440.

### Synthesis of Intermediate 2A

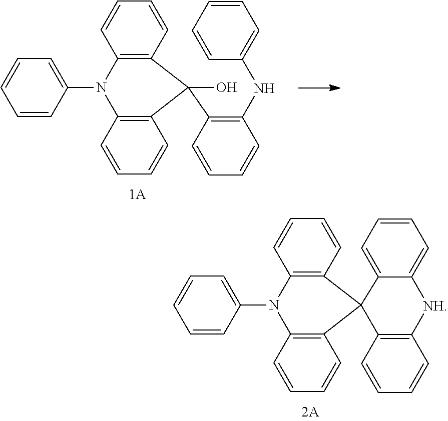 [SMILES: OC1(c2ccccc2Nc2ccccc2)c2ccccc2N(c2ccccc2)c2ccccc21 ; c1ccc(N2c3ccccc3C3(c4ccccc4Nc4ccccc43)c3ccccc32)cc1]

2.0 g of Compound 1A and 20 mL of toluene were added to a 100 mL, three-neck flask under an argon (Ar) atmosphere, and 0.73 g of methylsulfonic acid and 1.0 g of polyphosphoric acid were added thereto, followed by heating, refluxing, and stirring for about 4 hours. After the reaction was complete, water was added, an organic layer was isolated, and the solvents were removed via distillation. The crude product thus obtained was separated by silica gel chromatography (using toluene) to obtain 1.9 g (yield 95%) of Intermediate 2A as a white solid.

The molecular weight of Intermediate 2A, as measured by FAB-MS, was 422.

### Synthesis of Final Compound 8

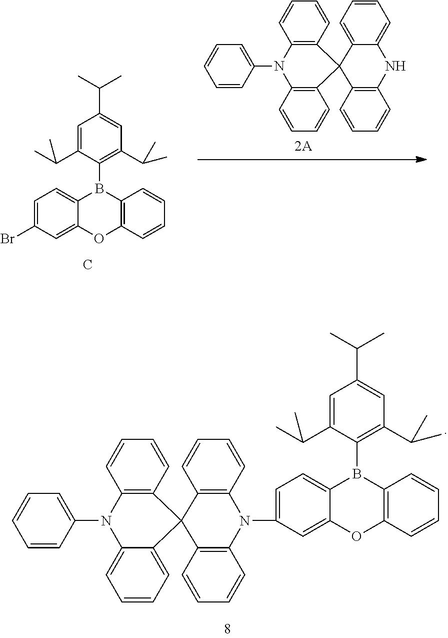

1.0 g of Compound C, 0.95 g of Intermediate 2A, 0.43 g of sodium tert-butoxide, 0.04 g of tris(dibenzylideneacetone)dipalladium(0), 0.05 g of tri-tert-butylphosphonium tetrafluoroborate, and 20 mL of toluene were added to a 100 mL, three-neck flask under an argon (Ar) atmosphere, followed by heating, refluxing, and stirring for about 8 hours. After the reaction was complete, water was added, an organic layer was isolated, and the solvents were removed via distillation. The crude product thus obtained was separated by silica gel chromatography (using a mixture of toluene and hexane) to obtain 1.7 g (yield 94%) of Compound 8 as a white solid.

The molecular weight of Compound 8, as measured by FAB-MS, was 802.

### 2. Synthesis of Compound 40

Compound 40 according to one or more embodiments of the present disclosure may be synthesized, for example, as follows.

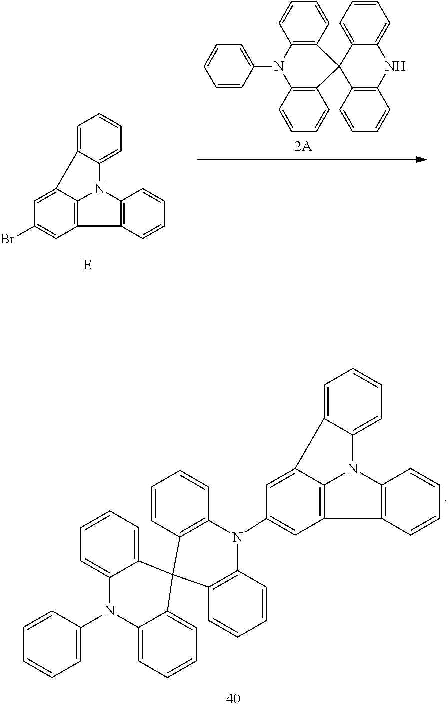

Compound 40 was obtained in 81% yield by conducting substantially the same procedure as described in the synthetic method of Compound 8, except for using Compound E instead of Compound C. The molecular weight of Compound 40, as measured by FAB-MS, was 661.

### 3. Synthesis of Compound 69

Compound 69 according to one or more embodiments of the present disclosure may be synthesized, for example, as follows.

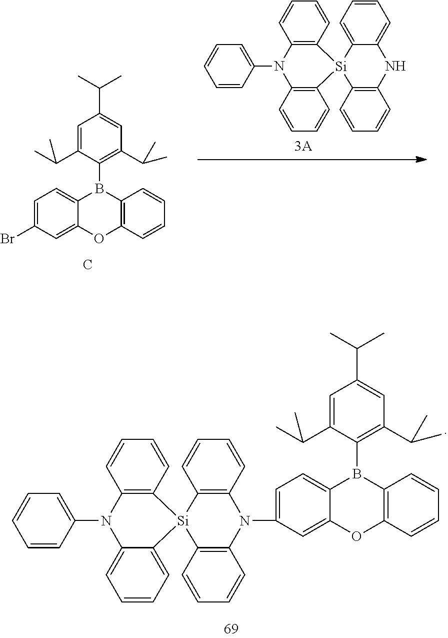

Compound 69 was obtained in 89% yield by conducting substantially the same procedure as described in the synthetic method of Compound 8, except for using Intermediate 3A instead of Intermediate 2A. The molecular weight of Compound 69, as measured by FAB-MS, was 818.

### 4. Synthesis of Compound 127

Compound 127 according to one or more embodiments of the present disclosure may be synthesized, for example, as follows.

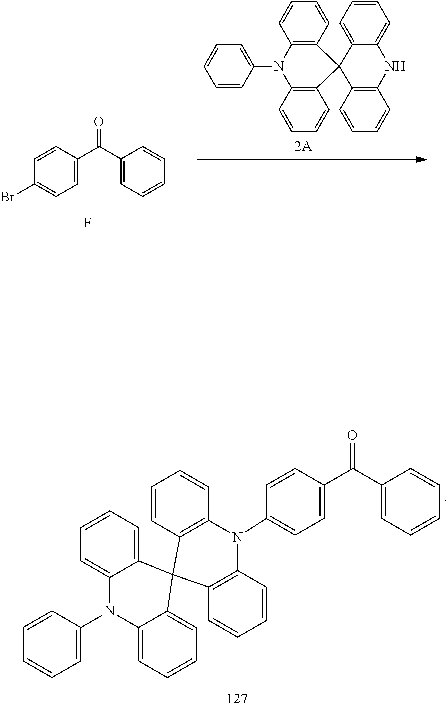

Compound 127 was obtained in 86% yield by conducting substantially the same procedure as described in the synthetic method of Compound 8, except for using Compound F instead of Compound C. The molecular weight of Compound 127, as measured by FAB-MS, was 602.

### 5. Synthesis of Compound 131

Compound 131 according to one or more embodiments of the present disclosure may be synthesized, for example, as follows.

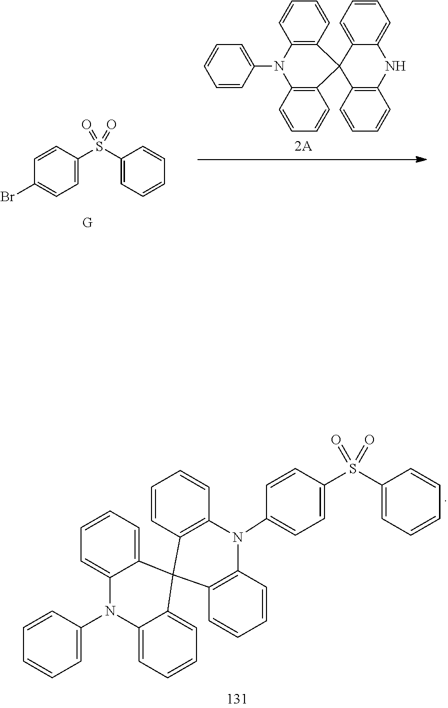

Compound 131 was obtained in 91% yield by conducting substantially the same procedure as described in the synthetic method of Compound 8, except for using Compound G instead of Compound C. The molecular weight of Compound 131, as measured by FAB-MS, was 638.

The above-described synthetic examples are illustrations, and reaction conditions may be changed as necessary. The compounds according to one or more embodiments of the present disclosure may be synthesized so as to include various suitable substituents using methods and materials available in the art. Such substituents may be introduced into a core structure represented by Formula 1 to yield properties suitable for application to an organic electroluminescence device.

### Device Manufacturing Examples

Organic electroluminescence devices of Examples 1 to 5 were manufactured using Compounds 8, 40, 69, 127 and 131, respectively, as dopant materials in the emission layer of each device.

### Example Compounds

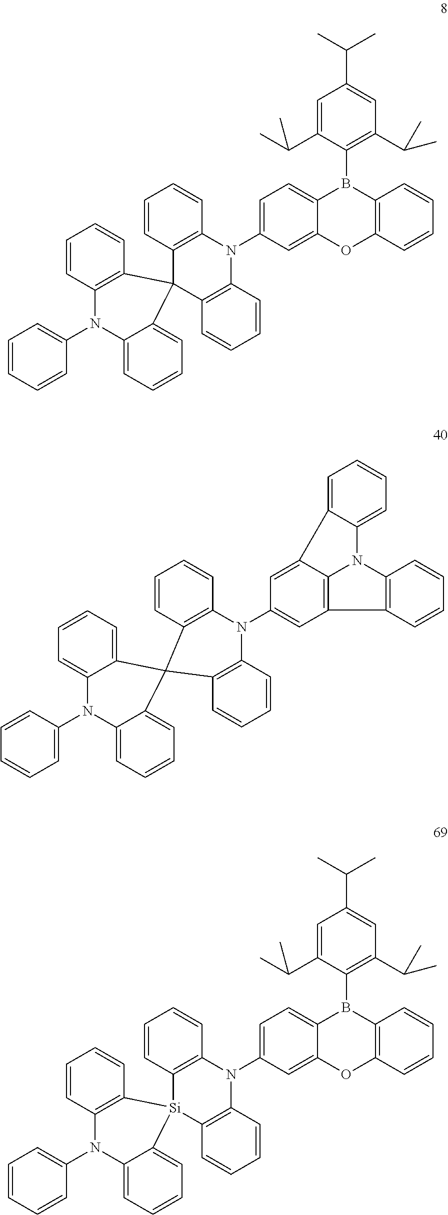 [SMILES: CC(C)c1cc(C(C)C)c(B2c3ccccc3Oc3cc(N4c5ccccc5C5(c6ccccc6N(c6ccccc6)c6ccccc65)c5ccccc54)ccc32)c(C(C)C)c1 ; c1ccc(N2c3ccccc3C3(c4ccccc42)c2ccccc2N(c2cc4c5ccccc5n5c6ccccc6c(c2)c45)c2ccccc23)cc1 ; CC(C)c1cc(C(C)C)c(B2c3ccccc3Oc3cc(N4c5ccccc5[Si]5(c6ccccc6N(c6ccccc6)c6ccccc65)c5ccccc54)ccc32)c(C(C)C)c1]

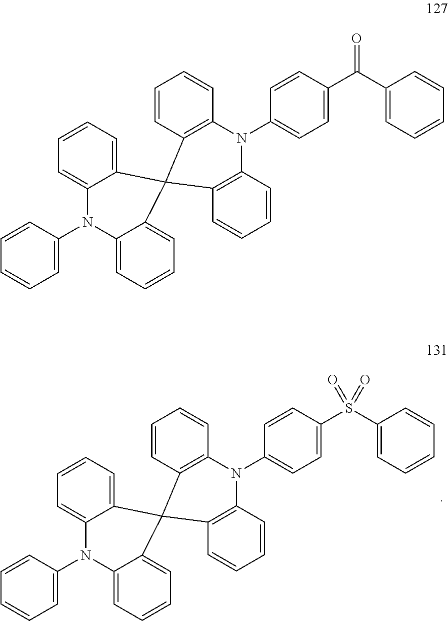 [SMILES: O=C(c1ccccc1)c1ccc(N2c3ccccc3C3(c4ccccc4N(c4ccccc4)c4ccccc43)c3ccccc32)cc1 ; O=S(=O)(c1ccccc1)c1ccc(N2c3ccccc3C3(c4ccccc4N(c4ccccc4)c4ccccc43)c3ccccc32)cc1]

Organic electroluminescence devices of Comparative Examples 1 to 10 were manufactured using Compounds C-1 to C-10, respectively, as dopant materials in the emission layer of each device.

### Comparative Compounds

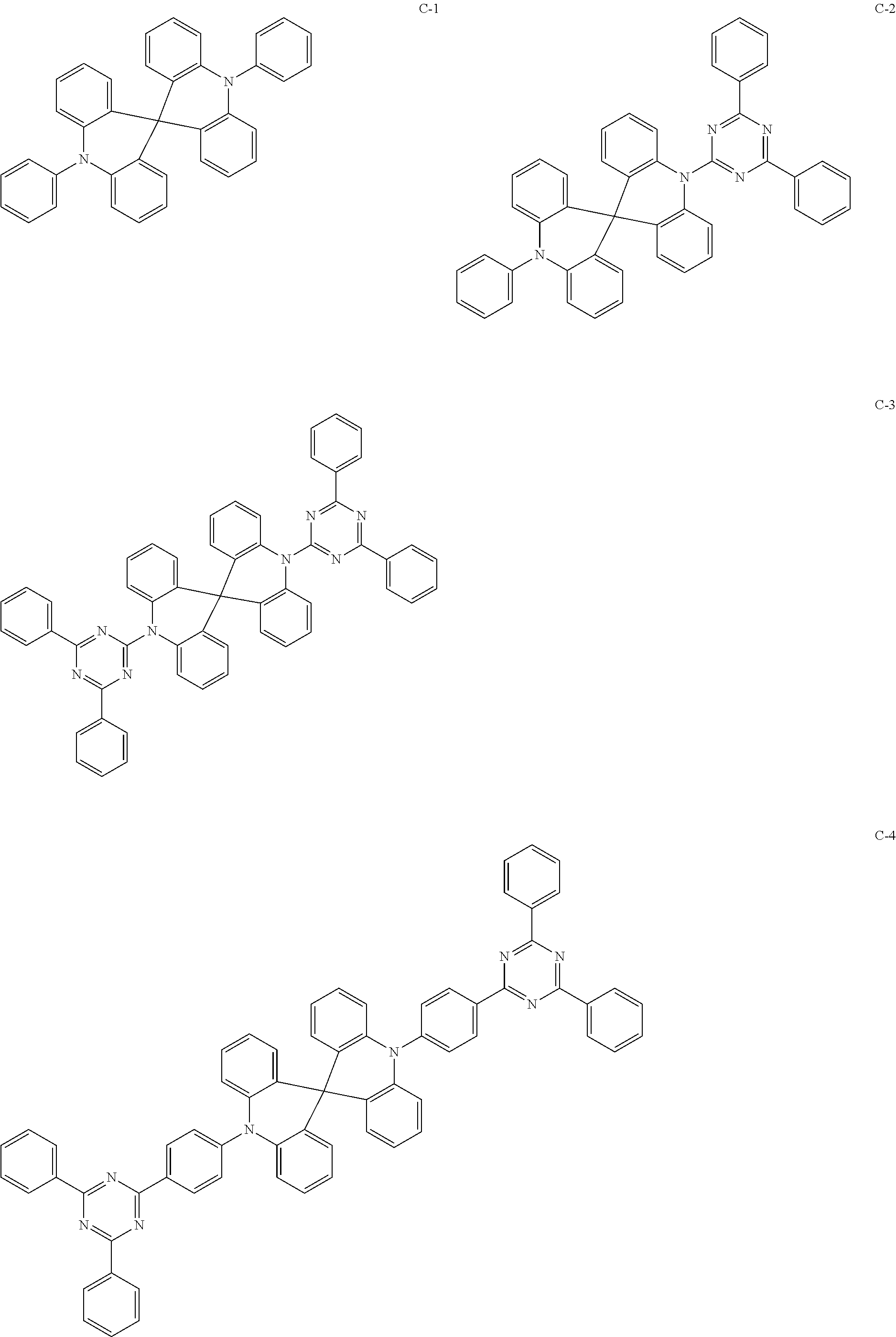 [SMILES: c1ccc(N2c3ccccc3C3(c4ccccc42)c2ccccc2N(c2ccccc2)c2ccccc23)cc1 ; c1ccc(-c2nc(-c3ccccc3)nc(N3c4ccccc4C4(c5ccccc5N(c5ccccc5)c5ccccc54)c4ccccc43)n2)cc1 ; c1ccc(-c2nc(-c3ccccc3)nc(N3c4ccccc4C4(c5ccccc53)c3ccccc3N(c3nc(-c5ccccc5)nc(-c5ccccc5)n3)c3ccccc34)n2)cc1 ; c1ccc(-c2nc(-c3ccccc3)nc(-c3ccc(N4c5ccccc5C5(c6ccccc64)c4ccccc4N(c4ccc(-c6nc(-c7ccccc7)nc(-c7ccccc7)n6)cc4)c4ccccc45)cc3)n2)cc1]

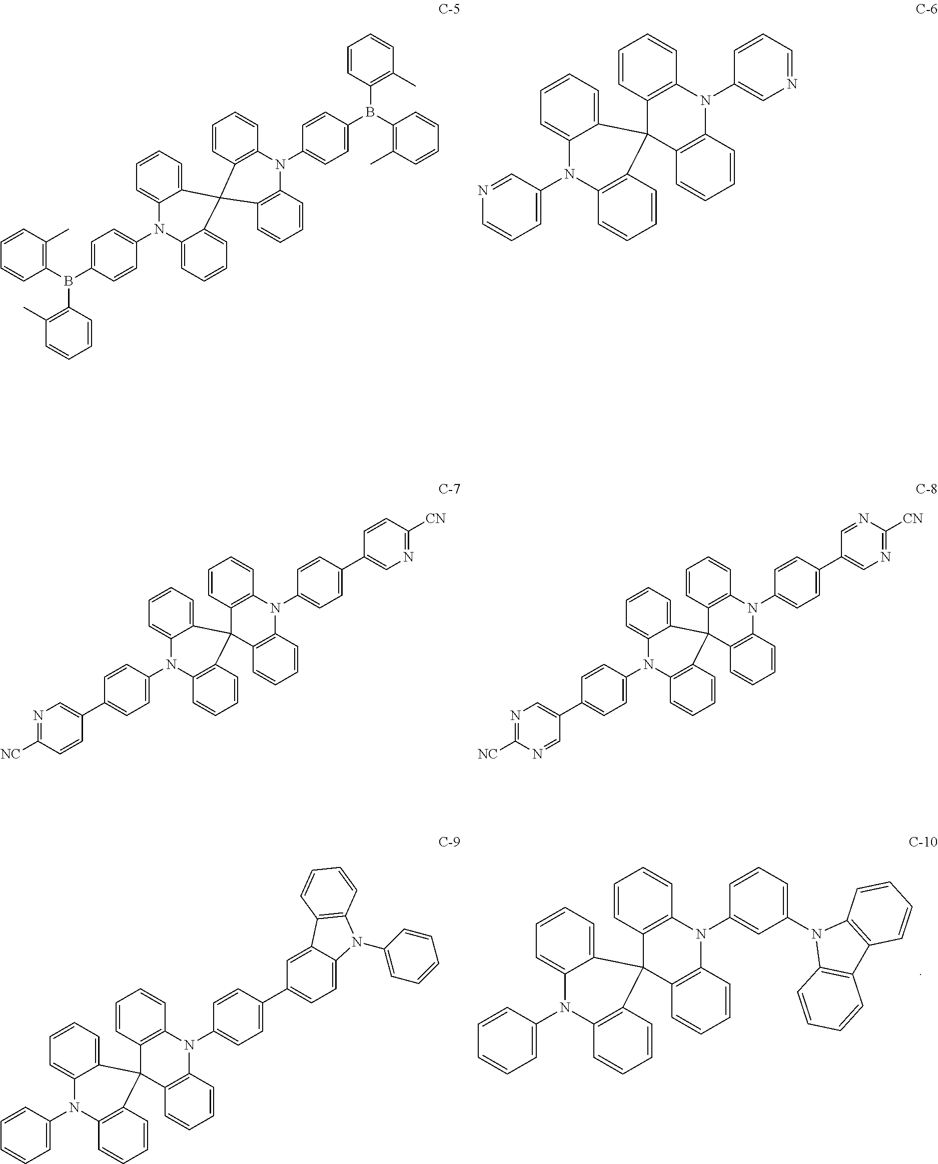

The S₁ (singlet) energy levels and T₁ (triplet) energy levels of the example compounds and the comparative compounds were calculated using a non-empirical molecular orbital method. For example, the calculations were conducted using B3LYP as a functional, and 6-31G(d) as a basis function in Gaussian 09 (Gaussian Co., Wallingford, Conn.). The results are shown in Table 1. ΔE_{ST} represents the difference between the singlet energy level and the triplet energy level.

TABLE 1

| | S₁ energy level | T₁ energy level | |
|---|---|---|---|
| | (eV) | (eV) | ΔE_{ST} |
| Example Compound 8 | 2.56 | 2.55 | 0.01 |
| Example Compound 40 | 2.75 | 2.72 | 0.03 |
| Example Compound 69 | 2.72 | 2.71 | 0.01 |
| Example Compound 127 | 2.52 | 2.52 | 0.00 |
| Example Compound 131 | 2.86 | 2.85 | 0.01 |
| Comparative Compound | 3.67 | 3.15 | 0.52 |
| C-1 | | | |
| Comparative Compound | 2.96 | 2.96 | 0.00 |
| C-2 | | | |
| Comparative Compound | 3.48 | 3.15 | 0.33 |
| C-3 | | | |
| Comparative Compound | 2.41 | 2.41 | 0.00 |
| C-4 | | | |
| Comparative Compound | 2.46 | 2.46 | 0.00 |
| C-5 | | | |
| Comparative Compound | 3.30 | 3.13 | 0.17 |
| C-6 | | | |
| Comparative Compound | 2.54 | 2.53 | 0.01 |
| C-7 | | | |
| Comparative Compound | 2.36 | 2.35 | 0.01 |
| C-8 | | | |
| Comparative Compound | 3.38 | 3.06 | 0.32 |
| C-9 | | | |
| Comparative Compound | 3.49 | 3.14 | 0.35 |
| C-10 | | | |

Referring to Table 1, Example Compounds 8, 40, 69, 127 and 131, and Comparative Compounds C-2 and C-4 to C-8 each showed low ΔE_{ST} values, and are thought to exhibit thermally activated delayed fluorescence. Comparative Compounds C-1, C-3, C-9 and C-10 showed high ΔE_{ST} values, and are not thought to exhibit thermally activated delayed fluorescence. Each organic electroluminescence device of Examples 1 to 5 and Comparative Examples 1 to 10 was manufactured by forming a first electrode using ITO to a thickness of about 150 nm, a hole injection layer using HAT-CN to a thickness of about 10 nm, a hole transport layer using NPB to a thickness of about 80 nm, an electron blocking layer using mCP to a thickness of about 5 nm, an emission layer using DPEPO doped with 20% of the Example Compound or the Comparative Compound to a thickness of about 20 nm, a hole blocking layer using DPEPO to a thickness of about 10 nm, an electron transport layer using TPBi to a thickness of about 30 nm, an electron injection layer using LiF to a thickness of about 0.5 nm, and a second electrode using Al to a thickness of about 100 nm. Each layer was formed using a vacuum deposition method.

The maximum emission wavelength and external quantum efficiency of each of the organic electroluminescence devices according to Examples 1 to 5 and Comparative Examples 1 to 10 were measured and are shown in Table 2. The EQE values in Table 2 were measured at about 10 mA/cm².

TABLE 2

| | Emission layer dopant | | EQE |
|---|---|---|---|
| | material | λₘₐₓ (nm) | (%) |
| Example 1 | Example Compound 8 | 468 | 23.8 |
| Example 2 | Example Compound 40 | 461 | 19.9 |
| Example 3 | Example Compound 69 | 459 | 22.5 |
| Example 4 | Example Compound 127 | 469 | 21.8 |
| Example 5 | Example Compound 131 | 459 | 20.5 |
| Comparative Example 1 | Comparative Compound | 413 | 1.2 |
| | C-1 | | |
| Comparative Example 2 | Comparative Compound | 489 | 3.2 |
| | C-2 | | |
| Comparative Example 3 | Comparative Compound | 496 | 2.8 |
| | C-3 | | |
| Comparative Example 4 | Comparative Compound | 501 | 17.5 |
| | C-4 | | |
| Comparative Example 5 | Comparative Compound | 498 | 15.1 |
| | C-5 | | |
| Comparative Example 6 | Comparative Compound | 420 | 2.1 |
| | C-6 | | |
| Comparative Example 7 | Comparative Compound | 470 | 5.5 |
| | C-7 | | |
| Comparative Example 8 | Comparative Compound | 512 | 8.2 |
| | C-8 | | |
| Comparative Example 9 | Comparative Compound | 415 | 1.1 |
| | C-9 | | |
| Comparative Example | Comparative Compound | 412 | 1.3 |
| 10 | C-10 | | |

Referring to the results of Table 2, Example Compounds 8, 40, 69, 127 and 131 all had light emission wavelengths of less than about 470 nm, and are suitable as deep blue dopants, thereby accomplishing high efficiency due to thermally activated delayed fluorescence. Comparative Examples 1, 6, 9 and 10 showed relatively low emission efficiencies and short emission wavelengths. Comparative Examples 4, 5 and 8 showed relatively high efficiencies but longer wavelengths, and deep blue light emission was not accomplished. Comparative Example 7 showed a wavelength near a deep blue color but with lower efficiency than the examples. Comparative Example 2 showed neither a deep blue color nor high efficiency. Though ΔE_{ST} of the calculated value of Comparative Example 2 was small, there was a chance that thermally activated delayed fluorescence was not shown. Comparative Example 3 showed a low efficiency and longer wavelength, and did not show a deep blue color.

The polycyclic compound according to one or more embodiments of the present disclosure may be utilized as a dopant of thermally activated delayed fluorescence for emitting deep blue color.

The organic electroluminescence device including the polycyclic compound according to one or more embodiments of the present disclosure accomplishes deep blue color and has excellent efficiency at the same time.

The organic electroluminescence device according to one or more embodiments of the present disclosure has excellent efficiency.

The polycyclic compound according to one or more embodiments of the present disclosure may be applied to an organic electroluminescence device and may contribute to high efficiency.

As used herein, expressions such as “at least one of”, “one of”, and “selected from”, when preceding a list of elements, modify the entire list of elements and do not modify the individual elements of the list. Further, the use of “may” when describing embodiments of the present disclosure refers to “one or more embodiments of the present disclosure”.

In addition, as used herein, the terms “use”, “using”, and “used” may be considered synonymous with the terms “utilize”, “utilizing”, and “utilized”, respectively.

As used herein, the terms “substantially”, “about”, and similar terms are used as terms of approximation and not as terms of degree, and are intended to account for the inherent deviations in measured or calculated values that would be recognized by those of ordinary skill in the art.

Also, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

Although example embodiments of the present disclosure have been described, it is understood that the present disclosure should not be limited to these example embodiments, but that various changes and modifications can be made by one ordinary skilled in the art within the spirit and scope of the present disclosure, as described in the following claims and equivalents thereof.

What is claimed is:

1. An organic electroluminescence device, comprising:
a first electrode;
a hole transport region on the first electrode;
an emission layer on the hole transport region;
an electron transport region on the emission layer; and
a second electrode on the electron transport region,
wherein the emission layer comprises a polycyclic compound represented by Formula 1: 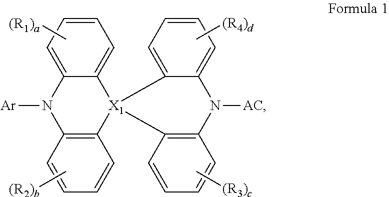
wherein, in Formula 1,
X₁ is C, Si, or Ge,
Ar is a substituted or unsubstituted aryl group having 6 to 30 carbon atoms for forming a ring, or a substituted or unsubstituted heteroaryl group having 2 to 30 carbon atoms for forming a ring,
R₁ to R₄ are each independently a hydrogen atom, a deuterium atom, a substituted or unsubstituted alkyl group having 1 to 10 carbon atoms, a substituted or unsubstituted aryl group having 6 to 30 carbon atoms for forming a ring, or a substituted or unsubstituted heteroaryl group having 2 to 30 carbon atoms for forming a ring,
“a” to “d” are each independently an integer of 0 to 4, and
AC is represented by one of Formulae 2 to 9: 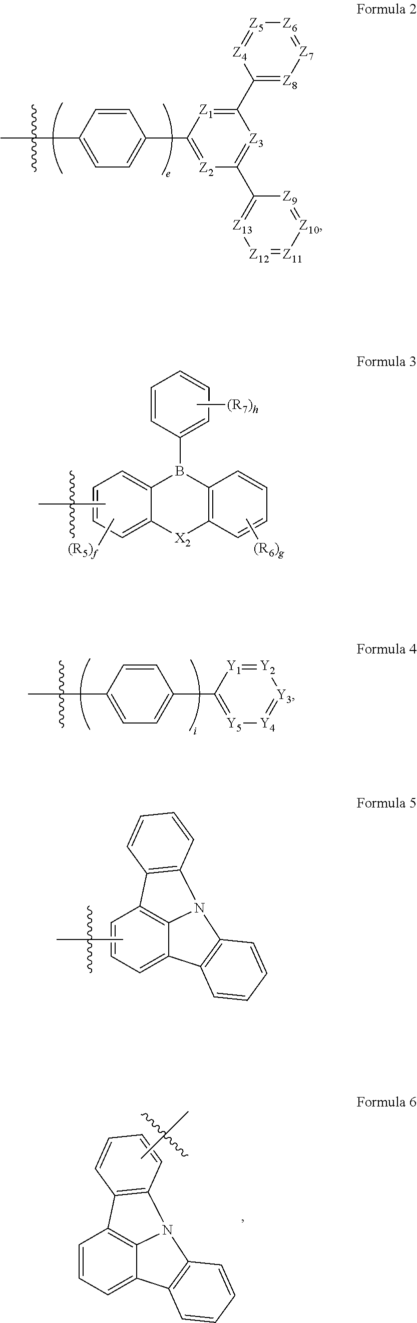 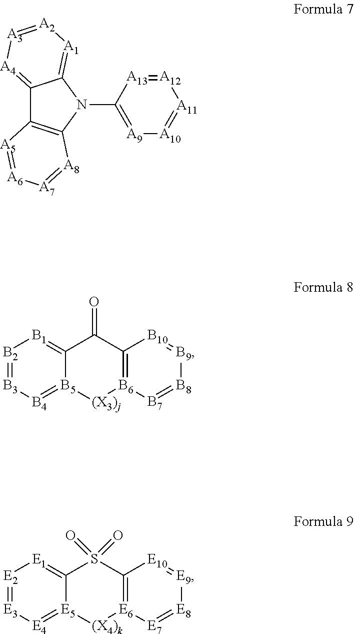
wherein, in Formula 2,
Z₁ to Z₁₃ are each independently CH or N, where two or three among Z₁ to Z₃ are each N, and at least one of Z₄ to Z₁₃ is N, and
“e” is an integer of 0 to 2,
wherein, in Formula 3,
X₂ is O, S or CR₈R₉,
R₅ to R₉ are each independently a hydrogen atom, a deuterium atom, or a substituted or unsubstituted alkyl group having 1 to 5 carbon atoms, or are combined with an adjacent group to form a hydrocarbon ring or a heterocycle,
“f” is an integer of 0 to 3,
“g” is an integer of 0 to 4, and
“h” is an integer of 0 to 5,
wherein, in Formula 4,
“i” is an integer of 0 to 2,
Y₁ is N or CR₁₀, Y₂ is N or CR₁₁, Y₃ is N or CR₁₂, Y₄ is N or CR₁₃, and Y₅ is N or CR₁₄, where at least one of Y₁ to Y₅ is N,
CR₁₀ to CR₁₄ are each independently a hydrogen atom, a deuterium atom, a cyano group, a substituted or unsubstituted alkyl group having 1 to 10 carbon atoms, or a trifluoromethyl group,
when X₁ of Formula 1 is C, at least one of R₁₀ to R₁₄ is not a hydrogen atom, and
when at least one of Y₂ and Y₄ is N, Y₃ is CR₁₂, and R₁₂ is not a cyano group,
wherein, in Formula 7,
A₁ to A₁₃ are each independently N or CR₁₅,
at least one of A₁ to A₈ is N, at least one of A₉ to A₁₃ is N, and at least one of A₁ to A₁₃ is a part that is connected with Formula 1, and
R₁₅ a hydrogen atom, a deuterium atom, a substituted or unsubstituted alkyl group having 1 to 10 carbon atoms, a substituted or unsubstituted aryl group having 6 to 30 carbon atoms for forming a ring, or a substituted or unsubstituted heteroaryl group having 2 to 30 carbon atoms for forming a ring,
wherein, in Formula 8,
B₁ to B₁₀ are each independently N or CR₁₆, and one of B₁ to B₁₀₀ is a part that is connected with Formula 1,
X₃ is a direct linkage, O, S, CR₁₇R₁₈, or SiR₁₉R₂₀,
“j” is 0 or 1, where if “j” is 1, B₅ and B₆ are C, and
R₁₆ to R₂₀ are each independently a hydrogen atom, a deuterium atom, a substituted or unsubstituted alkyl group having 1 to 10 carbon atoms, a substituted or unsubstituted aryl group having 6 to 30 carbon atoms for forming a ring, or a substituted or unsubstituted heteroaryl group having 2 to 30 carbon atoms for forming a ring, and
wherein, in Formula 9,
E₁ to E₁₀ are each independently N or CR₂₁, and one of E₁ to E₁₀ is a part that is connected with Formula 1,
X₄ is a direct linkage, O, S, CR₂₂R₂₃, or SiR₂₄R₂₅,
“k” is 0 or 1, where if “k” is 1, E₅ and E₆ are C, and
R₂₂ to R₂₅ are each independently a hydrogen atom, a deuterium atom, a substituted or unsubstituted alkyl group having 1 to 10 carbon atoms, a substituted or unsubstituted aryl group having 6 to 30 carbon atoms for forming a ring, or a substituted or unsubstituted heteroaryl group having 2 to 30 carbon atoms for forming a ring.

2. The organic electroluminescence device of claim 1, wherein Formula 3 is further represented by Formula 3-1 or 3-2: 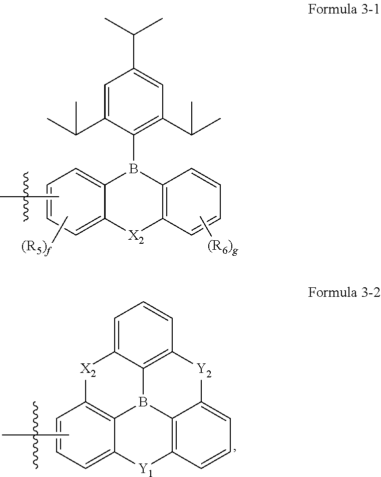
wherein, in Formulae 3-1 and 3-2,
X₂, R₅, R₆, “f” and “g” are each independently the same as defined in Formula 3,
Y₁ and Y₂ are each independently O, S, or CR₂₆R₂₇, and
R₂₆ and R₂₇ are each independently a hydrogen atom, a deuterium atom, or a substituted or unsubstituted alkyl group having 1 to 5 carbon atoms.

3. The organic electroluminescence device of claim 1, wherein Formula 4 is further represented by one of the following formulae:

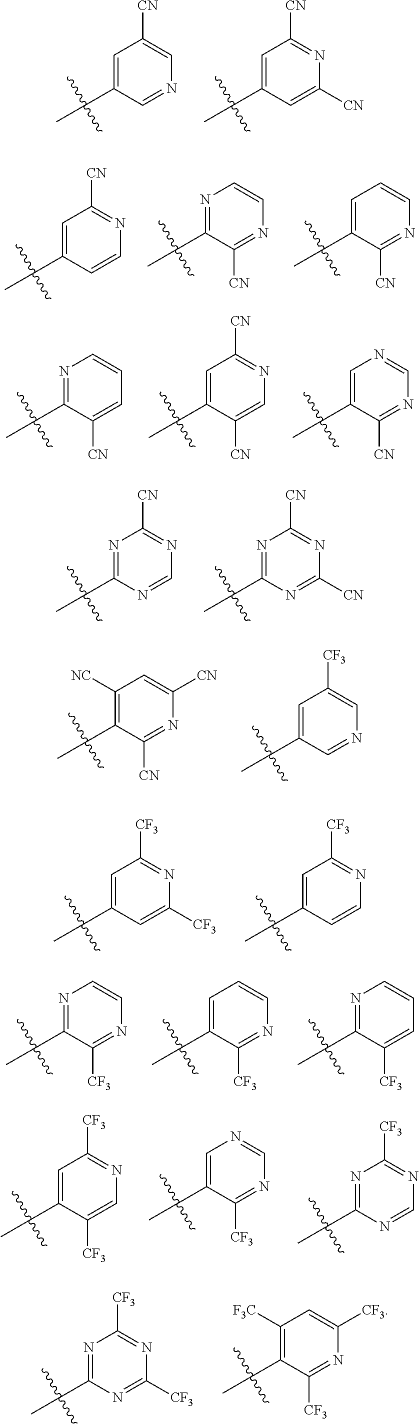

4. The organic electroluminescence device of claim 1, wherein Formula 7 is further represented by one of the following formulae:

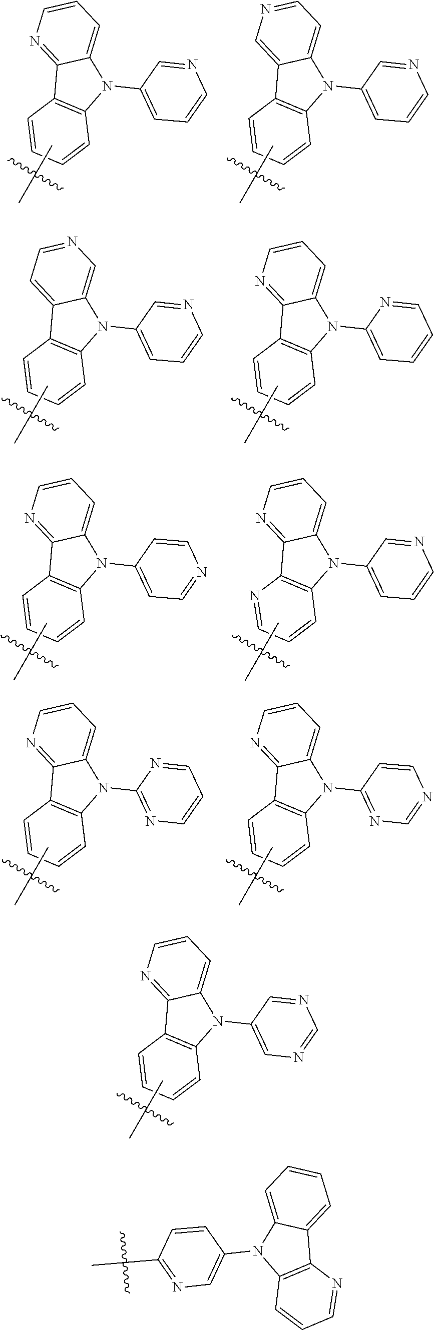

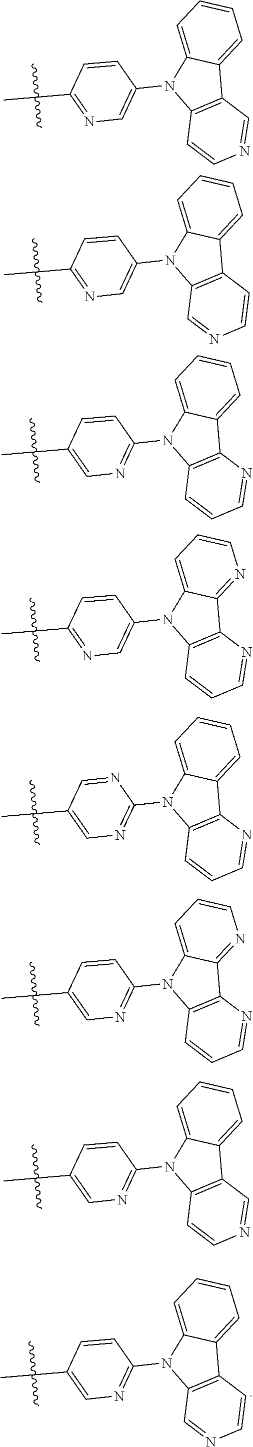

5. The organic electroluminescence device of claim 1, wherein Formula 8 is further represented by one of the following formulae:

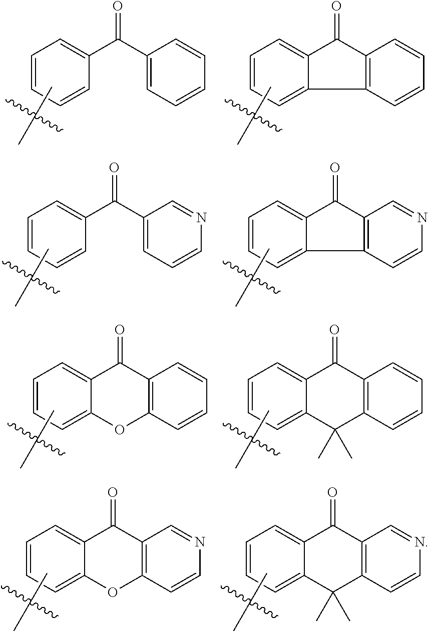

6. The organic electroluminescence device of claim 1, wherein Formula 9 is further represented by one of the following formulae:

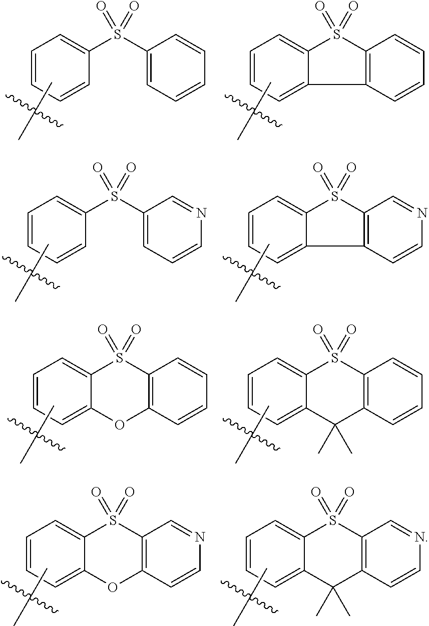

7. The organic electroluminescence device of claim 1, wherein Ar is a substituted or unsubstituted phenyl group.

8. The organic electroluminescence device of claim 1, wherein the emission layer comprises a host and a dopant, and
the dopant comprises the polycyclic compound.

9. The organic electroluminescence device of claim 8, wherein the polycyclic compound is a dopant for thermally activated delayed fluorescence.

10. The organic electroluminescence device of claim 8, wherein the polycyclic compound is a blue dopant having a wavelength of less than about 470 nm.

11. The organic electroluminescence device of claim 1, wherein the hole transport region comprises:
a hole injection layer on the first electrode;
a hole transport layer on the hole injection layer; and
an electron blocking layer on the hole transport layer.

12. The organic electroluminescence device of claim 1, wherein the electron transport region comprises:
a hole blocking layer on the emission layer;
an electron transport layer on the hole blocking layer; and
an electron injection layer on the electron transport layer.

13. The organic electroluminescence device of claim 1, wherein the polycyclic compound represented by Formula 1 is at least one selected from compounds represented in Compound Group 1:

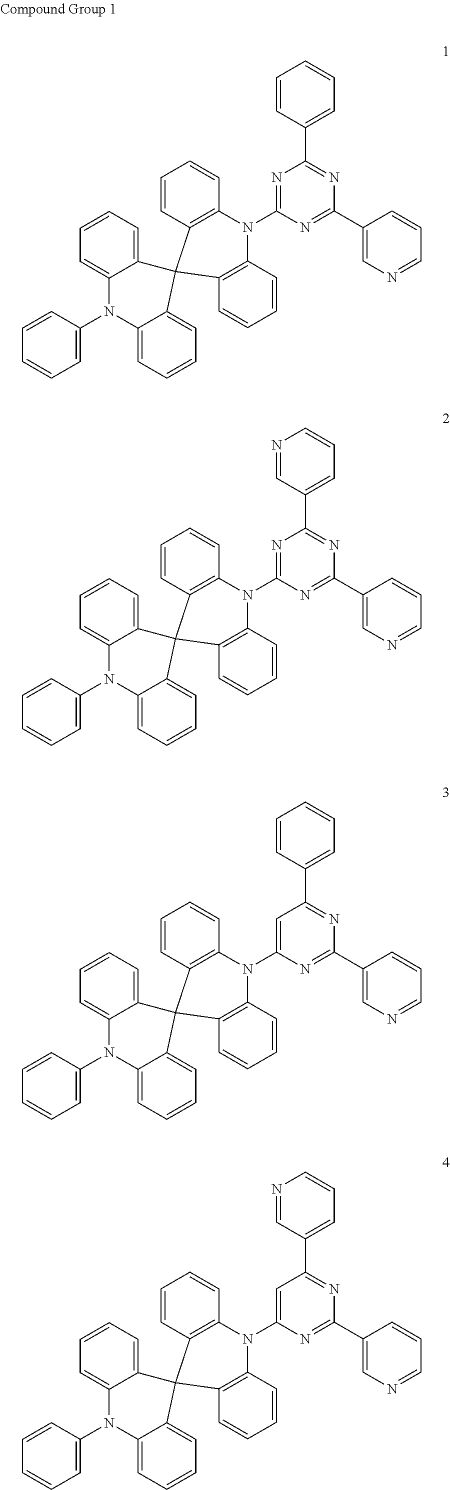 [SMILES: c1ccc(-c2nc(-c3cccnc3)nc(N3c4ccccc4C4(c5ccccc5N(c5ccccc5)c5ccccc54)c4ccccc43)n2)cc1 ; c1ccc(N2c3ccccc3C3(c4ccccc42)c2ccccc2N(c2nc(-c4cccnc4)nc(-c4cccnc4)n2)c2ccccc23)cc1 ; c1ccc(-c2cc(N3c4ccccc4C4(c5ccccc5N(c5ccccc5)c5ccccc54)c4ccccc43)nc(-c3cccnc3)n2)cc1 ; c1ccc(N2c3ccccc3C3(c4ccccc42)c2ccccc2N(c2cc(-c4cccnc4)nc(-c4cccnc4)n2)c2ccccc23)cc1]

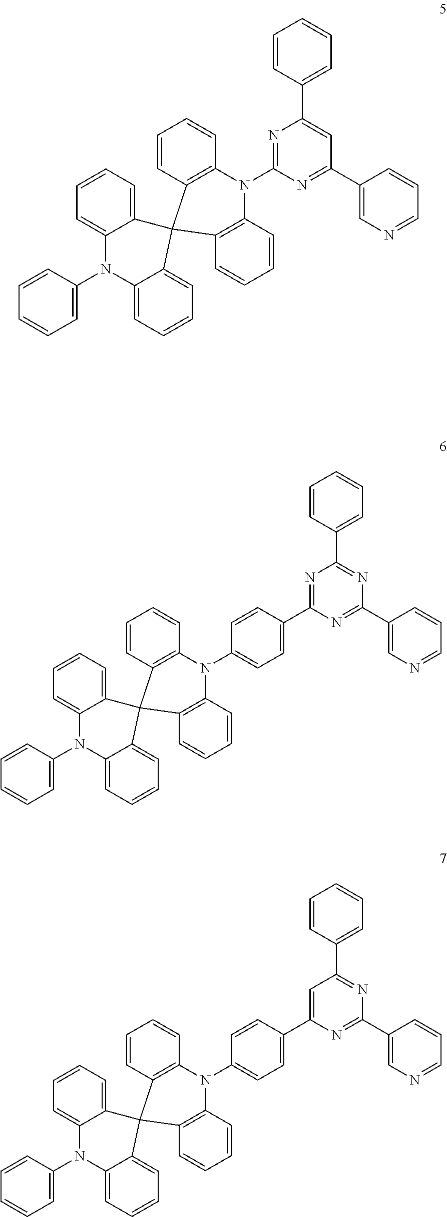 [SMILES: c1ccc(-c2cc(-c3cccnc3)nc(N3c4ccccc4C4(c5ccccc5N(c5ccccc5)c5ccccc54)c4ccccc43)n2)cc1 ; c1ccc(-c2nc(-c3ccc(N4c5ccccc5C5(c6ccccc6N(c6ccccc6)c6ccccc65)c5ccccc54)cc3)nc(-c3cccnc3)n2)cc1 ; c1ccc(-c2cc(-c3ccc(N4c5ccccc5C5(c6ccccc6N(c6ccccc6)c6ccccc65)c5ccccc54)cc3)nc(-c3cccnc3)n2)cc1]

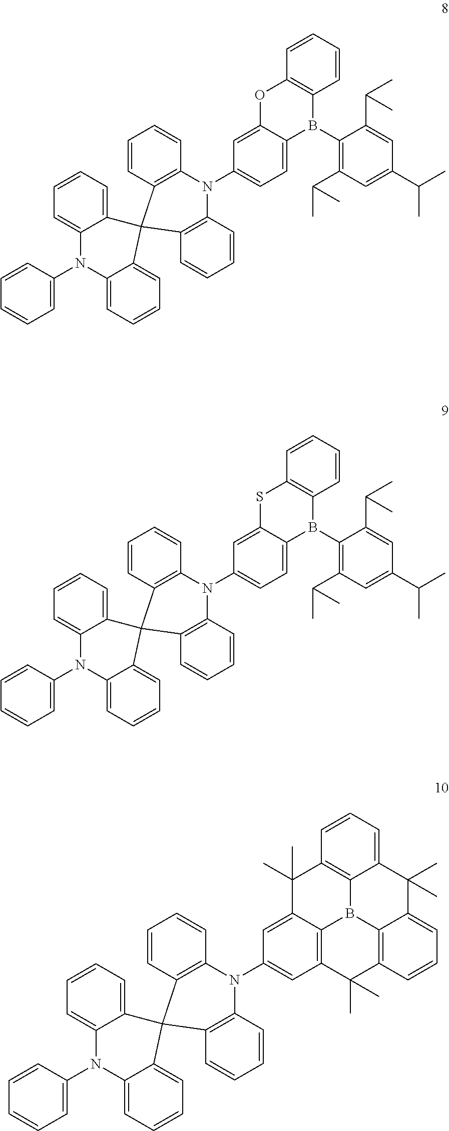 [SMILES: CC(C)c1cc(C(C)C)c(B2c3ccccc3Oc3cc(N4c5ccccc5C5(c6ccccc6N(c6ccccc6)c6ccccc65)c5ccccc54)ccc32)c(C(C)C)c1 ; CC(C)c1cc(C(C)C)c(B2c3ccccc3Sc3cc(N4c5ccccc5C5(c6ccccc6N(c6ccccc6)c6ccccc65)c5ccccc54)ccc32)c(C(C)C)c1 ; CC1(C)c2cccc3c2B2c4c1cccc4C(C)(C)c1cc(N4c5ccccc5C5(c6ccccc6N(c6ccccc6)c6ccccc65)c5ccccc54)cc(c12)C3(C)C]

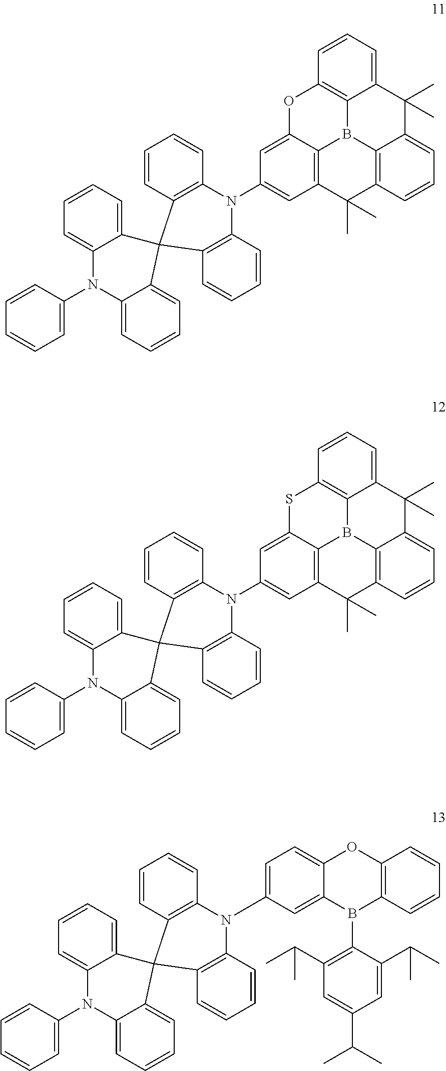 [SMILES: CC1(C)c2cccc3c2B2c4c(cc(N5c6ccccc6C6(c7ccccc7N(c7ccccc7)c7ccccc76)c6ccccc65)cc4C(C)(C)c4cccc1c42)O3 ; CC1(C)c2cccc3c2B2c4c(cc(N5c6ccccc6C6(c7ccccc7N(c7ccccc7)c7ccccc76)c6ccccc65)cc4C(C)(C)c4cccc1c42)S3 ; CC(C)c1cc(C(C)C)c(B2c3ccccc3Oc3ccc(N4c5ccccc5C5(c6ccccc6N(c6ccccc6)c6ccccc65)c5ccccc54)cc32)c(C(C)C)c1]

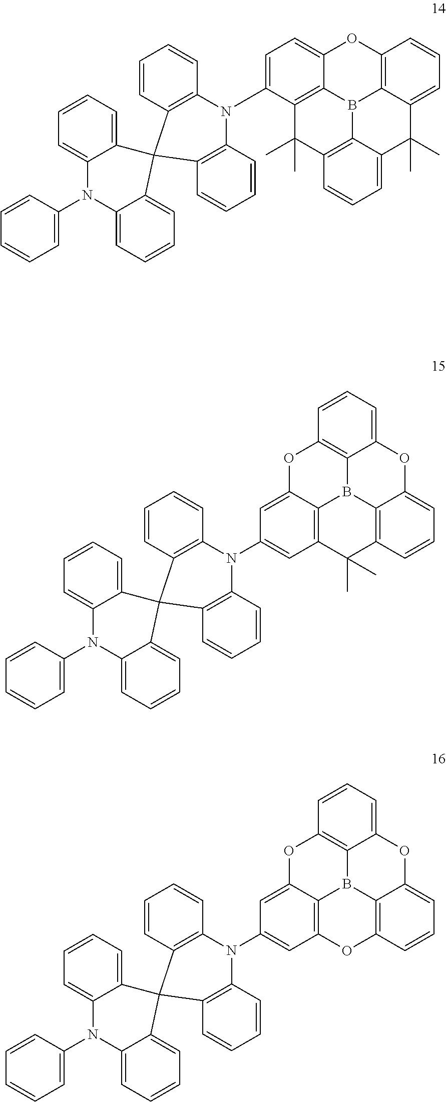 [SMILES: CC1(C)c2cccc3c2B2c4c1cccc4C(C)(C)c1c(N4c5ccccc5C5(c6ccccc6N(c6ccccc6)c6ccccc65)c5ccccc54)ccc(c12)O3 ; CC1(C)c2cccc3c2B2c4c(cccc4Oc4cc(N5c6ccccc6C6(c7ccccc7N(c7ccccc7)c7ccccc76)c6ccccc65)cc1c42)O3 ; c1ccc(N2c3ccccc3C3(c4ccccc42)c2ccccc2N(c2cc4c5c(c2)Oc2cccc6c2B5c2c(cccc2O4)O6)c2ccccc23)cc1]

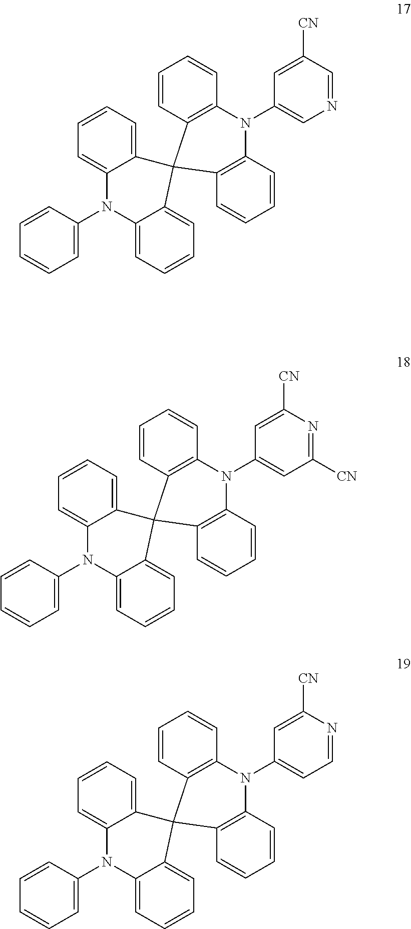 [SMILES: N#Cc1cncc(N2c3ccccc3C3(c4ccccc4N(c4ccccc4)c4ccccc43)c3ccccc32)c1 ; N#Cc1cc(N2c3ccccc3C3(c4ccccc4N(c4ccccc4)c4ccccc43)c3ccccc32)cc(C#N)n1 ; N#Cc1cc(N2c3ccccc3C3(c4ccccc4N(c4ccccc4)c4ccccc43)c3ccccc32)ccn1]

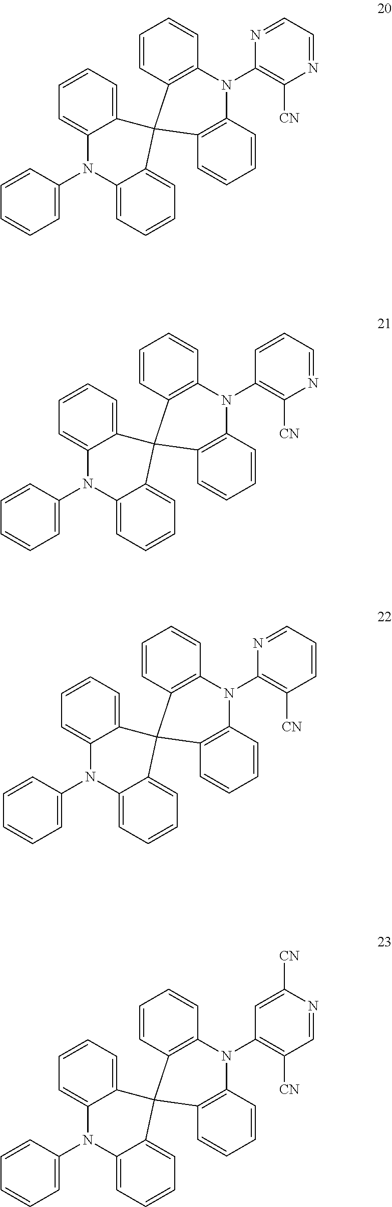 [SMILES: N#Cc1nccnc1N1c2ccccc2C2(c3ccccc3N(c3ccccc3)c3ccccc32)c2ccccc21 ; N#Cc1ncccc1N1c2ccccc2C2(c3ccccc3N(c3ccccc3)c3ccccc32)c2ccccc21 ; N#Cc1cccnc1N1c2ccccc2C2(c3ccccc3N(c3ccccc3)c3ccccc32)c2ccccc21 ; N#Cc1cc(N2c3ccccc3C3(c4ccccc4N(c4ccccc4)c4ccccc43)c3ccccc32)c(C#N)cn1]

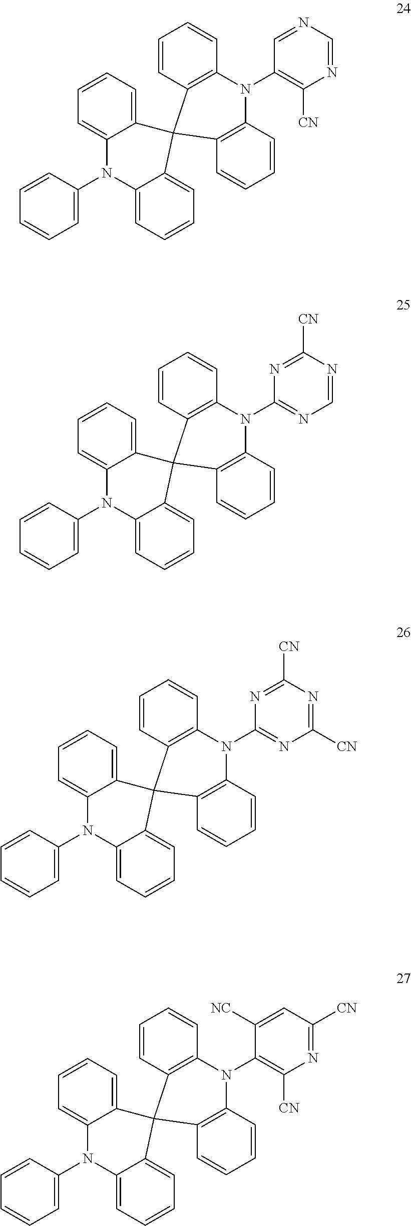 [SMILES: N#Cc1ncncc1N1c2ccccc2C2(c3ccccc3N(c3ccccc3)c3ccccc32)c2ccccc21 ; N#Cc1ncnc(N2c3ccccc3C3(c4ccccc4N(c4ccccc4)c4ccccc43)c3ccccc32)n1 ; N#Cc1nc(C#N)nc(N2c3ccccc3C3(c4ccccc4N(c4ccccc4)c4ccccc43)c3ccccc32)n1 ; N#Cc1cc(C#N)c(N2c3ccccc3C3(c4ccccc4N(c4ccccc4)c4ccccc43)c3ccccc32)c(C#N)n1]

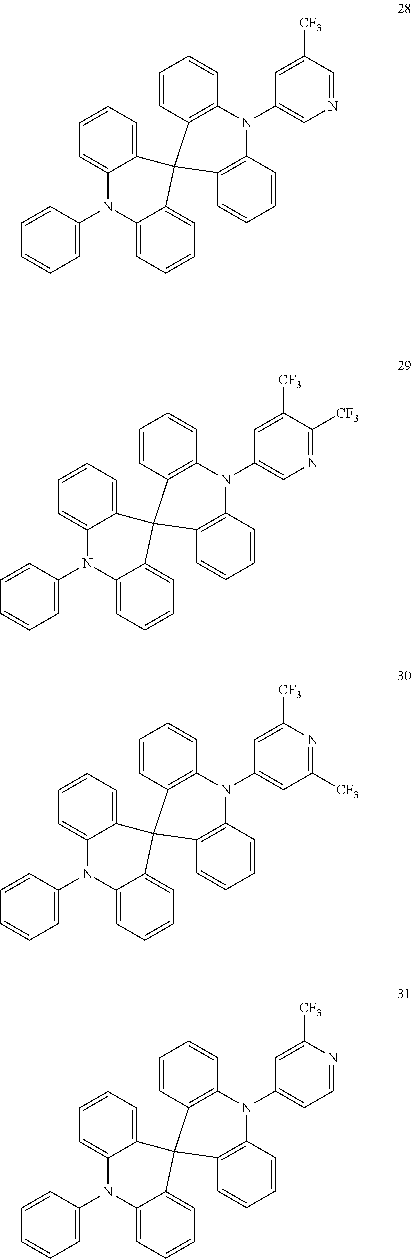 [SMILES: FC(F)(F)c1cncc(N2c3ccccc3C3(c4ccccc4N(c4ccccc4)c4ccccc43)c3ccccc32)c1 ; FC(F)(F)c1cc(N2c3ccccc3C3(c4ccccc4N(c4ccccc4)c4ccccc43)c3ccccc32)cnc1C(F)(F)F ; FC(F)(F)c1cc(N2c3ccccc3C3(c4ccccc4N(c4ccccc4)c4ccccc43)c3ccccc32)cc(C(F)(F)F)n1 ; FC(F)(F)c1cc(N2c3ccccc3C3(c4ccccc4N(c4ccccc4)c4ccccc43)c3ccccc32)ccn1]

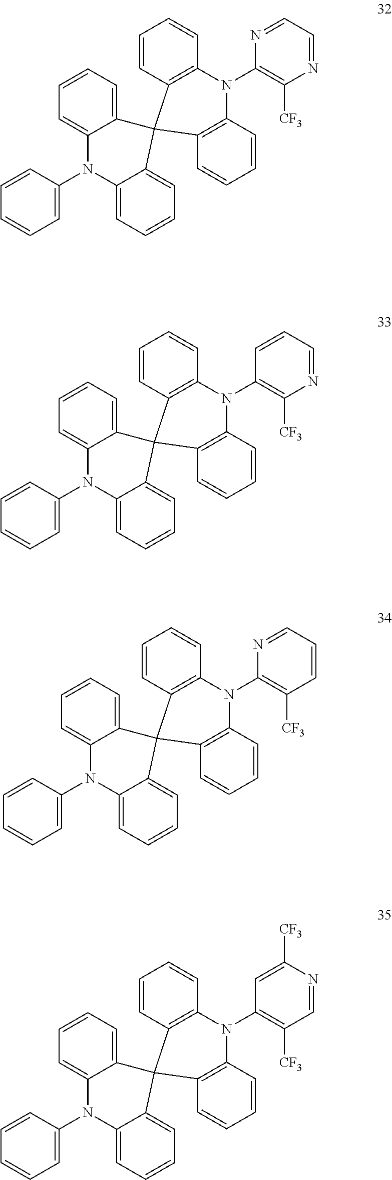 [SMILES: FC(F)(F)c1nccnc1N1c2ccccc2C2(c3ccccc3N(c3ccccc3)c3ccccc32)c2ccccc21 ; FC(F)(F)c1ncccc1N1c2ccccc2C2(c3ccccc3N(c3ccccc3)c3ccccc32)c2ccccc21 ; FC(F)(F)c1cccnc1N1c2ccccc2C2(c3ccccc3N(c3ccccc3)c3ccccc32)c2ccccc21 ; FC(F)(F)c1cc(N2c3ccccc3C3(c4ccccc4N(c4ccccc4)c4ccccc43)c3ccccc32)c(C(F)(F)F)cn1]

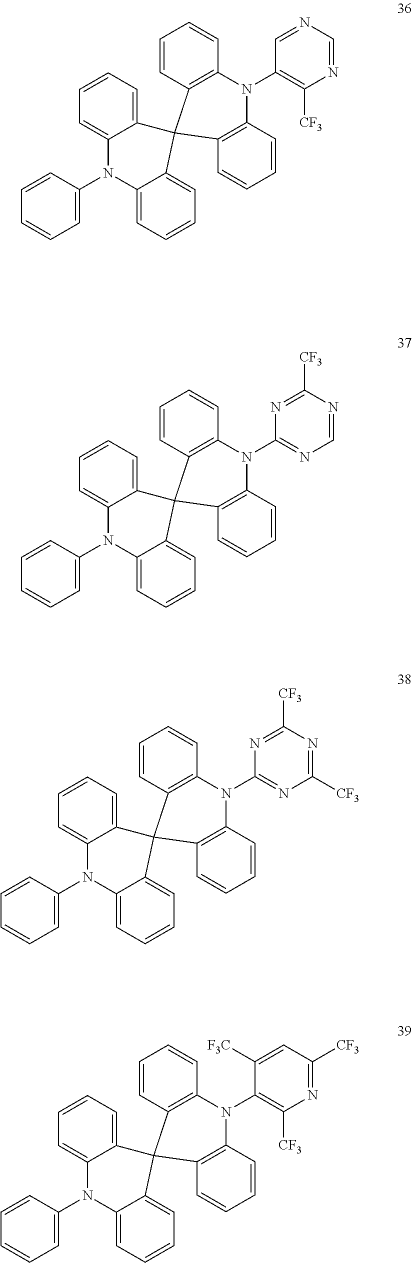 [SMILES: FC(F)(F)c1ncncc1N1c2ccccc2C2(c3ccccc3N(c3ccccc3)c3ccccc32)c2ccccc21 ; FC(F)(F)c1ncnc(N2c3ccccc3C3(c4ccccc4N(c4ccccc4)c4ccccc43)c3ccccc32)n1 ; FC(F)(F)c1nc(N2c3ccccc3C3(c4ccccc4N(c4ccccc4)c4ccccc43)c3ccccc32)nc(C(F)(F)F)n1 ; FC(F)(F)c1cc(C(F)(F)F)c(N2c3ccccc3C3(c4ccccc4N(c4ccccc4)c4ccccc43)c3ccccc32)c(C(F)(F)F)n1]

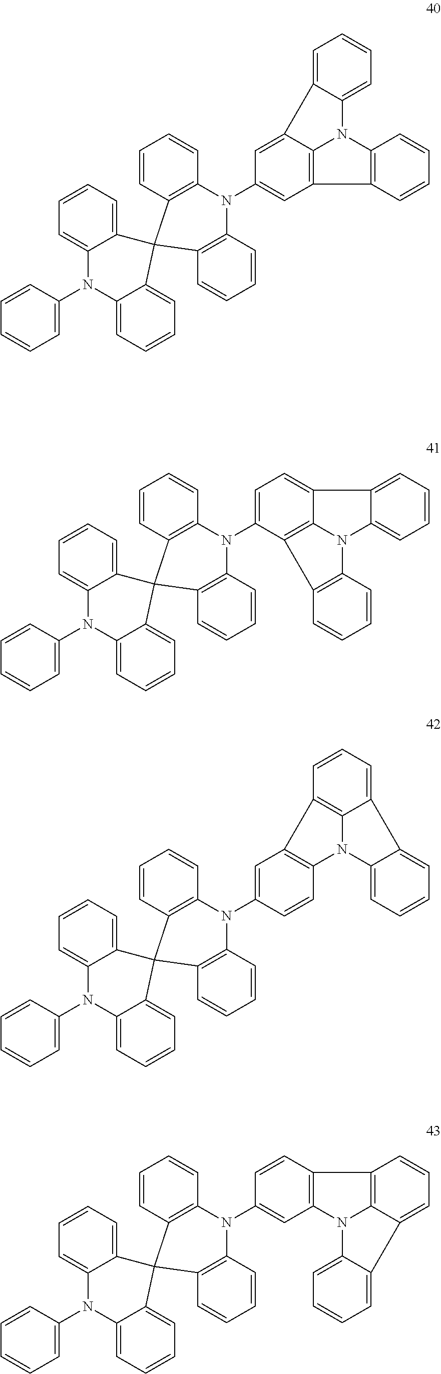 [SMILES: c1ccc(N2c3ccccc3C3(c4ccccc42)c2ccccc2N(c2cc4c5ccccc5n5c6ccccc6c(c2)c45)c2ccccc23)cc1 ; c1ccc(N2c3ccccc3C3(c4ccccc42)c2ccccc2N(c2ccc4c5ccccc5n5c6ccccc6c2c45)c2ccccc23)cc1 ; c1ccc(N2c3ccccc3C3(c4ccccc42)c2ccccc2N(c2ccc4c(c2)c2cccc5c6ccccc6n4c52)c2ccccc23)cc1 ; c1ccc(N2c3ccccc3C3(c4ccccc42)c2ccccc2N(c2ccc4c5cccc6c7ccccc7n(c4c2)c65)c2ccccc23)cc1]

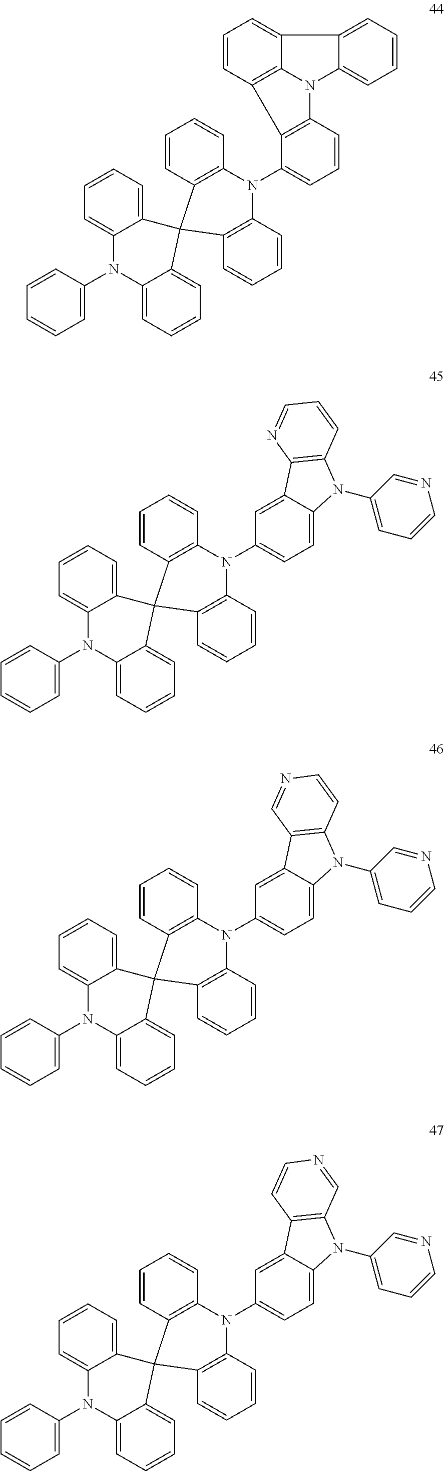 [SMILES: c1ccc(N2c3ccccc3C3(c4ccccc42)c2ccccc2N(c2cccc4c2c2cccc5c6ccccc6n4c52)c2ccccc23)cc1 ; c1ccc(N2c3ccccc3C3(c4ccccc42)c2ccccc2N(c2ccc4c(c2)c2ncccc2n4-c2cccnc2)c2ccccc23)cc1 ; c1ccc(N2c3ccccc3C3(c4ccccc42)c2ccccc2N(c2ccc4c(c2)c2cnccc2n4-c2cccnc2)c2ccccc23)cc1 ; c1ccc(N2c3ccccc3C3(c4ccccc42)c2ccccc2N(c2ccc4c(c2)c2ccncc2n4-c2cccnc2)c2ccccc23)cc1]

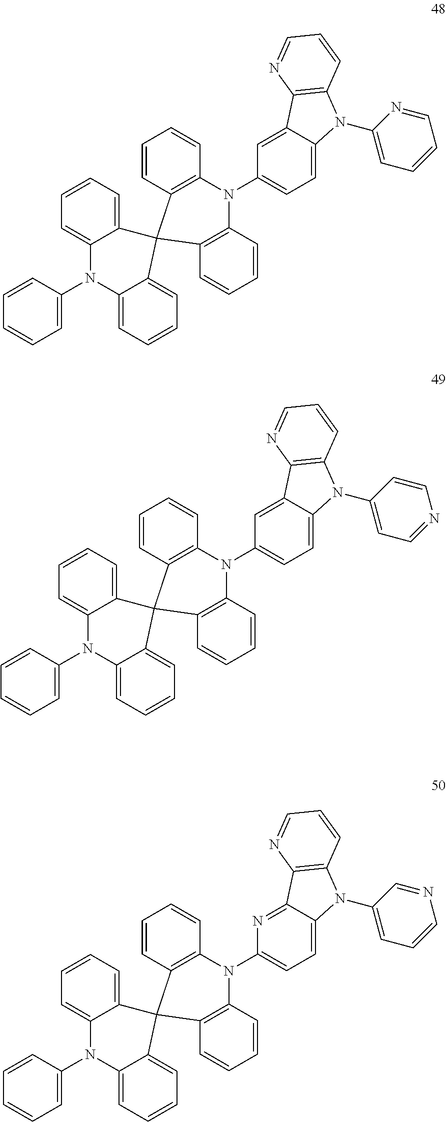 [SMILES: c1ccc(N2c3ccccc3C3(c4ccccc42)c2ccccc2N(c2ccc4c(c2)c2ncccc2n4-c2ccccn2)c2ccccc23)cc1 ; c1ccc(N2c3ccccc3C3(c4ccccc42)c2ccccc2N(c2ccc4c(c2)c2ncccc2n4-c2ccncc2)c2ccccc23)cc1 ; c1ccc(N2c3ccccc3C3(c4ccccc42)c2ccccc2N(c2ccc4c(n2)c2ncccc2n4-c2cccnc2)c2ccccc23)cc1]

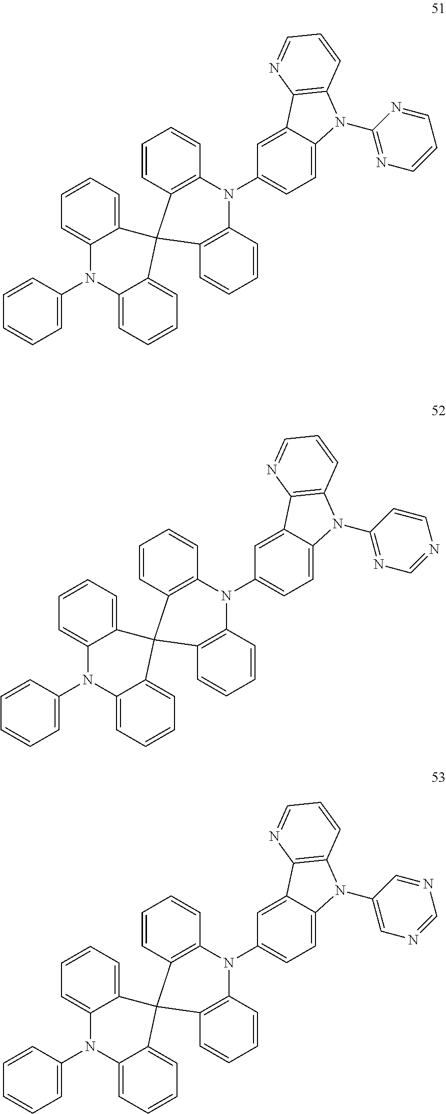 [SMILES: c1ccc(N2c3ccccc3C3(c4ccccc42)c2ccccc2N(c2ccc4c(c2)c2ncccc2n4-c2ncccn2)c2ccccc23)cc1 ; c1ccc(N2c3ccccc3C3(c4ccccc42)c2ccccc2N(c2ccc4c(c2)c2ncccc2n4-c2ccncn2)c2ccccc23)cc1 ; c1ccc(N2c3ccccc3C3(c4ccccc42)c2ccccc2N(c2ccc4c(c2)c2ncccc2n4-c2cncnc2)c2ccccc23)cc1]

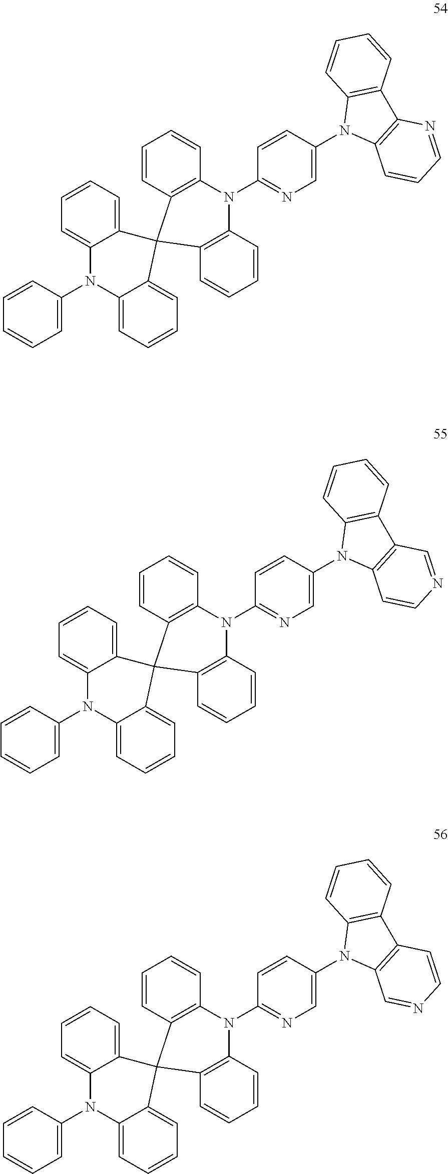 [SMILES: c1ccc(N2c3ccccc3C3(c4ccccc42)c2ccccc2N(c2ccc(-n4c5ccccc5c5ncccc54)cn2)c2ccccc23)cc1 ; c1ccc(N2c3ccccc3C3(c4ccccc42)c2ccccc2N(c2ccc(-n4c5ccccc5c5cnccc54)cn2)c2ccccc23)cc1 ; c1ccc(N2c3ccccc3C3(c4ccccc42)c2ccccc2N(c2ccc(-n4c5ccccc5c5ccncc54)cn2)c2ccccc23)cc1]

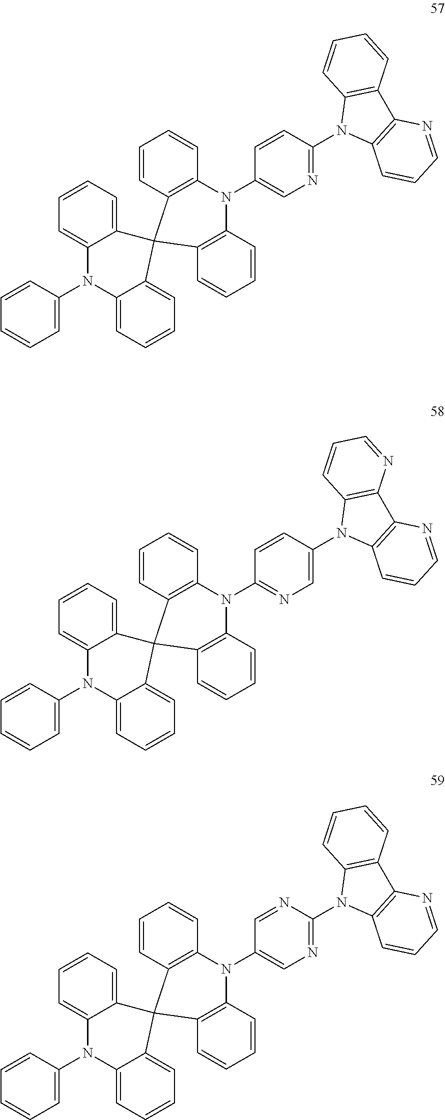 [SMILES: c1ccc(N2c3ccccc3C3(c4ccccc42)c2ccccc2N(c2ccc(-n4c5ccccc5c5ncccc54)nc2)c2ccccc23)cc1 ; c1ccc(N2c3ccccc3C3(c4ccccc42)c2ccccc2N(c2ccc(-n4c5cccnc5c5ncccc54)cn2)c2ccccc23)cc1 ; c1ccc(N2c3ccccc3C3(c4ccccc42)c2ccccc2N(c2cnc(-n4c5ccccc5c5ncccc54)nc2)c2ccccc23)cc1]

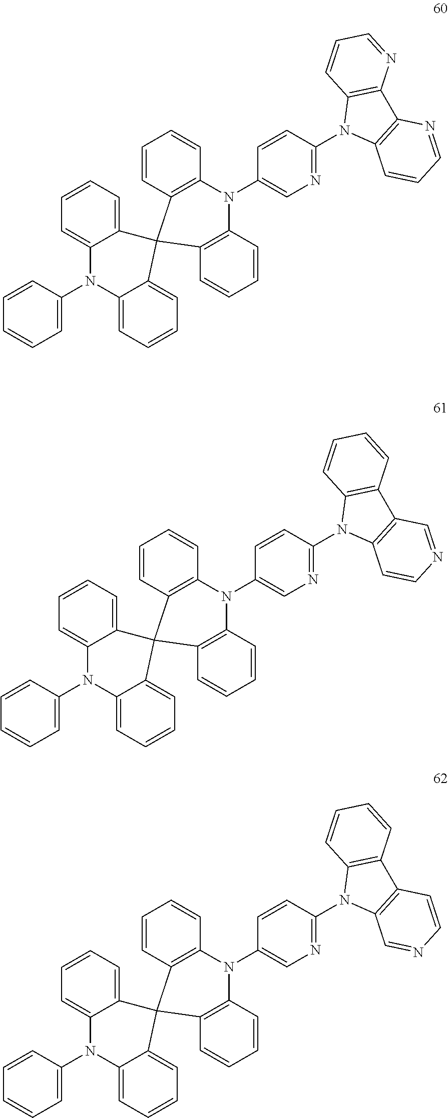 [SMILES: c1ccc(N2c3ccccc3C3(c4ccccc42)c2ccccc2N(c2ccc(-n4c5cccnc5c5ncccc54)nc2)c2ccccc23)cc1 ; c1ccc(N2c3ccccc3C3(c4ccccc42)c2ccccc2N(c2ccc(-n4c5ccccc5c5cnccc54)nc2)c2ccccc23)cc1 ; c1ccc(N2c3ccccc3C3(c4ccccc42)c2ccccc2N(c2ccc(-n4c5ccccc5c5ccncc54)nc2)c2ccccc23)cc1]

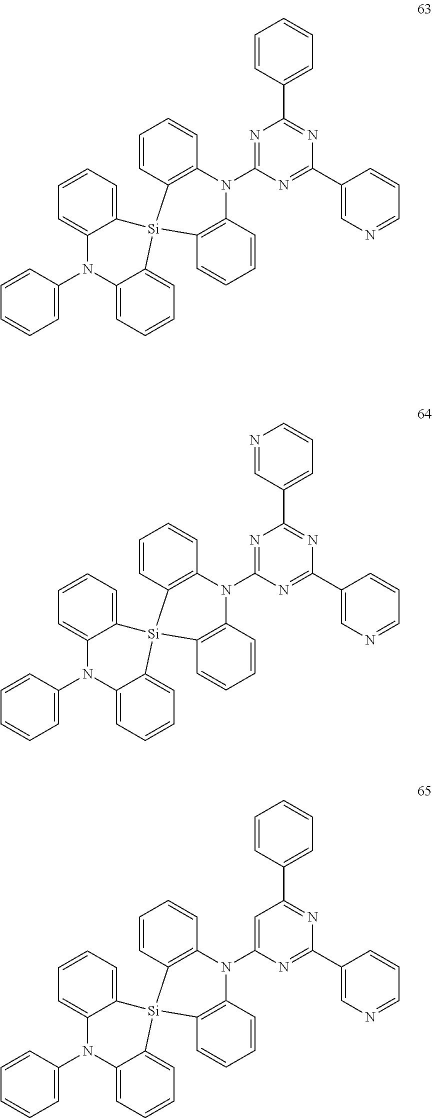 [SMILES: c1ccc(-c2nc(-c3cccnc3)nc(N3c4ccccc4[Si]4(c5ccccc5N(c5ccccc5)c5ccccc54)c4ccccc43)n2)cc1 ; c1ccc(N2c3ccccc3[Si]3(c4ccccc42)c2ccccc2N(c2nc(-c4cccnc4)nc(-c4cccnc4)n2)c2ccccc23)cc1 ; c1ccc(-c2cc(N3c4ccccc4[Si]4(c5ccccc5N(c5ccccc5)c5ccccc54)c4ccccc43)nc(-c3cccnc3)n2)cc1]

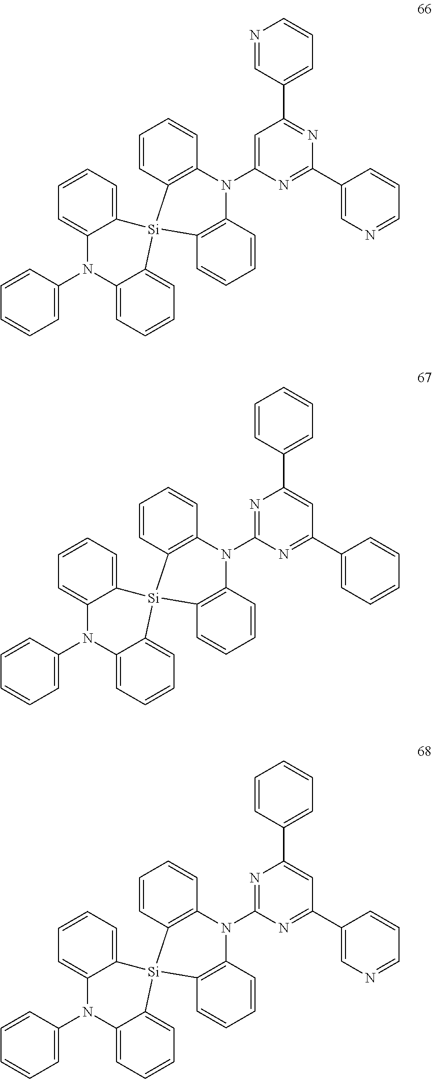 [SMILES: c1ccc(N2c3ccccc3[Si]3(c4ccccc42)c2ccccc2N(c2cc(-c4cccnc4)nc(-c4cccnc4)n2)c2ccccc23)cc1 ; c1ccc(-c2cc(-c3ccccc3)nc(N3c4ccccc4[Si]4(c5ccccc5N(c5ccccc5)c5ccccc54)c4ccccc43)n2)cc1 ; c1ccc(-c2cc(-c3cccnc3)nc(N3c4ccccc4[Si]4(c5ccccc5N(c5ccccc5)c5ccccc54)c4ccccc43)n2)cc1]

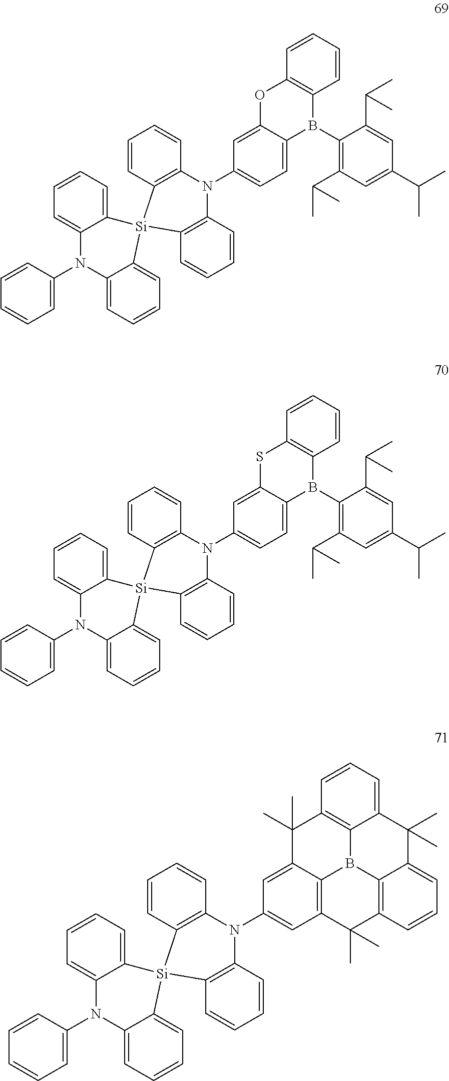 [SMILES: CC(C)c1cc(C(C)C)c(B2c3ccccc3Oc3cc(N4c5ccccc5[Si]5(c6ccccc6N(c6ccccc6)c6ccccc65)c5ccccc54)ccc32)c(C(C)C)c1 ; CC(C)c1cc(C(C)C)c(B2c3ccccc3Sc3cc(N4c5ccccc5[Si]5(c6ccccc6N(c6ccccc6)c6ccccc65)c5ccccc54)ccc32)c(C(C)C)c1 ; CC1(C)c2cccc3c2B2c4c1cccc4C(C)(C)c1cc(N4c5ccccc5[Si]5(c6ccccc6N(c6ccccc6)c6ccccc65)c5ccccc54)cc(c12)C3(C)C]

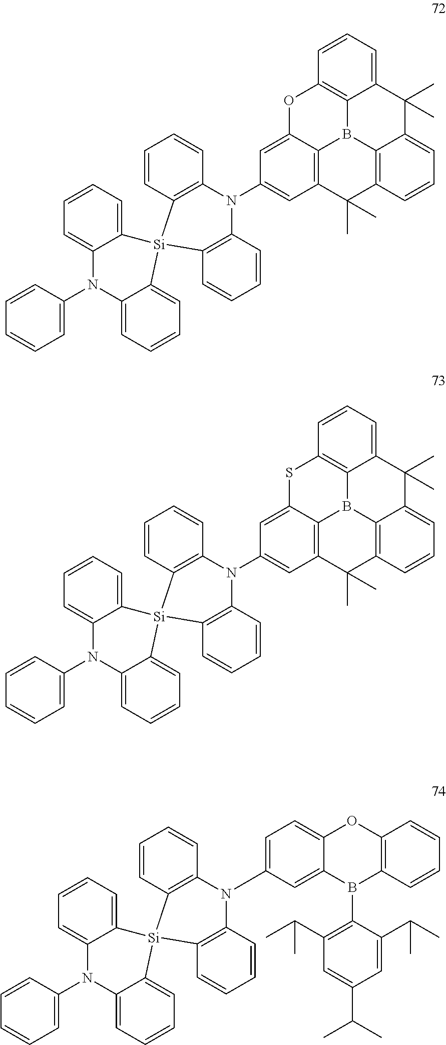 [SMILES: CC1(C)c2cccc3c2B2c4c(cc(N5c6ccccc6[Si]6(c7ccccc7N(c7ccccc7)c7ccccc76)c6ccccc65)cc4C(C)(C)c4cccc1c42)O3 ; CC1(C)c2cccc3c2B2c4c(cc(N5c6ccccc6[Si]6(c7ccccc7N(c7ccccc7)c7ccccc76)c6ccccc65)cc4C(C)(C)c4cccc1c42)S3 ; CC(C)c1cc(C(C)C)c(B2c3ccccc3Oc3ccc(N4c5ccccc5[Si]5(c6ccccc6N(c6ccccc6)c6ccccc65)c5ccccc54)cc32)c(C(C)C)c1]

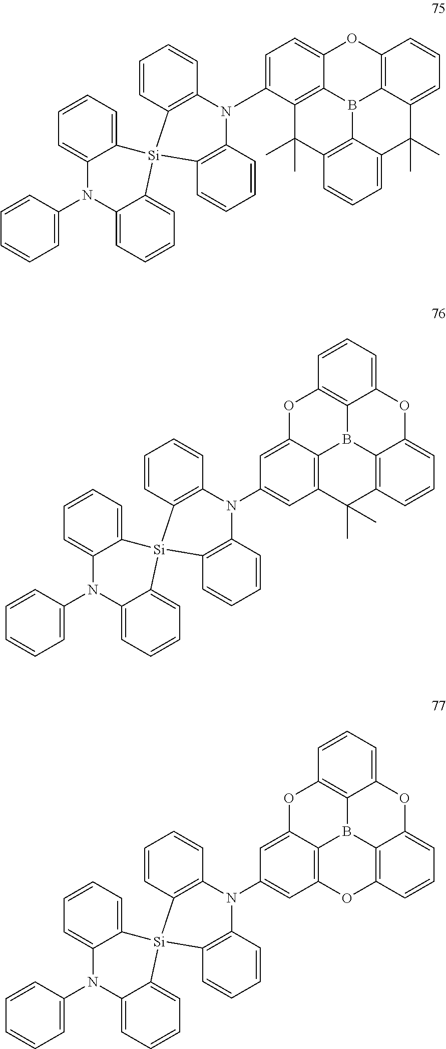 [SMILES: CC1(C)c2cccc3c2B2c4c1cccc4C(C)(C)c1c(N4c5ccccc5[Si]5(c6ccccc6N(c6ccccc6)c6ccccc65)c5ccccc54)ccc(c12)O3 ; CC1(C)c2cccc3c2B2c4c(cccc4Oc4cc(N5c6ccccc6[Si]6(c7ccccc7N(c7ccccc7)c7ccccc76)c6ccccc65)cc1c42)O3 ; c1ccc(N2c3ccccc3[Si]3(c4ccccc42)c2ccccc2N(c2cc4c5c(c2)Oc2cccc6c2B5c2c(cccc2O4)O6)c2ccccc23)cc1]

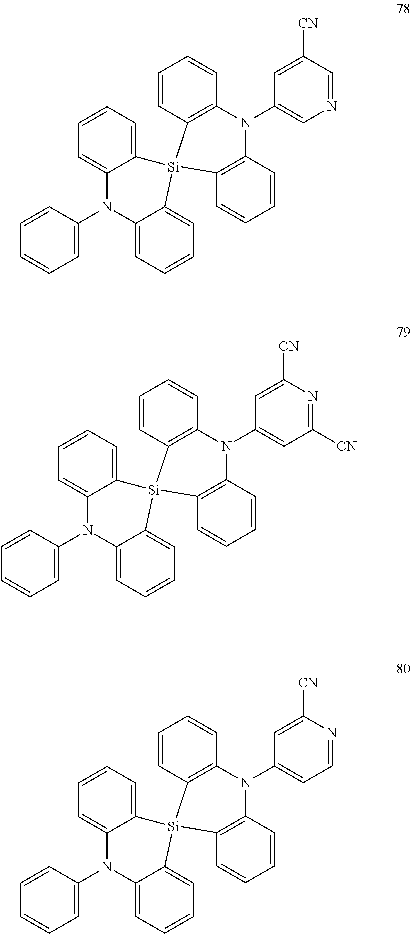 [SMILES: N#Cc1cncc(N2c3ccccc3[Si]3(c4ccccc4N(c4ccccc4)c4ccccc43)c3ccccc32)c1 ; N#Cc1cc(N2c3ccccc3[Si]3(c4ccccc4N(c4ccccc4)c4ccccc43)c3ccccc32)cc(C#N)n1 ; N#Cc1cc(N2c3ccccc3[Si]3(c4ccccc4N(c4ccccc4)c4ccccc43)c3ccccc32)ccn1]

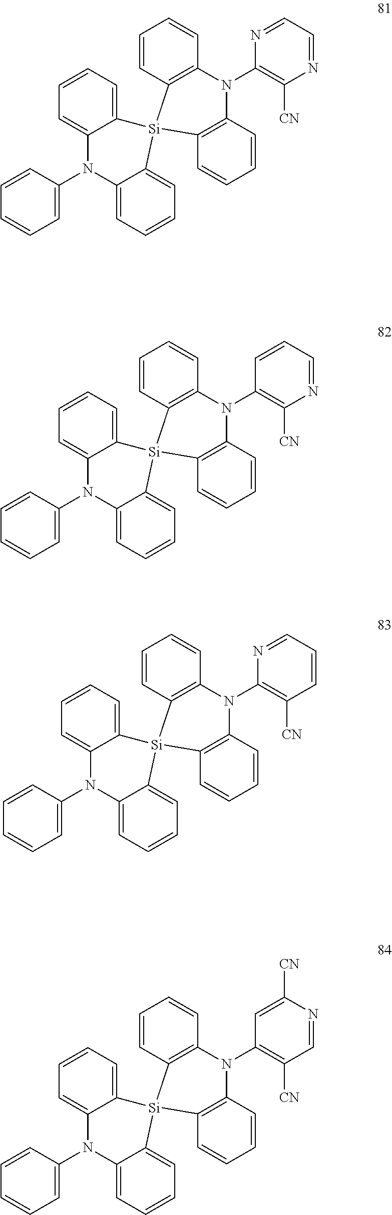 [SMILES: N#Cc1nccnc1N1c2ccccc2[Si]2(c3ccccc3N(c3ccccc3)c3ccccc32)c2ccccc21 ; N#Cc1ncccc1N1c2ccccc2[Si]2(c3ccccc3N(c3ccccc3)c3ccccc32)c2ccccc21 ; N#Cc1cccnc1N1c2ccccc2[Si]2(c3ccccc3N(c3ccccc3)c3ccccc32)c2ccccc21 ; N#Cc1cc(N2c3ccccc3[Si]3(c4ccccc4N(c4ccccc4)c4ccccc43)c3ccccc32)c(C#N)cn1]

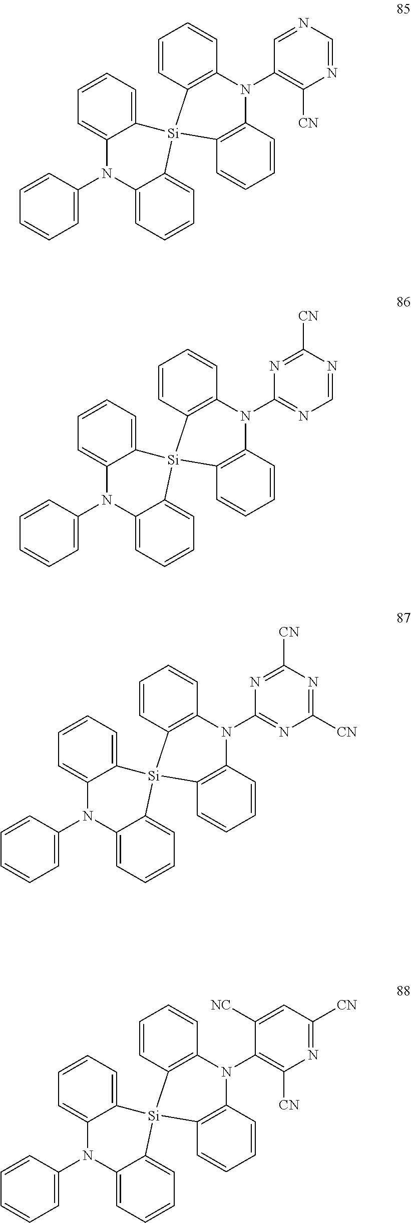 [SMILES: N#Cc1ncncc1N1c2ccccc2[Si]2(c3ccccc3N(c3ccccc3)c3ccccc32)c2ccccc21 ; N#Cc1ncnc(N2c3ccccc3[Si]3(c4ccccc4N(c4ccccc4)c4ccccc43)c3ccccc32)n1 ; N#Cc1nc(C#N)nc(N2c3ccccc3[Si]3(c4ccccc4N(c4ccccc4)c4ccccc43)c3ccccc32)n1 ; N#Cc1cc(C#N)c(N2c3ccccc3[Si]3(c4ccccc4N(c4ccccc4)c4ccccc43)c3ccccc32)c(C#N)n1]

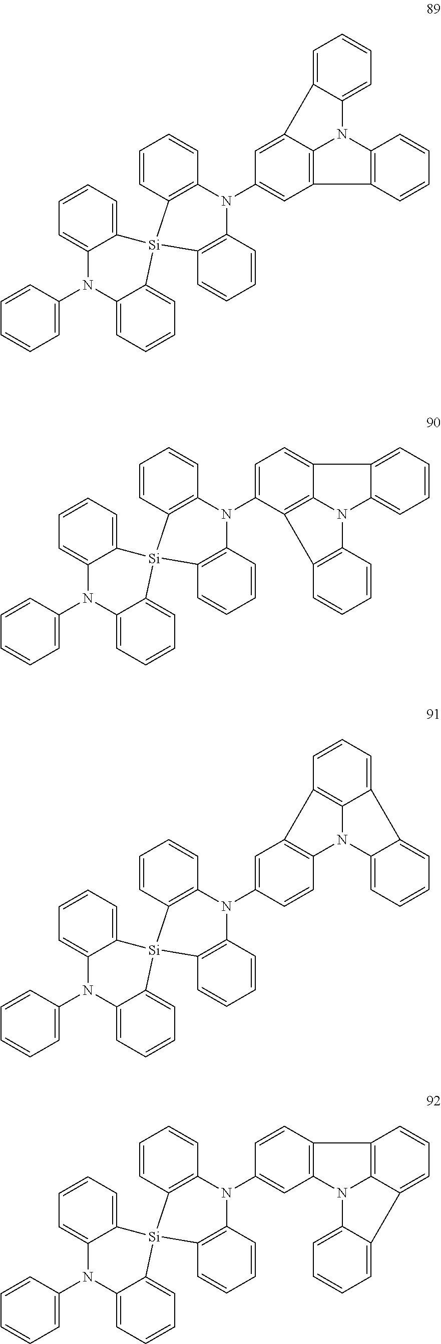 [SMILES: c1ccc(N2c3ccccc3[Si]3(c4ccccc42)c2ccccc2N(c2cc4c5ccccc5n5c6ccccc6c(c2)c45)c2ccccc23)cc1 ; c1ccc(N2c3ccccc3[Si]3(c4ccccc42)c2ccccc2N(c2ccc4c5ccccc5n5c6ccccc6c2c45)c2ccccc23)cc1 ; c1ccc(N2c3ccccc3[Si]3(c4ccccc42)c2ccccc2N(c2ccc4c(c2)c2cccc5c6ccccc6n4c52)c2ccccc23)cc1 ; c1ccc(N2c3ccccc3[Si]3(c4ccccc42)c2ccccc2N(c2ccc4c5cccc6c7ccccc7n(c4c2)c65)c2ccccc23)cc1]

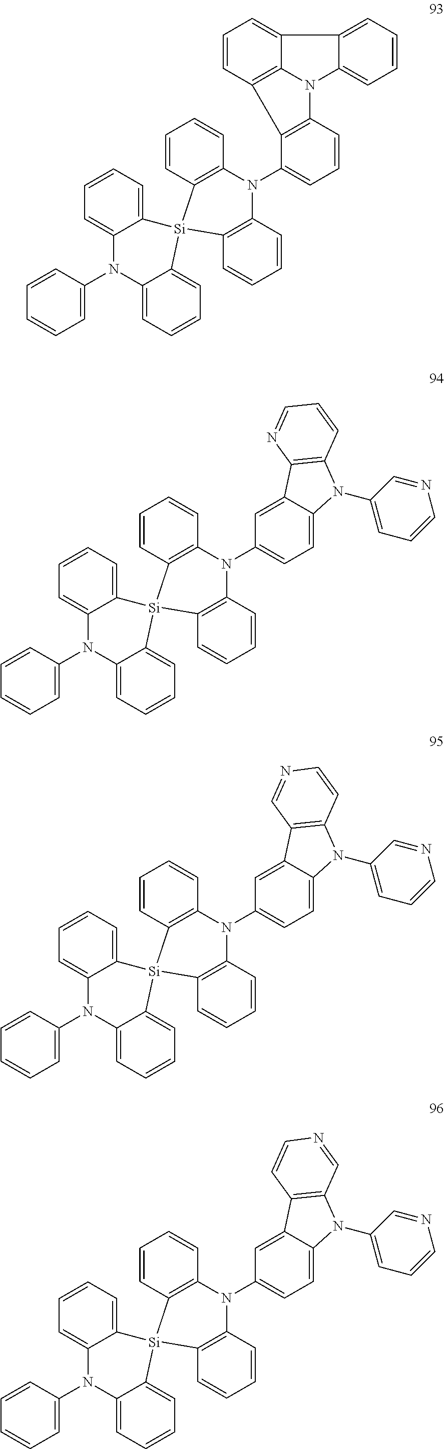 [SMILES: c1ccc(N2c3ccccc3[Si]3(c4ccccc42)c2ccccc2N(c2cccc4c2c2cccc5c6ccccc6n4c52)c2ccccc23)cc1 ; c1ccc(N2c3ccccc3[Si]3(c4ccccc42)c2ccccc2N(c2ccc4c(c2)c2ncccc2n4-c2cccnc2)c2ccccc23)cc1 ; c1ccc(N2c3ccccc3[Si]3(c4ccccc42)c2ccccc2N(c2ccc4c(c2)c2cnccc2n4-c2cccnc2)c2ccccc23)cc1 ; c1ccc(N2c3ccccc3[Si]3(c4ccccc42)c2ccccc2N(c2ccc4c(c2)c2ccncc2n4-c2cccnc2)c2ccccc23)cc1]

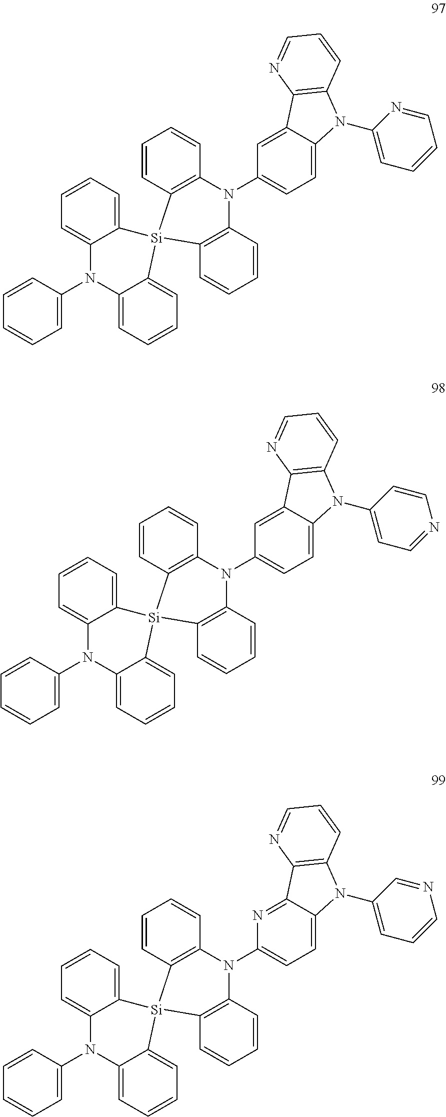 [SMILES: c1ccc(N2c3ccccc3[Si]3(c4ccccc42)c2ccccc2N(c2ccc4c(c2)c2ncccc2n4-c2ccccn2)c2ccccc23)cc1 ; c1ccc(N2c3ccccc3[Si]3(c4ccccc42)c2ccccc2N(c2ccc4c(c2)c2ncccc2n4-c2ccncc2)c2ccccc23)cc1 ; c1ccc(N2c3ccccc3[Si]3(c4ccccc42)c2ccccc2N(c2ccc4c(n2)c2ncccc2n4-c2cccnc2)c2ccccc23)cc1]

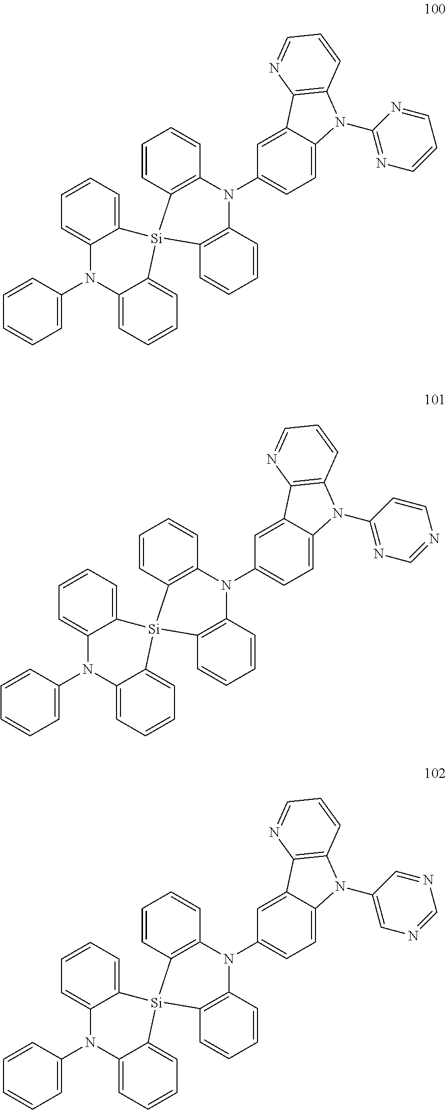 [SMILES: c1ccc(N2c3ccccc3[Si]3(c4ccccc42)c2ccccc2N(c2ccc4c(c2)c2ncccc2n4-c2ncccn2)c2ccccc23)cc1 ; c1ccc(N2c3ccccc3[Si]3(c4ccccc42)c2ccccc2N(c2ccc4c(c2)c2ncccc2n4-c2ccncn2)c2ccccc23)cc1 ; c1ccc(N2c3ccccc3[Si]3(c4ccccc42)c2ccccc2N(c2ccc4c(c2)c2ncccc2n4-c2cncnc2)c2ccccc23)cc1]

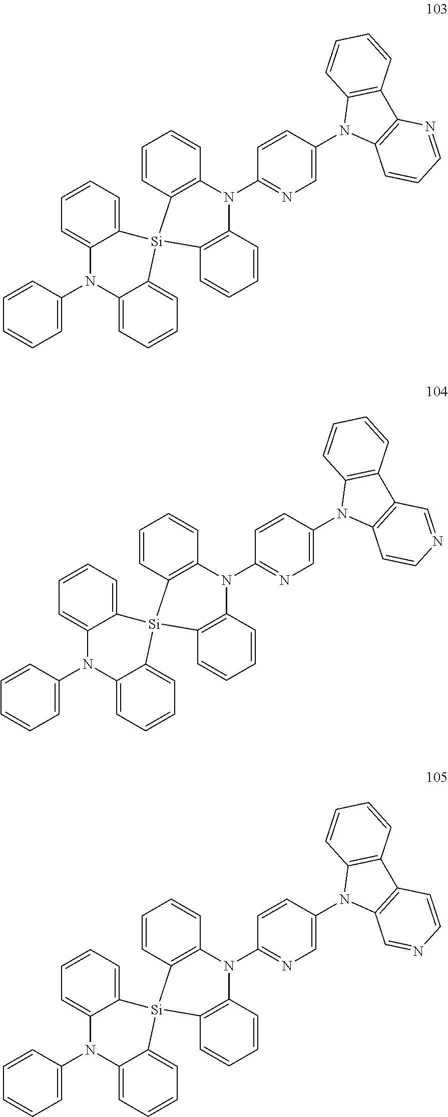 [SMILES: c1ccc(N2c3ccccc3[Si]3(c4ccccc42)c2ccccc2N(c2ccc(-n4c5ccccc5c5ncccc54)cn2)c2ccccc23)cc1 ; c1ccc(N2c3ccccc3[Si]3(c4ccccc42)c2ccccc2N(c2ccc(-n4c5ccccc5c5cnccc54)cn2)c2ccccc23)cc1 ; c1ccc(N2c3ccccc3[Si]3(c4ccccc42)c2ccccc2N(c2ccc(-n4c5ccccc5c5ccncc54)cn2)c2ccccc23)cc1]

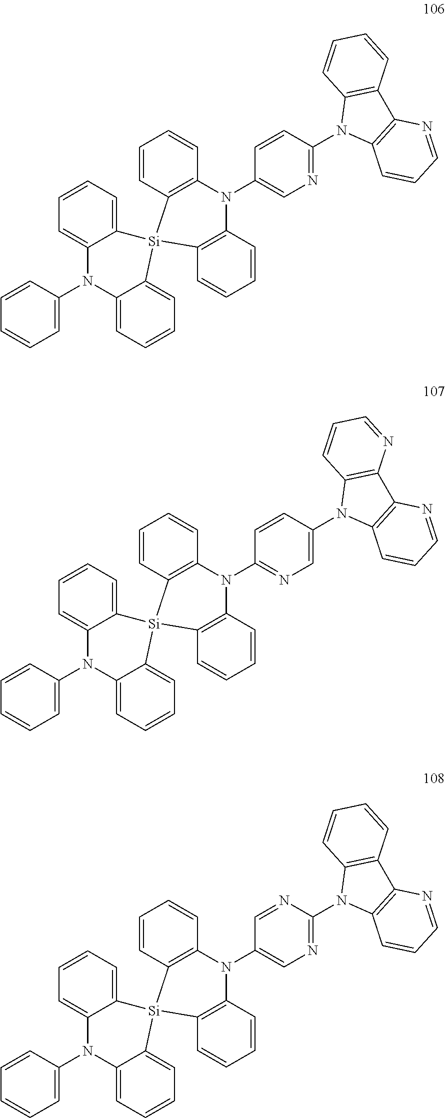 [SMILES: c1ccc(N2c3ccccc3[Si]3(c4ccccc42)c2ccccc2N(c2ccc(-n4c5ccccc5c5ncccc54)nc2)c2ccccc23)cc1 ; c1ccc(N2c3ccccc3[Si]3(c4ccccc42)c2ccccc2N(c2ccc(-n4c5cccnc5c5ncccc54)cn2)c2ccccc23)cc1 ; c1ccc(N2c3ccccc3[Si]3(c4ccccc42)c2ccccc2N(c2cnc(-n4c5ccccc5c5ncccc54)nc2)c2ccccc23)cc1]

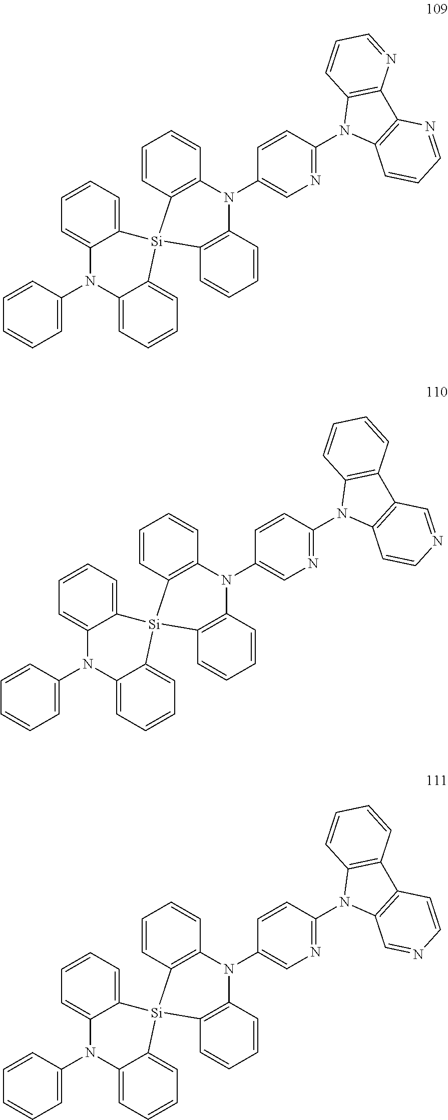 [SMILES: c1ccc(N2c3ccccc3[Si]3(c4ccccc42)c2ccccc2N(c2ccc(-n4c5cccnc5c5ncccc54)nc2)c2ccccc23)cc1 ; c1ccc(N2c3ccccc3[Si]3(c4ccccc42)c2ccccc2N(c2ccc(-n4c5ccccc5c5cnccc54)nc2)c2ccccc23)cc1 ; c1ccc(N2c3ccccc3[Si]3(c4ccccc42)c2ccccc2N(c2ccc(-n4c5ccccc5c5ccncc54)nc2)c2ccccc23)cc1]

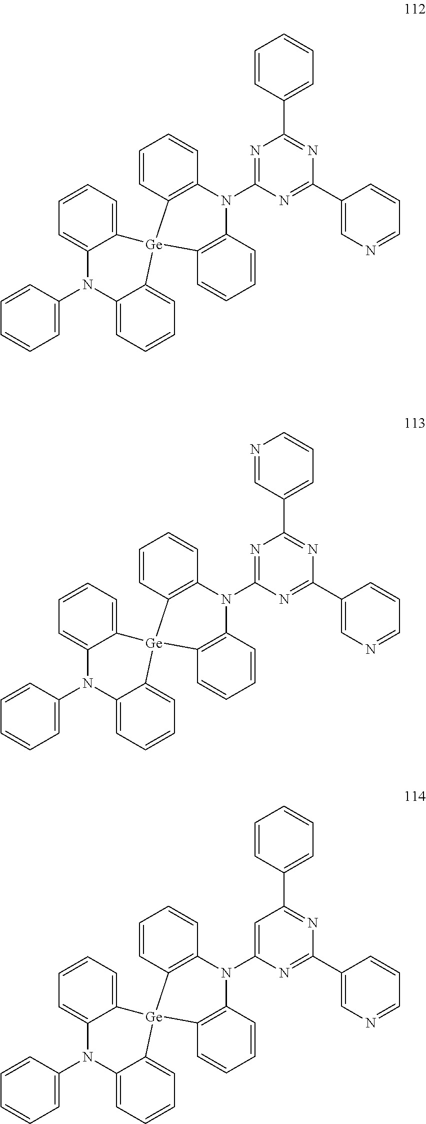

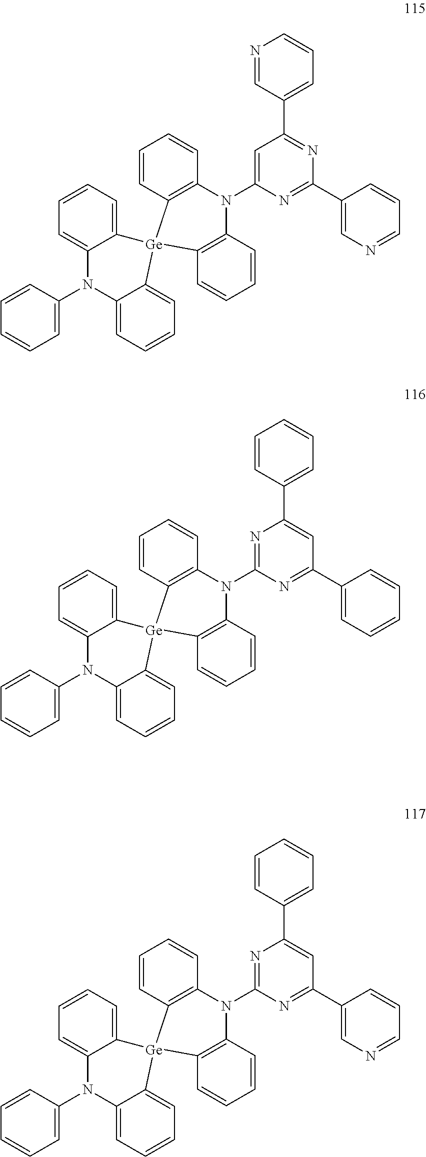

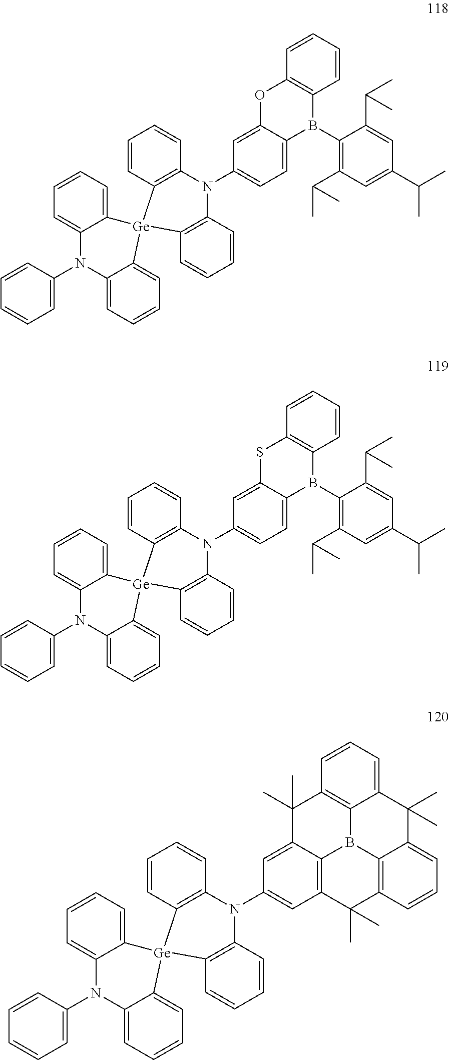

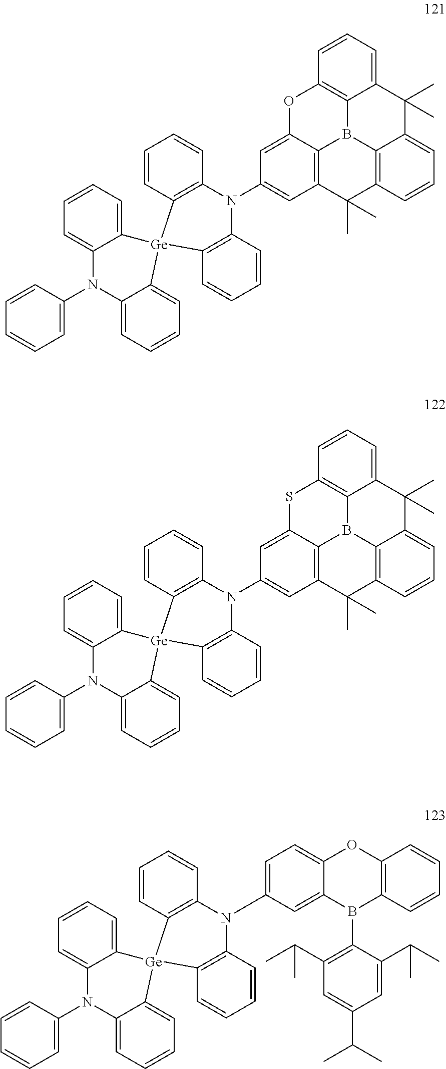

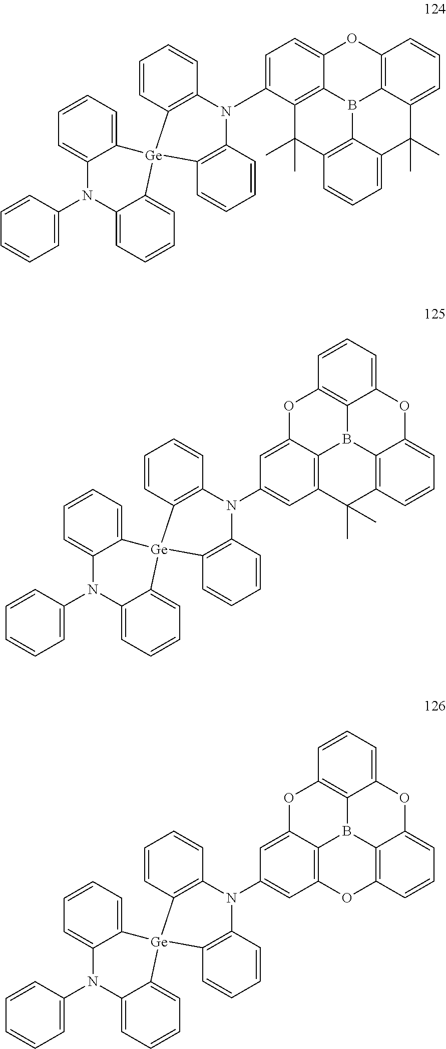

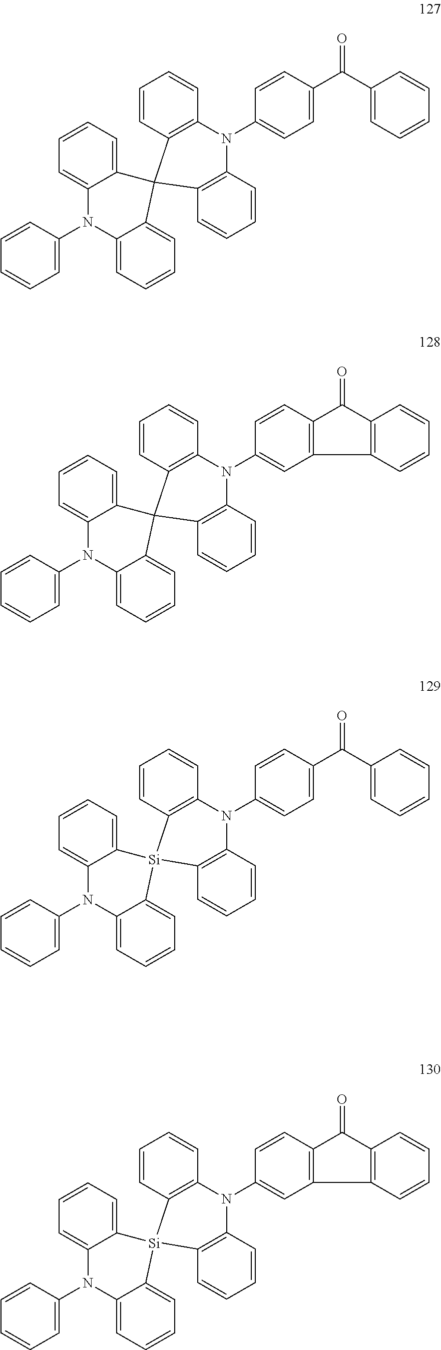 [SMILES: O=C(c1ccccc1)c1ccc(N2c3ccccc3C3(c4ccccc4N(c4ccccc4)c4ccccc43)c3ccccc32)cc1 ; O=C1c2ccccc2-c2cc(N3c4ccccc4C4(c5ccccc5N(c5ccccc5)c5ccccc54)c4ccccc43)ccc21 ; O=C(c1ccccc1)c1ccc(N2c3ccccc3[Si]3(c4ccccc4N(c4ccccc4)c4ccccc43)c3ccccc32)cc1 ; O=C1c2ccccc2-c2cc(N3c4ccccc4[Si]4(c5ccccc5N(c5ccccc5)c5ccccc54)c4ccccc43)ccc21]

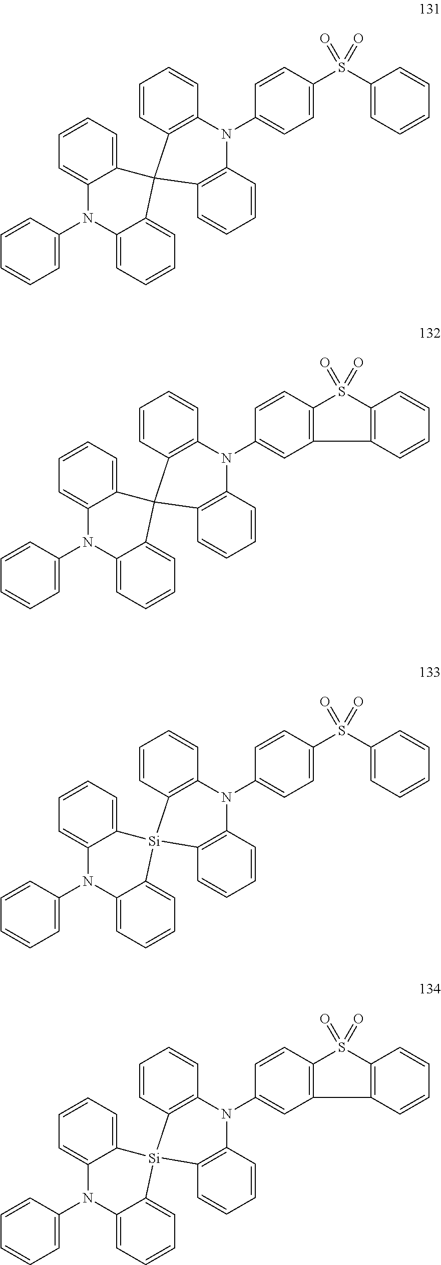 [SMILES: O=S(=O)(c1ccccc1)c1ccc(N2c3ccccc3C3(c4ccccc4N(c4ccccc4)c4ccccc43)c3ccccc32)cc1 ; O=S1(=O)c2ccccc2-c2cc(N3c4ccccc4C4(c5ccccc5N(c5ccccc5)c5ccccc54)c4ccccc43)ccc21 ; O=S(=O)(c1ccccc1)c1ccc(N2c3ccccc3[Si]3(c4ccccc4N(c4ccccc4)c4ccccc43)c3ccccc32)cc1 ; O=S1(=O)c2ccccc2-c2cc(N3c4ccccc4[Si]4(c5ccccc5N(c5ccccc5)c5ccccc54)c4ccccc43)ccc21]

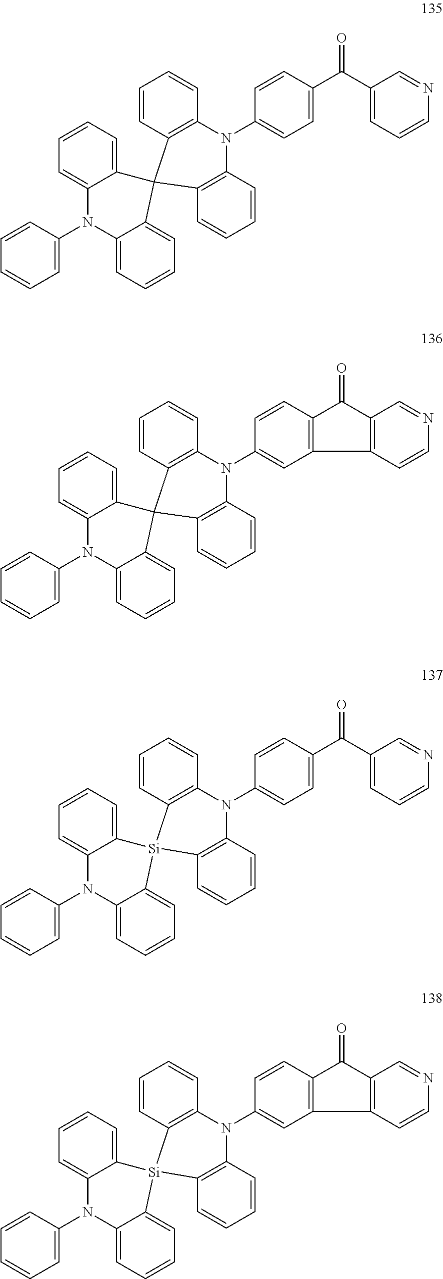 [SMILES: O=C(c1ccc(N2c3ccccc3C3(c4ccccc4N(c4ccccc4)c4ccccc43)c3ccccc32)cc1)c1cccnc1 ; O=C1c2cnccc2-c2cc(N3c4ccccc4C4(c5ccccc5N(c5ccccc5)c5ccccc54)c4ccccc43)ccc21 ; O=C(c1ccc(N2c3ccccc3[Si]3(c4ccccc4N(c4ccccc4)c4ccccc43)c3ccccc32)cc1)c1cccnc1 ; O=C1c2cnccc2-c2cc(N3c4ccccc4[Si]4(c5ccccc5N(c5ccccc5)c5ccccc54)c4ccccc43)ccc21]

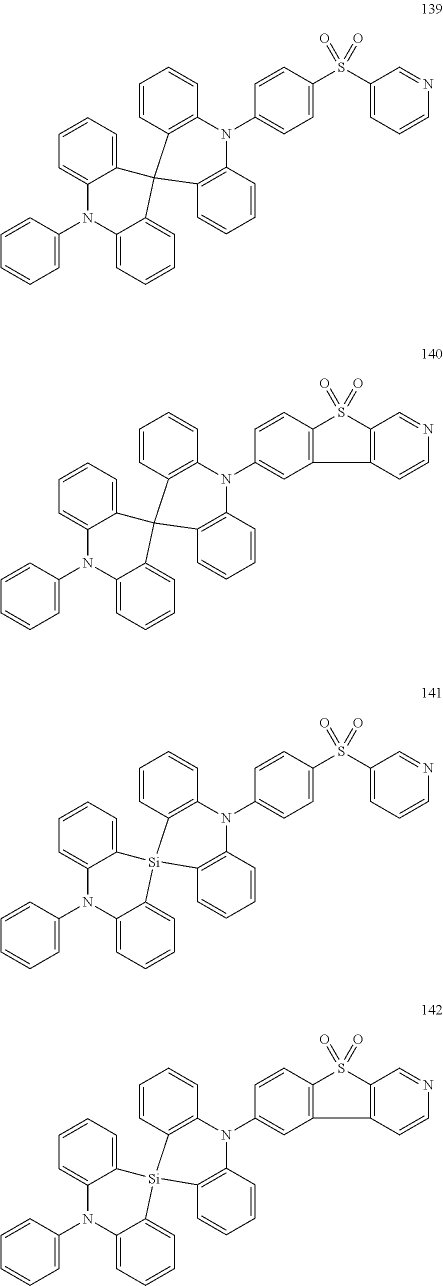 [SMILES: O=S(=O)(c1ccc(N2c3ccccc3C3(c4ccccc4N(c4ccccc4)c4ccccc43)c3ccccc32)cc1)c1cccnc1 ; O=S1(=O)c2ccc(N3c4ccccc4C4(c5ccccc5N(c5ccccc5)c5ccccc54)c4ccccc43)cc2-c2ccncc21 ; O=S(=O)(c1ccc(N2c3ccccc3[Si]3(c4ccccc4N(c4ccccc4)c4ccccc43)c3ccccc32)cc1)c1cccnc1 ; O=S1(=O)c2ccc(N3c4ccccc4[Si]4(c5ccccc5N(c5ccccc5)c5ccccc54)c4ccccc43)cc2-c2ccncc21]

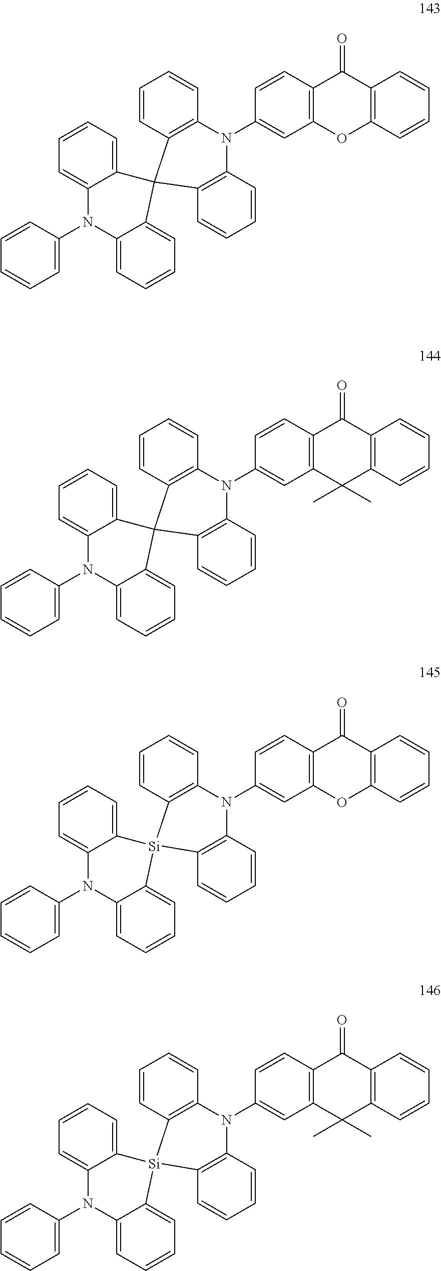 [SMILES: O=c1c2ccccc2oc2cc(N3c4ccccc4C4(c5ccccc5N(c5ccccc5)c5ccccc54)c4ccccc43)ccc12 ; CC1(C)c2ccccc2C(=O)c2ccc(N3c4ccccc4C4(c5ccccc5N(c5ccccc5)c5ccccc54)c4ccccc43)cc21 ; O=c1c2ccccc2oc2cc(N3c4ccccc4[Si]4(c5ccccc5N(c5ccccc5)c5ccccc54)c4ccccc43)ccc12 ; CC1(C)c2ccccc2C(=O)c2ccc(N3c4ccccc4[Si]4(c5ccccc5N(c5ccccc5)c5ccccc54)c4ccccc43)cc21]

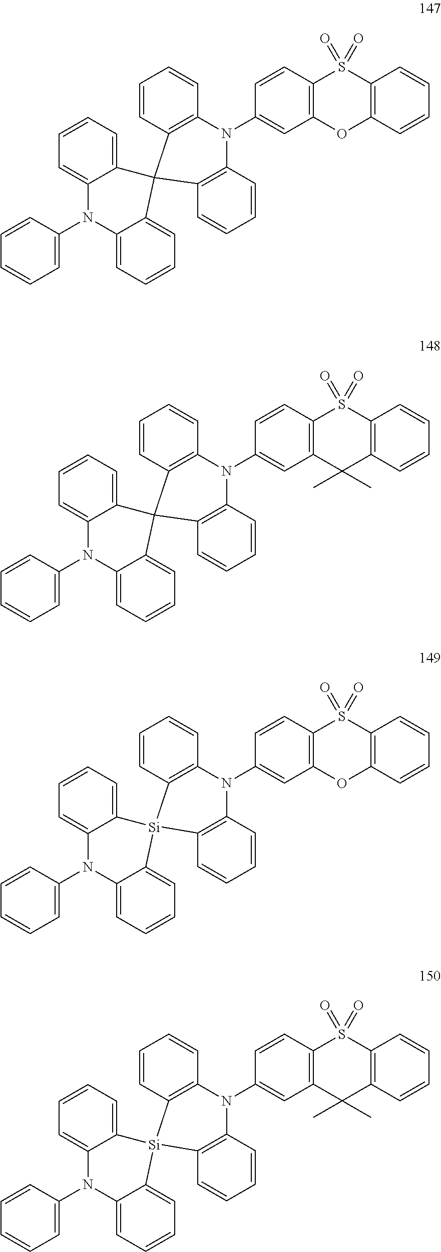 [SMILES: O=S1(=O)c2ccccc2Oc2cc(N3c4ccccc4C4(c5ccccc5N(c5ccccc5)c5ccccc54)c4ccccc43)ccc21 ; CC1(C)c2ccccc2S(=O)(=O)c2ccc(N3c4ccccc4C4(c5ccccc5N(c5ccccc5)c5ccccc54)c4ccccc43)cc21 ; O=S1(=O)c2ccccc2Oc2cc(N3c4ccccc4[Si]4(c5ccccc5N(c5ccccc5)c5ccccc54)c4ccccc43)ccc21 ; CC1(C)c2ccccc2S(=O)(=O)c2ccc(N3c4ccccc4[Si]4(c5ccccc5N(c5ccccc5)c5ccccc54)c4ccccc43)cc21]

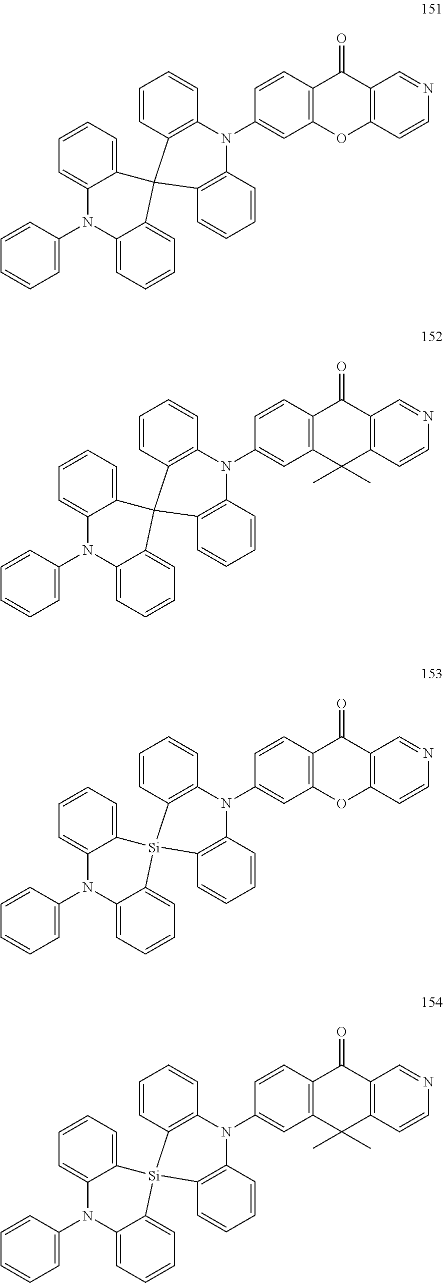 [SMILES: O=c1c2ccc(N3c4ccccc4C4(c5ccccc5N(c5ccccc5)c5ccccc54)c4ccccc43)cc2oc2ccncc12 ; CC1(C)c2ccncc2C(=O)c2ccc(N3c4ccccc4C4(c5ccccc5N(c5ccccc5)c5ccccc54)c4ccccc43)cc21 ; O=c1c2ccc(N3c4ccccc4[Si]4(c5ccccc5N(c5ccccc5)c5ccccc54)c4ccccc43)cc2oc2ccncc12 ; CC1(C)c2ccncc2C(=O)c2ccc(N3c4ccccc4[Si]4(c5ccccc5N(c5ccccc5)c5ccccc54)c4ccccc43)cc21]

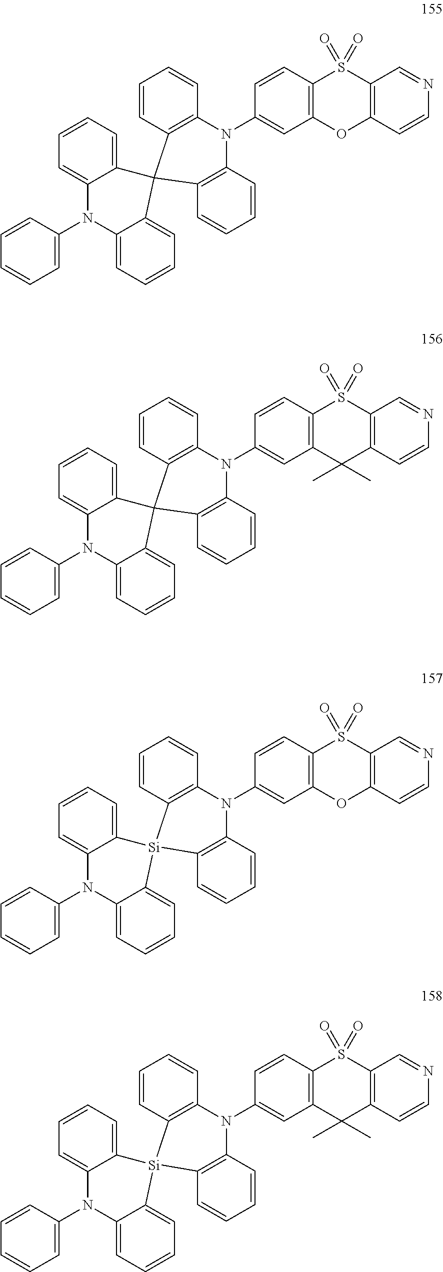 [SMILES: O=S1(=O)c2ccc(N3c4ccccc4C4(c5ccccc5N(c5ccccc5)c5ccccc54)c4ccccc43)cc2Oc2ccncc21 ; CC1(C)c2cc(N3c4ccccc4C4(c5ccccc5N(c5ccccc5)c5ccccc54)c4ccccc43)ccc2S(=O)(=O)c2cnccc21 ; O=S1(=O)c2ccc(N3c4ccccc4[Si]4(c5ccccc5N(c5ccccc5)c5ccccc54)c4ccccc43)cc2Oc2ccncc21 ; CC1(C)c2cc(N3c4ccccc4[Si]4(c5ccccc5N(c5ccccc5)c5ccccc54)c4ccccc43)ccc2S(=O)(=O)c2cnccc21]

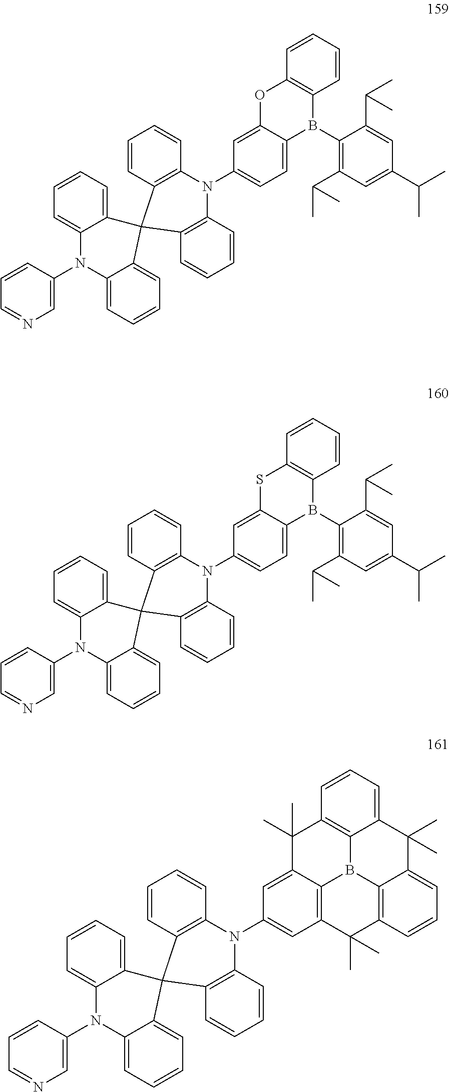 [SMILES: CC(C)c1cc(C(C)C)c(B2c3ccccc3Oc3cc(N4c5ccccc5C5(c6ccccc6N(c6cccnc6)c6ccccc65)c5ccccc54)ccc32)c(C(C)C)c1 ; CC(C)c1cc(C(C)C)c(B2c3ccccc3Sc3cc(N4c5ccccc5C5(c6ccccc6N(c6cccnc6)c6ccccc65)c5ccccc54)ccc32)c(C(C)C)c1 ; CC1(C)c2cccc3c2B2c4c1cccc4C(C)(C)c1cc(N4c5ccccc5C5(c6ccccc6N(c6cccnc6)c6ccccc65)c5ccccc54)cc(c12)C3(C)C]

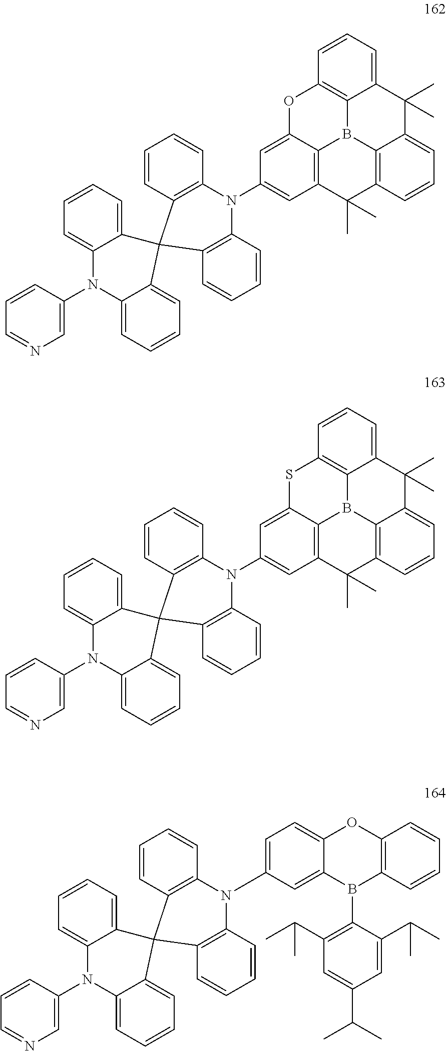 [SMILES: CC1(C)c2cccc3c2B2c4c(cc(N5c6ccccc6C6(c7ccccc7N(c7cccnc7)c7ccccc76)c6ccccc65)cc4C(C)(C)c4cccc1c42)O3 ; CC1(C)c2cccc3c2B2c4c(cc(N5c6ccccc6C6(c7ccccc7N(c7cccnc7)c7ccccc76)c6ccccc65)cc4C(C)(C)c4cccc1c42)S3 ; CC(C)c1cc(C(C)C)c(B2c3ccccc3Oc3ccc(N4c5ccccc5C5(c6ccccc6N(c6cccnc6)c6ccccc65)c5ccccc54)cc32)c(C(C)C)c1]

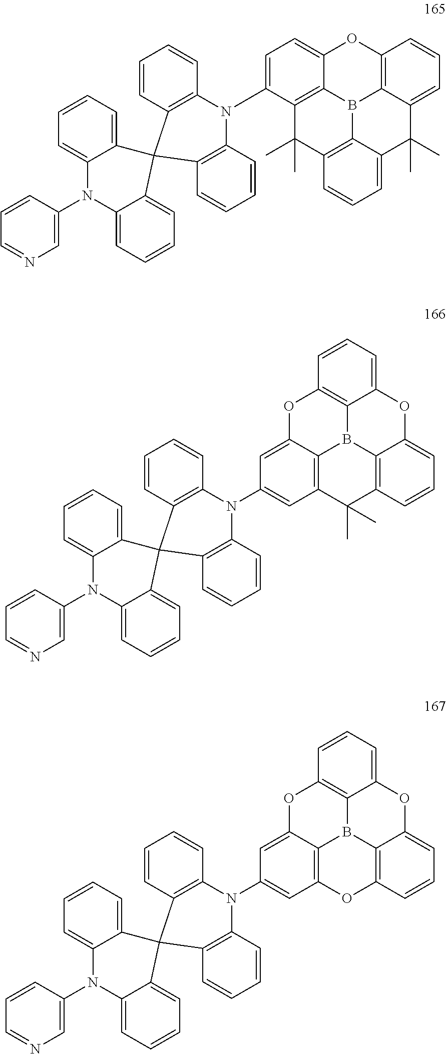 [SMILES: CC1(C)c2cccc3c2B2c4c1cccc4C(C)(C)c1c(N4c5ccccc5C5(c6ccccc6N(c6cccnc6)c6ccccc65)c5ccccc54)ccc(c12)O3 ; CC1(C)c2cccc3c2B2c4c(cccc4Oc4cc(N5c6ccccc6C6(c7ccccc7N(c7cccnc7)c7ccccc76)c6ccccc65)cc1c42)O3 ; c1cncc(N2c3ccccc3C3(c4ccccc42)c2ccccc2N(c2cc4c5c(c2)Oc2cccc6c2B5c2c(cccc2O4)O6)c2ccccc23)c1]

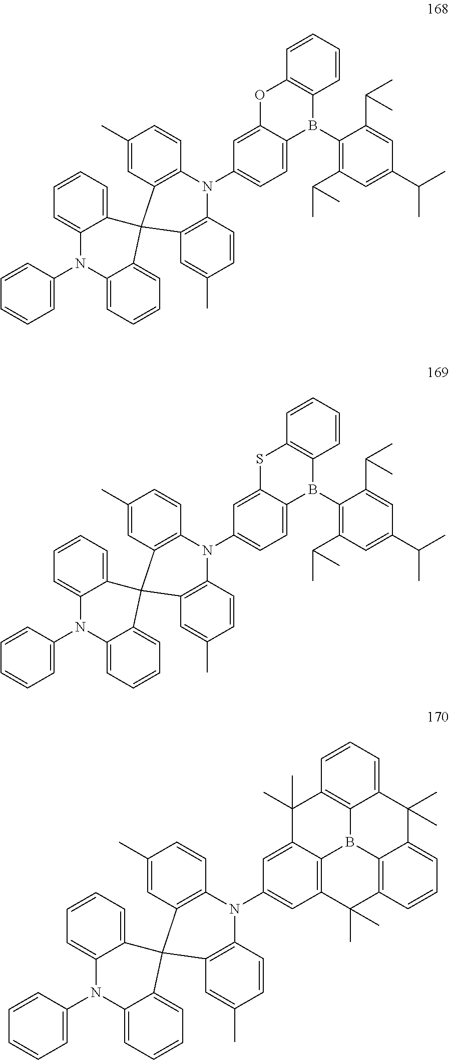 [SMILES: Cc1ccc2c(c1)C1(c3ccccc3N(c3ccccc3)c3ccccc31)c1cc(C)ccc1N2c1ccc2c(c1)Oc1ccccc1B2c1c(C(C)C)cc(C(C)C)cc1C(C)C ; Cc1ccc2c(c1)C1(c3ccccc3N(c3ccccc3)c3ccccc31)c1cc(C)ccc1N2c1ccc2c(c1)Sc1ccccc1B2c1c(C(C)C)cc(C(C)C)cc1C(C)C ; Cc1ccc2c(c1)C1(c3ccccc3N(c3ccccc3)c3ccccc31)c1cc(C)ccc1N2c1cc2c3c(c1)C(C)(C)c1cccc4c1B3c1c(cccc1C2(C)C)C4(C)C]

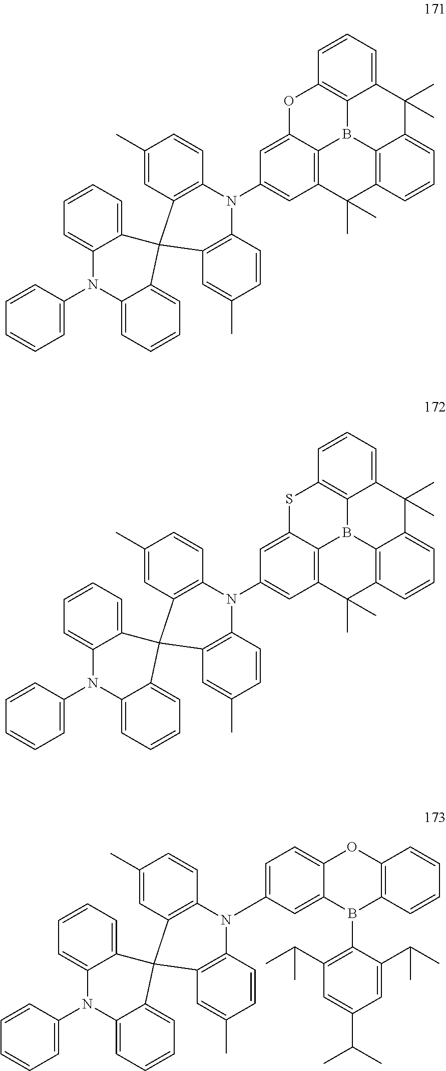 [SMILES: Cc1ccc2c(c1)C1(c3ccccc3N(c3ccccc3)c3ccccc31)c1cc(C)ccc1N2c1cc2c3c(c1)C(C)(C)c1cccc4c1B3c1c(cccc1C4(C)C)O2 ; Cc1ccc2c(c1)C1(c3ccccc3N(c3ccccc3)c3ccccc31)c1cc(C)ccc1N2c1cc2c3c(c1)C(C)(C)c1cccc4c1B3c1c(cccc1C4(C)C)S2 ; Cc1ccc2c(c1)C1(c3ccccc3N(c3ccccc3)c3ccccc31)c1cc(C)ccc1N2c1ccc2c(c1)B(c1c(C(C)C)cc(C(C)C)cc1C(C)C)c1ccccc1O2]

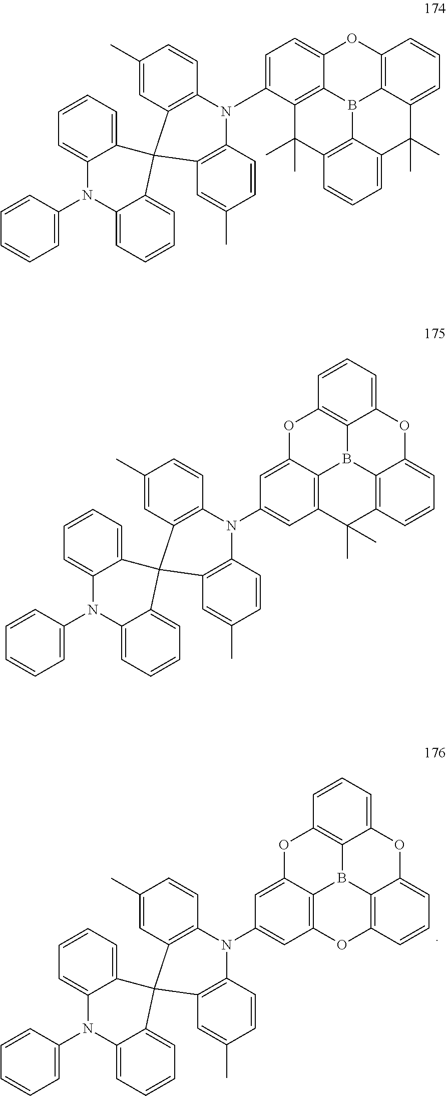 [SMILES: Cc1ccc2c(c1)C1(c3ccccc3N(c3ccccc3)c3ccccc31)c1cc(C)ccc1N2c1ccc2c3c1C(C)(C)c1cccc4c1B3c1c(cccc1C4(C)C)O2 ; Cc1ccc2c(c1)C1(c3ccccc3N(c3ccccc3)c3ccccc31)c1cc(C)ccc1N2c1cc2c3c(c1)C(C)(C)c1cccc4c1B3c1c(cccc1O2)O4 ; Cc1ccc2c(c1)C1(c3ccccc3N(c3ccccc3)c3ccccc31)c1cc(C)ccc1N2c1cc2c3c(c1)Oc1cccc4c1B3c1c(cccc1O2)O4]

14. A polycyclic compound represented by Formula 1 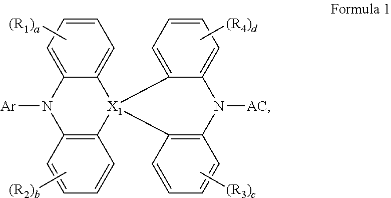
wherein, in Formula 1,
X₁ is C, Si, or Ge,
Ar is a substituted or unsubstituted aryl group having 6 to 30 carbon atoms for forming a ring, or a substituted or unsubstituted heteroaryl group having 2 to 30 carbon atoms for forming a ring,
R₁ to R₄ are each independently a hydrogen atom, a deuterium atom, a substituted or unsubstituted alkyl group having 1 to 10 carbon atoms, a substituted or unsubstituted aryl group having 6 to 30 carbon atoms for forming a ring, or a substituted or unsubstituted heteroaryl group having 2 to 30 carbon atoms for forming a ring,
“a” to “d” are each independently an integer of 0 to 4, and
AC is represented by one of Formulae 2 to 9: 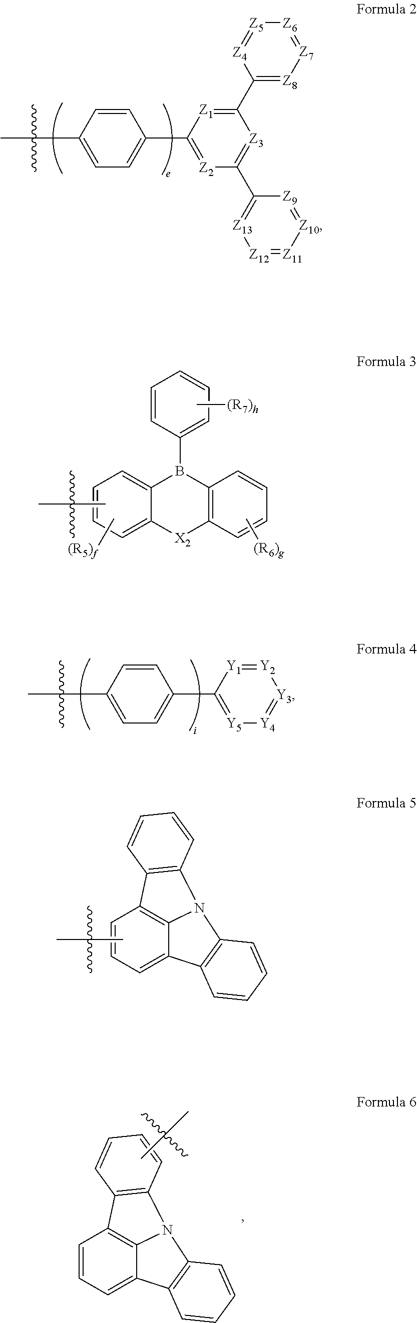 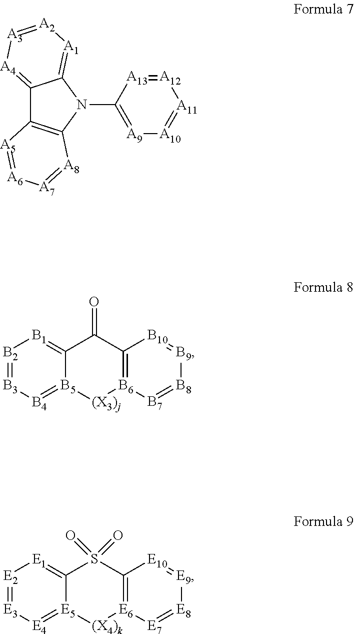
wherein, in Formula 2,
Z₁ to Z₁₃ are each independently CH or N, where two or three among Z₁ to Z₃ are each N, and at least one of Z₄ to Z₁₃ is N, and
“e” is an integer of 0 to 2,
wherein, in Formula 3,
X₂ is O, S or CR₈R₉,
R₅ to R₉ are each independently a hydrogen atom, a deuterium atom, or a substituted or unsubstituted alkyl group having 1 to 5 carbon atoms, or are combined with an adjacent group to form a hydrocarbon ring or a heterocycle,
“f” is an integer of 0 to 3,
“g” is an integer of 0 to 4, and
“h” is an integer of 0 to 5,
wherein, in Formula 4,
“i” is an integer of 0 to 2,
Y₁ is N or CR₁₀, Y₂ is N or CR₁₁, Y₃ is N or CR₁₂, Y₄ is N or CR₁₃, and Y₅ is N or CR₁₄, where at least one of Y₁ to Y₅ is N,
CR₁₀ to CR₁₄ are each independently a hydrogen atom, a deuterium atom, a cyano group, a substituted or unsubstituted alkyl group having 1 to 10 carbon atoms, or a trifluoromethyl group,
when X₁ of Formula 1 is C, at least one of R₁₀ to R₁₄ is not a hydrogen atom, and
when at least one of Y₂ and Y₄ is N, Y₃ is CR₁₂, and R₁₂ is not a cyano group,
wherein, in Formula 7,
A₁ to A₁₃ are each independently N or CR₁₅,
at least one of A₁ to A₈ is N, at least one of A₉ to A₁₃ is N, and at least one of A₁ to A₁₃ is a part that is connected with Formula 1, and
R₁₅ a hydrogen atom, a deuterium atom, a substituted or unsubstituted alkyl group having 1 to 10 carbon atoms, a substituted or unsubstituted aryl group having 6 to 30 carbon atoms for forming a ring, or a substituted or unsubstituted heteroaryl group having 2 to 30 carbon atoms for forming a ring, wherein, in Formula 8,
B₁ to B₁₀ are each independently N or CR₁₆, and one of B₁ to B₁₀ is a part that is connected with Formula 1,
X₃ is a direct linkage, O, S, CR₁₇R₁₈, or SiR₁₉R₂₀,
“j” is 0 or 1, where if “j” is 1, B₅ and B₆ are C, and
R₁₆ to R₂₀ are each independently a hydrogen atom, a deuterium atom, a substituted or unsubstituted alkyl group having 1 to 10 carbon atoms, a substituted or unsubstituted aryl group having 6 to 30 carbon atoms for forming a ring, or a substituted or unsubstituted heteroaryl group having 2 to 30 carbon atoms for forming a ring, and
wherein, in Formula 9,
E₁ to E₁₀ are each independently N or CR₂₁, and one of E₁ to E₁₀ is a part that is connected with Formula 1,
X₄ is a direct linkage, O, S, CR₂₂R₂₃, or SiR₂₄R₂₅,
“k” is 0 or 1, where if “k” is 1, E₅ and E₆ are C, and
R₂₂ to R₂₅ are each independently a hydrogen atom, a deuterium atom, a substituted or unsubstituted alkyl group having 1 to 10 carbon atoms, a substituted or unsubstituted aryl group having 6 to 30 carbon atoms for forming a ring, or a substituted or unsubstituted heteroaryl group having 2 to 30 carbon atoms for forming a ring.

15. The polycyclic compound of claim 14, wherein Formula 3 is further represented by Formula 3-1 or 3-2: 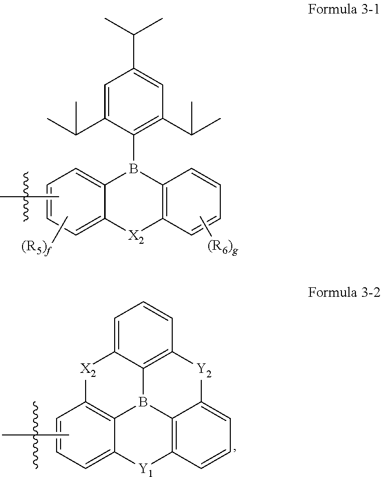
wherein, in Formulae 3-1 and 3-2,
X₂, R₅, R₆, “f” and “g” are each independently the same as defined in Formula 3,
Y₁ and Y₂ are each independently O, S or CR₂₆R₂₇, and
R₂₆ and R₂₇ are each independently a hydrogen atom, a deuterium atom, or a substituted or unsubstituted alkyl group having 1 to 5 carbon atoms.

16. The polycyclic compound of claim 14, wherein Formula 4 is further represented by one of the following formulae:

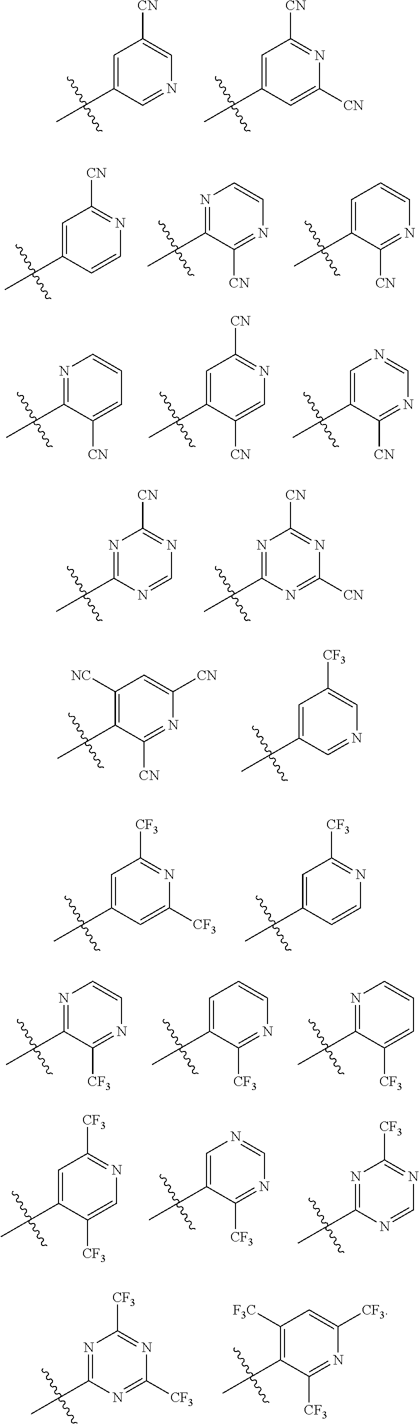

17. The polycyclic compound of claim 14, wherein Formula 7 is further represented by one of the following formulae:

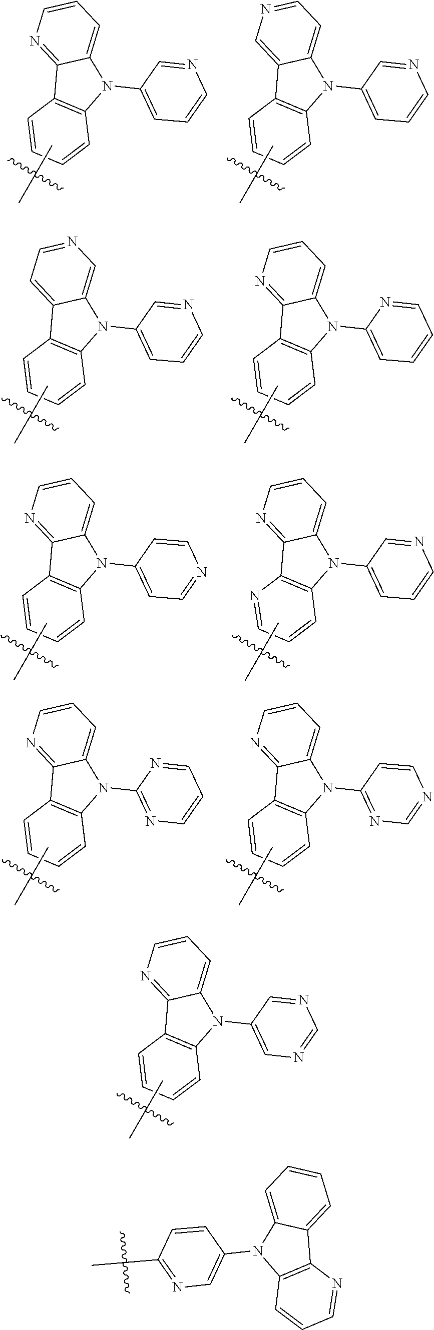

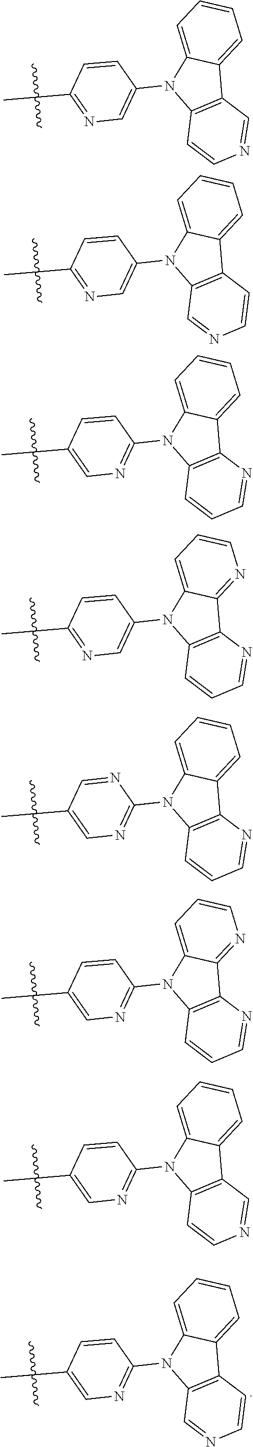

18. The polycyclic compound of claim 14, wherein Formula 8 is further represented by one of the following formulae:

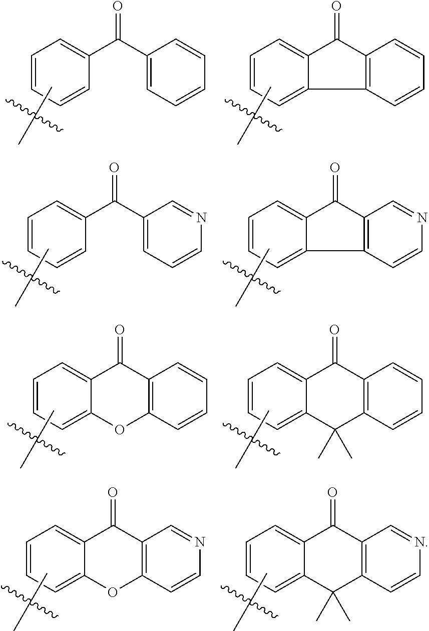

19. The polycyclic compound of claim 14, wherein Formula 9 is further represented by one of the following formulae:

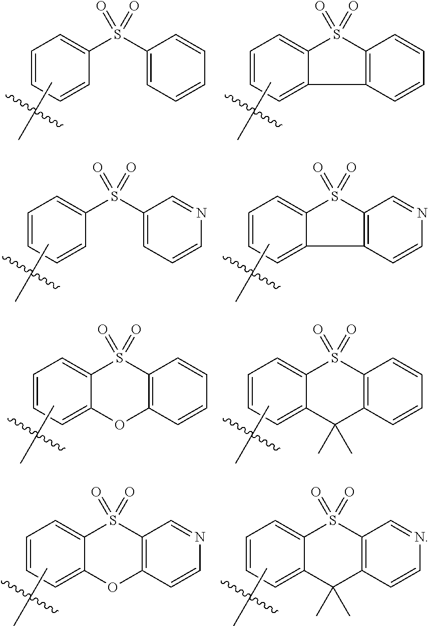

20. The polycyclic compound of claim 14, wherein the polycyclic compound represented by Formula 1 is at least one selected from compounds represented in Compound Group 1: 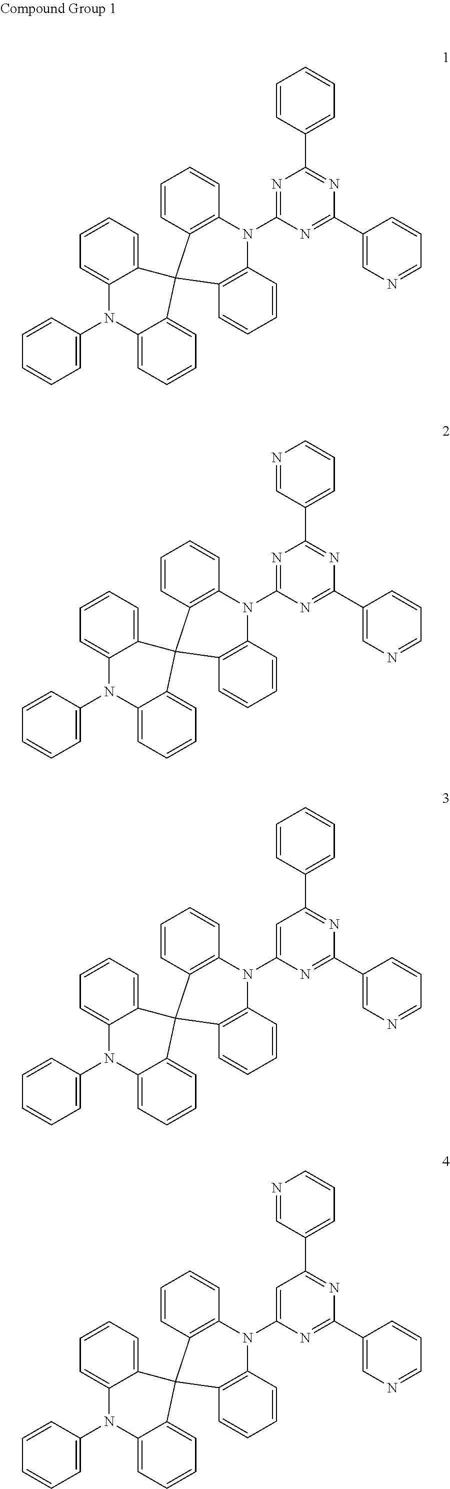 [SMILES: c1ccc(-c2nc(-c3cccnc3)nc(N3c4ccccc4C4(c5ccccc5N(c5ccccc5)c5ccccc54)c4ccccc43)n2)cc1 ; c1ccc(N2c3ccccc3C3(c4ccccc42)c2ccccc2N(c2nc(-c4cccnc4)nc(-c4cccnc4)n2)c2ccccc23)cc1 ; c1ccc(-c2cc(N3c4ccccc4C4(c5ccccc5N(c5ccccc5)c5ccccc54)c4ccccc43)nc(-c3cccnc3)n2)cc1 ; c1ccc(N2c3ccccc3C3(c4ccccc42)c2ccccc2N(c2cc(-c4cccnc4)nc(-c4cccnc4)n2)c2ccccc23)cc1] 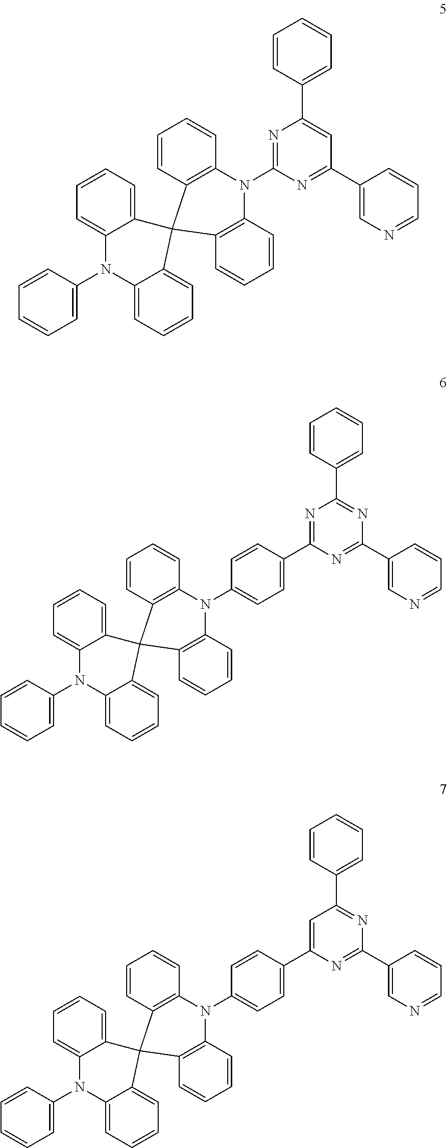 [SMILES: c1ccc(-c2cc(-c3cccnc3)nc(N3c4ccccc4C4(c5ccccc5N(c5ccccc5)c5ccccc54)c4ccccc43)n2)cc1 ; c1ccc(-c2nc(-c3ccc(N4c5ccccc5C5(c6ccccc6N(c6ccccc6)c6ccccc65)c5ccccc54)cc3)nc(-c3cccnc3)n2)cc1 ; c1ccc(-c2cc(-c3ccc(N4c5ccccc5C5(c6ccccc6N(c6ccccc6)c6ccccc65)c5ccccc54)cc3)nc(-c3cccnc3)n2)cc1] 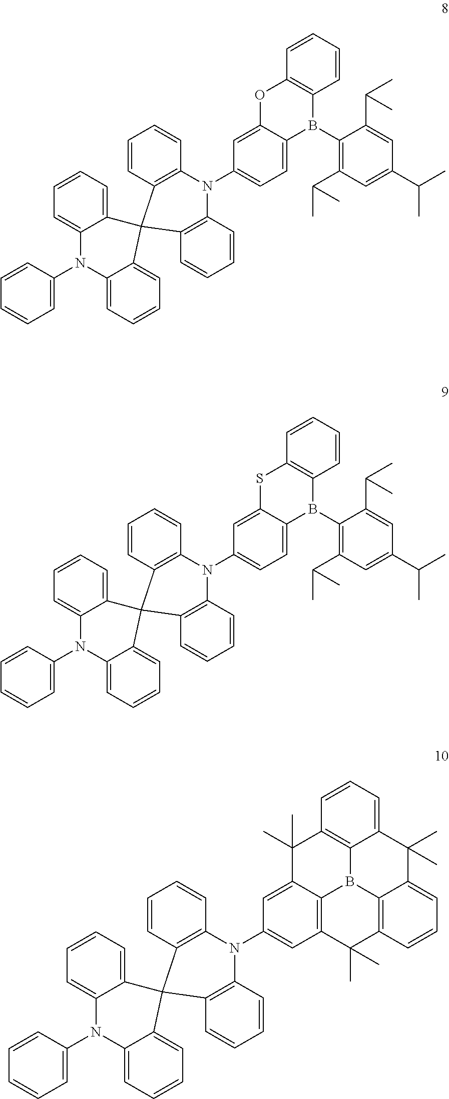 [SMILES: CC(C)c1cc(C(C)C)c(B2c3ccccc3Oc3cc(N4c5ccccc5C5(c6ccccc6N(c6ccccc6)c6ccccc65)c5ccccc54)ccc32)c(C(C)C)c1 ; CC(C)c1cc(C(C)C)c(B2c3ccccc3Sc3cc(N4c5ccccc5C5(c6ccccc6N(c6ccccc6)c6ccccc65)c5ccccc54)ccc32)c(C(C)C)c1 ; CC1(C)c2cccc3c2B2c4c1cccc4C(C)(C)c1cc(N4c5ccccc5C5(c6ccccc6N(c6ccccc6)c6ccccc65)c5ccccc54)cc(c12)C3(C)C] 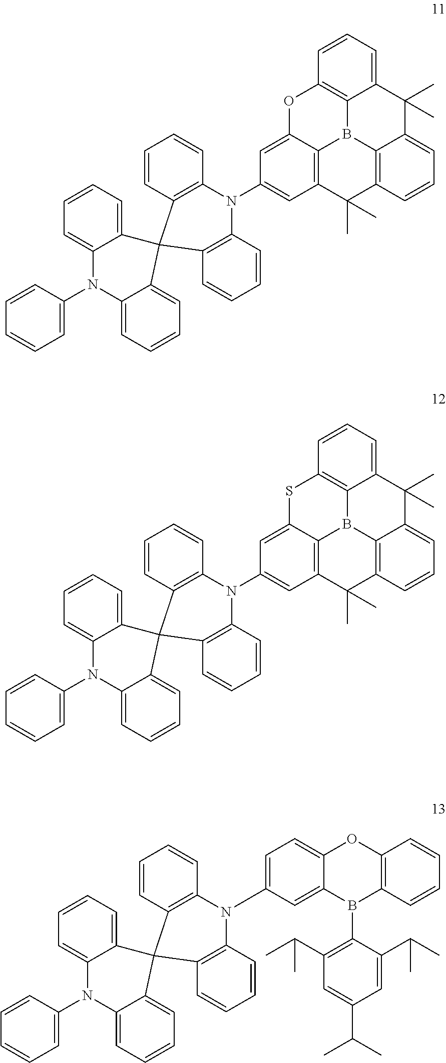 [SMILES: CC1(C)c2cccc3c2B2c4c(cc(N5c6ccccc6C6(c7ccccc7N(c7ccccc7)c7ccccc76)c6ccccc65)cc4C(C)(C)c4cccc1c42)O3 ; CC1(C)c2cccc3c2B2c4c(cc(N5c6ccccc6C6(c7ccccc7N(c7ccccc7)c7ccccc76)c6ccccc65)cc4C(C)(C)c4cccc1c42)S3 ; CC(C)c1cc(C(C)C)c(B2c3ccccc3Oc3ccc(N4c5ccccc5C5(c6ccccc6N(c6ccccc6)c6ccccc65)c5ccccc54)cc32)c(C(C)C)c1] 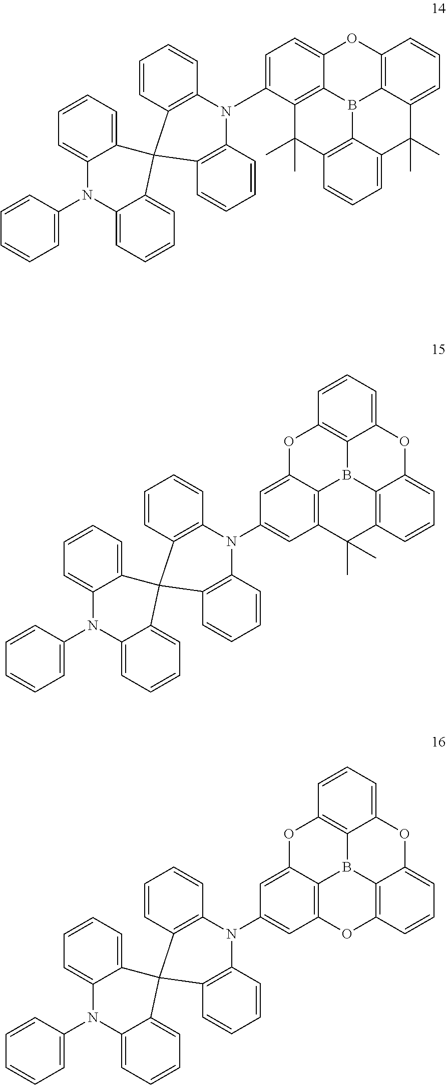 [SMILES: CC1(C)c2cccc3c2B2c4c1cccc4C(C)(C)c1c(N4c5ccccc5C5(c6ccccc6N(c6ccccc6)c6ccccc65)c5ccccc54)ccc(c12)O3 ; CC1(C)c2cccc3c2B2c4c(cccc4Oc4cc(N5c6ccccc6C6(c7ccccc7N(c7ccccc7)c7ccccc76)c6ccccc65)cc1c42)O3 ; c1ccc(N2c3ccccc3C3(c4ccccc42)c2ccccc2N(c2cc4c5c(c2)Oc2cccc6c2B5c2c(cccc2O4)O6)c2ccccc23)cc1] 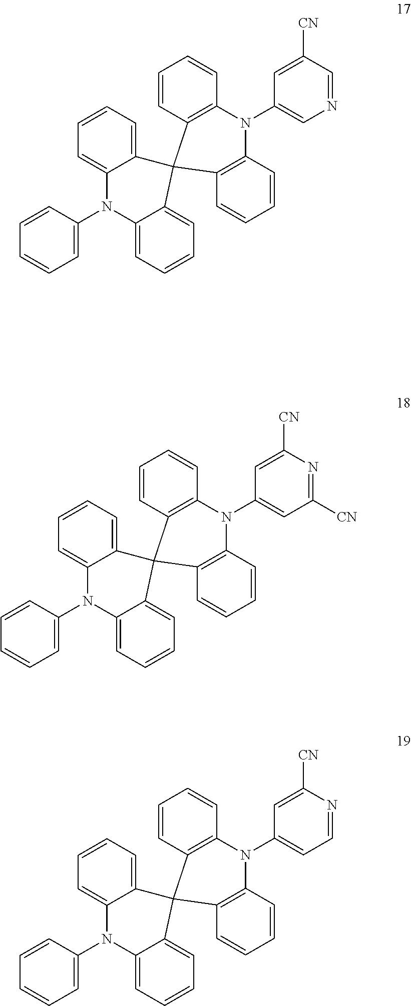 [SMILES: N#Cc1cncc(N2c3ccccc3C3(c4ccccc4N(c4ccccc4)c4ccccc43)c3ccccc32)c1 ; N#Cc1cc(N2c3ccccc3C3(c4ccccc4N(c4ccccc4)c4ccccc43)c3ccccc32)cc(C#N)n1 ; N#Cc1cc(N2c3ccccc3C3(c4ccccc4N(c4ccccc4)c4ccccc43)c3ccccc32)ccn1] 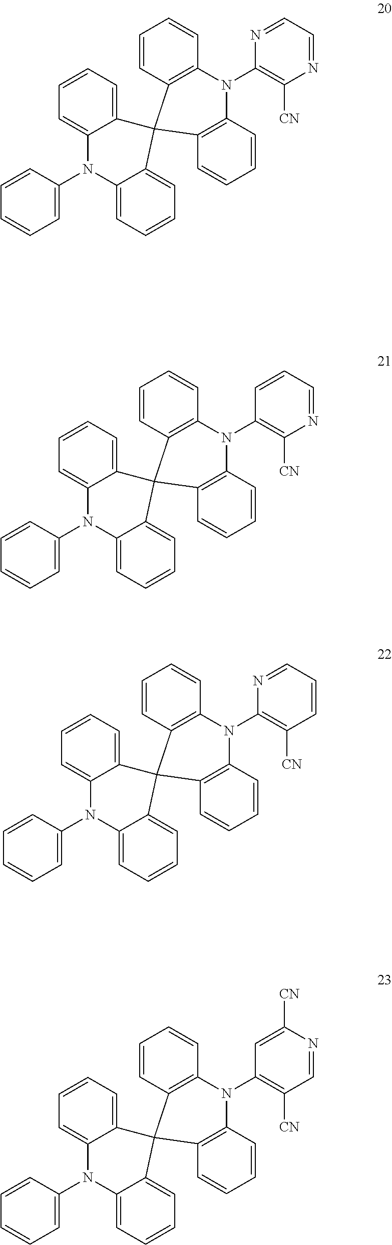 [SMILES: N#Cc1nccnc1N1c2ccccc2C2(c3ccccc3N(c3ccccc3)c3ccccc32)c2ccccc21 ; N#Cc1ncccc1N1c2ccccc2C2(c3ccccc3N(c3ccccc3)c3ccccc32)c2ccccc21 ; N#Cc1cccnc1N1c2ccccc2C2(c3ccccc3N(c3ccccc3)c3ccccc32)c2ccccc21 ; N#Cc1cc(N2c3ccccc3C3(c4ccccc4N(c4ccccc4)c4ccccc43)c3ccccc32)c(C#N)cn1] 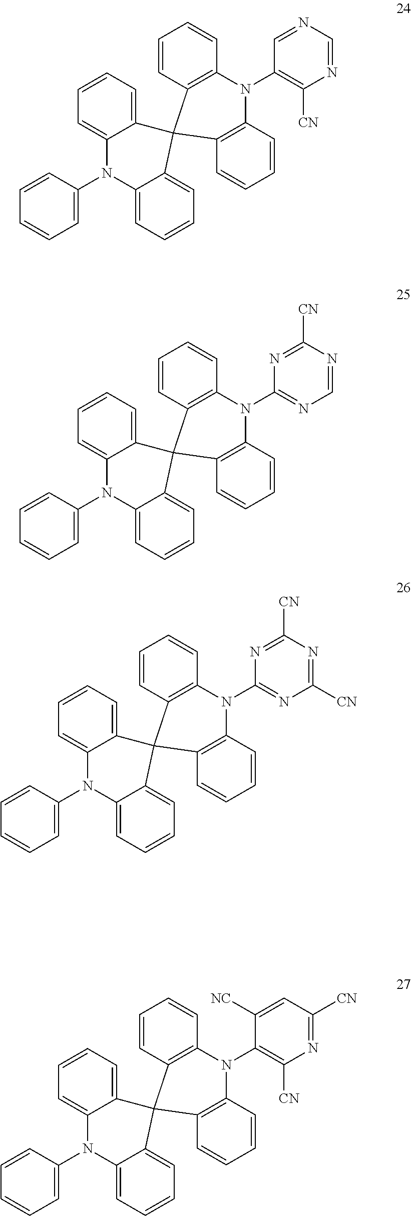 [SMILES: N#Cc1ncncc1N1c2ccccc2C2(c3ccccc3N(c3ccccc3)c3ccccc32)c2ccccc21 ; N#Cc1ncnc(N2c3ccccc3C3(c4ccccc4N(c4ccccc4)c4ccccc43)c3ccccc32)n1 ; N#Cc1nc(C#N)nc(N2c3ccccc3C3(c4ccccc4N(c4ccccc4)c4ccccc43)c3ccccc32)n1 ; N#Cc1cc(C#N)c(N2c3ccccc3C3(c4ccccc4N(c4ccccc4)c4ccccc43)c3ccccc32)c(C#N)n1] 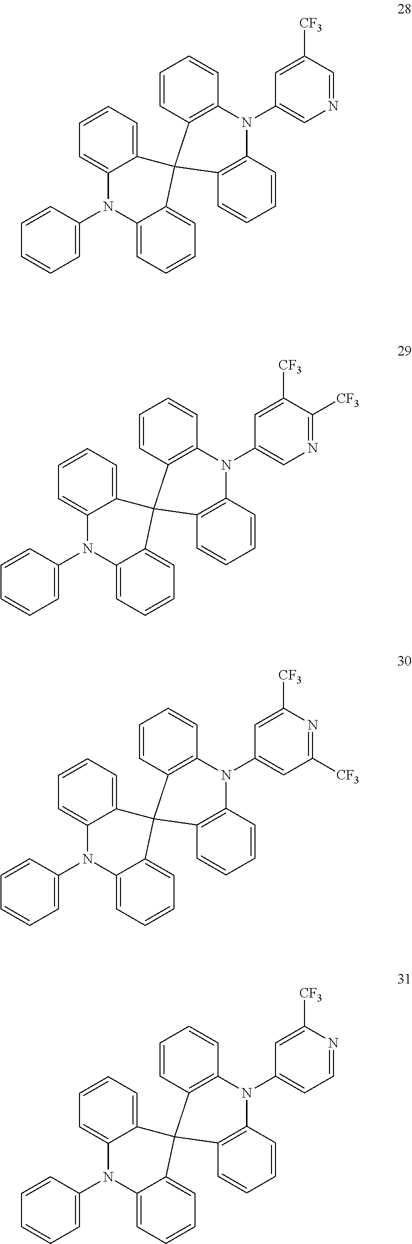 [SMILES: FC(F)(F)c1cncc(N2c3ccccc3C3(c4ccccc4N(c4ccccc4)c4ccccc43)c3ccccc32)c1 ; FC(F)(F)c1cc(N2c3ccccc3C3(c4ccccc4N(c4ccccc4)c4ccccc43)c3ccccc32)cnc1C(F)(F)F ; FC(F)(F)c1cc(N2c3ccccc3C3(c4ccccc4N(c4ccccc4)c4ccccc43)c3ccccc32)cc(C(F)(F)F)n1 ; FC(F)(F)c1cc(N2c3ccccc3C3(c4ccccc4N(c4ccccc4)c4ccccc43)c3ccccc32)ccn1] 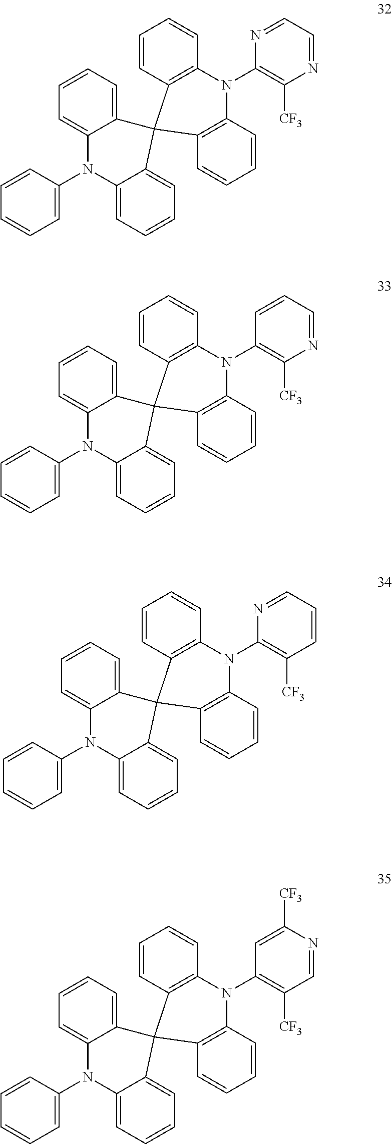 [SMILES: FC(F)(F)c1nccnc1N1c2ccccc2C2(c3ccccc3N(c3ccccc3)c3ccccc32)c2ccccc21 ; FC(F)(F)c1ncccc1N1c2ccccc2C2(c3ccccc3N(c3ccccc3)c3ccccc32)c2ccccc21 ; FC(F)(F)c1cccnc1N1c2ccccc2C2(c3ccccc3N(c3ccccc3)c3ccccc32)c2ccccc21 ; FC(F)(F)c1cc(N2c3ccccc3C3(c4ccccc4N(c4ccccc4)c4ccccc43)c3ccccc32)c(C(F)(F)F)cn1] 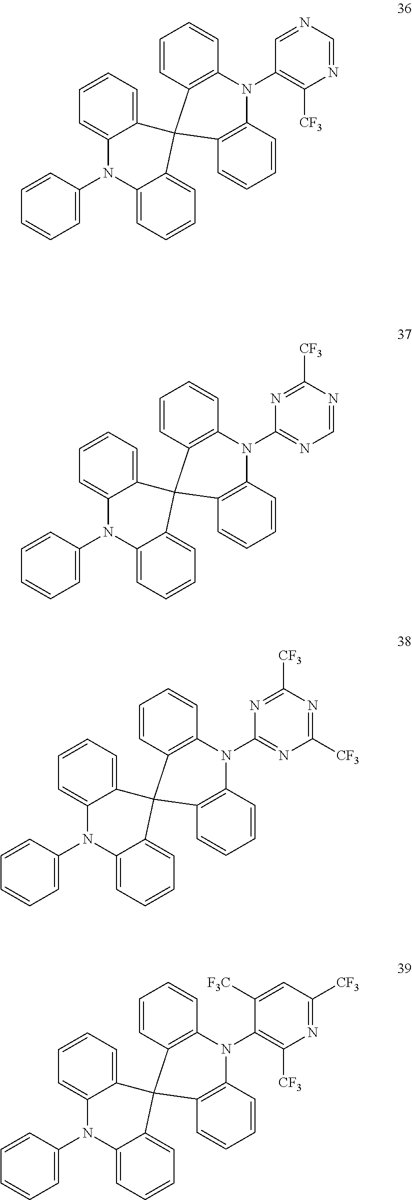 [SMILES: FC(F)(F)c1ncncc1N1c2ccccc2C2(c3ccccc3N(c3ccccc3)c3ccccc32)c2ccccc21 ; FC(F)(F)c1ncnc(N2c3ccccc3C3(c4ccccc4N(c4ccccc4)c4ccccc43)c3ccccc32)n1 ; FC(F)(F)c1nc(N2c3ccccc3C3(c4ccccc4N(c4ccccc4)c4ccccc43)c3ccccc32)nc(C(F)(F)F)n1 ; FC(F)(F)c1cc(C(F)(F)F)c(N2c3ccccc3C3(c4ccccc4N(c4ccccc4)c4ccccc43)c3ccccc32)c(C(F)(F)F)n1] 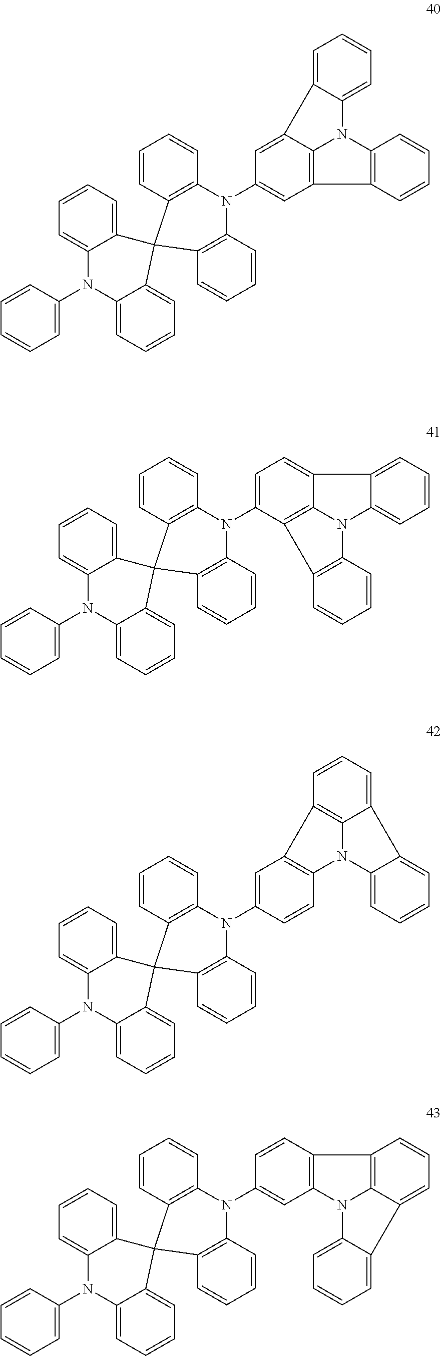 [SMILES: c1ccc(N2c3ccccc3C3(c4ccccc42)c2ccccc2N(c2cc4c5ccccc5n5c6ccccc6c(c2)c45)c2ccccc23)cc1 ; c1ccc(N2c3ccccc3C3(c4ccccc42)c2ccccc2N(c2ccc4c5ccccc5n5c6ccccc6c2c45)c2ccccc23)cc1 ; c1ccc(N2c3ccccc3C3(c4ccccc42)c2ccccc2N(c2ccc4c(c2)c2cccc5c6ccccc6n4c52)c2ccccc23)cc1 ; c1ccc(N2c3ccccc3C3(c4ccccc42)c2ccccc2N(c2ccc4c5cccc6c7ccccc7n(c4c2)c65)c2ccccc23)cc1] 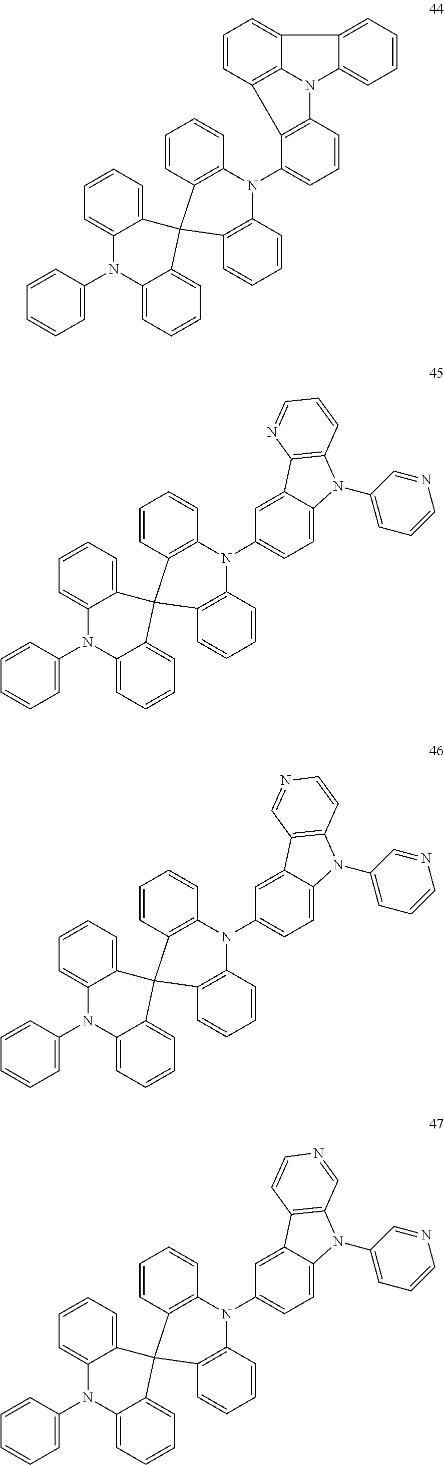 [SMILES: c1ccc(N2c3ccccc3C3(c4ccccc42)c2ccccc2N(c2cccc4c2c2cccc5c6ccccc6n4c52)c2ccccc23)cc1 ; c1ccc(N2c3ccccc3C3(c4ccccc42)c2ccccc2N(c2ccc4c(c2)c2ncccc2n4-c2cccnc2)c2ccccc23)cc1 ; c1ccc(N2c3ccccc3C3(c4ccccc42)c2ccccc2N(c2ccc4c(c2)c2cnccc2n4-c2cccnc2)c2ccccc23)cc1 ; c1ccc(N2c3ccccc3C3(c4ccccc42)c2ccccc2N(c2ccc4c(c2)c2ccncc2n4-c2cccnc2)c2ccccc23)cc1] 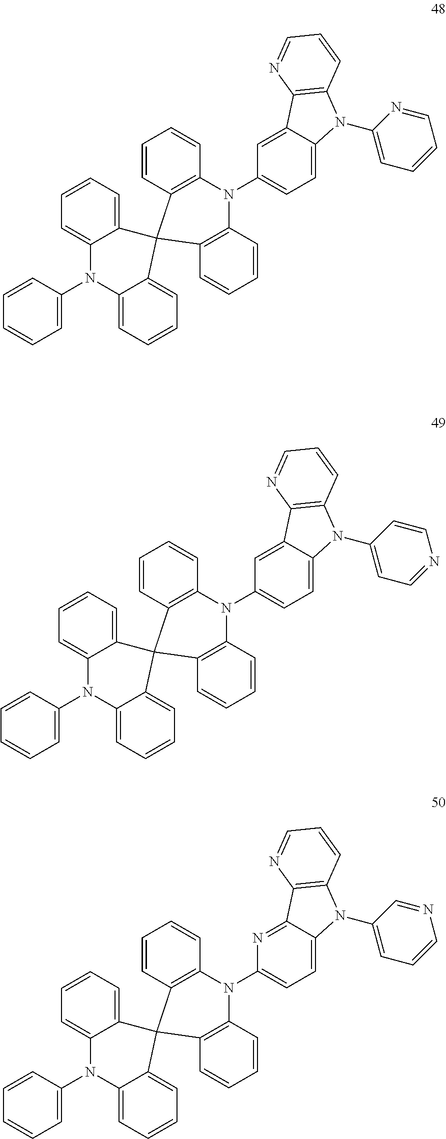 [SMILES: c1ccc(N2c3ccccc3C3(c4ccccc42)c2ccccc2N(c2ccc4c(c2)c2ncccc2n4-c2ccccn2)c2ccccc23)cc1 ; c1ccc(N2c3ccccc3C3(c4ccccc42)c2ccccc2N(c2ccc4c(c2)c2ncccc2n4-c2ccncc2)c2ccccc23)cc1 ; c1ccc(N2c3ccccc3C3(c4ccccc42)c2ccccc2N(c2ccc4c(n2)c2ncccc2n4-c2cccnc2)c2ccccc23)cc1] 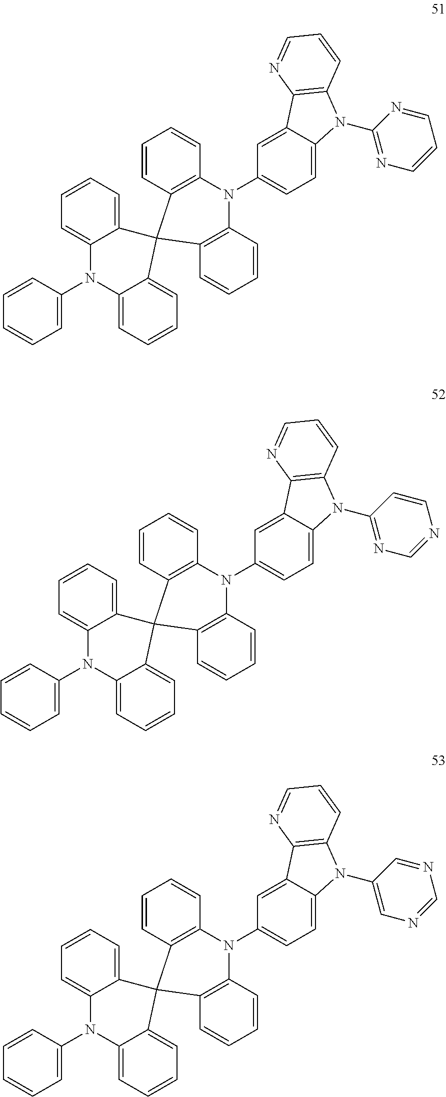 [SMILES: c1ccc(N2c3ccccc3C3(c4ccccc42)c2ccccc2N(c2ccc4c(c2)c2ncccc2n4-c2ncccn2)c2ccccc23)cc1 ; c1ccc(N2c3ccccc3C3(c4ccccc42)c2ccccc2N(c2ccc4c(c2)c2ncccc2n4-c2ccncn2)c2ccccc23)cc1 ; c1ccc(N2c3ccccc3C3(c4ccccc42)c2ccccc2N(c2ccc4c(c2)c2ncccc2n4-c2cncnc2)c2ccccc23)cc1] 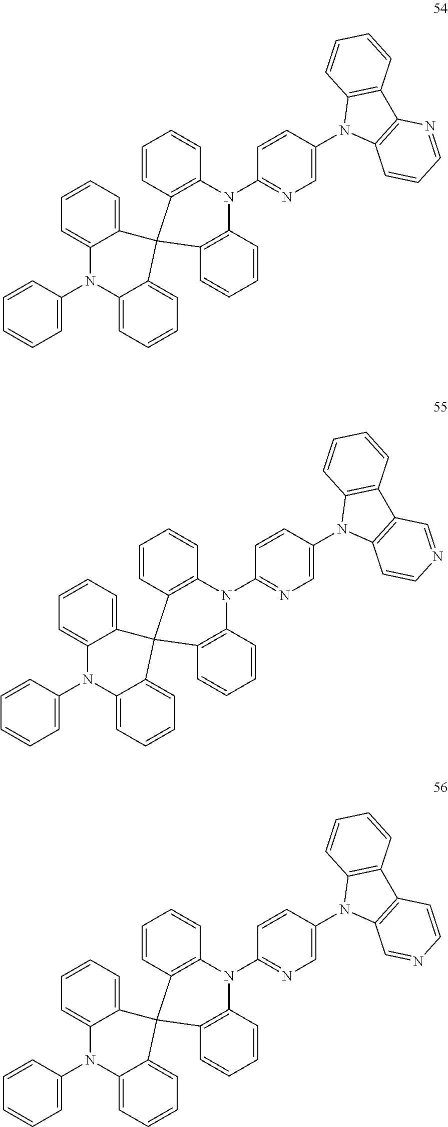 [SMILES: c1ccc(N2c3ccccc3C3(c4ccccc42)c2ccccc2N(c2ccc(-n4c5ccccc5c5ncccc54)cn2)c2ccccc23)cc1 ; c1ccc(N2c3ccccc3C3(c4ccccc42)c2ccccc2N(c2ccc(-n4c5ccccc5c5cnccc54)cn2)c2ccccc23)cc1 ; c1ccc(N2c3ccccc3C3(c4ccccc42)c2ccccc2N(c2ccc(-n4c5ccccc5c5ccncc54)cn2)c2ccccc23)cc1] 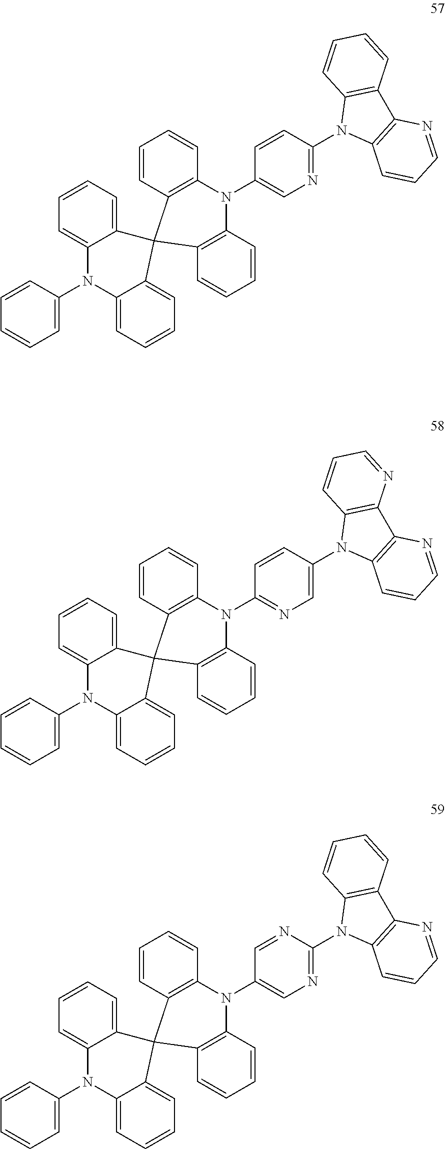 [SMILES: c1ccc(N2c3ccccc3C3(c4ccccc42)c2ccccc2N(c2ccc(-n4c5ccccc5c5ncccc54)nc2)c2ccccc23)cc1 ; c1ccc(N2c3ccccc3C3(c4ccccc42)c2ccccc2N(c2ccc(-n4c5cccnc5c5ncccc54)cn2)c2ccccc23)cc1 ; c1ccc(N2c3ccccc3C3(c4ccccc42)c2ccccc2N(c2cnc(-n4c5ccccc5c5ncccc54)nc2)c2ccccc23)cc1] 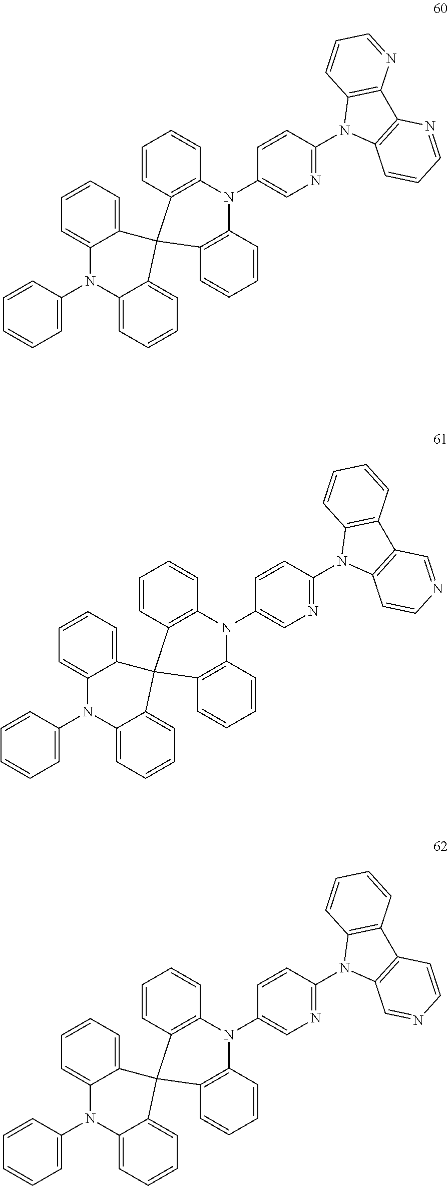 [SMILES: c1ccc(N2c3ccccc3C3(c4ccccc42)c2ccccc2N(c2ccc(-n4c5cccnc5c5ncccc54)nc2)c2ccccc23)cc1 ; c1ccc(N2c3ccccc3C3(c4ccccc42)c2ccccc2N(c2ccc(-n4c5ccccc5c5cnccc54)nc2)c2ccccc23)cc1 ; c1ccc(N2c3ccccc3C3(c4ccccc42)c2ccccc2N(c2ccc(-n4c5ccccc5c5ccncc54)nc2)c2ccccc23)cc1] 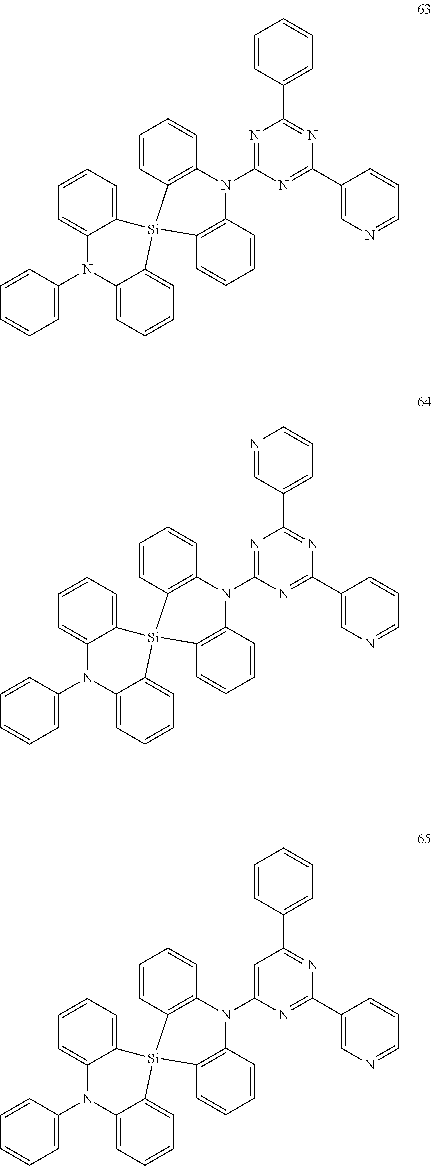 [SMILES: c1ccc(-c2nc(-c3cccnc3)nc(N3c4ccccc4[Si]4(c5ccccc5N(c5ccccc5)c5ccccc54)c4ccccc43)n2)cc1 ; c1ccc(N2c3ccccc3[Si]3(c4ccccc42)c2ccccc2N(c2nc(-c4cccnc4)nc(-c4cccnc4)n2)c2ccccc23)cc1 ; c1ccc(-c2cc(N3c4ccccc4[Si]4(c5ccccc5N(c5ccccc5)c5ccccc54)c4ccccc43)nc(-c3cccnc3)n2)cc1] 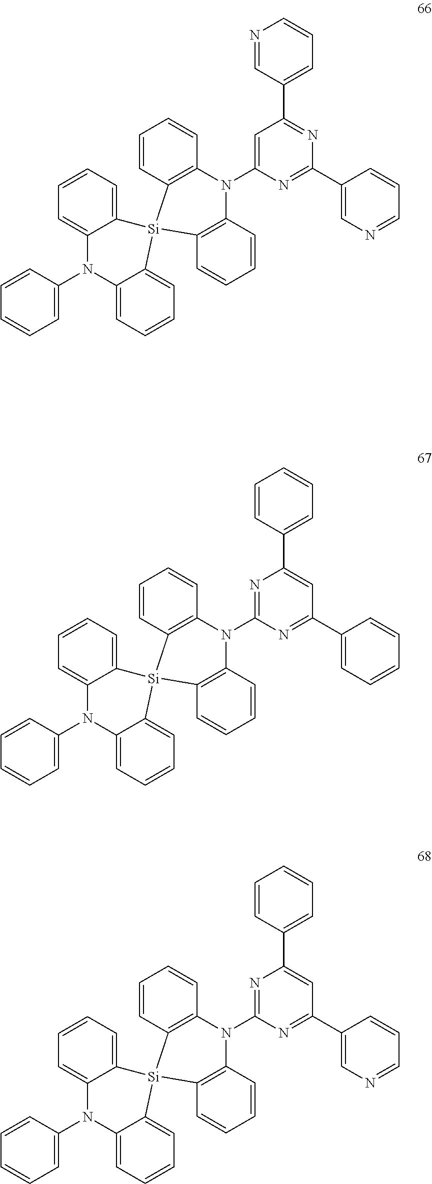 [SMILES: c1ccc(N2c3ccccc3[Si]3(c4ccccc42)c2ccccc2N(c2cc(-c4cccnc4)nc(-c4cccnc4)n2)c2ccccc23)cc1 ; c1ccc(-c2cc(-c3ccccc3)nc(N3c4ccccc4[Si]4(c5ccccc5N(c5ccccc5)c5ccccc54)c4ccccc43)n2)cc1 ; c1ccc(-c2cc(-c3cccnc3)nc(N3c4ccccc4[Si]4(c5ccccc5N(c5ccccc5)c5ccccc54)c4ccccc43)n2)cc1] 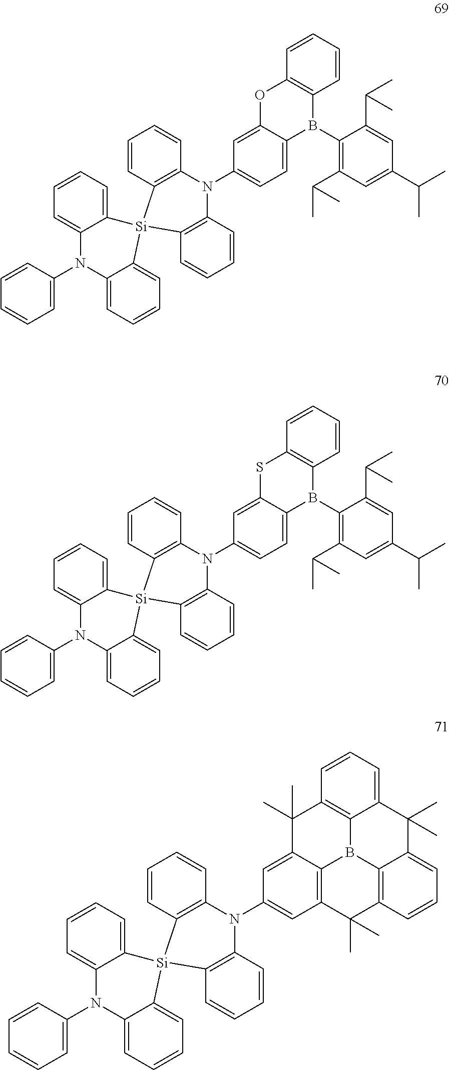 [SMILES: CC(C)c1cc(C(C)C)c(B2c3ccccc3Oc3cc(N4c5ccccc5[Si]5(c6ccccc6N(c6ccccc6)c6ccccc65)c5ccccc54)ccc32)c(C(C)C)c1 ; CC(C)c1cc(C(C)C)c(B2c3ccccc3Sc3cc(N4c5ccccc5[Si]5(c6ccccc6N(c6ccccc6)c6ccccc65)c5ccccc54)ccc32)c(C(C)C)c1 ; CC1(C)c2cccc3c2B2c4c1cccc4C(C)(C)c1cc(N4c5ccccc5[Si]5(c6ccccc6N(c6ccccc6)c6ccccc65)c5ccccc54)cc(c12)C3(C)C] 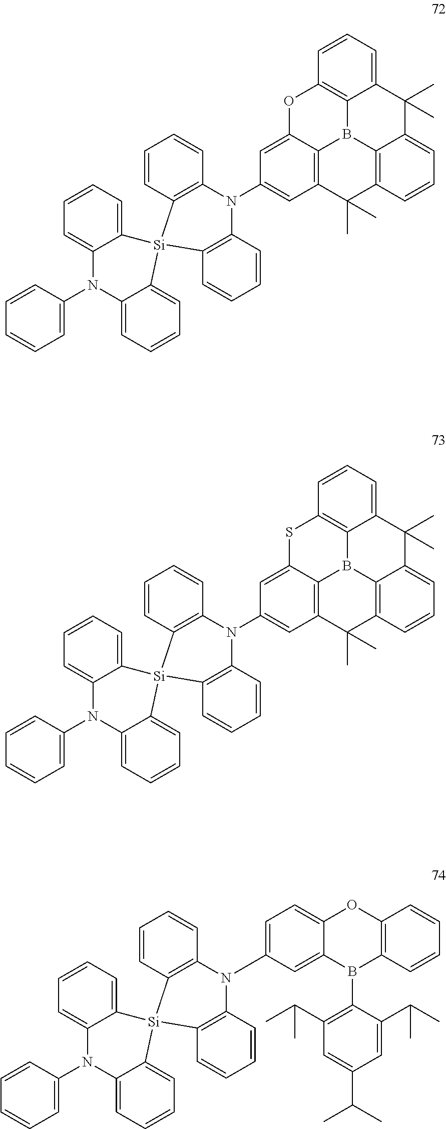 [SMILES: CC1(C)c2cccc3c2B2c4c(cc(N5c6ccccc6[Si]6(c7ccccc7N(c7ccccc7)c7ccccc76)c6ccccc65)cc4C(C)(C)c4cccc1c42)O3 ; CC1(C)c2cccc3c2B2c4c(cc(N5c6ccccc6[Si]6(c7ccccc7N(c7ccccc7)c7ccccc76)c6ccccc65)cc4C(C)(C)c4cccc1c42)S3 ; CC(C)c1cc(C(C)C)c(B2c3ccccc3Oc3ccc(N4c5ccccc5[Si]5(c6ccccc6N(c6ccccc6)c6ccccc65)c5ccccc54)cc32)c(C(C)C)c1] 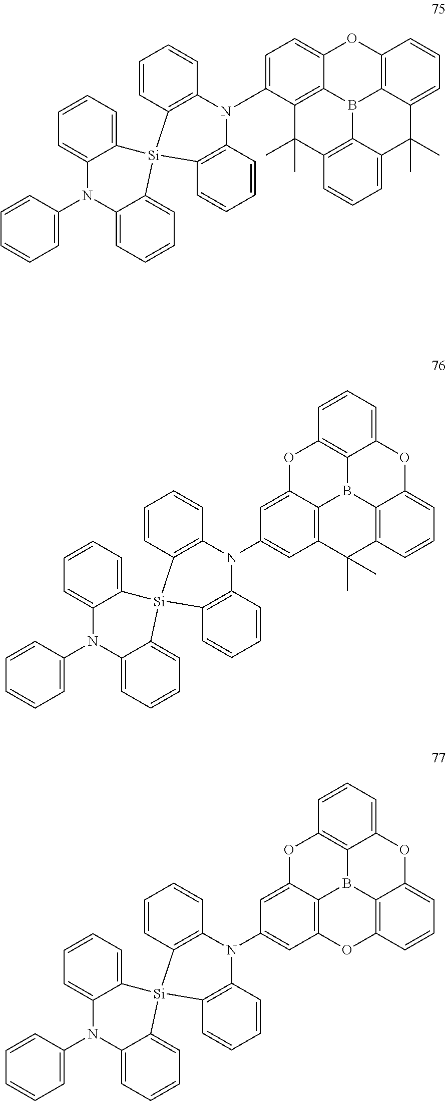 [SMILES: CC1(C)c2cccc3c2B2c4c1cccc4C(C)(C)c1c(N4c5ccccc5[Si]5(c6ccccc6N(c6ccccc6)c6ccccc65)c5ccccc54)ccc(c12)O3 ; CC1(C)c2cccc3c2B2c4c(cccc4Oc4cc(N5c6ccccc6[Si]6(c7ccccc7N(c7ccccc7)c7ccccc76)c6ccccc65)cc1c42)O3 ; c1ccc(N2c3ccccc3[Si]3(c4ccccc42)c2ccccc2N(c2cc4c5c(c2)Oc2cccc6c2B5c2c(cccc2O4)O6)c2ccccc23)cc1] 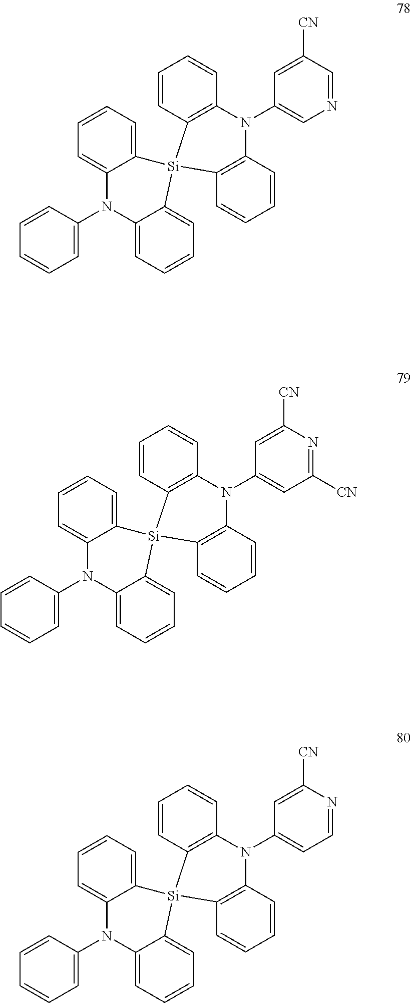 [SMILES: N#Cc1cncc(N2c3ccccc3[Si]3(c4ccccc4N(c4ccccc4)c4ccccc43)c3ccccc32)c1 ; N#Cc1cc(N2c3ccccc3[Si]3(c4ccccc4N(c4ccccc4)c4ccccc43)c3ccccc32)cc(C#N)n1 ; N#Cc1cc(N2c3ccccc3[Si]3(c4ccccc4N(c4ccccc4)c4ccccc43)c3ccccc32)ccn1] 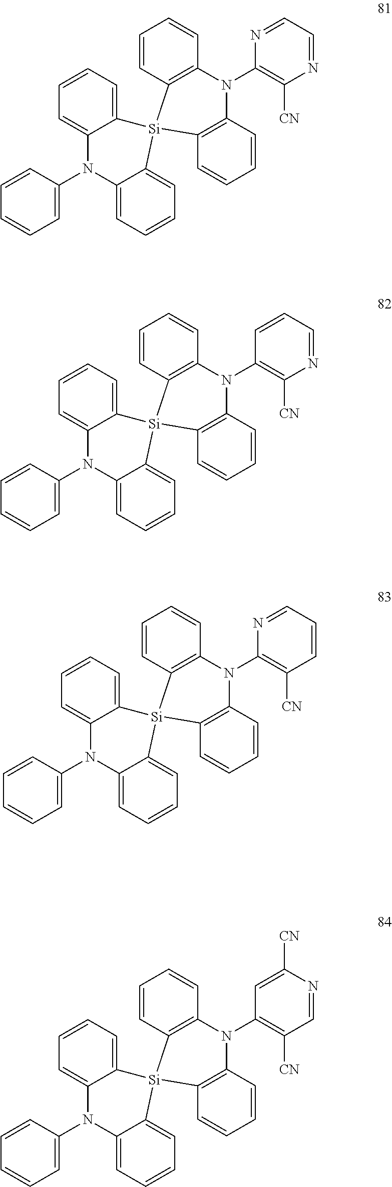 [SMILES: N#Cc1nccnc1N1c2ccccc2[Si]2(c3ccccc3N(c3ccccc3)c3ccccc32)c2ccccc21 ; N#Cc1ncccc1N1c2ccccc2[Si]2(c3ccccc3N(c3ccccc3)c3ccccc32)c2ccccc21 ; N#Cc1cccnc1N1c2ccccc2[Si]2(c3ccccc3N(c3ccccc3)c3ccccc32)c2ccccc21 ; N#Cc1cc(N2c3ccccc3[Si]3(c4ccccc4N(c4ccccc4)c4ccccc43)c3ccccc32)c(C#N)cn1] 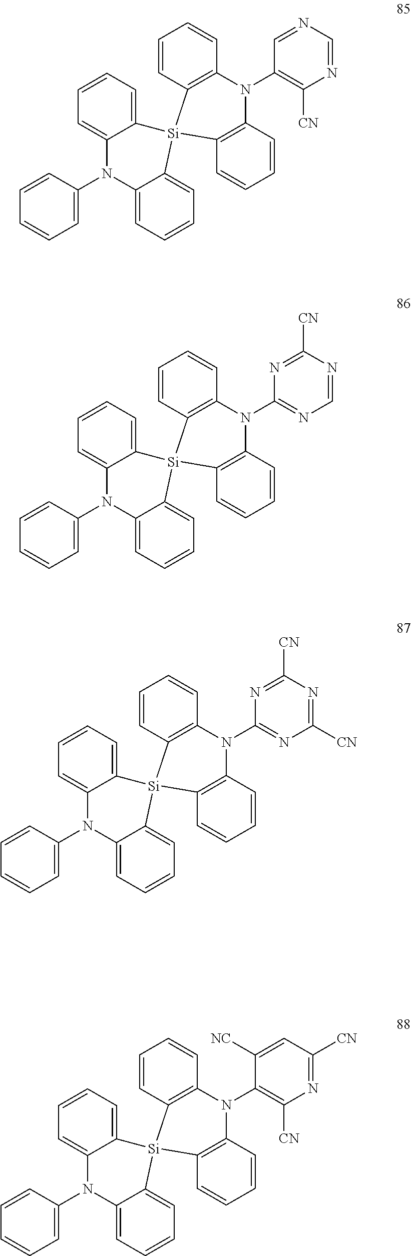 [SMILES: N#Cc1ncncc1N1c2ccccc2[Si]2(c3ccccc3N(c3ccccc3)c3ccccc32)c2ccccc21 ; N#Cc1ncnc(N2c3ccccc3[Si]3(c4ccccc4N(c4ccccc4)c4ccccc43)c3ccccc32)n1 ; N#Cc1nc(C#N)nc(N2c3ccccc3[Si]3(c4ccccc4N(c4ccccc4)c4ccccc43)c3ccccc32)n1 ; N#Cc1cc(C#N)c(N2c3ccccc3[Si]3(c4ccccc4N(c4ccccc4)c4ccccc43)c3ccccc32)c(C#N)n1] 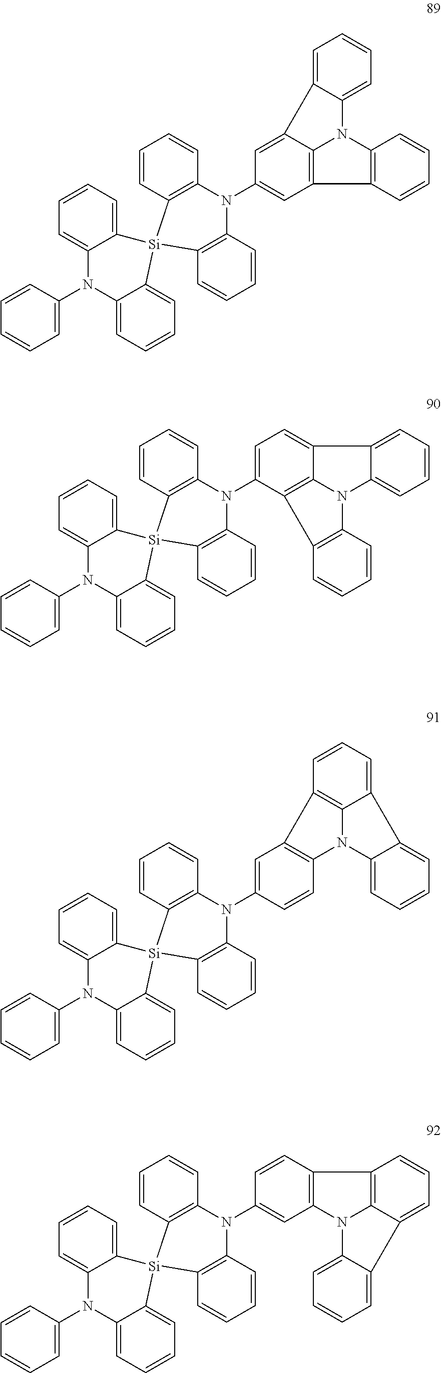 [SMILES: c1ccc(N2c3ccccc3[Si]3(c4ccccc42)c2ccccc2N(c2cc4c5ccccc5n5c6ccccc6c(c2)c45)c2ccccc23)cc1 ; c1ccc(N2c3ccccc3[Si]3(c4ccccc42)c2ccccc2N(c2ccc4c5ccccc5n5c6ccccc6c2c45)c2ccccc23)cc1 ; c1ccc(N2c3ccccc3[Si]3(c4ccccc42)c2ccccc2N(c2ccc4c(c2)c2cccc5c6ccccc6n4c52)c2ccccc23)cc1 ; c1ccc(N2c3ccccc3[Si]3(c4ccccc42)c2ccccc2N(c2ccc4c5cccc6c7ccccc7n(c4c2)c65)c2ccccc23)cc1] 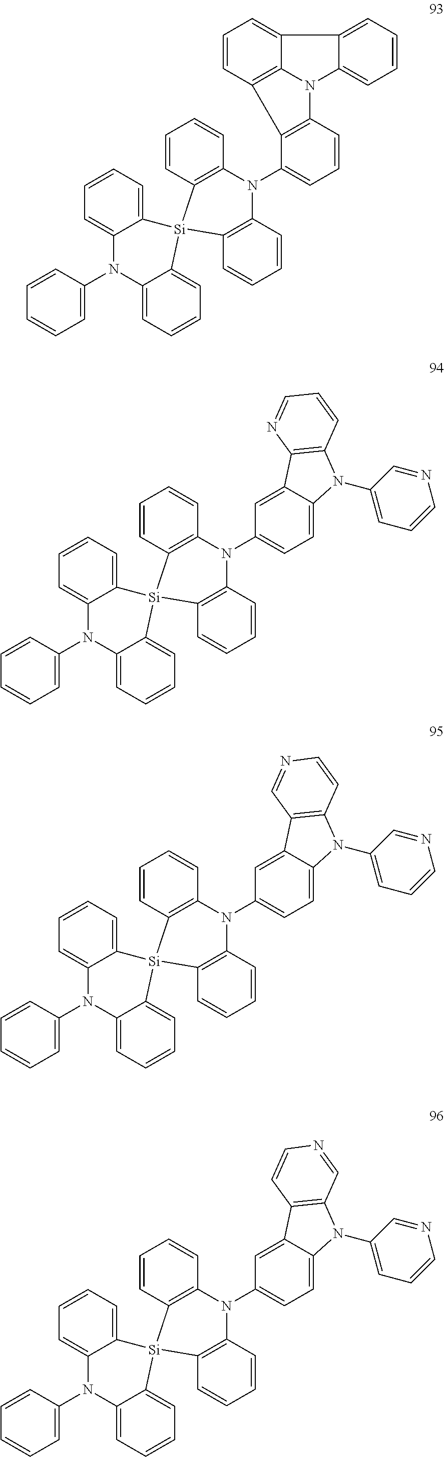 [SMILES: c1ccc(N2c3ccccc3[Si]3(c4ccccc42)c2ccccc2N(c2cccc4c2c2cccc5c6ccccc6n4c52)c2ccccc23)cc1 ; c1ccc(N2c3ccccc3[Si]3(c4ccccc42)c2ccccc2N(c2ccc4c(c2)c2ncccc2n4-c2cccnc2)c2ccccc23)cc1 ; c1ccc(N2c3ccccc3[Si]3(c4ccccc42)c2ccccc2N(c2ccc4c(c2)c2cnccc2n4-c2cccnc2)c2ccccc23)cc1 ; c1ccc(N2c3ccccc3[Si]3(c4ccccc42)c2ccccc2N(c2ccc4c(c2)c2ccncc2n4-c2cccnc2)c2ccccc23)cc1] 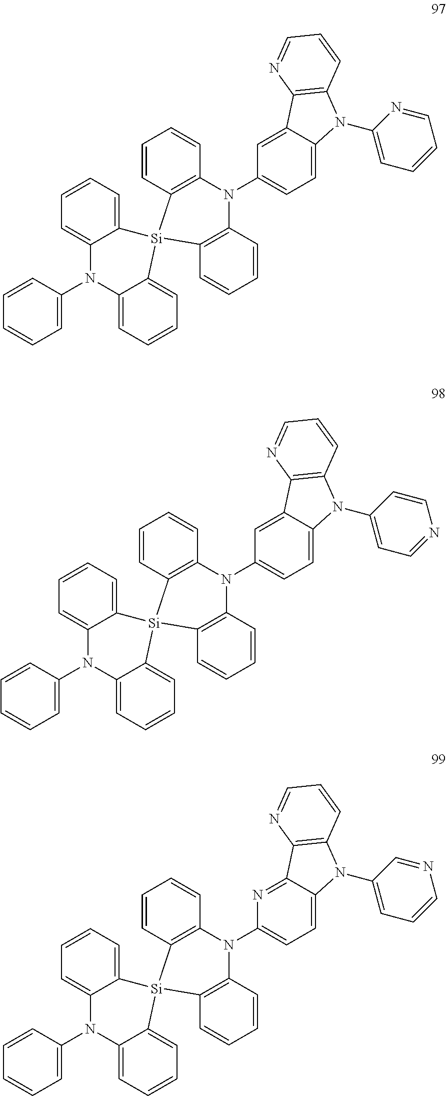 [SMILES: c1ccc(N2c3ccccc3[Si]3(c4ccccc42)c2ccccc2N(c2ccc4c(c2)c2ncccc2n4-c2ccccn2)c2ccccc23)cc1 ; c1ccc(N2c3ccccc3[Si]3(c4ccccc42)c2ccccc2N(c2ccc4c(c2)c2ncccc2n4-c2ccncc2)c2ccccc23)cc1 ; c1ccc(N2c3ccccc3[Si]3(c4ccccc42)c2ccccc2N(c2ccc4c(n2)c2ncccc2n4-c2cccnc2)c2ccccc23)cc1] 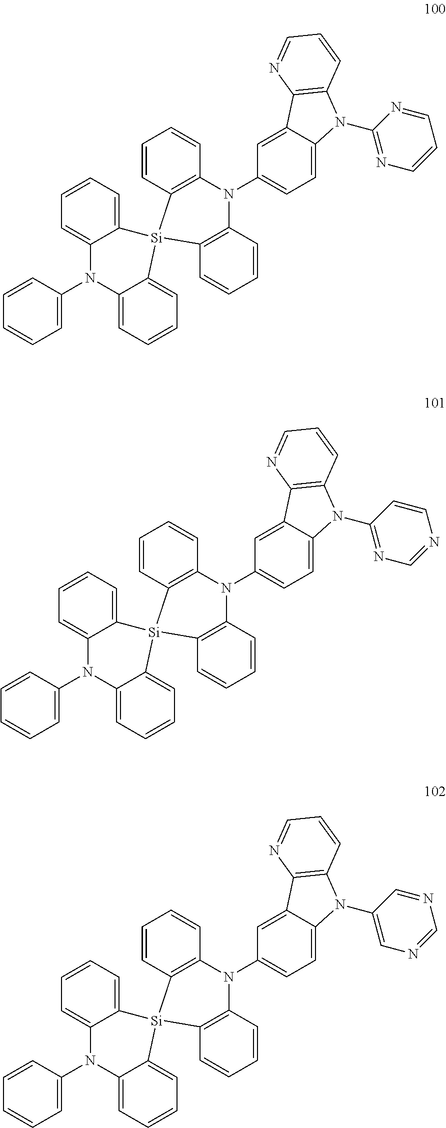 [SMILES: c1ccc(N2c3ccccc3[Si]3(c4ccccc42)c2ccccc2N(c2ccc4c(c2)c2ncccc2n4-c2ncccn2)c2ccccc23)cc1 ; c1ccc(N2c3ccccc3[Si]3(c4ccccc42)c2ccccc2N(c2ccc4c(c2)c2ncccc2n4-c2ccncn2)c2ccccc23)cc1 ; c1ccc(N2c3ccccc3[Si]3(c4ccccc42)c2ccccc2N(c2ccc4c(c2)c2ncccc2n4-c2cncnc2)c2ccccc23)cc1] 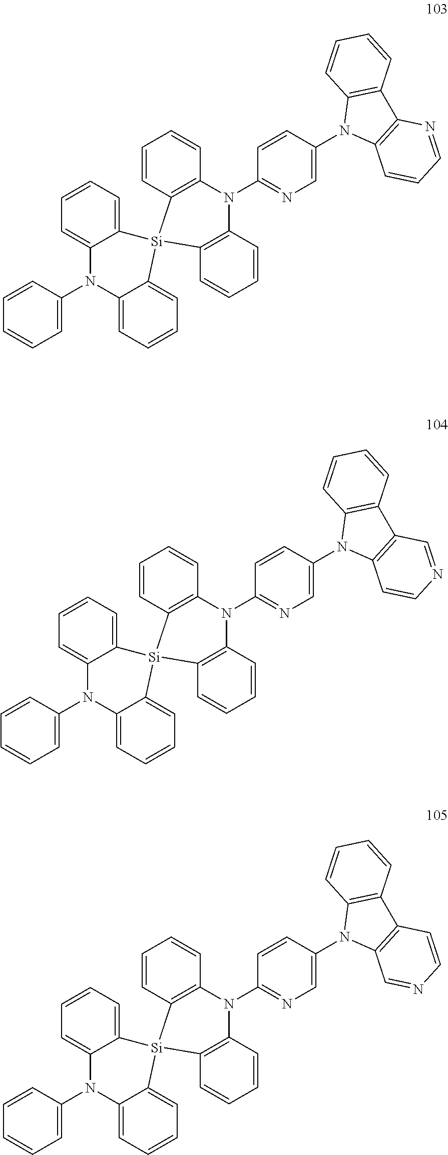 [SMILES: c1ccc(N2c3ccccc3[Si]3(c4ccccc42)c2ccccc2N(c2ccc(-n4c5ccccc5c5ncccc54)cn2)c2ccccc23)cc1 ; c1ccc(N2c3ccccc3[Si]3(c4ccccc42)c2ccccc2N(c2ccc(-n4c5ccccc5c5cnccc54)cn2)c2ccccc23)cc1 ; c1ccc(N2c3ccccc3[Si]3(c4ccccc42)c2ccccc2N(c2ccc(-n4c5ccccc5c5ccncc54)cn2)c2ccccc23)cc1] 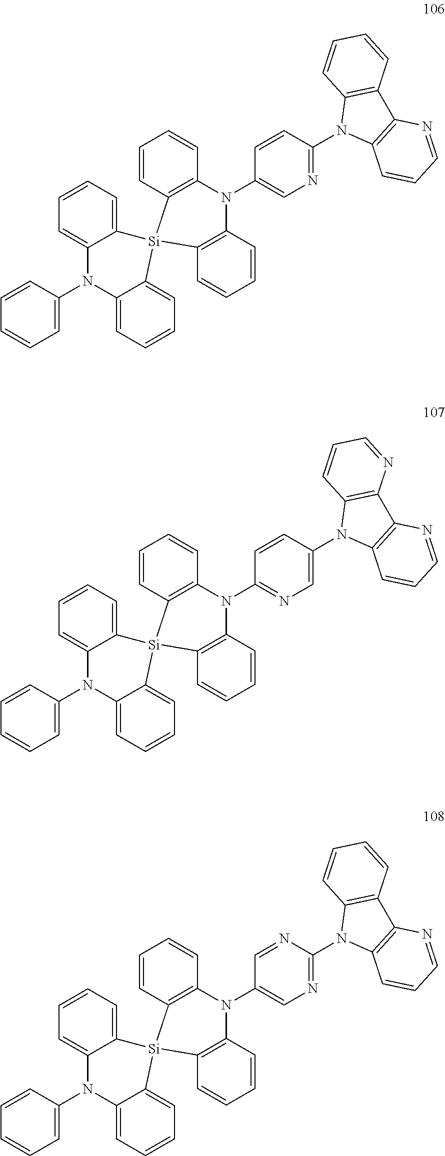 [SMILES: c1ccc(N2c3ccccc3[Si]3(c4ccccc42)c2ccccc2N(c2ccc(-n4c5ccccc5c5ncccc54)nc2)c2ccccc23)cc1 ; c1ccc(N2c3ccccc3[Si]3(c4ccccc42)c2ccccc2N(c2ccc(-n4c5cccnc5c5ncccc54)cn2)c2ccccc23)cc1 ; c1ccc(N2c3ccccc3[Si]3(c4ccccc42)c2ccccc2N(c2cnc(-n4c5ccccc5c5ncccc54)nc2)c2ccccc23)cc1] 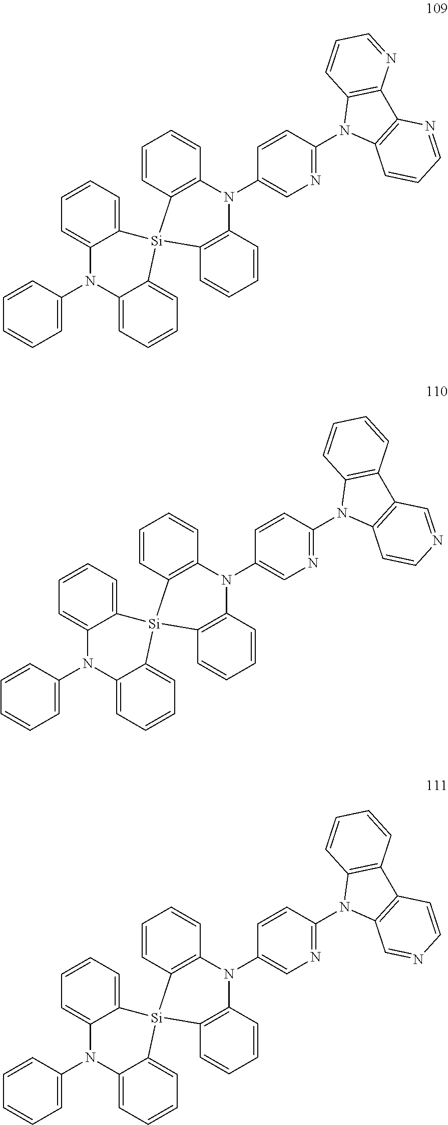 [SMILES: c1ccc(N2c3ccccc3[Si]3(c4ccccc42)c2ccccc2N(c2ccc(-n4c5cccnc5c5ncccc54)nc2)c2ccccc23)cc1 ; c1ccc(N2c3ccccc3[Si]3(c4ccccc42)c2ccccc2N(c2ccc(-n4c5ccccc5c5cnccc54)nc2)c2ccccc23)cc1 ; c1ccc(N2c3ccccc3[Si]3(c4ccccc42)c2ccccc2N(c2ccc(-n4c5ccccc5c5ccncc54)nc2)c2ccccc23)cc1] 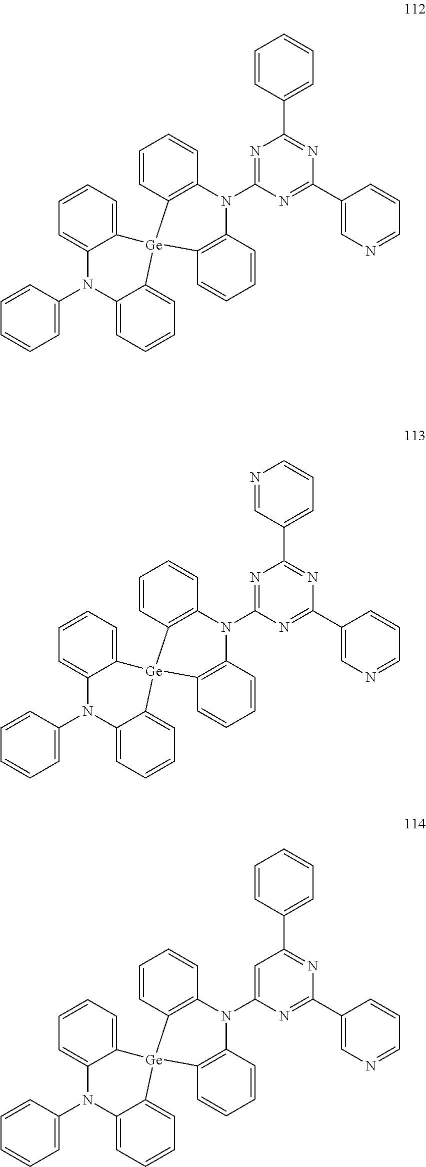 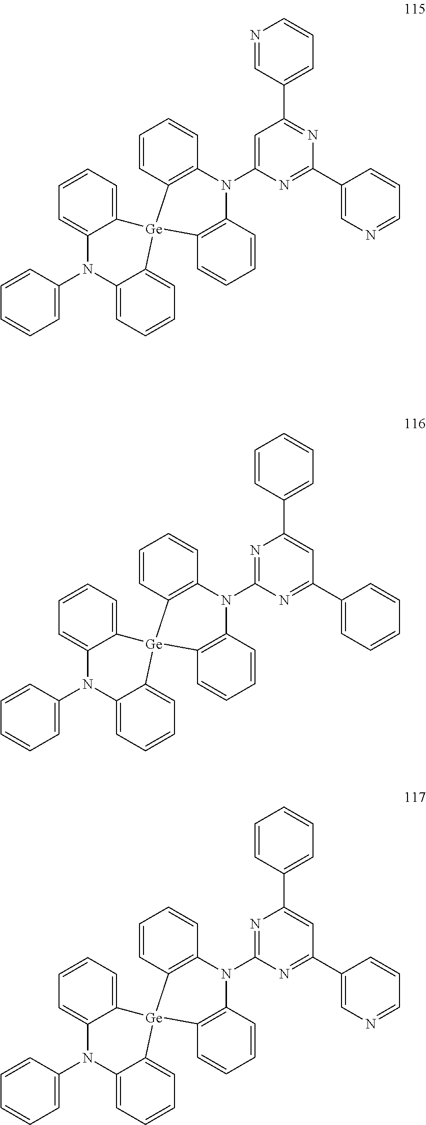 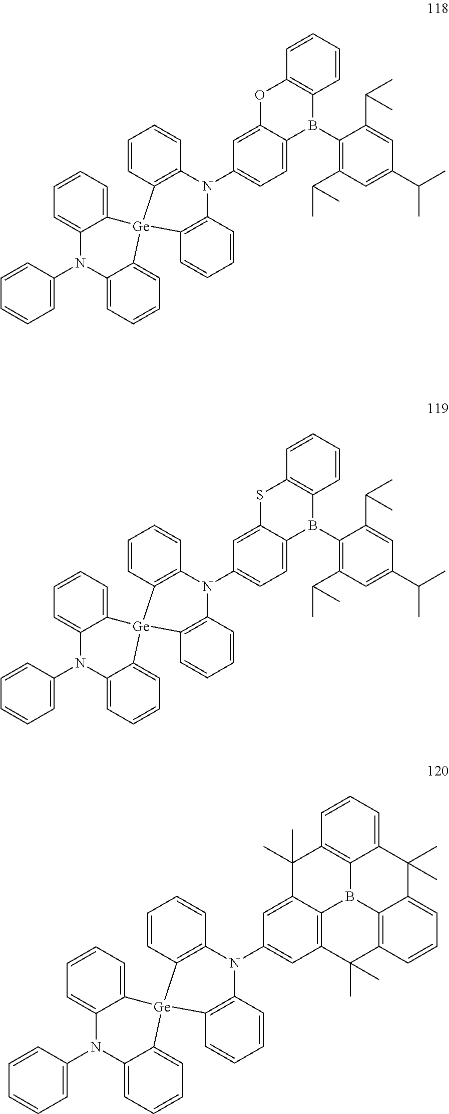 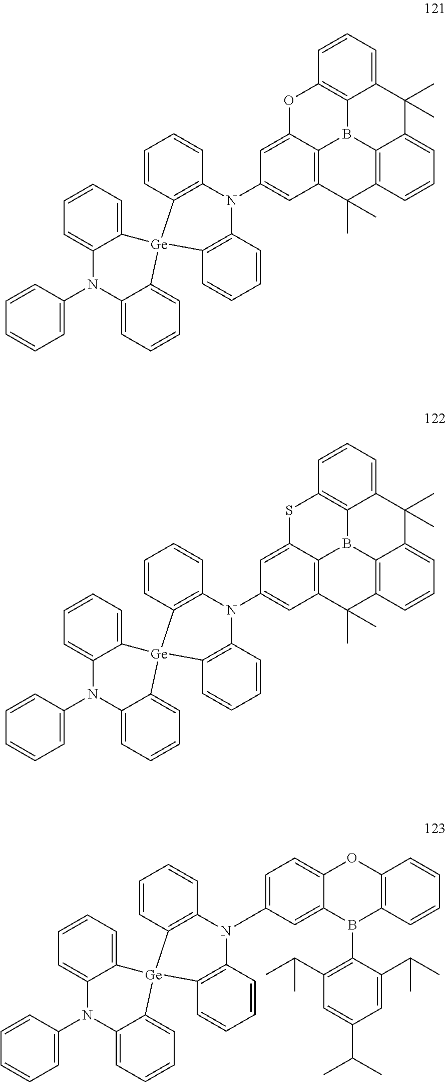 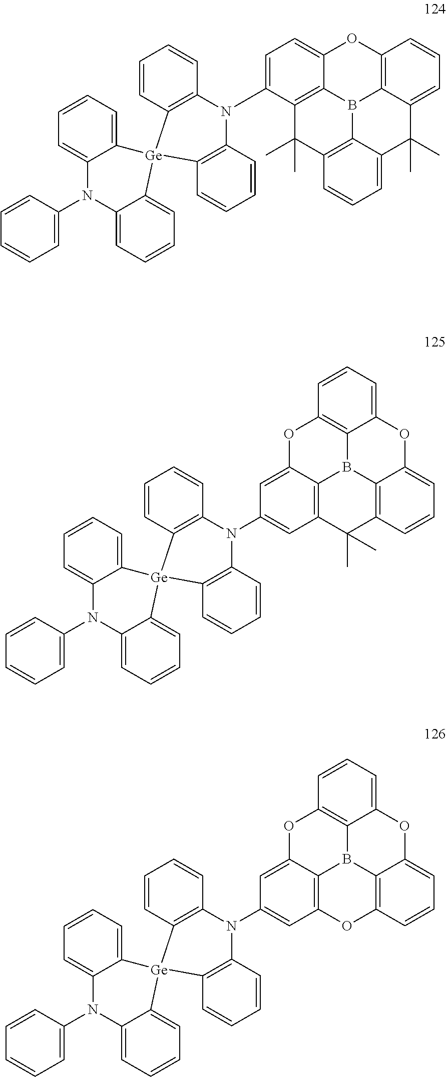 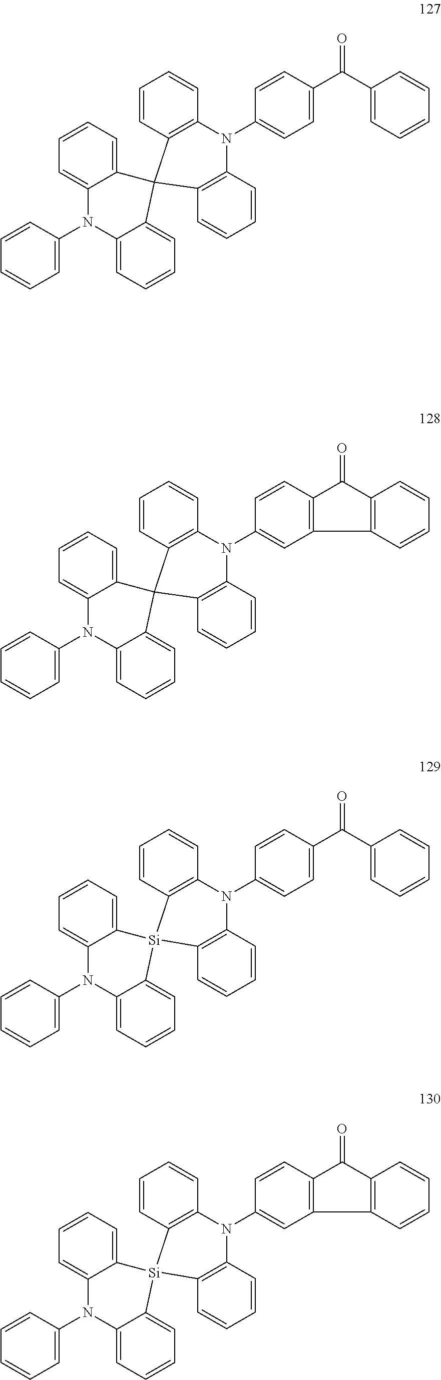 [SMILES: O=C(c1ccccc1)c1ccc(N2c3ccccc3C3(c4ccccc4N(c4ccccc4)c4ccccc43)c3ccccc32)cc1 ; O=C1c2ccccc2-c2cc(N3c4ccccc4C4(c5ccccc5N(c5ccccc5)c5ccccc54)c4ccccc43)ccc21 ; O=C(c1ccccc1)c1ccc(N2c3ccccc3[Si]3(c4ccccc4N(c4ccccc4)c4ccccc43)c3ccccc32)cc1 ; O=C1c2ccccc2-c2cc(N3c4ccccc4[Si]4(c5ccccc5N(c5ccccc5)c5ccccc54)c4ccccc43)ccc21] 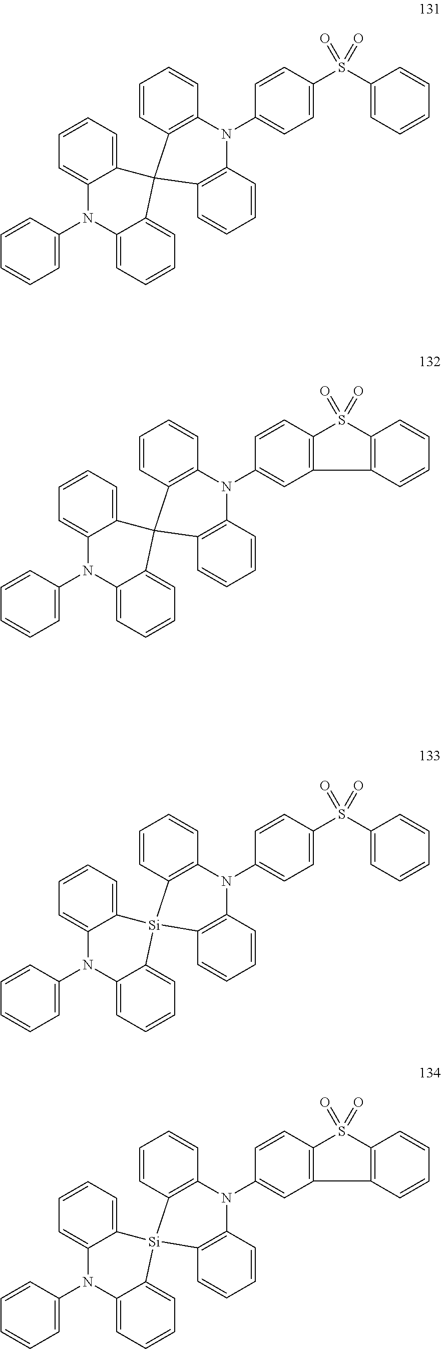 [SMILES: O=S(=O)(c1ccccc1)c1ccc(N2c3ccccc3C3(c4ccccc4N(c4ccccc4)c4ccccc43)c3ccccc32)cc1 ; O=S1(=O)c2ccccc2-c2cc(N3c4ccccc4C4(c5ccccc5N(c5ccccc5)c5ccccc54)c4ccccc43)ccc21 ; O=S(=O)(c1ccccc1)c1ccc(N2c3ccccc3[Si]3(c4ccccc4N(c4ccccc4)c4ccccc43)c3ccccc32)cc1 ; O=S1(=O)c2ccccc2-c2cc(N3c4ccccc4[Si]4(c5ccccc5N(c5ccccc5)c5ccccc54)c4ccccc43)ccc21] 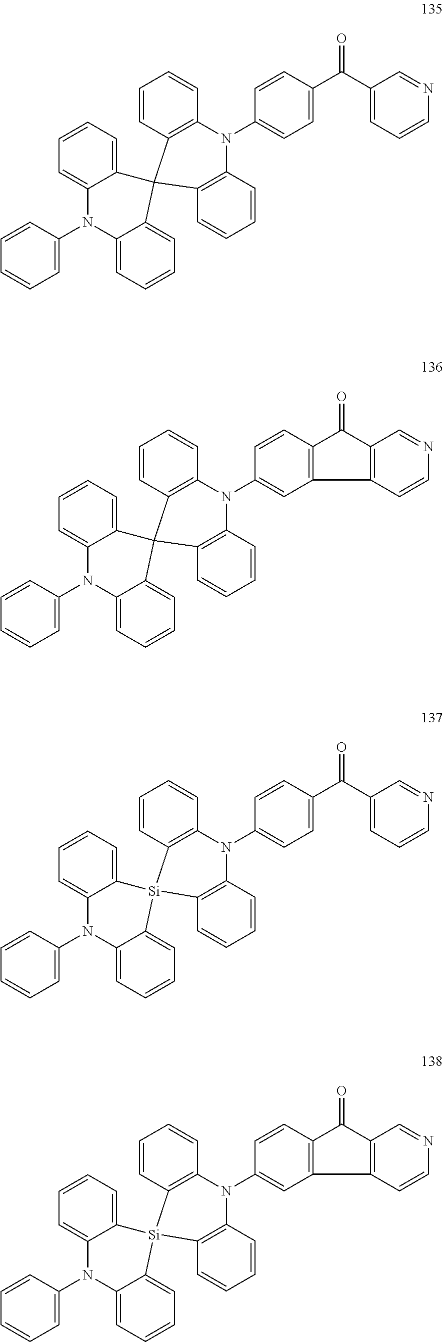 [SMILES: O=C(c1ccc(N2c3ccccc3C3(c4ccccc4N(c4ccccc4)c4ccccc43)c3ccccc32)cc1)c1cccnc1 ; O=C1c2cnccc2-c2cc(N3c4ccccc4C4(c5ccccc5N(c5ccccc5)c5ccccc54)c4ccccc43)ccc21 ; O=C(c1ccc(N2c3ccccc3[Si]3(c4ccccc4N(c4ccccc4)c4ccccc43)c3ccccc32)cc1)c1cccnc1 ; O=C1c2cnccc2-c2cc(N3c4ccccc4[Si]4(c5ccccc5N(c5ccccc5)c5ccccc54)c4ccccc43)ccc21] 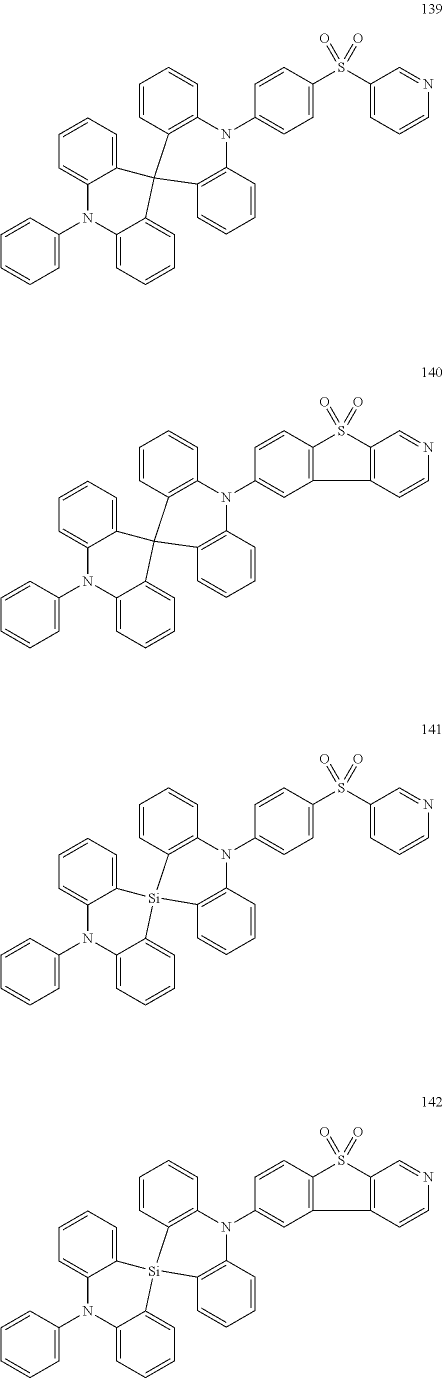 [SMILES: O=S(=O)(c1ccc(N2c3ccccc3C3(c4ccccc4N(c4ccccc4)c4ccccc43)c3ccccc32)cc1)c1cccnc1 ; O=S1(=O)c2ccc(N3c4ccccc4C4(c5ccccc5N(c5ccccc5)c5ccccc54)c4ccccc43)cc2-c2ccncc21 ; O=S(=O)(c1ccc(N2c3ccccc3[Si]3(c4ccccc4N(c4ccccc4)c4ccccc43)c3ccccc32)cc1)c1cccnc1 ; O=S1(=O)c2ccc(N3c4ccccc4[Si]4(c5ccccc5N(c5ccccc5)c5ccccc54)c4ccccc43)cc2-c2ccncc21] 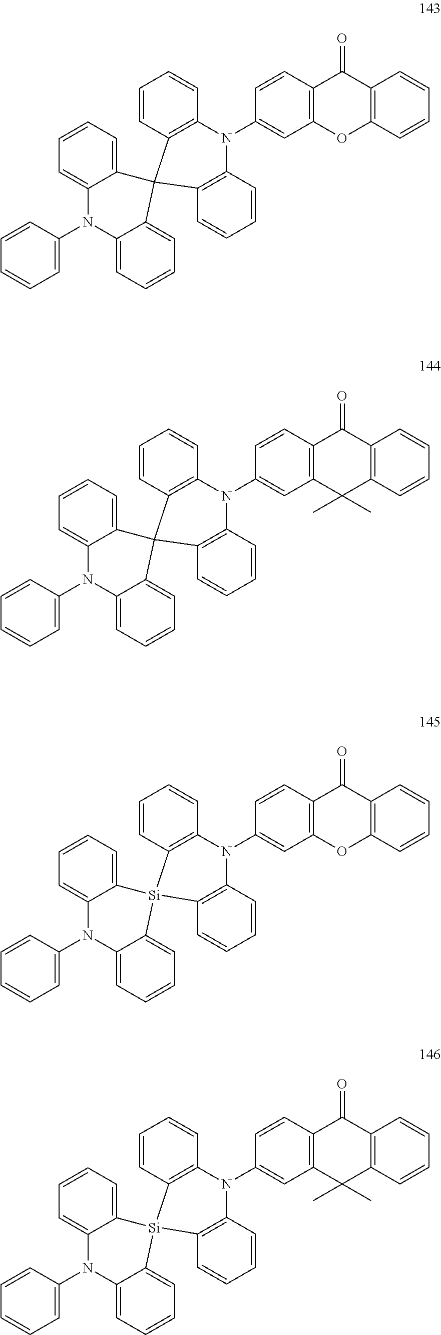 [SMILES: O=c1c2ccccc2oc2cc(N3c4ccccc4C4(c5ccccc5N(c5ccccc5)c5ccccc54)c4ccccc43)ccc12 ; CC1(C)c2ccccc2C(=O)c2ccc(N3c4ccccc4C4(c5ccccc5N(c5ccccc5)c5ccccc54)c4ccccc43)cc21 ; O=c1c2ccccc2oc2cc(N3c4ccccc4[Si]4(c5ccccc5N(c5ccccc5)c5ccccc54)c4ccccc43)ccc12 ; CC1(C)c2ccccc2C(=O)c2ccc(N3c4ccccc4[Si]4(c5ccccc5N(c5ccccc5)c5ccccc54)c4ccccc43)cc21] 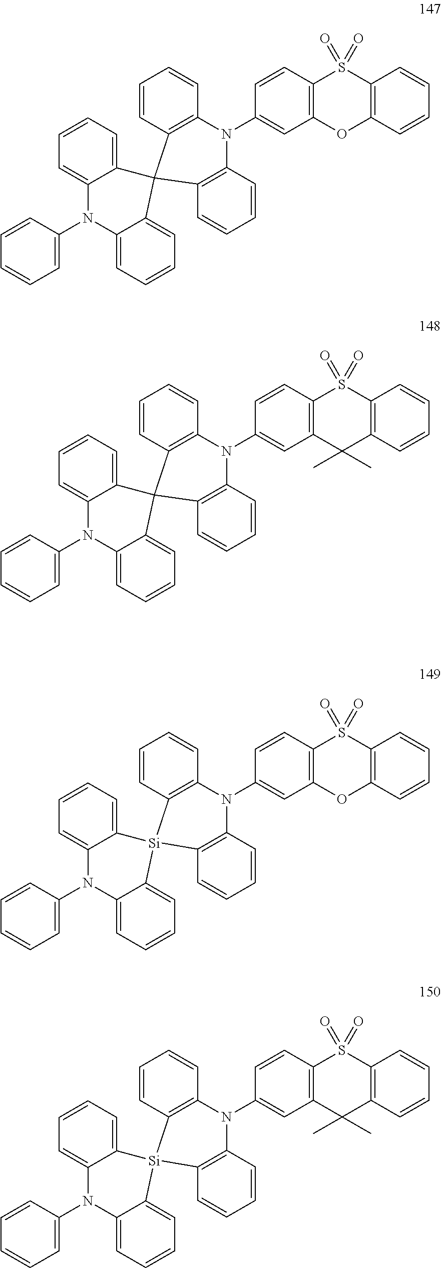 [SMILES: O=S1(=O)c2ccccc2Oc2cc(N3c4ccccc4C4(c5ccccc5N(c5ccccc5)c5ccccc54)c4ccccc43)ccc21 ; CC1(C)c2ccccc2S(=O)(=O)c2ccc(N3c4ccccc4C4(c5ccccc5N(c5ccccc5)c5ccccc54)c4ccccc43)cc21 ; O=S1(=O)c2ccccc2Oc2cc(N3c4ccccc4[Si]4(c5ccccc5N(c5ccccc5)c5ccccc54)c4ccccc43)ccc21 ; CC1(C)c2ccccc2S(=O)(=O)c2ccc(N3c4ccccc4[Si]4(c5ccccc5N(c5ccccc5)c5ccccc54)c4ccccc43)cc21] 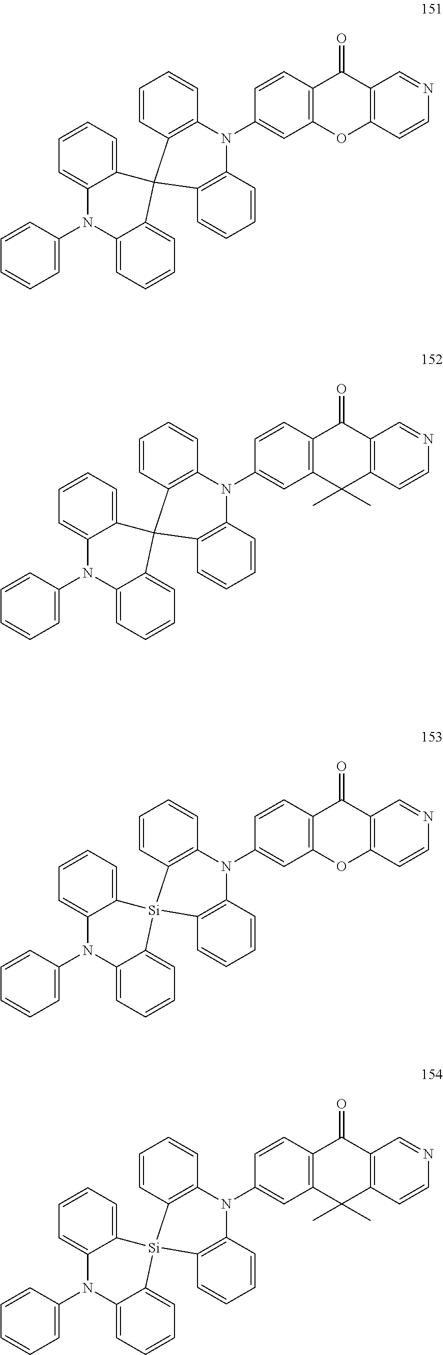 [SMILES: O=c1c2ccc(N3c4ccccc4C4(c5ccccc5N(c5ccccc5)c5ccccc54)c4ccccc43)cc2oc2ccncc12 ; CC1(C)c2ccncc2C(=O)c2ccc(N3c4ccccc4C4(c5ccccc5N(c5ccccc5)c5ccccc54)c4ccccc43)cc21 ; O=c1c2ccc(N3c4ccccc4[Si]4(c5ccccc5N(c5ccccc5)c5ccccc54)c4ccccc43)cc2oc2ccncc12 ; CC1(C)c2ccncc2C(=O)c2ccc(N3c4ccccc4[Si]4(c5ccccc5N(c5ccccc5)c5ccccc54)c4ccccc43)cc21] 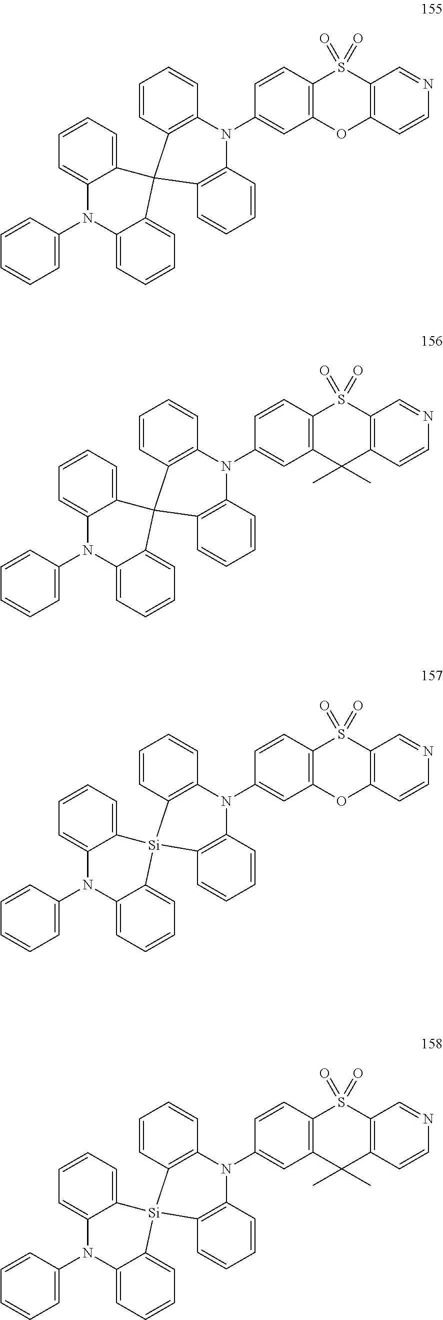 [SMILES: O=S1(=O)c2ccc(N3c4ccccc4C4(c5ccccc5N(c5ccccc5)c5ccccc54)c4ccccc43)cc2Oc2ccncc21 ; CC1(C)c2cc(N3c4ccccc4C4(c5ccccc5N(c5ccccc5)c5ccccc54)c4ccccc43)ccc2S(=O)(=O)c2cnccc21 ; O=S1(=O)c2ccc(N3c4ccccc4[Si]4(c5ccccc5N(c5ccccc5)c5ccccc54)c4ccccc43)cc2Oc2ccncc21 ; CC1(C)c2cc(N3c4ccccc4[Si]4(c5ccccc5N(c5ccccc5)c5ccccc54)c4ccccc43)ccc2S(=O)(=O)c2cnccc21] 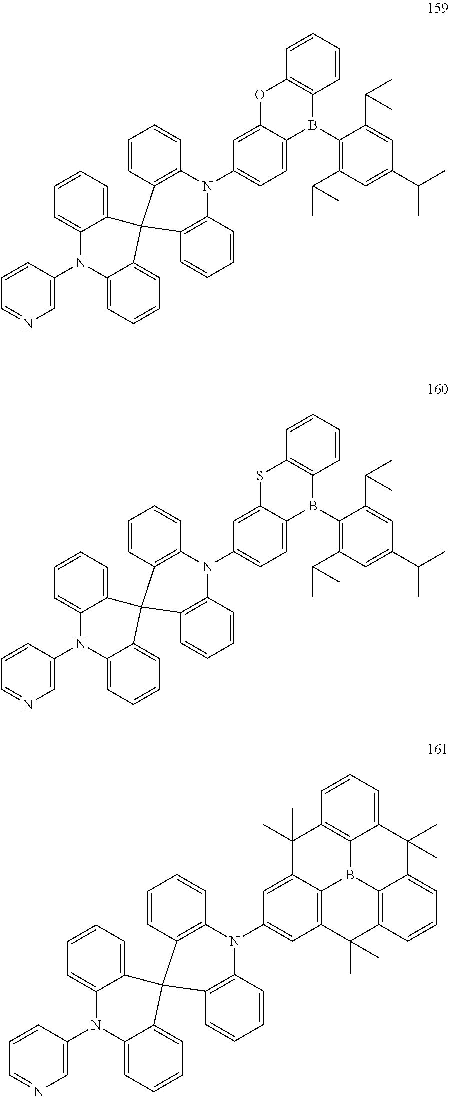 [SMILES: CC(C)c1cc(C(C)C)c(B2c3ccccc3Oc3cc(N4c5ccccc5C5(c6ccccc6N(c6cccnc6)c6ccccc65)c5ccccc54)ccc32)c(C(C)C)c1 ; CC(C)c1cc(C(C)C)c(B2c3ccccc3Sc3cc(N4c5ccccc5C5(c6ccccc6N(c6cccnc6)c6ccccc65)c5ccccc54)ccc32)c(C(C)C)c1 ; CC1(C)c2cccc3c2B2c4c1cccc4C(C)(C)c1cc(N4c5ccccc5C5(c6ccccc6N(c6cccnc6)c6ccccc65)c5ccccc54)cc(c12)C3(C)C] 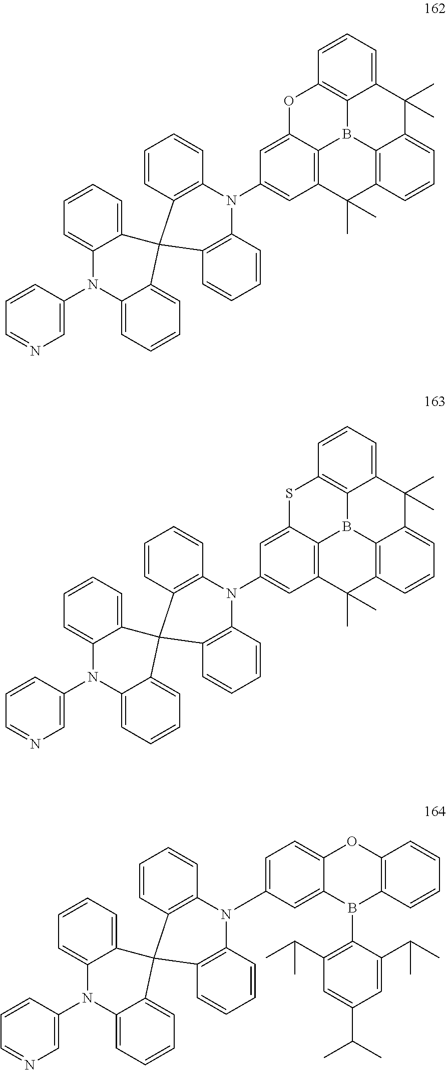 [SMILES: CC1(C)c2cccc3c2B2c4c(cc(N5c6ccccc6C6(c7ccccc7N(c7cccnc7)c7ccccc76)c6ccccc65)cc4C(C)(C)c4cccc1c42)O3 ; CC1(C)c2cccc3c2B2c4c(cc(N5c6ccccc6C6(c7ccccc7N(c7cccnc7)c7ccccc76)c6ccccc65)cc4C(C)(C)c4cccc1c42)S3 ; CC(C)c1cc(C(C)C)c(B2c3ccccc3Oc3ccc(N4c5ccccc5C5(c6ccccc6N(c6cccnc6)c6ccccc65)c5ccccc54)cc32)c(C(C)C)c1] 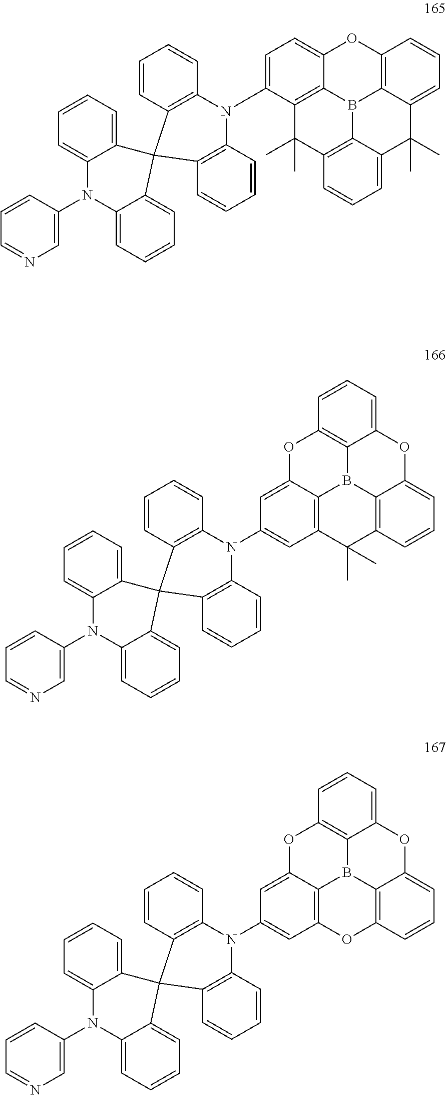 [SMILES: CC1(C)c2cccc3c2B2c4c1cccc4C(C)(C)c1c(N4c5ccccc5C5(c6ccccc6N(c6cccnc6)c6ccccc65)c5ccccc54)ccc(c12)O3 ; CC1(C)c2cccc3c2B2c4c(cccc4Oc4cc(N5c6ccccc6C6(c7ccccc7N(c7cccnc7)c7ccccc76)c6ccccc65)cc1c42)O3 ; c1cncc(N2c3ccccc3C3(c4ccccc42)c2ccccc2N(c2cc4c5c(c2)Oc2cccc6c2B5c2c(cccc2O4)O6)c2ccccc23)c1] 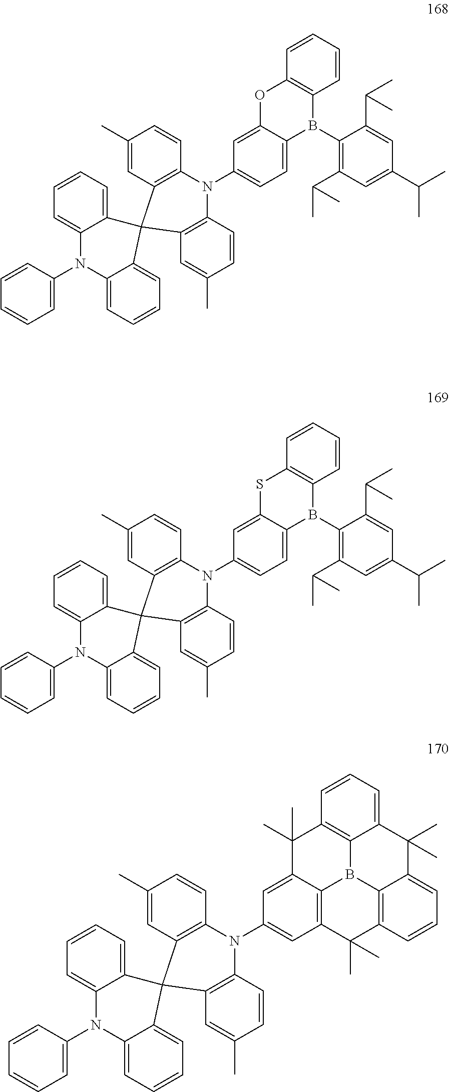 [SMILES: Cc1ccc2c(c1)C1(c3ccccc3N(c3ccccc3)c3ccccc31)c1cc(C)ccc1N2c1ccc2c(c1)Oc1ccccc1B2c1c(C(C)C)cc(C(C)C)cc1C(C)C ; Cc1ccc2c(c1)C1(c3ccccc3N(c3ccccc3)c3ccccc31)c1cc(C)ccc1N2c1ccc2c(c1)Sc1ccccc1B2c1c(C(C)C)cc(C(C)C)cc1C(C)C ; Cc1ccc2c(c1)C1(c3ccccc3N(c3ccccc3)c3ccccc31)c1cc(C)ccc1N2c1cc2c3c(c1)C(C)(C)c1cccc4c1B3c1c(cccc1C2(C)C)C4(C)C] 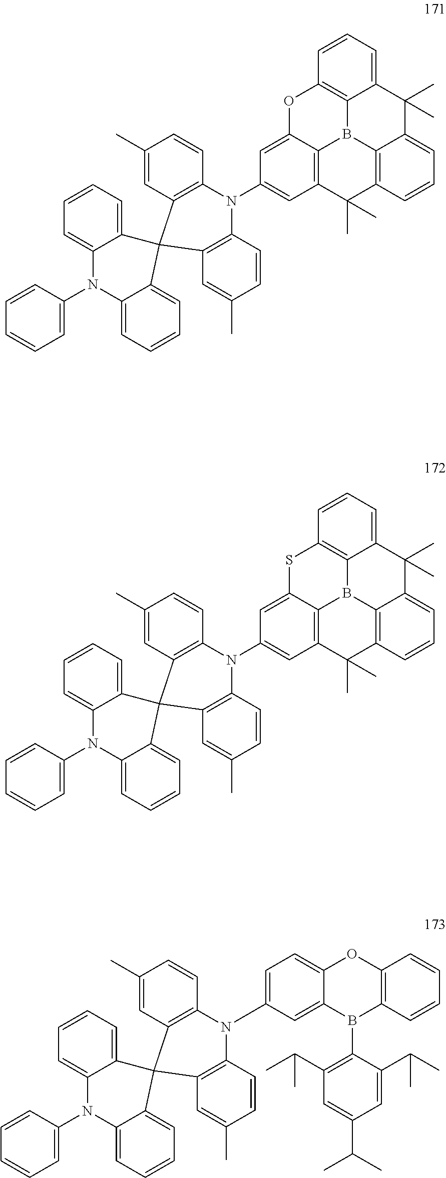 [SMILES: Cc1ccc2c(c1)C1(c3ccccc3N(c3ccccc3)c3ccccc31)c1cc(C)ccc1N2c1cc2c3c(c1)C(C)(C)c1cccc4c1B3c1c(cccc1C4(C)C)O2 ; Cc1ccc2c(c1)C1(c3ccccc3N(c3ccccc3)c3ccccc31)c1cc(C)ccc1N2c1cc2c3c(c1)C(C)(C)c1cccc4c1B3c1c(cccc1C4(C)C)S2 ; Cc1ccc2c(c1)C1(c3ccccc3N(c3ccccc3)c3ccccc31)c1cc(C)ccc1N2c1ccc2c(c1)B(c1c(C(C)C)cc(C(C)C)cc1C(C)C)c1ccccc1O2] 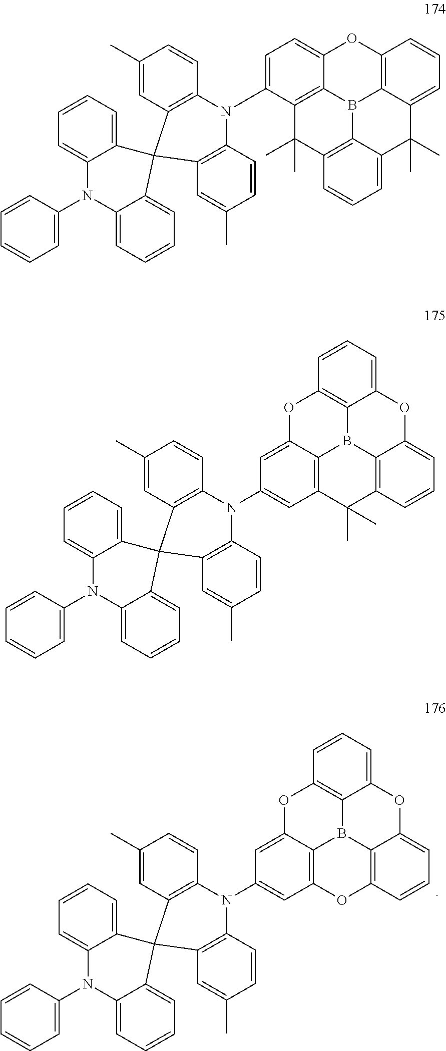 [SMILES: Cc1ccc2c(c1)C1(c3ccccc3N(c3ccccc3)c3ccccc31)c1cc(C)ccc1N2c1ccc2c3c1C(C)(C)c1cccc4c1B3c1c(cccc1C4(C)C)O2 ; Cc1ccc2c(c1)C1(c3ccccc3N(c3ccccc3)c3ccccc31)c1cc(C)ccc1N2c1cc2c3c(c1)C(C)(C)c1cccc4c1B3c1c(cccc1O2)O4 ; Cc1ccc2c(c1)C1(c3ccccc3N(c3ccccc3)c3ccccc31)c1cc(C)ccc1N2c1cc2c3c(c1)Oc1cccc4c1B3c1c(cccc1O2)O4]
